(19) United States
(12) Patent Application Publication
(10) Pub. No.: US 2019/0206520 A1
(43) Pub. Date: Jul. 4, 2019

(54) MOBILE-NATIVE CLINICAL TRIAL OPERATIONS SERVICE SUITE

(71) Applicant: QZ Labs, RSF, CA (US)

(72) Inventors: Isaac Eshagh Eteminan, Rancho Santa Fe, CA (US); Oscar Rangel, San Marcos, CA (US); Joe Waldron, San Diego, CA (US); Nicholas Warner, San Diego, CA (US); James William Bishop, Reno, NV (US); Marco Carosi, Roma (IT)

(21) Appl. No.: 16/100,094

(22) Filed: Aug. 9, 2018

Related U.S. Application Data
(60) Provisional application No. 62/612,495, filed on Dec. 31, 2017.

(51) Int. Cl. G16H 10/20; G16H 10/60

(52) U.S. Cl. CPC G16H 10/20; G16H 80/00; G16H 10/60

(57) ABSTRACT

A trial operations service suite in a clinical trial operations system includes: patient, clinician, investigator, and coordinator portals supporting corresponding applications and providing specific services for each type of participant, where the coordinator portal allows a coordinator user to develop, build, customize, and establish trial protocol and participant interaction using an application factory system embedded within a trial design aspect of the coordinator portal; security configuration and management authentication databases; operational databases including trial data including provisioning and authorization data describing trial participants, access logs recording participant activity, protected and de-identified health information and/or non-health provided by patients, clinicians, and associated sensors; a notification relay service and corresponding external notifications gateway providing essential communication between the system and users, between clinicians and patients, and between coordinators and all participants; and a secure communication relay service providing multi-media communication among participants, constrained by permissions as established by a particular coordinator user.

### CROSS-REFERENCE TO RELATED APPLICATIONS

This application claims priority to U.S. Provisional Patent Application Ser. No. 62/612,495, filed on Dec. 31, 2017.

### BACKGROUND

Healthcare applications of information and communication technology are growing rapidly, with many new developments appearing constantly in such areas as electronic medical records, personal fitness trackers, doctor-patient communication, diagnostic sensors, and many more. Too many product and service offerings exist to enumerate even a fraction of them here. In fact, new offerings are emerging so rapidly that it is quite impossible to know about everything that exists. In such an environment, all that can be done is to describe a new solution on its own terms without attempting to compare it to others. What can be stated, however, is that it would be a rare for a system to integrate all these elements together such that the needs of all participants in drug or device clinical trials are incorporated in a balanced and thorough manner, covering not only the coordinators running the trial, but also the investigators analyzing the data and producing conclusions, the patients and their doctors on the front lines of health experience and data collection, and the oversight staff—risk managers, compliance officers, regulators, etc.—who ensure traceability of results and accountability of responsible parties.

Therefore, there exists a need for a clinical trial operations system to support the aforementioned participants with capabilities: such that the patients and their doctors who participate in a drug or device clinical trial benefit from automatic secure collection and storage of relevant data; such that each trial's investigators may securely receive appropriately anonymized (also referred to as de-identified) versions of that data, potentially in real time if allowed by the trial protocol, with automatic secure logging of data access, and may securely record corresponding research activities, observations, and conclusions in a fully traceable lab notebook tied to the trial, its data, and the data access logs; such that each trial's operational coordinators may design trial enrollment (including qualification and consent), engagement, data collection, and other information flow protocols quickly and easily within hours, and automatically create and deploy corresponding web and mobile device applications within minutes, without having to hire an engineering team to spend weeks or months translating the design into software; such that each trial's risk and compliance coordinators may analyze and extract traceability and accountability records as required; and such that all participants are able, if and as permitted by the trial protocol, to communicate with one another securely.

### SUMMARY

Some embodiments provide ways to manage clinical trials among various entities. Such entities may include, for instance, clinicians, patients, coordinators, investigators, trial management enterprises, etc. The various entities may interact via a set of applications provided by some embodiments. Such applications may be executed by various appropriate user devices (e.g., smartphones, tablets, personal computers, workstations, etc.) and/or facility devices (e.g., medical testing equipment, servers, etc.). Such user devices may be associated with various user types (e.g., patient devices, clinician devices, coordinator devices, investigator devices, etc.).

In addition, some embodiments may include various sensors (e.g., biometric sensors, health sensors, dosage monitors, environmental sensors, etc.). Such sensors may be associated with a particular patient and/or clinician (or facility). The sensors may automatically collect information related to the particular patient and/or clinician.

Various users (e.g., patient-users, clinician-users, coordinator-users, investigator-users, manager-users, etc.) may access a system of some embodiments via the set of applications, a set of corresponding portals, and an appropriate user device. For instance, a patient-user may utilize a patient application running on a device such as a smartphone and a patient portal provided by a system server or other appropriate resource. The applications and/or portals may be provided as network-based resources (e.g., via a web browser or application programming interface).

Some embodiments may provide secure communication pathways among users. Such communication may include automatically generated notifications. The pathways may allow for communication of user-specific information and/or provision of anonymous or de-identified data (e.g., a patient may be able to send information to a clinician for review and discussion, whereas a trial coordinator may only be able to receive such information without reference to the specific patient).

Collected data may be stored and/or analyzed at various appropriate system resources (e.g., one or more servers). Such analysis may include identification of health events or results. Such analysis may be used to trigger automatic notifications and/or updates, as appropriate.

The preceding Summary is intended to serve as a brief introduction to various features of some exemplary embodiments. Other embodiments may be implemented in other specific forms without departing from the scope of the disclosure.

### BRIEF DESCRIPTION OF THE SEVERAL VIEWS OF THE DRAWINGS

The exemplary features of the disclosure are set forth in the appended claims. However, for purpose of explanation, several embodiments are illustrated in the following drawings.

FIG. 1 illustrates a schematic block diagram of a clinical trial operations system according to an exemplary embodiment of the invention;

FIG. 2 illustrates a schematic block diagram of a trial operations service suite in a clinical trial operations system according to an exemplary embodiment of the invention;

FIG. 3 illustrates additional detail within the service suite of FIG. 2, according to an exemplary embodiment of the invention;

FIG. 4 illustrates a schematic block diagram of a group of federated trial operations service suites in a clinical trial operations system according to an exemplary embodiment of the invention;

FIG. 5-FIG. 21 illustrate a group of processes distributed among a patient application, patient portal, and other elements of a trial operations service suite in a clinical trial operations system according to an exemplary embodiment of the invention;

FIG. 22-FIG. 40 illustrate a group of processes distributed among a clinician application, clinician portal, and other elements of a trial operations service suite in a clinical trial operations system according to an exemplary embodiment of the invention;

FIG. 41-FIG. 53 illustrate a group of processes distributed among an investigator application, investigator portal, and other elements of a trial operations service suite in a clinical trial operations system according to an exemplary embodiment of the invention;

FIG. 54-FIG. 69 illustrate a group of processes distributed among a coordinator application, coordinator portal, and other elements of a trial operations service suite in a clinical trial operations system according to an exemplary embodiment of the invention;

FIG. 70-FIG. 77 illustrate a group of autonomous processes performed by elements of a trial operations service suite in a clinical trial operations system according to an exemplary embodiment of the invention; and

FIG. 78 illustrates a schematic block diagram of an exemplary computer system used to implement some embodiments.

### DETAILED DESCRIPTION

The following detailed description describes currently contemplated modes of carrying out exemplary embodiments. The description is not to be taken in a limiting sense but is made merely for the purpose of illustrating the general principles of some embodiments, as the scope of the disclosure is best defined by the appended claims.

Various features are described below that can each be used independently of one another or in combination with other features. Broadly, some embodiments generally provide mobile-native clinical trial operations systems and algorithms.

A first exemplary embodiment provides a trial operations service suite in a clinical trial operations system comprising: patient, clinician, investigator, and coordinator portals supporting corresponding applications and providing specific portal services for each type of participant, wherein the coordinator portal allows a coordinator user to develop, build, customize, and establish trial protocol and participant interaction using an application factory system embedded within a trial design aspect of the coordinator portal; security configuration and management authentication databases; operational databases, partitioned for security, comprising trial data including provisioning and authorization data describing trial participants, access logs recording participant activity, protected and de-identified health information provided by patients, clinicians, and associated sensors, and non-health information provided by patients, clinicians, and associated sensors; a notification relay service and corresponding external notifications gateway providing essential communication between the system and users, between clinicians and patients, and between coordinators and all participants; and a secure communication relay service providing multi-media communication among participants, constrained by permissions as established by a particular coordinator user.

A second exemplary embodiment provides a method of operating a trial operations service suite in a clinical trial operations system, the method comprising: serving a mobile application factory composition studio to a coordinator application via a coordinator portal; accepting from the coordinator application a set of customizations of trial protocol and participant interaction screens and logic; building and distributing customized patient, clinician, and investigator applications for use with corresponding patient, clinician, and investigator portals of the trial operations service suite; registering and enrolling patient, clinician, investigator, and coordinator users; inviting patients and clinicians to register and enroll as users; sending and receiving notifications among users; recording patient health information provided by patients, clinicians, and associated sensors; protecting patient health information according to Health Insurance Portability and Accountability Act (HIPAA) regulations; presenting de-identified patient health information to investigator users for analysis; and supporting secure multi-media communication among participants, constrained by permissions as established by a particular coordinator user.

The preceding is intended to serve as a brief introduction to various features of some exemplary embodiments of the invention. Other embodiments may be implemented in other specific forms within the scope of the invention.

### I. Hardware Architecture

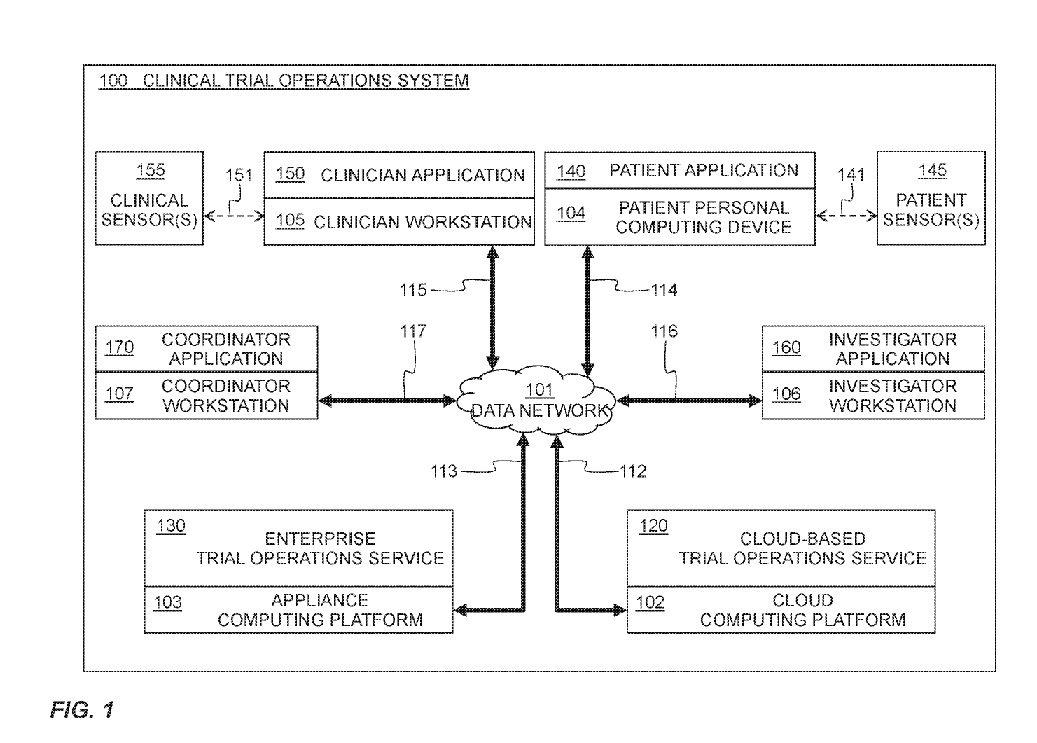

FIG. 1 illustrates a schematic block diagram of a clinical trial operations system 100 according to an exemplary embodiment of the invention. Data network 101 may interconnect the various elements of system 100. The network 101 may include one or more private networks as well as one or more public networks including the Internet. Constituent networks may include cellular mobile radio networks using various standards such as LTE; Wi-Fi networks serving public facilities such as airports, shopping centers, hospitals; Wi-Fi networks serving private facilities such as homes, labs, offices, and clinics; wired networks such as Ethernets within private facilities such as homes, labs, offices, and clinics; cable or DSL access networks; and any other form of data network.

Each category of participant may be considered to be a user of corresponding elements in system 100. A patient who participates in a clinical trial supported by system 100 may use a patient personal computing device 104, which may be a mobile device such as a tablet or smartphone, or a traditional computer such as a laptop or desktop, and which may connect to data network 101 via an interface 114 that may be implemented using any of numerous technologies as listed above or otherwise.

Patient application 140 may include software executed by the patient personal computing device 104 that may provide user interface (UI) and operational functions supporting the patient's interaction with system 100. Patient workstation 104 may also execute other software applications that fall outside system 100, in addition to, or in place of, patient application 140. For certain types of study, it may be necessary to incorporate health and environment information from sensors such as blood pressure monitors, heart rate monitors, personal fitness bands, smart watches, smart home products, and/or other similar devices.

For certain kinds of study, it may also be appropriate to incorporate health, event, and environment information from study-specific sensors provided to the patient, such as a custom drug dispenser that detects the time and conditions when a patient consumes a dose. Such devices are represented in the diagram by patient sensor(s) 145. In addition to study-specific sensors, such patient sensors may include biometric sensors (e.g., heart rate monitors, blood pressure sensors, blood sugar sensors, thermometers, etc.), environmental sensors (e.g., humidity sensors, temperature sensors, etc.), and/or event other event sensors. Furthermore, such sensors may include data received from a patient (e.g., indication of pain level or other appropriate factor, logging medication times and/or amounts, etc.).

Patient sensor(s) 145 may communicate with the patient personal computing device 104 via an interface 141 in order to provide sensor readings and related information. For each device represented by patient sensor(s) 145, interface 141 may include a wired connection such as a USB cable, and/or a wireless connection such as Bluetooth or Wi-Fi.

A doctor, advanced practitioner, or nurse who treats or oversees treatment of a patient participating in a clinical trial supported by system 100 may use a clinician workstation 105, which may be a mobile device such as a tablet or smartphone, or a traditional computer such as a laptop or desktop, and which may connect to data network 101 via an interface 115 that may be implemented using any of numerous technologies as listed above or otherwise.

Clinician application 150 may include software executed by clinician workstation 105 that provides UI and operational functions supporting the clinician's interaction with system 100. Clinician workstation 105 may also execute other software applications that fall outside system 100, in addition to, or in place of, clinician application 150.

For certain kinds of study, it may be necessary to incorporate health and environment information from sensors such as blood pressure monitors, heart rate monitors, thermometers, CT scanners, MRI scanners, and/or other diagnostic devices. Such devices are represented in the diagram by clinical sensor(s) 155. Clinical sensor(s) 155 may communicate with clinician workstation 105 via an interface 151 in order to provide sensor readings and related information. For each device represented by clinical sensor(s) 155, interface 151 may include a wired connection such as a USB cable or Ethernet network, a wireless connection such as Bluetooth or a Wi-Fi network, or a combination of such connections.

A technician, scientist, or other researcher who analyzes data and draws conclusions regarding the results of a clinical trial supported by system 100 may use an investigator workstation 106, which may be a mobile device such as a tablet or smartphone, or a traditional computer such as a laptop or desktop, and which may connect to data network 101 via an interface 116 that may be implemented using any of numerous technologies as listed above or otherwise.

Investigator application 160 may include software executed by investigator workstation 106 that provides UI and operational functions supporting the investigator's interaction with system 100. Investigator workstation 106 may also execute other software applications that fall outside system 100, in addition to, or in place of, investigator application 160.

A supervisor, designer, trial manager, site manager, risk manager, compliance manager, or other administrator who oversees one or more aspects of a clinical trial supported by system 100 may use a coordinator workstation 107, which may be a mobile device such as a tablet or smartphone, or a traditional computer such as a laptop or desktop, and which may connect to data network 101 via an interface 117 that may be implemented using any of numerous technologies as listed above or otherwise.

Coordinator application 170 may include software executed by coordinator workstation 107 that provides user interface and operational functions supporting the coordinator's interaction with system 100. Coordinator workstation 107 may also execute other software applications that fall outside system 100, in addition to or in place of coordinator application 170.

The system 100 may utilize a trial operations service that provides capabilities with which each participant may interact via the respective application. A distinct trial operations service may be used for each separate clinical trial in order to ensure strong data privacy protection, even when a single organization is operating multiple trials and therefore multiple trial operations services. Such an approach also prevents propagating any faults that may occur in one instance to other instances, thereby ensuring that no single fault compromises multiple trials.

The example of system 100 includes two kinds of trial operations service, but for any particular trial only one of those types is to be deployed, depending on the preference of the organization operating the trial.

A cloud-based trial operations service 120, which may include software functions executing in a cloud computing platform 102, may be selected by an organization that prefers to outsource its information technology operations to an expert in that field. For example, a biopharmaceutical research lab may prefer not to distract itself from its core competency, or a government research lab may require implementation using the services of a Federal Risk and Authorization Management Program (FedRamp)-approved provider.

An enterprise trial operations service 130, which may include software functions executing in an appliance computing platform 103, may be selected by an organization such as a large company with multiple capabilities that prefers to keep information technology operations in its own facility and network. For example, a medical device manufacturer may already have information technology competency due to the electronic and software-driven nature of its devices. Other reasons may also exist for choosing one or the other deployment option for the trial operations service.

One of ordinary skill in the art will recognize that system 100 may be implemented in various different ways without departing from the scope of the disclosure. For instance, the various elements of the system may be arranged in various different ways. As another example, various devices or elements may be implemented using multiple devices or sub-elements. Likewise, in some embodiments multiple devices or elements may be combined into a single device or element. In addition, various other elements may be included and/or various listed elements may be omitted in some embodiments.

### II. Services

Many clinical trials take place in multiple locations, with separate personnel who may or may not interact with one another. In each of the participant categories described above, individual participants may be associated with one or more such locations, or sites, such that data access and communication permissions may be constrained to remain within each site. For example, a coordinator in the trial manager role may have permission to access data for all sites, while a coordinator in a site manager role may only have permission to access data for the assigned site. However, in clinical trial operations system 100 all sites associated with a particular clinical trial are supported by the same trial operations support service 120 or 130, thus ensuring commonality of trial data and procedures across all sites.

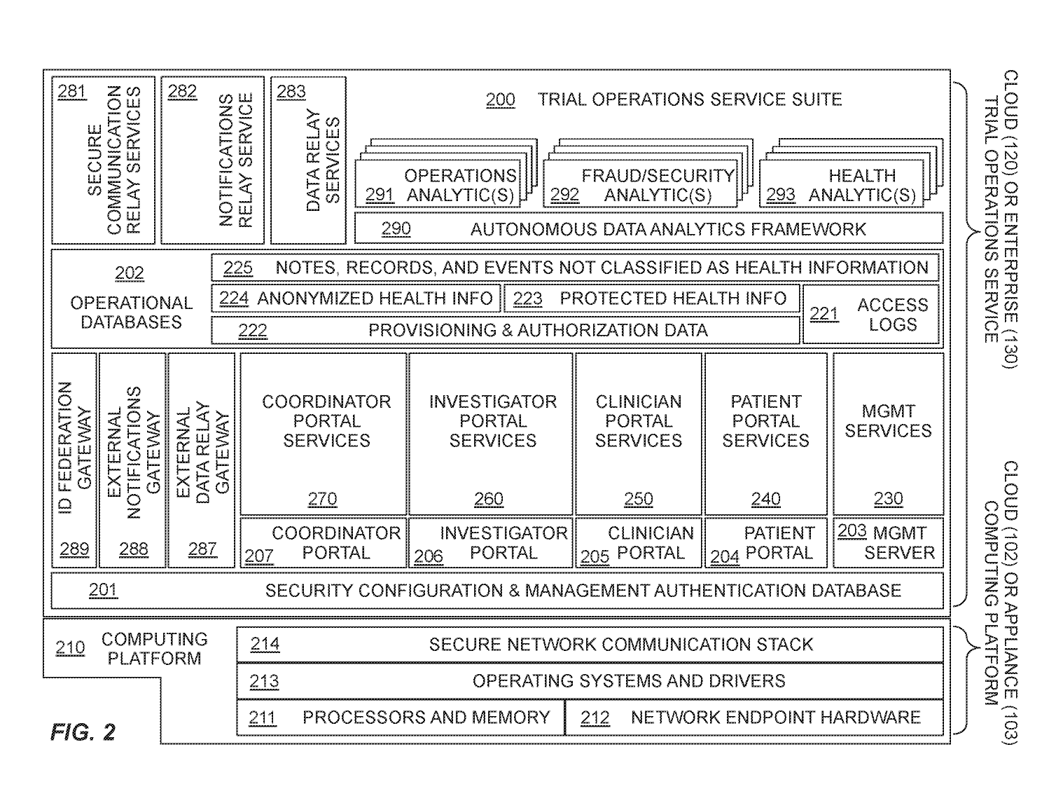

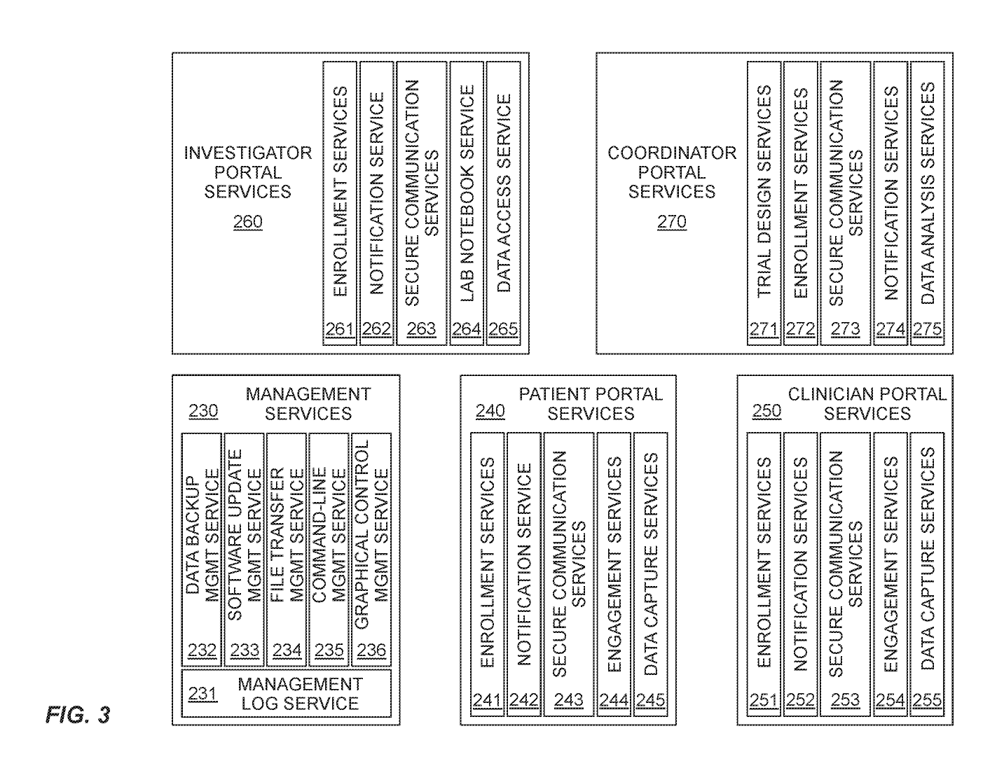

Enterprise trial operations service 130 and cloud-based trial operations service 120 may be similar, and both appliance computing platform 103 and cloud computing platform 102 provide similar capabilities. A unified view detailing these is provided in FIG. 2 and FIG. 3, which together illustrate a schematic block diagram for a trial operations service suite 200 and computing platform 210 in a clinical trial operations system according to an exemplary embodiment of the invention.

The mapping of trial operations service suite 200 modules to both cloud trial operations service 120 and enterprise trial operations service 130 is represented by the long upper bracket. The mapping of computing platform 210 modules to both cloud computing platform 102 and enterprise computing platform 103 is represented by the short lower bracket.

Platform elements may include processors and memory 211, network endpoint hardware 212, operating systems and drivers 213, and a secure network communication stack 214. In any particular deployment, computing platform 210 may include multiple physical or virtual computing machines, including multiple instances of the depicted modules, according to capacity, availability, and security requirements of the particular clinical trial to which the corresponding trial operations service suite 200 instance is applied, as is usual for such platforms.

Secure network communication stack 214 may provide standard protocols for interconnection with other elements of system 100, as well as external entities that may also be connected to data network 101 but aren't shown as part of system 100. Protocols may include TCP/IP, AMQP, HTTP, TLS, SMTP, IMAP, and/or other upper-layer protocols well known to information technology specialists. External entities may include physician billing or medical records systems, laboratory data processing systems, compliance-monitoring systems, data analytics systems, payment systems, external email networks, and others that may become useful over the life of a particular clinical trial.

Because clinical trial operations are subject to strict regulatory requirements regarding information security and privacy, computing platform 210 must be carefully selected and configured with features that accommodate those requirements. Encryption of data at rest and in motion, role-based data access permission capabilities, and bullet-proof communication stacks are all important. A cloud computing platform 102 may be used only if it explicitly supports HIPAA compliance, which in addition to the features listed above also requires physical security and strong partitioning among customers so unrelated applications cannot interact with one another either intentionally or unintentionally. An organization choosing to deploy trial operations service suite 200 in an appliance computing platform 103 is obliged to satisfy these physical security and related requirements itself.

Security configuration and management authentication database 201 may provide a data repository specifically for information about the particular trial operations service suite 200 and its deployment in computing platform 210. Configuration data may include, but is not limited to, such persistent knowledge as how it relates to the network in which it resides, how the various function modules are deployed across potentially multiple physical and/or virtual computing machines, and licensed capacity. Security configuration data in particular may provide the mathematical basis of trust for establishing communication paths at the level of attaching the services hosted in trial operations service suite 200 to other services elsewhere in the network. Management authentication data may provide the credentials and permissions necessary to protect access to management functions.

Operational databases 202 may provide multiple data repositories specific to the clinical trial supported by trial operations service suite 200. This group of databases is architecturally distinct from security configuration and management authentication databases 201 for reasons of information privacy, data capacity differences, and transaction capacity differences. A more detailed description of the specific data modules within operational databases 202 follows the descriptions of the services themselves, in order for the reader to have sufficient context.

Beyond that foundation, the functions of trial operations service suite 200 naturally fall into a few major groups. The first of these includes the management functions, which may provide ways to configure and control the trial operations service suite 200 and the underlying computing platform 210. Management server 203 may form the basis of the management function group, providing a network interface and an operating environment for the various management services. Management server 203 may include such components as a web server, a secure shell interface, a file transfer protocol server, and related capabilities typically used in the management of information technology systems. The following additional details of management services 230 are shown in FIG. 3.

Management log service 231 may ensure that every access of management server 203 from a management user (often also called a system administrator in the information technology industry), whether successfully authenticated or rejected, is recorded. Actions commanded by authenticated management users, as well as automatic actions performed by the various management services may also be recorded or logged. The placement of management log service 231 parallel to management server 203 indicates that service 231 may act as a support function for all the management services, enabling the responsibility of the service to log everything that happens.

The remaining management services may provide the usual functionality one finds in network element management. Certain key features are identified explicitly here, but others may be included as well.

Data backup management service 232 may provide ways to routinely capture consistent snapshots of all data in trial operations service suite 200, including the contents of security configuration and management authentication database 201 as well as the contents of operational databases 202, and securely convey each snapshot to an external repository.

Software update management service 233 may provide ways for securely receiving, authenticating, validating, and activating new software loads and configuration data for trial operations service suite 200.

File transfer management service 234 may provide ways for securely sending and receiving general data files, and may be used both independently and as a support function for data backup management service 232 as well as software update management service 233.

Command-line management service 235 may receive typed user commands that may change state or display information in a line-by-line terminal mode, while graphical control management service 236 may provide buttons and menu items that may change state or display information on a graphical interface mode and are able to be selected by a user.

The next major group of functions may provide access for various types of user in the trial community served by trial operations service suite 200. Each user category may be provided with a respective portal 204, 205, 206, or 207, which may include a web server dedicated to that category of users and which may support highly secure information presentation and interaction for multiple users within that category.

Multiple user roles may also be supported within each category, as well as site assignments for each user, depending on data access permissions encoded in provisioning and authorization data 222 of operational databases 202. For each user category, support for a variety of capabilities may be encapsulated within a corresponding set of services 240, 250, 260, or 270 built atop the corresponding portal 204, 205, 206, or 207, partitioned from the others to prevent inappropriate information crossover, and interacting with the corresponding application 140, 150, 160, or 170 of system 100. Each user category's portal and services are described below.

Patient portal 204 and patient portal services 240 may provide access for patients participating in the supported clinical trial, interacting with patient application 140. The patient user category may include user accounts not only for a particular patient participating in a clinical trial, but also any of that patient's family members or representatives such as a formally declared HIPAA agents or a holder of healthcare power of attorney who the patient has designated to have access to the system. Each patient account may have specific permissions to view data, update data, create diary and journal entries, receive notifications, and the like, depending on the corresponding records in provisioning and authorization data 222 as established by the patient, or the trial protocol as established by a coordinator. Patient portal 204 may provide, through patient application 140, a base view whereby the patient user can see account status, see overviews of each available service such as whether unviewed notifications exist, and select an available service to activate.

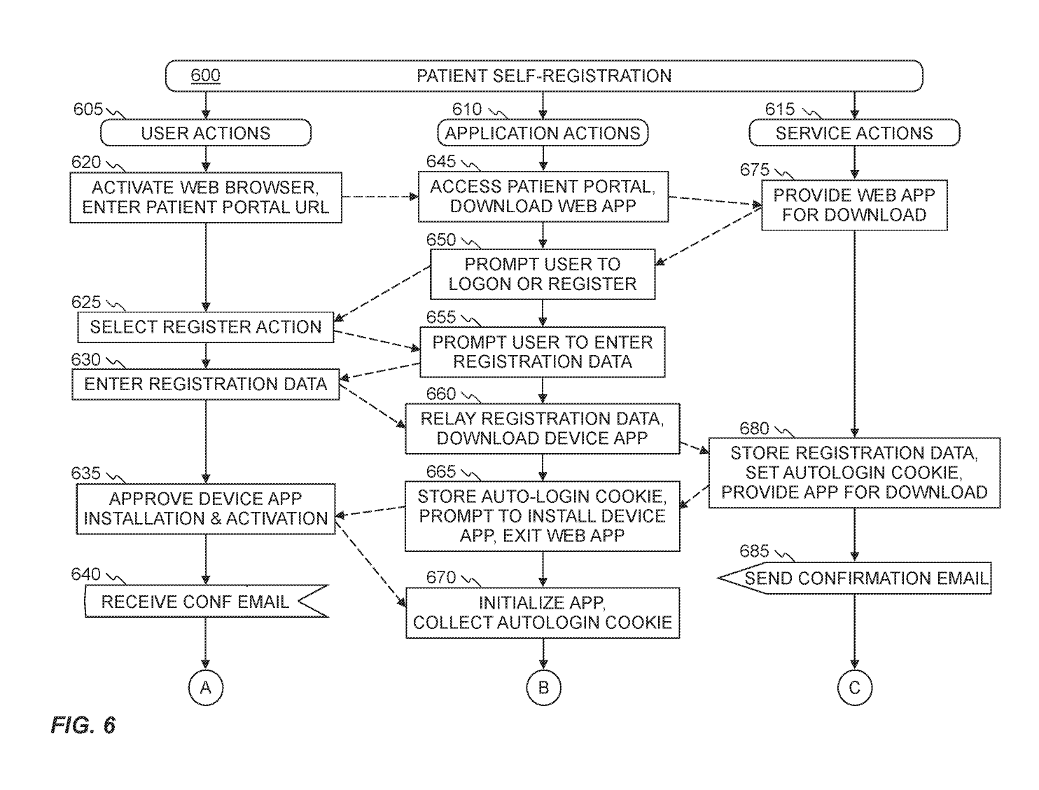

Patient portal services 240 are detailed further in FIG. 3 and may include any subset of the services shown depending on the requirements of the particular clinical trial being supported by trial operations service suite 200. Additional services not shown or described may also be created within this service framework. Procedural details for certain key capabilities of services 240 may be found in FIG. 6-FIG. 21.

Patient portal services 240 may include a group of enrollment services 241, which may support patient account management including registration of personal identifying and contact information, qualification and informed consent for trial participation, invitation of related clinicians and other patients to participate in the trial, invitation of family members or representatives to create a linked patient account, and control of corresponding data and service access permissions for linked patient accounts.

Patient portal services 240 may include a notification service 242, which may support receiving targeted or broadcast messages from coordinators or from automatic aspects of the system. Notifications may include status updates such as general trial progress information or system availability information, instructions such as requests for referrals or to complete a survey, reminders for timed events such as doctor appointments or drug doses, information about missed communication attempts, or any other similar information.

Notification service 242 may display a list of received notifications and may provide the ability to delete those that have been read and understood if permitted by the trial design. Alternatively, the trial design may not permit deletion, in which case notification service 242 would not offer the capability and all active notifications would remain available even after having been read. In a preferred embodiment, notification service 242 may be implemented, for example, using standard email client technology. In another preferred embodiment when patient application 140 is a mobile device application, notification service 242 may use a capability specific to a particular mobile device operating system, such as Firebase Cloud Messaging, Apple Push Notification Services, or Windows Push Notification Services.

Patient portal services 240 may include a group of secure communication services 243, which may act as a secure client supporting one or more of email, one-on-one chat, closed-group chat, open-group conversation forum, multimedia messaging, voice calling, and video calling between a patient and other users as permitted by the trial design. Additional communication modes may be incorporated into secure communication services 243 as necessary. Communication pairings likely to be permitted in a typical trial design may include a patient with his or her clinicians, and a patient with his or her family members within the same patient account group. For certain types of trials, it may also be appropriate to let all patients communicate with one another. Permission for other combinations may be unlikely, such as a patient with a coordinator or a patient with an investigator, but they may be supported if permitted. For each communication mode, standard protocols and client formats may be implemented in a preferred embodiment, but confined to the closed system 100 in order to ensure privacy and security of health-related information.

Patient portal services 240 may include a group of engagement services 244, which may provide a variety of capabilities allowing patients to record observations, answer surveys which may or may not have been announced or requested via a notification, report status either unsolicited or in response to a request in a notification, schedule events and associated reminders, keep a diary of medication dosing or device usage according to forms provided by the trial design, keep a journal of general observations or feelings in a free text format, and generally track progress and prompt activity to keep the patient participating through the trial.

Engagement services 244, with support from patient application 140, may also capture readings from non-medical sensors embedded natively in patient personal computing device 104. For example, a patient's location may be continuously tracked and used to measure as well as encourage compliance with doctor visit schedules or exercise regimens associated with the trial. Location data collected in this manner may also be used by analytics algorithms to detect multi-user patterns of compliance or non-compliance, location-related health effects, promising areas for trial participant recruiting, and other indications. Depending on trial design, engagement services 244 may be presented in a game-like fashion (also known as “gamification”), such that a patient receives encouragement and recognition for completing entries, responding to surveys and status queries, or showing up at scheduled appointments, for example; more desirable prizes may be issued for repeated instances of such desirable behaviors as well. Any information provided by a patient through engagement services 244 may be stored by patient portal 204 in operational databases 202.

Patient portal services 240 may include a group of data capture services 245, which may provide a variety of capabilities allowing patients to record observations, keep a diary of medication dosing or device usage according to forms provided by the trial design, and keep a journal of general observations or feelings in a free text format. Data capture services 245 may also operate in the background of patient portal 204 and patient application 140 to collect readings from patient sensors 145 and store them in operational databases 202. Data capture services 245 may vary according to what specific sensors are present and attached to patient personal computing device 104, as detected by patient application 140. A specific trial may require that one or more specific sensor types be present at all times or available on occasion, a detail to be expressed in trial design. Data capture services 245 may then enforce such a requirement and continuously or periodically or occasionally collect sensor readings according to the trial design. Also depending on trial design, a patient informed consent protocol enacted by enrollment services 241 may collect a patient's consent for access to patient sensors 145, in advance of actually enabling data capture services 245, so that data capture services 245 is absolved of any need for repeatedly requesting consent.

Clinician portal 205 and clinician portal services 250 may provide access for clinicians with one or more patients participating in the supported clinical trial, interacting with clinician application 150. The clinician user category may include user accounts not only for a doctor treating a particular patient participating in a clinical trial, but also any of that doctor's staff, partners, associated advanced practitioners, or nurses who also participate in treatment or care of a participating patient. Each clinician account may have specific permissions to view data, update data, create diary entries, receive notifications, and the like, depending on the corresponding records in provisioning and authorization data 222 as established by the primary clinician, or the trial protocol as established by a coordinator.

Clinician portal 205 may provide, through clinician application 150, a base view whereby the clinician user can see account status, see overviews of each available clinician-specific service such as whether unviewed notifications exist and select an available service to activate, view a list of associated patients participating in the supported clinical trial and corresponding overviews of patient-related services, and select a patient and service from that list.

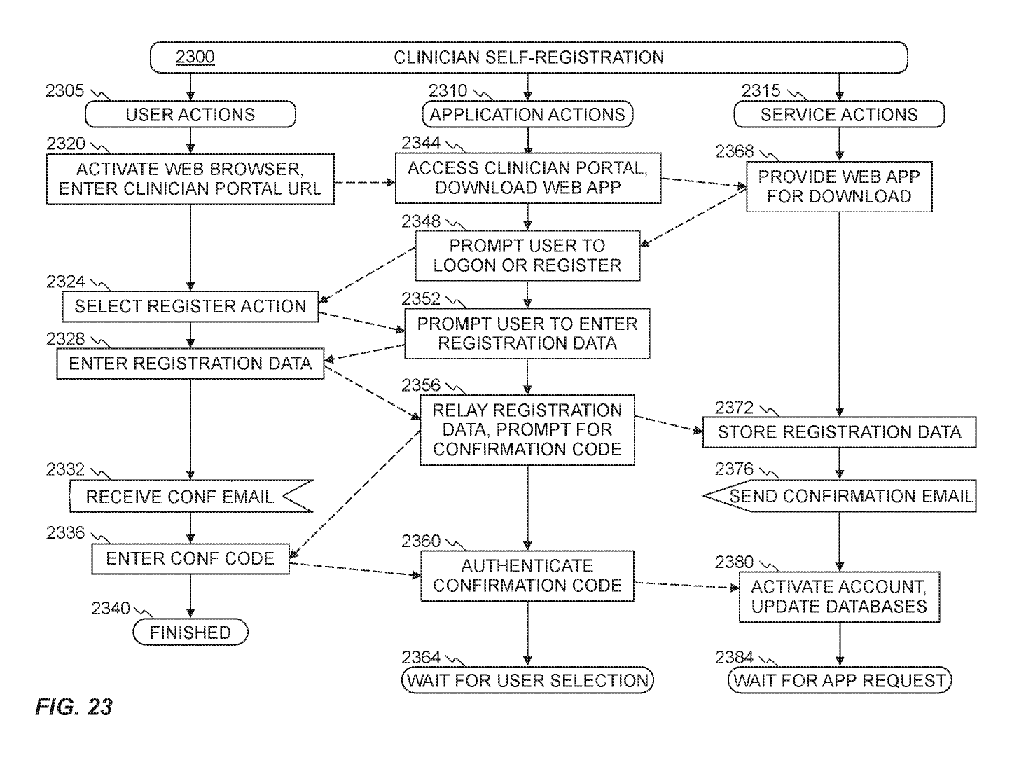

Clinician portal services 250 are detailed further in FIG. 3 and may include any subset of the services shown depending on the requirements of the particular clinical trial being supported by trial operations service suite 200; additional services not shown or described may also be created within this service framework. Procedural details for certain key capabilities of services 250 may be found in FIG. 23-FIG. 40.

Clinician portal services 250 may include a group of enrollment services 251, which may support clinician account management including registration of personal identifying and contact information, qualification and informed consent for trial participation, invitation of related clinicians and other patients to participate in the trial, control of corresponding data and service access permissions for linked clinician accounts, and status views of associated patients participating in the trial. Examples of how a primary clinician may control permissions for associated clinician's accounts may include: the medical assistants and advanced practitioners on a physician's team may each serve a separate subset of the patients under that physician's care, and therefore only have access to their respective subset; a nurse may have access to a patient's medical data but not the corresponding demographic information, while an administrative medical assistant may have access to the demographic information but not the medical data. Other combinations may be configurable as well.

Clinician portal services 250 may include a group of notification services 252, which may support receiving targeted or broadcast messages from coordinators or from automatic aspects of the system, as well as sending targeted or broadcast messages to associated patients. Received notifications may include status updates such as general trial progress information or system availability information, instructions such as requests for referrals or to complete a survey, reminders for timed events such as patient appointments or procedures, information about missed communication attempts, or any other similar information.

Notification services 252 may display a list of received notifications, and it may provide the ability to delete those that have been read and understood if permitted by the trial design. Alternatively, the trial design may not permit deletion, in which case notification services 252 would not offer the capability and all active notifications would remain available even after having been read. Notification services 252 may also offer the ability to send notifications, if permitted by the trial design, in which case a composition and recipient selection capability may be present. In a preferred embodiment, notification services 252 may be implemented, for example, using standard email client technology. In another preferred embodiment when clinician application 150 is a mobile device application, notification services 252 may use a capability specific to a particular mobile device operating system, such as Firebase Cloud Messaging, Apple Push Notification Services, or Windows Push Notification Services.

Clinician portal services 250 may include a group of secure communication services 253, which may act as a secure client supporting one or more of email, one-on-one chat, closed-group chat, open-group conversation forum, multimedia messaging, voice calling, and video calling between a clinician and other users as permitted by the trial design.

Additional communication modes may be incorporated into secure communication services 253 as necessary. Communication pairings likely to be permitted in a typical trial design may include a clinician with his or her patients, and a clinician with his or her associated clinicians within the same account group. For certain types of trials, it may also be appropriate to let all clinicians communicate with one another, with investigators, and with coordinators. For each communication mode, standard protocols and client formats may be implemented in a preferred embodiment but confined to the closed system 100 in order to ensure privacy and security of health-related information.

Clinician portal services 250 may include a group of engagement services 254, which may provide a variety of capabilities allowing clinicians to answer surveys which may or may not have been announced or requested via a notification, report status either unsolicited or in response to a request in a notification, schedule events and associated reminders, and generally prompt activity to keep the clinician participating through the trial.

Depending on trial design, engagement services 254 may be presented in a game-like fashion (also known as “gamification”), such that a clinician receives encouragement and recognition for completing entries, responding to surveys and status queries, or performing specific procedures during scheduled appointments, for example; more desirable prizes may be issued for repeated instances of such desirable behaviors as well. Any information provided by a clinician through engagement services 254 may be stored by clinician portal 205 in operational databases 202.

Clinician portal services 250 may include data capture services 255, which may provide capabilities for a clinician to record procedures, observations, and sensor readings from clinical sensors 155 during a clinical appointment with or for a particular patient participating in the trial and store them in operational databases 202. Sensor aspects of data capture services 255 may vary according to what specific sensors are present and attached to clinician workstation 105, as detected by clinician application 150. Diary aspects of data capture services 255 may vary according to trial design to focus on observations and procedures that are pertinent to the trial.

Investigator portal 206 and investigator portal services 260 may provide access for investigators handling the research and analysis responsibilities in the supported clinical trial, interacting with investigator application 160. The investigator user category may include user accounts for numerous scientists, technicians, and others who access anonymized/de-identified health information and reports to assess progress, success, and failure of various trial goals. Each investigator account may have specific permissions to view data, create lab notebook entries, receive notifications, and the like, depending on the corresponding records in provisioning and authorization data 222 as established by the principal investigator, or the trial protocol as established by a coordinator. Investigator portal 206 may provide, through investigator application 160, a base view whereby the investigator user can see account status, see overviews of each available service such as whether unviewed notifications exist, and select an available service to activate.

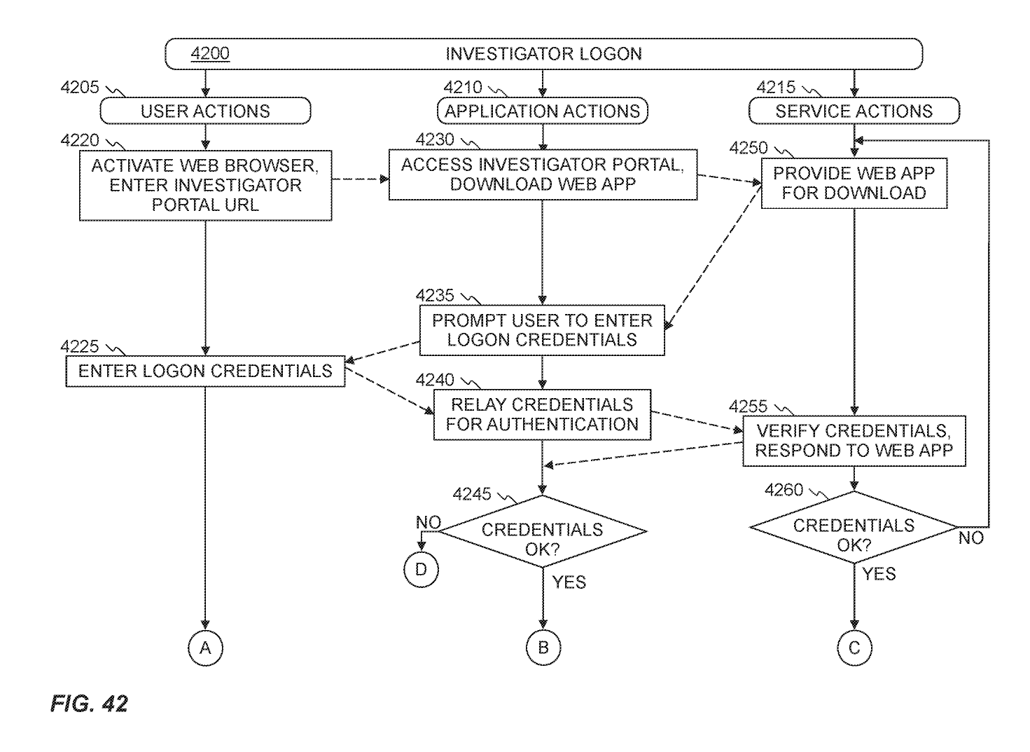

Investigator portal services 260 are detailed further in FIG. 3 and may include any subset of the services shown depending on the requirements of the particular clinical trial being supported by trial operations service suite 200; additional services not shown or described may also be created within this service framework. Procedural details for certain key capabilities of services 250 may be found in FIG. 42-FIG. 53.

Investigator portal services 260 may include a group of enrollment services 261, which may support investigator account management including registration of personal identifying and contact information, and examination of anonymized/de-identified patient and clinician statistics.

Investigator portal services 260 may include a notification service 262, which may support receiving targeted or broadcast messages from coordinators or from automatic aspects of the system. Received notifications may include status updates such as general trial progress information or system availability information, information about missed communication attempts, or any other similar information.

Notification service 262 may display a list of received notifications, and it may provide the ability to delete those that have been read and understood if permitted by the trial design. Alternatively, the trial design may not permit deletion, in which case notification service 262 would not offer the capability and all active notifications would remain available even after having been read. Notification service 262 may be implemented using standard email client technology in a preferred embodiment.

Investigator portal services 260 may include a group of secure communication services 263, which may act as a secure client supporting one or more of email, one-on-one chat, closed-group chat, open-group conversation forum, multimedia messaging, voice calling, and video calling between an investigator and other users as permitted by the trial design. Additional communication modes may be incorporated into secure communication services 263 as necessary. Communication pairings likely to be permitted in a typical trial design may include an investigator with his or her peer investigators, and an investigator with a coordinator. For certain types of trials, it may also be appropriate to let an investigator communicate with clinicians. Communication between investigators and patients may be unlikely, but such a capability may be supported if permitted. For each communication mode, standard protocols and client formats may be implemented in a preferred embodiment but confined to the closed system 100 in order to ensure privacy and security of health-related information.

Investigator portal services 260 may include an electronic lab notebook service 264, which may provide a variety of capabilities allowing investigators to record observations and experiment results regarding the trial. Electronic lab notebook service 264 may support importation of analysis results, notes, and other pertinent information created using tools that exist outside system 100. Electronic lab notebook service may support free-form data entry, or it may structure data entry according to requirements of the trial as expressed in the trial design. Electronic lab notebook service 264 may authenticate and render unalterable all entries so as to provide an auditable and legally admissible chain of evidence regarding the trial research results, as may be necessary for regulatory actions related to the trial and its goals. Any information provided by an investigator through electronic lab notebook service 264 may be stored by investigator portal 206 in operational databases 202.

Investigator portal services 260 may include a data access service 265, which may provide capabilities for an investigator to retrieve anonymized/de-identified health information and non-health information from operational databases 202. Arbitrary queries and filtering may be supported. Predefined queries provided by the trial design may be supported as well. Data access service 265 may also log every data access request and its results in a form interoperable with electronic lab notebook service 264, so as to enhance the auditable experiment record. Data analysis tools may exist outside of system 100; it would be the investigator's responsibility to import corresponding results, notes, and other pertinent information using electronic lab notebook service 264.

Coordinator portal 207 and coordinator portal services 270 may provide access for people in charge of managing various aspects of the trial, interacting with coordinator application 170. The coordinator user category may include user accounts for various individuals with distinct or overlapping responsibilities, including but not limited to trial design, everyday trial operations, and compliance. Each coordinator account may have specific permissions to view data, update data, change the trial design, send notifications, and the like, depending on the corresponding records in provisioning and authorization data 222 as established by the primary coordinator. Coordinator portal 207 may provide, through coordinator application 170, a base view whereby the coordinator user can see account status, see overviews of each available coordinator-specific service, and select an available service to activate.

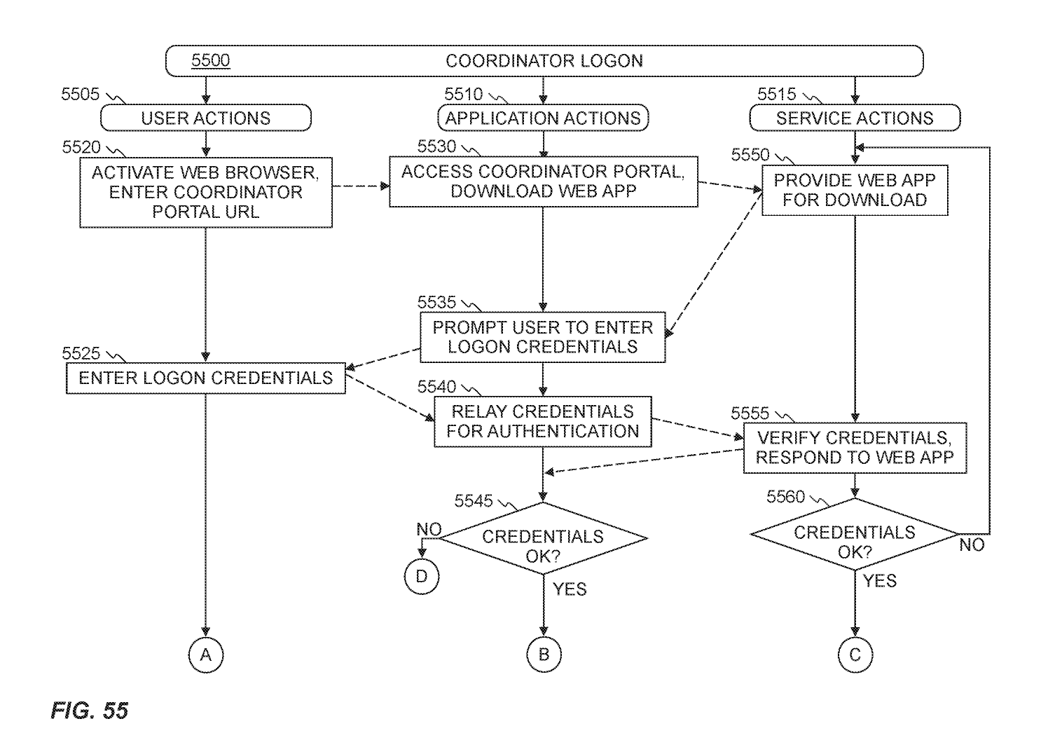

Coordinator portal services 270 are detailed further in FIG. 3 and may include any subset of the services shown depending on the requirements of the particular clinical trial being supported by trial operations service suite 200; additional services not shown or described may also be created within this service framework. Procedural details for certain key capabilities of services 250 may be found in FIG. 55-FIG. 69.

Coordinator portal services 270 may include a group of trial design services 271, which may support configuration of trial operations service suite 200 for the particular clinical trial being supported. Trial design services 271 may be used both at the beginning of a trial to set its initial design, and during the trial to change aspects of its design that may need adjustment as the trial progresses. Trial design services 271 may provide selections to configure whether specific services are available in each group of portal services 240, 250, and 260, such as which communication modes may be activated in secure communication services 243, 253, 263, and 273. Other feature selections may include without limitation whether identity federation gateway 289 is active and if so with what other trials it may interoperate, whether external data relay gateway 287 is active and if so what external connections it may make, what kind of inter-user communication, notification, and invitation pairings are permitted, and what kind of data analytics are activated.

Trial design services 271 may also provide capabilities for designing detailed user interaction procedures such as screen layouts, order of presentation and text for participant qualification and informed consent protocols, supported sensors both mandatory and optional, sensor data collection schedules, branding imagery and wording, wording of internal and external notification templates, wording of invitation templates, structure of surveys and status queries, structure and emphasis of diary or journal entry templates, structure and emphasis of lab notebook entry templates, supported data access query templates, data access result display formats, and every other aspect of user interaction supported by applications 140, 150, and 160. Trial design services 271 may even alter the look and capabilities of coordinator application 170.

In a preferred embodiment, trial design services 271 may be implemented by embedding in trial operations service suite 200 an enhanced version of the Application Factory System disclosed in U.S. Pat. No. 8,719,776, which is incorporated herein by reference.

Numerous enhancements have been made to the Application Factory System since its disclosure in the referenced patent. For example, the Build Engine is now able to create not only native mobile applications for deployment to all existing mobile device types as previously disclosed, but also web applications for deployment to a web browser via a web server. In trial operations service suite 200, this supports creation and per-trial tuning of the various user portals 204, 205, 206, and 207, which as has been stated are effectively web servers, along with their corresponding portal services 240, 250, 260, and 270, and their corresponding user applications 140, 150, 160, and 170. Each user application may be deployed as either a web application or a mobile application, or both, at the discretion of the coordinator designing the trial through trial design services 271.

In another important enhancement of the Application Factory System, which is already presaged in its patent as “formalized enhanced concepts” incorporated in the Data and Logic Canvas at “higher . . . Levels” of “complexity or capability,” the Visual Representation Language has been extended to incorporate multiple additional modes of expression covering multiple additional types of logic and data structure. The Composition Studio is now able to support these new language modes, and the Build Engine is now able to incorporate corresponding code, including trained neural networks, into generated applications, services, and data structures.

Examples of new visual language constructs include the decision-tree language of Flowable, which may be used in trial design services 271 for generation of user surveys, participant qualification protocols, and informed consent protocols; multiple screen-design wireframe tools including InVision, Sketch, and Photoshop, which may be used in trial design services 271 for generation of screen layouts and branding details; the database schema construction tools from DbDesigner.net, which may be used in trial design services 271 for tuning operational databases 202 to the specific requirements of a particular clinical trial; and a novel visual language specifically for designing machine learning algorithms, selecting artificial neural network parameters (depth, density, feedback propagation distance, etc.), and feeding datasets to the resulting machine for training, which may be used in trial design services 271 to, for example, generate trial-specific analytics modules to deploy on autonomous data analytics framework 290.

Also extended is the ability of the Service Palette to represent, and resulting applications to employ and interact with, not only peripherals, sensors, and interfaces embedded in a unitary mobile device that is executing an application, but external peripherals and sensors connected via those interfaces as well; this connectivity middleware becomes a library included in every application produced by the Build Engine and deployed as patient application 140 or clinician application 150.

Finally, the Application Factory System has been enhanced to compose, build, and distribute complete client-server applications that include both front-end elements deployed via web browser or as mobile device applications, and back-end elements deployed as services into infrastructure such as cloud computing platform 102 and appliance computing platform 103. Further, the Composition Studio, Build Engine, and Distribution Center of the Application Factory System have been enhanced to support service deployment to multiple types of cloud computing platform 102, including both a monolithic multi-server style of back-end acting as an always-on data center enabling economical operation under constant load, and a “microservices” or “serverless” style of back-end enabling economical operation under light usage or intermittent load. A coordinator may use these distribution model capabilities in trial design services 271 to tune the entire trial operations service suite 200 package for economical deployment according to the expected usage pattern for the clinical trial being supported.

Coordinator portal services 270 may include a group of enrollment services 272, which may support coordinator account management as well as account management for other user classes, including registration with or without personal identifying and contact information, invitation of preselected clinicians and patients to participate in the trial (if such are known and their contact information is available), control of corresponding data and service access permissions for all user accounts, and status views of investigators, clinicians, and patients participating in the trial.

Enrollment services 272 may also provide links to tools for managing external advertising, enabling recruitment of unknown and therefore non-invitable trial participants, both patients and clinicians. Even different users within the coordinator class may have different permissions, depending on each individual's role with respect to the specific trial. For example, one coordinator may have only trial design responsibility and therefore access to trial design services 271 but not other services, while another coordinator may have compliance responsibility and have access to data analysis services 275 but not other services. Any combination of permissions may be constructed within enrollment services 272 in order to support a variety of separate responsibilities.

Coordinator portal services 270 may include a group of secure communication services 273, which may act as a secure client supporting one or more of email, one-on-one chat, closed-group chat, open-group conversation forum, multimedia messaging, voice calling, and video calling between a coordinator and other users as permitted by the trial design. Additional communication modes may be incorporated into secure communication services 273 as necessary. Communication pairings likely to be permitted in a typical trial design may include a coordinator with other coordinators, and a coordinator with investigators. For certain types of trials, it may also be appropriate to let coordinators communicate with clinicians. Communication between coordinators and patients may be unlikely, but such a capability may be supported if permitted. For each communication mode, standard protocols and client formats may be implemented in a preferred embodiment but confined to the closed system 100 in order to ensure privacy and security of health-related information.

Coordinator portal services 270 may include a group of notification services 274, which may support receiving targeted or broadcast messages from other coordinators or from automatic aspects of the system, as well as sending targeted or broadcast messages to other coordinators, investigators, clinicians, and patients either individually or as a group. Received notifications may include status updates such as general trial progress information or system availability information, reminders for timed events such as compliances audits, information about missed communication attempts, or any other similar information. Notification services 274 may display a list of received notifications, and it may provide the ability to delete those that have been read and understood if permitted by the trial design. Alternatively, the trial design may not permit deletion, in which case notification services 274 would not offer the capability and all active notifications would remain available even after having been read. Notification services 274 may also offer the ability to send notifications, if permitted by the trial design, in which case a composition and recipient selection capability may be present. Notification services 274 may be implemented using standard email client technology in a preferred embodiment.

Coordinator portal services 270 may include a group of data analysis services 275, which may provide a variety of capabilities allowing coordinators to retrieve various statistics as well as, if permitted, anonymized/de-identified health information and non-health information from operational databases 202. If permitted, a coordinator may also use data analysis services 275 to retrieve protected health information from operational databases 202. Arbitrary queries and filtering may be supported. Predefined queries provided by the trial design may be supported as well.

Data analysis services 275 may also provide tools for analyzing retrieved data, including for example display formatting and filtering capabilities, and statistical curve-fitting capabilities. Data analysis services 275 may incorporate an equivalent of the investigator's electronic lab notebook service 264, so as to ensure data access is auditable and to enhance the experiment record. Data analysis services 275 may provide capabilities for exporting data to external tools that may exist outside of system 100; it would be the coordinator's responsibility to import corresponding results, notes, and other pertinent information using the electronic lab notebook service equivalent, or to log into the system as an investigator and use the corresponding electronic lab notebook service 264.

The next major group of functions are the internal relay services and external gateways. Relay services provide capabilities for interaction among services of trial operations service suite 200. Gateways provide capabilities for communication and data access between trial operations service suite 200 and affiliated external systems, or among multiple related trial operations service suites 200; these affiliations and relations may generally be driven by common business control, for example.

Secure communications relay service 281 may provide standards-based server roles associated with the email, one-on-one chat, closed-group chat, open-group conversation forum, multimedia messaging, voice calling, and video calling communication modes for which secure communications services 243, 253, 263, and 273 may provide corresponding standards-based client roles. For each communication mode, standard protocols and server formats may be implemented in a preferred embodiment but confined to the closed system 100 in order to ensure privacy and security of health-related information.

Notifications relay service 282 may provide a standards-based email server role corresponding to the standards-based email client role provided in a preferred embodiment by notification services 242, 252, 262, and 274. Use of standard email technology for notification relay service 282 and notification services 242, 252, 262, and 274 is only one option for implementation of these services; a non-standard messaging technology may be used in an alternate embodiment, as may standard chat, news, RSS, or blog technology. In any case, notifications may be confined to the closed system 100 in order to ensure privacy and security of health-related information, except for notifications to users' external contact addresses via external notifications gateway 288, which may be filtered to ensure no leakage of protected health information.

External notifications gateway 288 may provide a connection to external networks for the purpose of forwarding notifications with no protected health information, such as notice of a missed or available communication attempt, to an external contact address associated with a particular user. In a preferred embodiment, external notifications may be limited to using email or cellular short message service (SMS) technology in order to contact people via common and publicly accessible networks using one or more well-known address formats, regardless of what technology is used internally for notifications. In an alternate embodiment, external notifications may be provided via interoperation with a mobile device push notification network such as Firebase Cloud Messaging, Apple Push Notification Services, or Windows Push Notification Services. However, additional technologies may be supported in an alternate embodiment if they are common enough to justify implementation; for example, it may become appropriate to interoperate with widely used closed networks such as Facebook Messenger and others.

Data relay services 283 may provide an internal database access client accessible by external data relay gateway 287. External data relay gateway 287 may provide an entry point to trial operations service suite 200 for external users associated with external systems such as physician billing or medical records systems, laboratory data processing systems, compliance-monitoring systems, data analytics systems, or others, allowing them access to data in operational databases 202 if provisioned in, and as authorized in, provisioning and authorization data 222.

Identity federation gateway 289 may support a standard single-sign-on protocol among multiple trial operations service suites 200 operated by a single trial specialist or sponsor, or among affiliated trial specialists or sponsors. If configured for a particular trial and permitted for a particular user, identity federation gateway 289 allows coordinator portal 207, investigator portal 206, clinician portal 205, and possibly patient portal 204 to present a logged-on user with a list of affiliated trials whose trial operations service suite 200 may be accessed directly without re-entering log-on credentials. In a preferred embodiment, standard federated identity technology may be used to accomplish this.

In each case, the external gateway and internal relay roles may be separated for security architecture purposes, to ensure that a process in contact with an open external network port does not have direct access to an internal database or other internal data flow. This implies that user portals 204, 205, 206, and 207, as well as identity federation gateway 289, may also utilize data relay services 283 for access to provisioning and authorization data when authenticating a user logon attempt. However, after successful authentication and establishment of a secure connection between said portals and corresponding user applications 140, 150, 160, and 170, portal services 240, 250, 260, and 270 may securely access operations databases 202 without the assistance of data relay services 283.

The final major group of functions in trial operations service suite 200 is the analytics. Autonomous data analytics framework 290 may provide an environment for automatic processes that analyze different types or multiple types of data as it comes into the system, looking for correlations indicating some important actionable fact. Framework 290 may include utility functions for examining swaths of data from operational databases 202, for monitoring streams of events from the various services and gateways, and for passing new data and events back into the databases, services, and gateways as appropriate. Each analytic algorithm may be embodied in an actor working in the context of framework 290, and may be specifically programmed to examine a particular kind of data or event, and may create new data or events as a result, or initiate a notification if appropriate. Throughout this disclosure, the term “actor” may refer to sets of hardware and/or software modules that may provide the functionalities described herein.

Three classes of analytic actor are depicted in this example, and additional classes may be devised in the context of framework 290. Operations analytics 291 may be designed to recognize certain data as indicating lack of progress or achievement of a milestone relevant to the clinical trial operation overall, or to recognize a pattern of significance in patient enrollments such as excess location clustering or insufficient age distribution. Fraud/security analytics 292 may be designed to recognize certain data as indicating an intrusion from incorrectly authorized users; a pattern of information abuse by a coordinator, clinician, or investigator; a pattern of insufficient observation recording by an investigator; or a variety of other possible symptoms of system misuse or attack. Health analytics 293 may be designed to recognize certain data as indicating a significant health event for a particular patient that might otherwise go unnoticed, for example via correlation of long-term sensor readings or correlation of information from multiple clinicians.

Analytic actors may be selected from a menu of available algorithms during trial design or designed specifically for a trial using the composition studio tools provided by trial design services 271. Through the ability of trial design services 271 to change the trial design as the trial proceeds, additional analytic actors may be selected for activation, or new ones designed, throughout the life of the trial.

Now that all the major function groups have been described, the reader has sufficient context to learn the details of operational databases 202. As has been stated previously, operational databases 202 may provide multiple data repositories specific to the clinical trial supported by trial operations service suite 200. Data is partitioned within operational databases 202 in order to provide security and privacy for legally protected information separately from information that is not necessarily legally protected but that should still be secured, and in particular in order to ensure corresponding access controls are enforced. Every data record in every module of operational databases 202 may be encrypted for privacy and security protection, accompanied by cryptologic authentication so as to be immutable, and duplicated in multiple locations to ensure no data is lost in the event of equipment failure. Encryption algorithms and keys may change over time as necessary to remain ahead of technological advances for compromising any particular generation of protection.

First among the modules of operational databases 202 may be access logs 221, which records all access by any service to any piece of data within any module of operational databases 202. Each log entry includes a record of what data item was accessed, by which service within trial operations service suite 200, on behalf of what user, and when the access occurred. Other attributes associated with the particulars of a data access event may also be recorded, depending on the specific nature of the access. Blockchain techniques may be used to protect the integrity of event log ordering.

Provisioning and authorization data 222 may store information regarding users, their relationships, and their permissions. Provisioning data for each represented participant may include, but is not limited to, identifying information and other registration data such as enrollment (including qualification and consent) status if the participant is a patient or a clinician, logon credentials within the corresponding portal, and external addresses for use by external notifications gateway 288. Provisioning data may also include interoperable credentials for use by identity federation gateway 289, if supported by the trial design and appropriate for the particular user. External users with data access rights through external data relay gateway 287 may also be represented in provisioning data by registration data such as access credentials, again if supported by the trial design.

Authorization data may include the set of records representing relationships between patients and clinicians, between patients and their affiliates (family members and representatives), and between primary clinicians and their affiliates (partners, nurses, etc.). Authorization data may also include data and service access permissions for each user, effectively defining roles and controlling what each can do within trial operations service suite 200.

Protected health information 223 may store detailed medical and identifying information regarding each patient participating in the clinical trial, as collected from patients and clinicians by patient application 140 and clinician application 150 in cooperation with patient portal 204 and clinician portal 205 and the modules of patient portal services 240 and clinician portal services 250. This database module may contain legally protected information covered under various privacy laws such as HIPAA, and therefore requires partitioned access and access control. Users with a need to access this data, which may include the patient or the patient's attending clinician, may be permitted to read and write to protected health info 223.

Anonymized health information 224 may store detailed medical information regarding each patient participating in the clinical trial, collected from patients and clinicians as described above for protected health information 223. However, anonymized health information 224 omits patient identifying information that might otherwise accompany detailed medical information. For example, patient data may be distinguished by a unique anonymous patient code that cannot be correlated to the patient's actual identity. Further, detailed information that might contribute to de-anonymizing (or re-identifying) the medical information is omitted or altered to reduce its precision. For example, location tracking information may be stored with reduced latitude/longitude precision or limited to zip code, city, or state if necessary in anonymized health information 224, where it may be as precise as a GPS reading in a corresponding record in protected health information 223. Users with no need to access protected health information 223, which may include investigators and coordinators, may instead be granted access to anonymized health information 224.

Notes, records, and events not classified as health information 225 may store everything else recorded within trial operations service suite 200. Such information may include, without limitation, communication event records created by the various secure communication services 243, 253, 263, and 273, as well as secure communications relay services 281; notification event records created by the various notification services 242, 252, 262, and 274, as well as notifications relay service 282 and external notifications gateway 288; correlations and other events detected by the various analytics operating within autonomous data analytics framework 290; and research notes collected by electronic lab notebook service 264 and data analysis services 275.

While the examples shown may illustrate many individual modules as separate elements, one of ordinary skill in the art would recognize that these modules may be combined into a single functional block or element. One of ordinary skill in the art would also recognize that a single module may be divided into multiple modules.

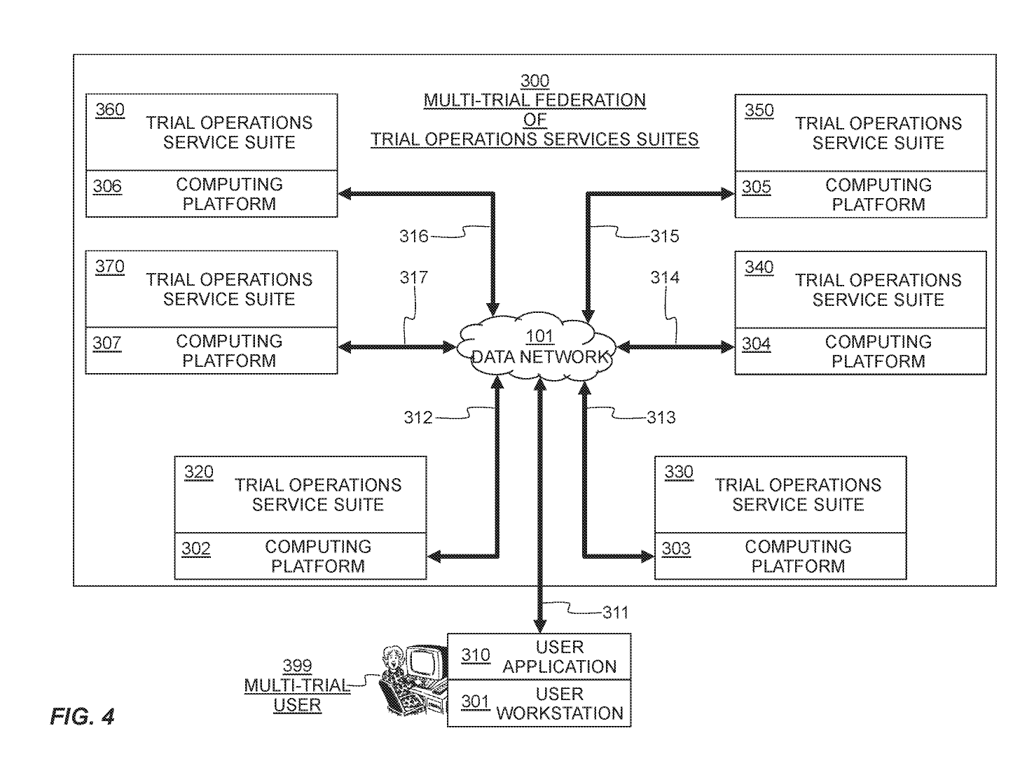

FIG. 4 illustrates a schematic block diagram for a group of federated trial operations service suites in a clinical trial operations system according to an exemplary embodiment of the invention. In the example multi-trial federation of trial operations suites 300, six trial operations service suites 200 and corresponding computing platforms 210 are designated trial operations service suites 320, 330, 340, 350, 360, and 370, hosted in computing platforms 302, 303, 304, 305, 306, and 307, respectively. Each one supports a separate clinical trial, but because all 6 clinical trials may be operated by the same contract research organization (CRO) or other entity such as a pharmaceutical developer or government research lab, for example, they may share investigators and coordinators and desire to use federated identity to simplify the lives of those individuals by allowing single-sign-on among the trials. Each trial operations service suite would be configured to have an active identity federation gateway 289, and their corresponding security configuration in security configuration and management authentication database 201 would provide credentials for secure identity federation connections between each pair of them, facilitated by data network 101 via transport interfaces 312, 313, 314, 315, 316, and 317 respectively.

With such a network established, multi-trial user 399, who by way of example may be a coordinator employed by the CRO or other entity to oversee the design of all six trials, or perhaps an investigator contracted by the CRO or other entity to research related aspects of all 6 trials, or even a clinician with patients participating in three of the trials, may user his or her user workstation 301 (which may be a clinician workstation 105, an investigator workstation 106, or a coordinator workstation 107, depending on which role user 399 performs) and user application 310 (which may respectively be the clinician application 150, investigator application 160, or coordinator application 170) to sign on via one trial's trial operations service suite, and proceed to any of the others, as authorized, via single sign-on—that is, without having to keep track of and enter separate credentials for every authorized trial.

### III. Methods of Operation

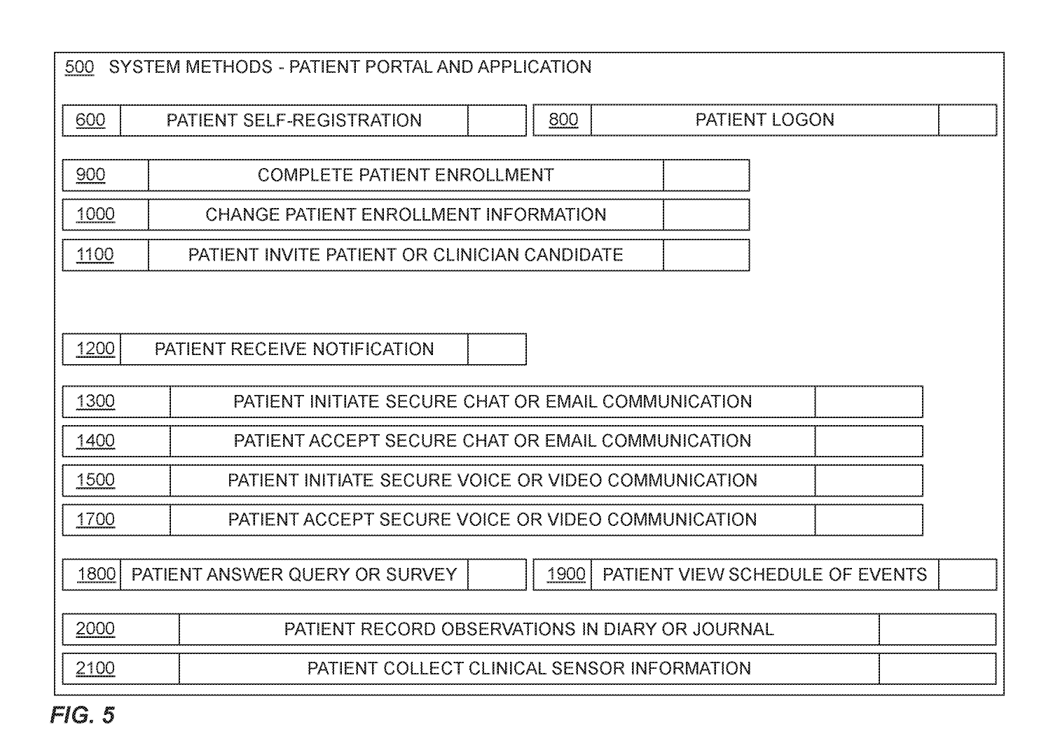

FIG. 5 illustrates a group of processes distributed among a patient application, patient portal, and other elements of a trial operations service suite in a clinical trial operations system according to an exemplary embodiment of the invention. The processes may include self-registration, logon, enrollment completion, personal data update, participant invitations, notification receipt, secure communication, survey completion, calendar utilization, diary and journal entries, and sensor data collection. FIG. 6-FIG. 21 illustrate additional detail for each of the processes in FIG. 5.

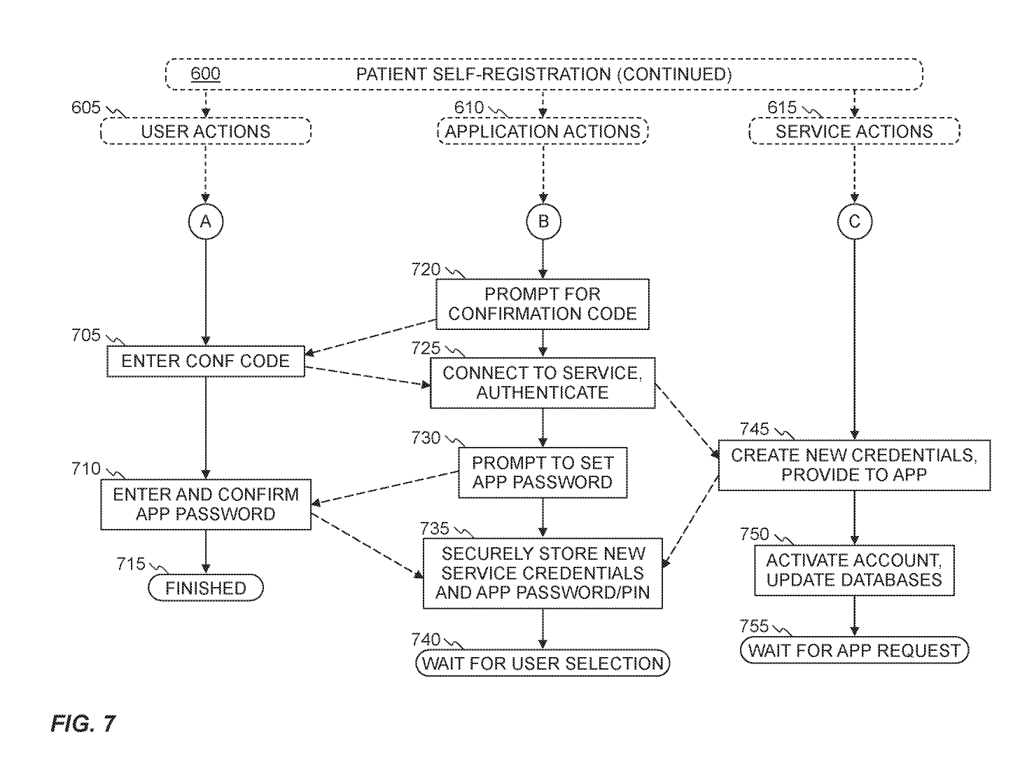

FIG. 6 and FIG. 7 provide detail of process 600, patient self-registration. Three primary actors participate in this process: a user, which in this case is the patient; an application, which in this case is patient application 140 in both its web app and device app implementations, as well as a web browser in which to run the web app; and a service, which in this case is the various elements of trial operations service suite 200, including in particular patient portal 204. In the figure, the operations performed by these actors are shown as part of interacting sub-processes: user actions 605, application actions 610, and service actions 615. Solid arrows represent temporal ordering and information flow within a sub-process, and dashed arrows represent temporal ordering and information flow between sub-processes. Note that the sub-process flows cross from FIG. 6 to FIG. 7 via the connection points shown as labeled circles.

Patient self-registration process 600 begins at operation 620, in which the user may activate a web browser installed in patient personal computing device 104 and enter a URL to access patient portal 204. The patient may have acquired this URL from any of several possible sources advertising the clinical trial of interest, including but not limited to: general news media; a website describing the trial; an invitation email, text message, or fax sent by the trial operations service at the request of another patient, a clinician, or a coordinator using their respective invitation processes 1100, 2800, or 6000. At this point, the web browser should at operation 645 access patient portal 204 to download and execute the patient application 140 web app. Patient portal 204 facilitates this download at operation 675. Upon loading, at operation 650 patient application 140 prompts the user to logon or register, so at operation 625 the user selects the register action, whereupon at operation 655 patient application 140 prompts the user to enter registration data. Registration data may include identifying information for the patient, as well as external contact information such as email address or phone number. The patient may enter values for these data in operation 630, and if so then at operation 660 patient application 140 relays them to patient portal 204 for processing and proceeds to download the patient application 140 device app installer package. At operation 680, patient portal 204 stores the registration data in operational databases 202, creating a new entry for the patient in provisioning and authorization data 222 and partially populating it. Also, in operation 680 patient portal provides the patient application 140 device app installer package for download and sets a cookie in the patient application 140 web app containing initial credentials for automatic login when the device app starts, as well as a confirmation code the patient will enter at a later operation to demonstrate access to the provided contact address. At operation 685, this notification is constructed to contain the same confirmation code and a description of its use; patient portal 204 submits it to notifications relay service 282 so that it may be sent to the patient's external contact address, facilitated by process 7400 notifications relay service autonomous operation and process 7450 external notifications gateway autonomous operation. Note that operation 685 refers to that notification as an email message, but it may also be an SMS text message, fax, voice call, or other contact medium according to the type of external contact address provided by the patient at operation 630. At operation 665, then, the patient application 140 web app stores the automatic logon credentials cookie, prompts the patient to install the patient application 140 device app, and exits. At operation 635, the patient may approve installation and activation of the patient application 140 device app, using techniques specific to the operating system of patient personal computing device 104, whereupon the patient application 140 device app will initialize at operation 670 and collect the cookie containing automatic logon credentials and confirmation code, which was left for it by the patient application 140 web app at operation 665. Once initialized, patient application 140 will at operation 720 prompt the patient to enter the confirmation code created at operation 745.

In parallel, at operation 640 the patient should receive on the provided contact address the confirmation message that was constructed and sent at operation 685. When ready, at operation 705 the patient may enter the confirmation code thus received, allowing patient application 140 to connect securely and authenticate with patient portal 204 at operation 725. The confirmation code may also be conveyed as part of this authentication for a secondary confirmation by patient portal 204. Though not shown in the Figure, if for some reason either authentication or confirmation fails, the entire process must be restarted. Since this initial authentication is performed with the original auto-logon credentials and confirmation code that were exposed, respectively, via a cookie in order to communicate between the web app and device app forms of patient application 140 and via an email or other medium in order to communicate between patient portal 204 and the patient, at operation 745 patient portal 204 creates new credentials and provides them to patient application 140 device app directly via the secure connection. Patient portal 204 also updates databases 202, specifically provisioning and authorization data 222, with the new credentials and an indication that the patient's user account is now active; these actions are embodied in operation 750. In the meantime, patient application 140 prompts the patient at operation 730 to set a password that must be entered to allow user access to the patient application 140 device app, thereby protecting the service credentials from abuse by others. The patient enters and confirms this app password at operation 710. At operation 735, upon receiving these new credentials from patient portal 204 and app password from the patient, patient application 140 device app stores them securely in protected storage using facilities provided by the operating system of patient personal computing device 104.

At this point, process 600 is complete, with each sub-process quiescing in its own way. User sub-process 605 simply finishes at operation 715, with nothing further for the patient to do. Patient application 140 remains running in patient personal computing device 104, so its quiescent state is waiting for the user's next selection at operation 740. Similarly, patient portal 204 has an established session for this user, with patient application 140 connected and authenticated, so it too quiesces in an active state waiting for the next application request at operation 755.

Note that at any point after registration or any other activity, the patient may choose to log off by deactivating the patient application 140 device app, which may mean closing it, quitting it, or performing some other specific action on it according to the norms of the operating system for patient personal computing device 104. If this happens, in order to perform any other activity in the system the patient must log back on using process 800, patient logon.

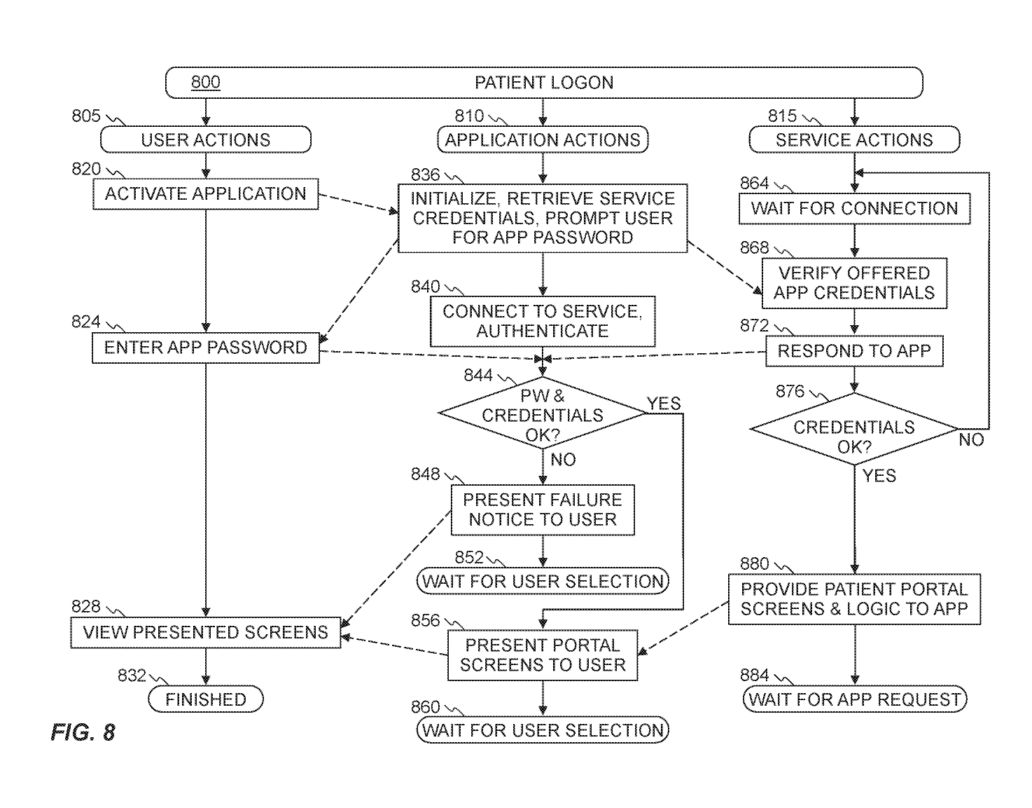

Patient logon process 800 is detailed in FIG. 8 and is a prerequisite for as well as an embedded operation in every other patient process that follows. Three primary actors participate in this process: a user, which in this case is the patient; an application, which in this case is the patient application 140 device app; and a service, which in this case is the various elements of trial operations service suite 200, including in particular patient portal 204. In the figure, the operations performed by these actors are shown as part of interacting sub-processes: user actions 805, application actions 810, and service actions 815. Solid arrows represent temporal ordering and information flow within a sub-process, and dashed arrows represent temporal ordering and information flow between sub-processes.

Patient logon process 800 begins at operation 820, in which the user may activate the patient application 140 device app previously installed in patient personal computing device 104 via patient self-registration process 600. At operation 836, the patient application 140 device app will initialize and retrieve its service credentials and app password from protected storage, prompt the user to enter the app password, then use the service credentials at operation 840 to connect and authenticate with patient portal 204. In parallel, the patient at operation 824 may enter the app password. In the meantime, patient portal 204 has been waiting for this connection, represented as operation 864; see also portals autonomous operation process 7200. Upon arrival of the connection attempt, patient portal 204 will at operation 868 verify the offered app credentials and respond accordingly at operation 872. If the credentials were not accepted, the decision at operation 876 causes server sub-process 815 to return to operation 864 and await another connection attempt. Similarly, application sub-process 810 checks at operation 844 whether the response indicated acceptance of the credentials, as well as whether the user entered the correct app password. If either is not accepted, patient application 140 presents a failure notice to the user at operation 848 and waits for a user selection at operation 852. If the credentials and password were both accepted, then patient portal 204 provides patient application 140 with screens and logic associated with patient portal services 240 at operation 880, and patient application 140 in turn presents the initial screen of that view to the user at operation 856. The patient then may view the presented screen, whether the failure notice of operation 848 or the portal view of operation 856, at operation 828.

At this point, patient logon process 800 is complete, with each sub-process quiescing in its own way. User sub-process 805 simply finishes at operation 832, with nothing further for the patient to do. In application sub-process 810, patient application 140 remains running in patient personal computing device 104, so its quiescent state is waiting for the user's next selection at operation 860. Similarly, patient portal 204 has an established session for this user, with patient application 140 connected and authenticated, so it too quiesces in an active state waiting for the next application request at operation 884. Any of the other patient methods may be selected in this state.

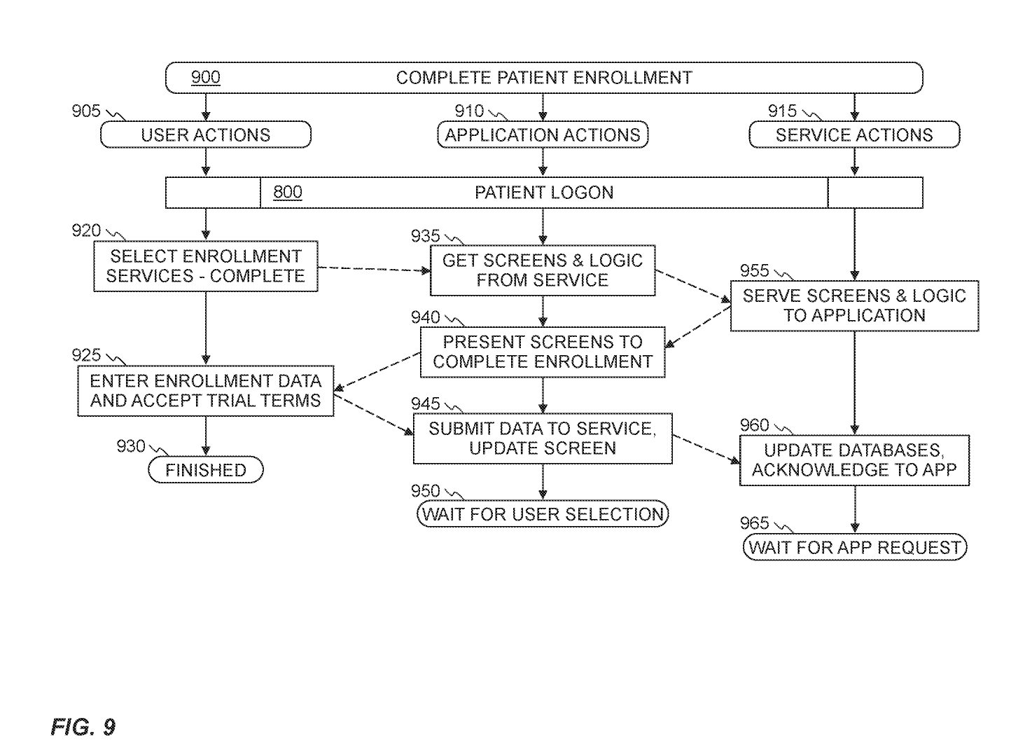

If a patient has not yet accepted the trial terms and provided all the demographic, health status, and other information required for the trial, one of the first actions to occur after patient self-registration process 600 is complete patient enrollment process 900, which is depicted in FIG. 9. Three primary actors participate in this process: a user, which in this case is the patient; an application, which in this case is the patient application 140 device app; and a service, which in this case is the various elements of trial operations service suite 200, including in particular patient portal 204 and patient portal services 240. In the figure, the operations performed by these actors are shown as part of interacting sub-processes: user actions 905, application actions 910, and service actions 915. Solid arrows represent temporal ordering and information flow within a sub-process, and dashed arrows represent temporal ordering and information flow between sub-processes.

Complete patient enrollment process 900 begins with the prerequisite of patient logon process 800, in which all three actors participate. After that, at operation 920 the patient may select enrollment services 241 from the menu of patient portal services 240. This may include explicit selection of an action to complete enrollment, although in a preferred embodiment patient application 140 may be aware from its patient portal services logic that enrollment is incomplete and must be the first action to take. Either way, at operation 935 patient application 140 may get the corresponding additional screens and logic from patient portal 204 if they are not already loaded, and patient portal 204 would provide them at operation 955. At operation 940, then, the application presents the appropriate screens for completing enrollment to the user. Depending on the trial design established by a coordinator, the patient may be presented a number of screens and options and data entry opportunities in order to qualify for participation in the trial, then review and consent to the terms of the trial and provide enrollment information required by the trial protocol such as health status, clinician relationships, and others. Operation 925 embodies the patient's activity of reading this material and entering the requested data. This may take the form of several interactions between user and app, but in the end when all the entries have been made, patient application 140 at operation 945 may submit the entire data package to patient portal 204 enrollment services 241, which in turn at operation 960 would update operational databases 202 with this new information about the patient. Some of the information may be recorded in provisioning and authorization data 222, while other information may be categorized such that it must be recorded in protected health information 223; if appropriate, some of the latter may be also de-identified and recorded in anonymized health information 224. When all the data is safely stored, still within operation 960, patient portal 204 enrollment services 241 should provide an acknowledgement to patient application 140 so it knows enrollment is complete and does not present the option to do so again.

At this point, complete patient enrollment process 900 is complete, with each sub-process quiescing in its own way. User sub-process 905 simply finishes at operation 930, with nothing further for the patient to do. In application sub-process 910, patient application 140 remains running in patient personal computing device 104, so its quiescent state is waiting for the user's next selection at operation 950. Similarly, patient portal 204 has an established session for this user, with patient application 140 connected and authenticated, so it too quiesces in an active state waiting for the next application request at operation 965. Any of the other patient methods may be selected in this state.

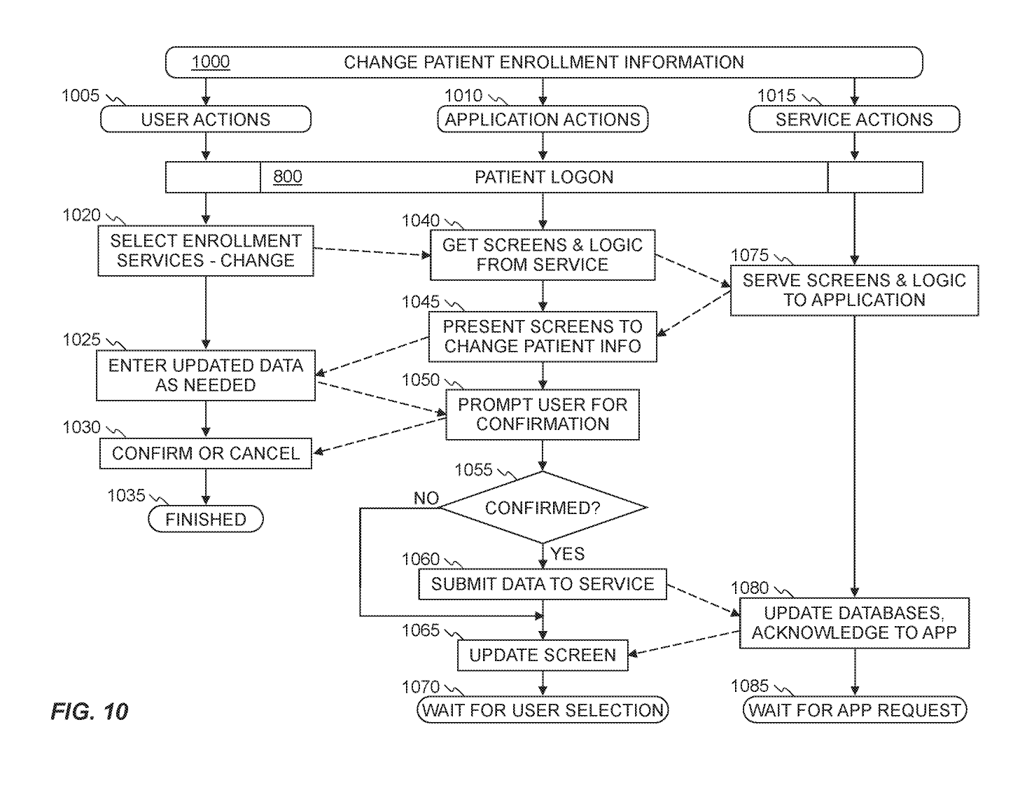

When a patient needs to change enrollment information, such as contact address or health status, or even to withdraw from the trial by deleting enrollment, the change patient enrollment information process 1000 may be used, as depicted in FIG. 10. The same three-actor model applies here, with the same temporal/informational interaction conventions. The sub-processes in this case are labeled user actions 1005, application actions 1010, and service actions 1015.

Change patient enrollment information process 1000 begins with the prerequisite of patient logon process 800, in which all three actors participate. After that, at operation 1020 the patient may select the change option under enrollment services 241 from the menu of patient portal services 240. At operation 1040 patient application 140 may get the corresponding additional screens and logic from patient portal 204 if they are not already loaded, and patient portal 204 would provide them at operation 1075. At operation 1045, then, the application presents the appropriate screens for changing enrollment information. Depending on the trial design established by a coordinator, the patient may be presented one or more screens of options and data entry opportunities in order to update the information as needed, all embodied in operation 1025. This may take the form of several interactions between user and app, but in the end when all the changes have been made, patient application 140 at operation 1050 may prompt the patient for confirmation, and the patient at operation 1030 may choose to confirm or cancel the changes. If patient application 140 determines at operation 1055 that the patient confirmed the changes, it may at operation 1060 submit the change package to patient portal 204 enrollment services 241, which in turn at operation 1080 would update operational databases 202 with this new information about the patient. Some of the information may be updated in provisioning and authorization data 222, while other information may be categorized such that it must be updated in protected health information 223; if appropriate, some of the latter may also be de-identified and recorded in anonymized health information 224. When all the data is safely stored, still within operation 1080, patient portal 204 enrollment services 241 should provide an acknowledgement to patient application 140 so it knows the change was successful. Confirmed or not, changed successfully or not, patient application 140 updates the screen to reflect the appropriate state of affairs at operation 1065.

At this point, change patient enrollment information process 1000 is complete, with each sub-process quiescing in its own way. User sub-process 1005 simply finishes at operation 1035, with nothing further for the patient to do. In application sub-process 1010, patient application 140 remains running in patient personal computing device 104, so its quiescent state is waiting for the user's next selection at operation 1070. Similarly, patient portal 204 has an established session for this user, with patient application 140 connected and authenticated, so it too quiesces in an active state waiting for the next application request at operation 1085. Any of the other patient methods may be selected in this state.

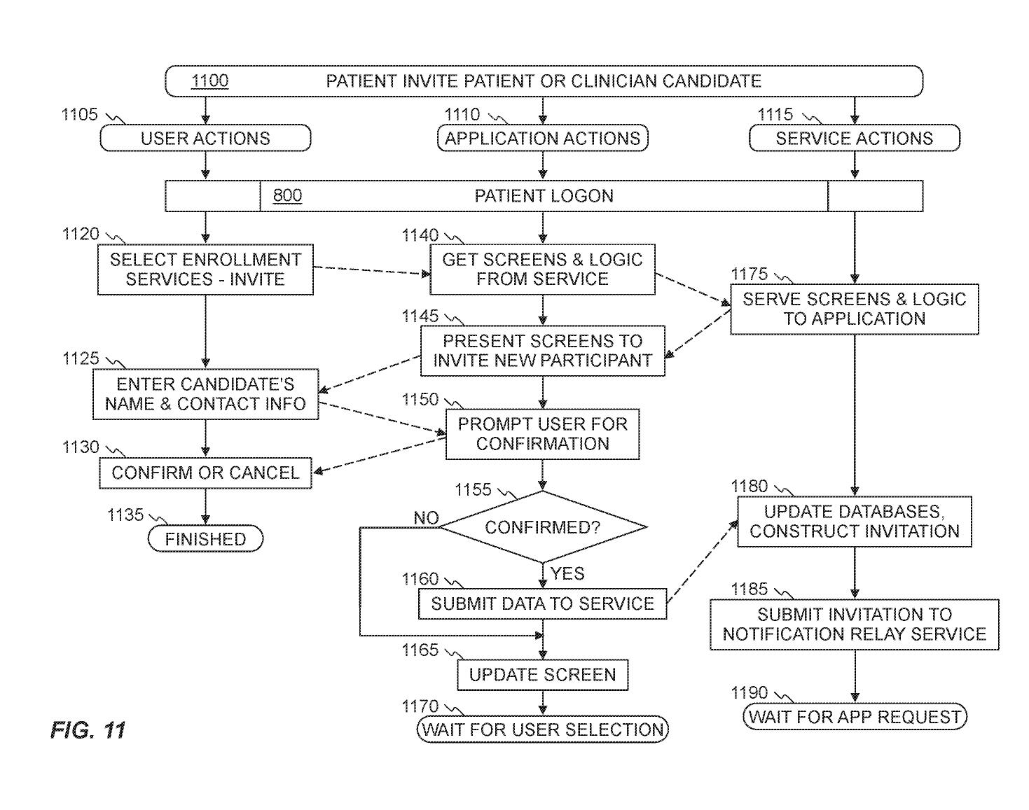

One of the ways a patient or clinician such as a doctor may learn about a particular trial and choose to self-register for it (process 600 for patients, process 2300 for clinicians) is by invitation from a current participant, based on real-life interactions. This “social networking” aspect of trial participation is facilitated by process 1100, patient invite patient or clinician candidate, which is depicted in FIG. 11. The same three-actor model applies here, with the same temporal/informational interaction conventions. The sub-processes in this case are labeled user actions 1105, application actions 1110, and service actions 1115.

Patient invite patient or clinician candidate process 1100 begins with the prerequisite of patient logon process 800, in which all three actors participate. After that, at operation 1120 the patient may select the invite option under enrollment services 241 from the menu of patient portal services 240. At operation 1140 patient application 140 may get the corresponding additional screens and logic from patient portal 204 if they are not already loaded, and patient portal 204 would provide them at operation 1175. At operation 1145, then, the application presents the appropriate screens for inviting new participants. Depending on the trial design established by a coordinator, the patient may be presented one or more screens of options and data entry opportunities in order to provide name and contact information for the patient or clinician candidate to be invited, all embodied in operation 1125. This may take the form of several interactions between user and app, but in the end when the information has been entered, patient application 140 at operation 1150 may prompt the patient for confirmation, and the patient at operation 1130 may choose to confirm or cancel the invitation. If patient application 140 determines at operation 1155 that the patient confirmed the invitation, it may at operation 1160 submit the invitation package to patient portal 204 enrollment services 241, which in turn at operation 1180 would update operational databases 202 with this new information about the invitee and construct the actual invitation message. In general, only provisioning and authorization data 222 would be created during this operation, because the invitee would have to provide the remaining enrollment data in his or her own execution of self-registration process 600/2300 and enrollment completion process 900/2600. When all the data is safely stored, patient portal 204 enrollment services 241 should submit the invitation to notification relay services 282 within trial operations service suite 200, in order to have the invitation relayed externally; refer to process 7400, notifications relay service autonomous operation, for details of how this occurs. Finally, whether the invitation was confirmed or not, patient application 140 may update the screen to reflect the appropriate state of affairs at operation 1165. Note that this process is depicted as inviting only a single candidate at a time, which is one possible embodiment. In an alternate embodiment, multiple candidates may be invited at the same time, in which case the screens presented at operation 1145 and the information entered by the user at operation 1125 may include options for more than one name and contact address, while the service actions at operations 1180 and 1185 may process multiple entries and create multiple invitations.

At this point, patient invite patient or clinician candidate process 1100 is complete, with each sub-process quiescing in its own way. User sub-process 1105 simply finishes at operation 1135, with nothing further for the patient to do. In application sub-process 1110, patient application 140 remains running in patient personal computing device 104, so its quiescent state is waiting for the user's next selection at operation 1170. Similarly, patient portal 204 has an established session for this user, with patient application 140 connected and authenticated, so it too quiesces in an active state waiting for the next application request at operation 1190. Any of the other patient methods may be selected in this state.

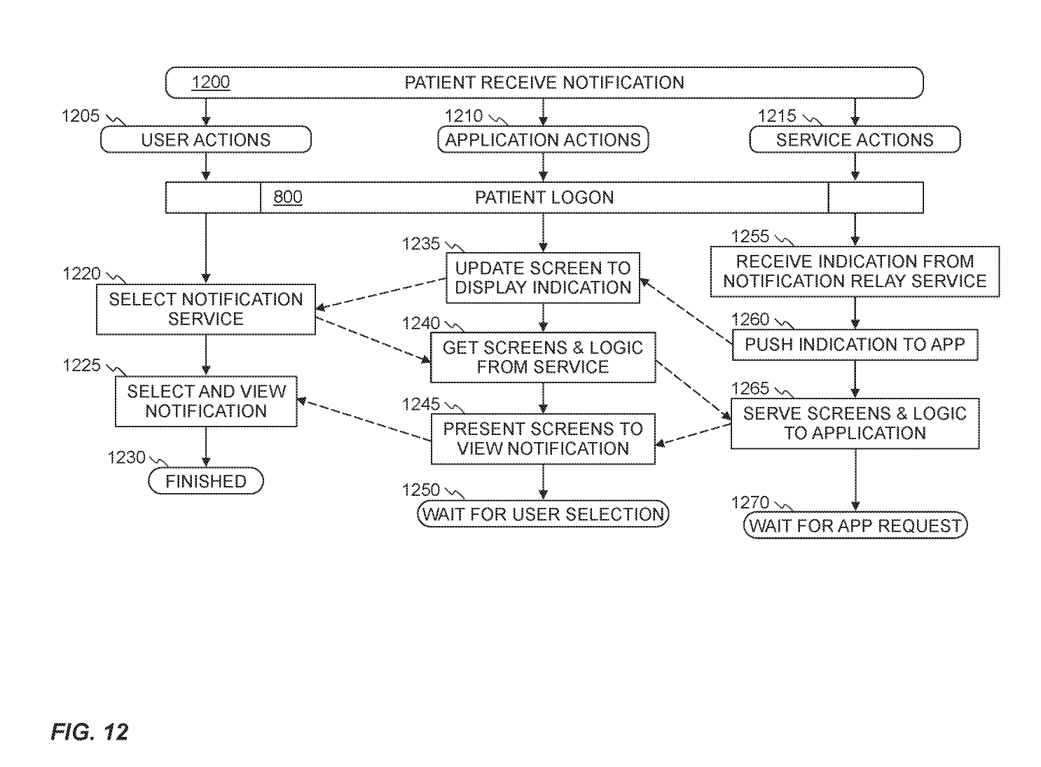

A clinician or coordinator may send patients a notification conveying, for example, news about the trial, a dose reminder, or a request for an appointment; see the corresponding send notification processes 3100 and 6800 described later. Patient receive notification process 1200, which is depicted in FIG. 12, enables the patient to view such notifications. Once again, the three-actor model and temporal/informational interaction conventions apply. The sub-processes in this case are labeled user actions 1205, application actions 1210, and service actions 1215.

Patient receive notification process 1200 begins with the prerequisite of patient logon process 800, in which all three actors participate. Note that external notification mechanisms described in processes 7400 and 7450, respectively notifications relay service autonomous operation and external notifications gateway autonomous operation, may prompt a patient who is not already logged on to do so. Once patient logon process 800 has established a portal session for the patient, whether prompted anew or already in place from previous activity, patient portal 204 may at operation 1255 receive an indication of the notification from notification relay service 282, and then at operation 1260 push that indication to patient application 140. Patient application 140 in turn may at operation 1235 update whatever screen it is displaying to include a form of the indication observable by the user.

After that, at operation 1220 the patient may select notification service 242 from the menu of patient portal services 240. This may involve explicit selection of a command to view notifications, or simply touching or clicking on the indication itself. Either way, at operation 1240 patient application 140 may get the corresponding additional screens and logic from patient portal 204 if they are not already loaded, and patient portal 204 would provide them at operation 1265. At operation 1245, then, the application presents the appropriate screens for viewing the notification to the user. Operation 1225 embodies the patient's activity of viewing the presented notification, which may also include selecting a specific notification from a list of notifications that may have been sent to the patient. Note that, although not shown in FIG. 12, if the selected notification includes a survey or health status query, or is an event reminder, patient application 140 may also in this operation switch automatically to the corresponding section of engagement services 244, as specified in patient answer query or survey process 1800 and patient view schedule of events process 1900.

At this point, patient receive notification process 1200 is complete, with each sub-process quiescing in its own way. User sub-process 1205 simply finishes at operation 1230, with nothing further for the patient to do. In application sub-process 1210, patient application 140 remains running in patient personal computing device 104, so its quiescent state is waiting for the user's next selection at operation 1250. Similarly, patient portal 204 has an established session for this user, with patient application 140 connected and authenticated, so it too quiesces in an active state waiting for the next application request at operation 1270. Any of the other patient methods may be selected in this state.

Trial operations service suite 200 may include support for communication between a patient and other trial participants, potentially including other patients, clinicians, investigators, and coordinators. Which other participants a patient is allowed to select for communication may be constrained by the trial design as established by a coordinator. For example, a patient may be permitted to communicate only with a single clinician overseeing that patient's treatment and trial participation, or a patient may be permitted to communicate with other patients in the trial. This capability, if permitted by the trial design, may enable a social networking aspect across the community of trial participants, thereby enhancing participants' engagement in the trial protocol. Four modes of communication may be supported, which are divided into two categories for the purpose of explaining their operation. Communication that is oriented toward exchanging written messages, documents, images, and other related information is categorized as messaging, and typified as chat mode or email mode. Communication that is oriented toward verbal or visual conversation over a live full-duplex connection is categorized as calling and typified as voice mode or video mode. The processes supporting these capabilities are specified for each category, and within each category for initiating and accepting communications of the corresponding category, yielding four separate processes to be described hereinafter.

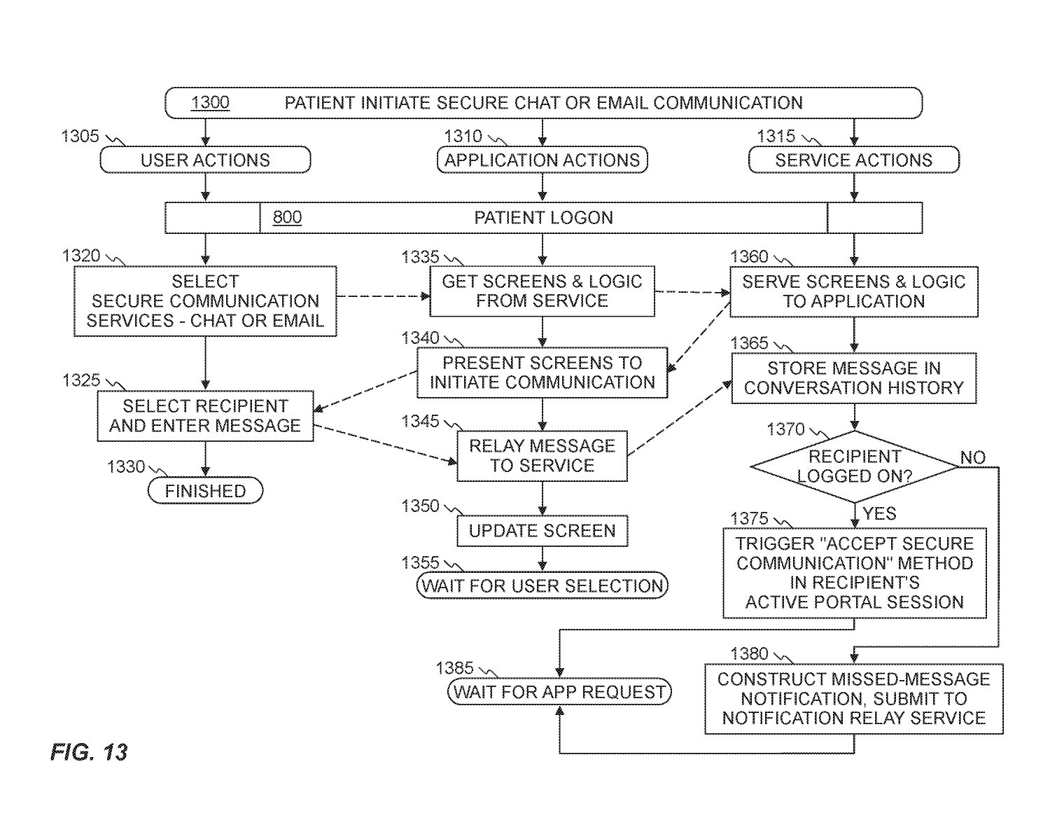

The first communication process is patient initiate secure chat or email communication process 1300, which is depicted in FIG. 13. As usual, the three-actor model and temporal/informational interaction conventions apply. The sub-processes in this case are labeled user actions 1305, application actions 1310, and service actions 1315; the primary service actor is secure communication services 243, with help from secure communication relay services 281.

Patient initiate secure chat or email communication process 1300 begins with the prerequisite of patient logon process 800, in which all three actors participate. After that, at operation 1320 the patient may select secure communication services 243 and the appropriate communication mode—in this case either chat or email—from the menu of patient portal services 240. At operation 1335 patient application 140 may get the corresponding additional screens and logic from patient portal 204 if they are not already loaded, and patient portal 204 would provide them at operation 1360. At operation 1340, then, the application presents the appropriate screens for initiating communication. Depending on the trial design established by a coordinator, the patient may select one or more recipients to which a chat or email message should be sent, with the selection mechanism and message entry embodied in operation 1325, whereupon patient application 140 should at operation 1345 relay the entered message to secure communication services 243 of patient portal 204, and at operation 1350 update the presented screen accordingly. Meanwhile, secure communication relay services 281 at operation 1365 may record the message in a conversation history associated with both the sending patient and the recipient, which may preferably be stored in the notes, records, and events 225 section of operational databases 202. Note that while only one recipient is depicted in the figure, this and other operations may be repeated for each of multiple recipients if the sender selected more than one. Then, at operation 1370 secure communication relay services 281 may determine whether the recipient is currently logged on in an active portal session. If so, at operation 1375 secure communication relay services 281 may trigger the recipient's accept secure chat or email communication process 1400, 3300, 4800, or 6300 depending on the recipient's role in the system, respectively another patient, a clinician, an investigator, or a coordinator. If the recipient does not have a current active portal session, then at operation 1380 secure communication relay services 281 may construct a missed-message notification and submit it to notifications relay service 282 so that it may be sent to the recipient's external contact address, facilitated by process 7400 notifications relay service autonomous operation and process 7450 external notifications gateway autonomous operation.

At this point, patient initiate secure chat or email communication process 1300 is complete, with each sub-process quiescing in its own way. User sub-process 1305 simply finishes at operation 1330, with nothing further for the patient to do. In application sub-process 1310, patient application 140 remains running in patient personal computing device 104, so its quiescent state is waiting for the user's next selection at operation 1355. Similarly, patient portal 204 has an established session for this user, with patient application 140 connected and authenticated, so it too quiesces in an active state waiting for the next application request at operation 1385. Any of the other patient methods may be selected in this state.

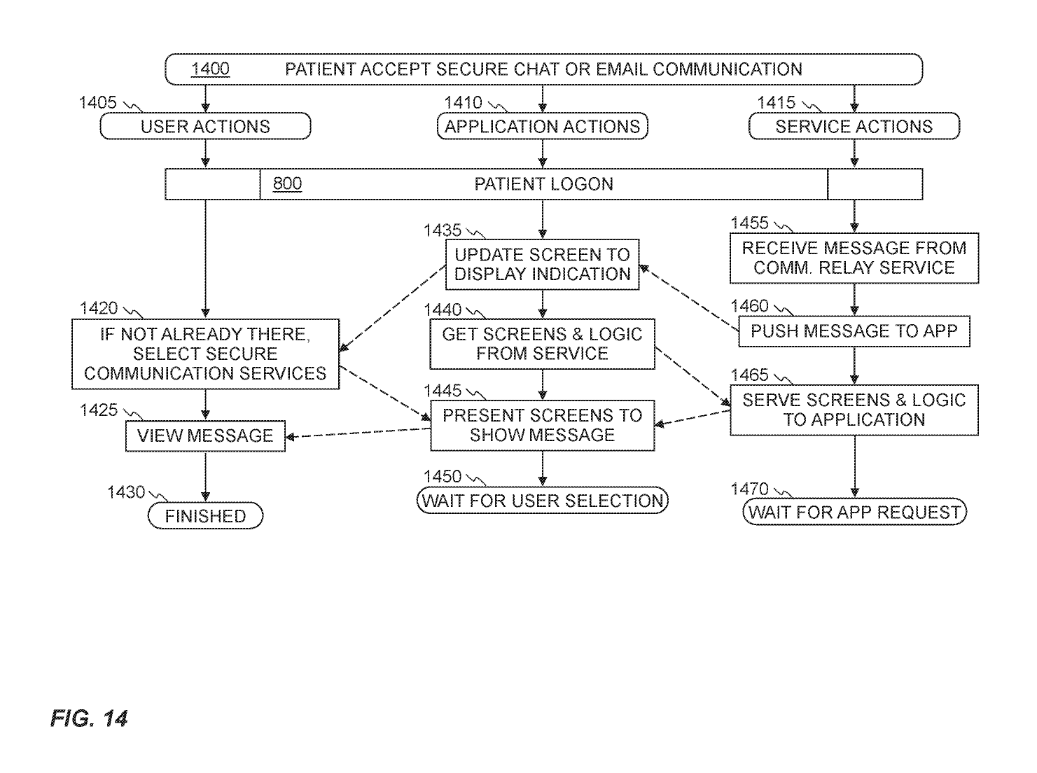

The next communication process is patient accept secure chat or email communication process 1400, which is depicted in FIG. 14. As usual, the three-actor model and temporal/informational interaction conventions apply. The sub-processes in this case are labeled user actions 1405, application actions 1410, and service actions 1415; the primary service actor is secure communication services 243, with help from secure communication relay services 281.

Patient accept secure chat or email communication process 1400 begins with the prerequisite of patient logon process 800, in which all three actors participate. Note that external notification mechanisms described in processes 7400 and 7450, respectively notifications relay service autonomous operation and external notifications gateway autonomous operation, may prompt a patient who is not already logged on to do so. Once patient logon process 800 has established a portal session for the patient, whether prompted anew or already in place from previous activity, at operation 1455 secure communication services 243 of patient portal 204 may receive a copy of the message from secure communication relay services 281. After that, at operation 1460 the patient portal 204 should push the message to patient application 140, which would in turn at operation 1435 update whatever screen it is displaying to include an indication of the message's arrival. At operation 1440 patient application 140 may predictively retrieve the corresponding additional screens and logic from patient portal 204 if they are not already loaded, and patient portal 204 would provide them at operation 1465.

When the patient notices the indication thus displayed, at operation 1420 the patient may select secure communication services 243 and the appropriate communication mode—in this case either chat or email—from the menu of patient portal services 240. This may involve explicit selection of a command to enter the corresponding service, or simply touching or clicking on the indication itself; alternatively, patient application 140 may switch to the corresponding service mode automatically, or already be in the corresponding service mode due to previous activity. However it arrived in the appropriate state, at operation 1445 patient application 140 presents the appropriate screens for viewing the received message. Operation 1425 embodies the patient's activity of viewing the presented message. If the communication mode is email, this may also include selecting a specific message from a list of messages that may have been sent to the patient. If the communication mode is chat, this may also include viewing other messages both sent and received in the context of a conversation.

At this point, patient accept secure chat or email communication process 1400 is complete, with each sub-process quiescing in its own way. User sub-process 1405 simply finishes at operation 1430, with nothing further for the patient to do. In application sub-process 1410, patient application 140 remains running in patient personal computing device 104, so its quiescent state is waiting for the user's next selection at operation 1450. Similarly, patient portal 204 has an established session for this user, with patient application 140 connected and authenticated, so it too quiesces in an active state waiting for the next application request at operation 1470. Any of the other patient methods may be selected in this state.

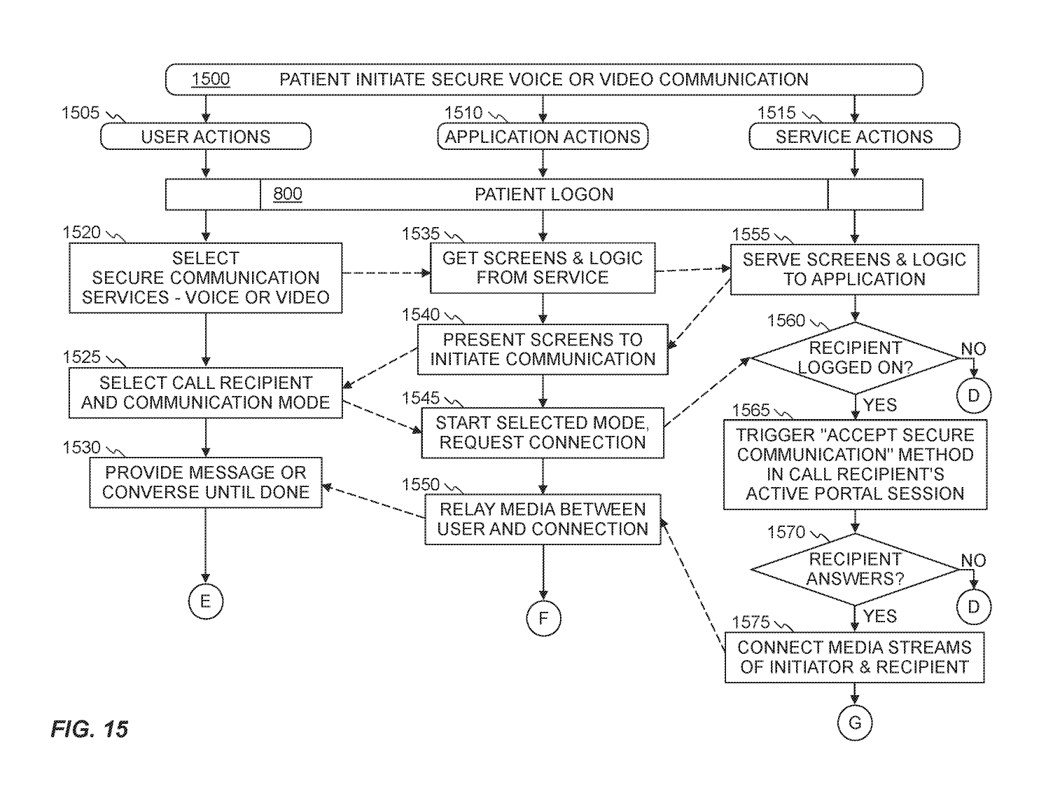

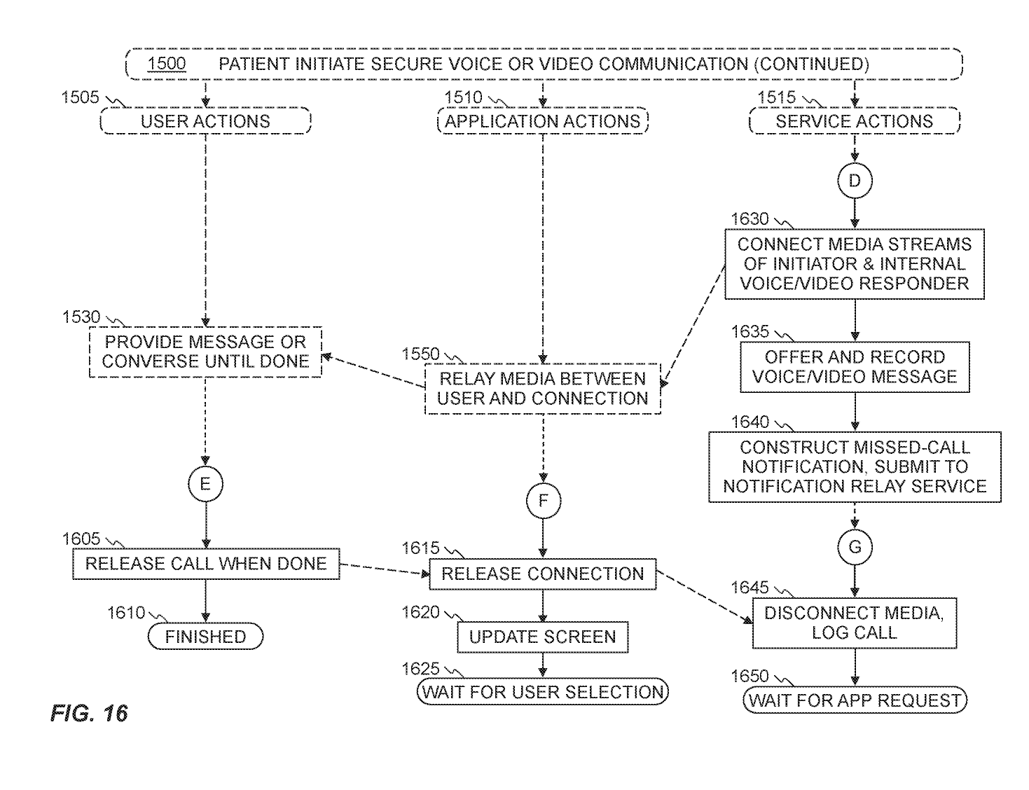

The next communication process is patient initiate secure voice or video communication process 1500, which is depicted in FIG. 15 and FIG. 16. As usual, the three-actor model and temporal/informational interaction conventions apply. The sub-processes in this case are labeled user actions 1505, application actions 1510, and service actions 1515; the primary service actor is secure communication services 243, with help from secure communication relay services 281. Note that the sub-process flows cross from FIG. 15 to FIG. 16 via the connection points shown as labeled circles.

Patient initiate secure voice or video communication process 1500 begins with the prerequisite of patient logon process 800, in which all three actors participate. After that, at operation 1520 the patient may select secure communication services 243 and the appropriate communication mode—in this case either voice or video—from the menu of patient portal services 240. At operation 1535 patient application 140 may get the corresponding additional screens and logic from patient portal 204 if they are not already loaded, and patient portal 204 would provide them at operation 1555. At operation 1540, then, the application presents the appropriate screens for initiating communication. Depending on the trial design established by a coordinator, the patient may select another trial participant with which a voice or video call should be placed, with the selection mechanism and message entry embodied in operation 1525, whereupon patient application 140 should at operation 1545 start the selected communication mode and request secure communication services 243 to have secure communication relay services 281 make a connection supporting the call. At operation 1560 secure communication relay services 281 may determine whether the called participant, referred to as recipient in the Figure, is currently logged on in an active portal session. If so, at operation 1565 secure communication relay services 281 may trigger the recipient's accept secure voice or video communication process 1700, 3600, 5100, or 6600 depending on the recipient's role in the system, respectively another patient, a clinician, an investigator, or a coordinator. During that process, the recipient may choose to accept (answer), reject, or ignore the call. At operation 1570, secure communication relay services 281 determines this outcome. If the recipient answers, at operation 1575 secure communication services 243 and secure communication relay services 281 connect the media streams of the initiator and the recipient so that they may converse.

Whether the call is answered by the recipient or the message recorder, at operation 1650 clinician application 150 relays the media streams between the calling user and the connection, so that the clinician may at operation 1630 either converse with the called participant or leave a voice or video message, as appropriate. Note that these two operations appear on both FIG. 15 and FIG. 16 and represent the same actions in both. When the clinician is finished conversing with or providing a voice/video message for the recipient, he or she will release the call at operation 1610. This may take the form of clicking or touching a hang-up control on the screen of, or pressing a button on, clinician workstation 105.

If the recipient does not have a current active portal session (operation 1560), or if the recipient does not answer the call (operation 1570), then at operation 1630 secure communication services 243 may connect the initiator's media streams to those of an internal voice or video responder—effectively an answering machine—provided by secure communication relay services 281, which at operation 1635 may offer prompts to record a voice or video message and then do so if one is provided by the initiator. After the voice or video message is finished, which may be determined by detecting a configured period of silence, or by detecting an explicit indication from the calling user such as a key word, tone, or disconnect, or if the caller chooses not to leave a message at all, at operation 1640 the secure communication relay services 281 may construct a missed-call notification and submit it to notifications relay service 282 so that it may be sent to the recipient's external contact address, facilitated by process 7400 notifications relay service autonomous operation and process 7450 external notifications gateway autonomous operation.

Whether the call is answered by the recipient or the message recorder, at operation 1550 patient application 140 relays the media streams between the calling user and the connection, so that the patient may at operation 1530 either converse with the called participant or leave a voice or video message, as appropriate. Note that these two operations appear on both FIG. 15 and FIG. 16 and represent the same actions in both. When the patient is finished conversing with or providing a voice/video message for the recipient, he or she will release the call at operation 1605. This may take the form of clicking or touching a hang-up control on the screen of, or pressing a button on, patient personal computing device 104. Alternatively, patient application 140, secure communication services 243, or secure communication relay services 281 may detect a predetermined spoken command or visible gesture from the patient as indicating the call is over, or detect that a disconnect indication of some sort was detected on the other side of the call. However call termination may be detected, patient application 140 will at operation 1615 release the connection and at operation 1620 update the screen to reflect this change of state. At operation 1645 secure communication services 243 will disconnect the media streams between the initiator's and recipient's portal sessions and record the event in a conversation history associated with both the calling patient and the recipient, which may preferably be stored in the notes, records, and events 225 section of operational databases 202.

At this point, patient initiate secure voice or video communication process 1500 is complete, with each sub-process quiescing in its own way. User sub-process 1505 simply finishes at operation 1610, with nothing further for the patient to do. In application sub-process 1510, patient application 140 remains running in patient personal computing device 104, so its quiescent state is waiting for the user's next selection at operation 1625. Similarly, patient portal 204 has an established session for this user, with patient application 140 connected and authenticated, so it too quiesces in an active state waiting for the next application request at operation 1650. Any of the other patient methods may be selected in this state.

Note that patient initiate secure voice or video communication process 1500 may also be used to listen to a voice message or view a video message left by another participant when a call from that other participant was missed by the patient. The details of this usage are not depicted, but they are effectively identical if the “answering machine” function of secure communication relay services 281 is considered the call recipient and the patient's interactions with that function are considered the conversation.

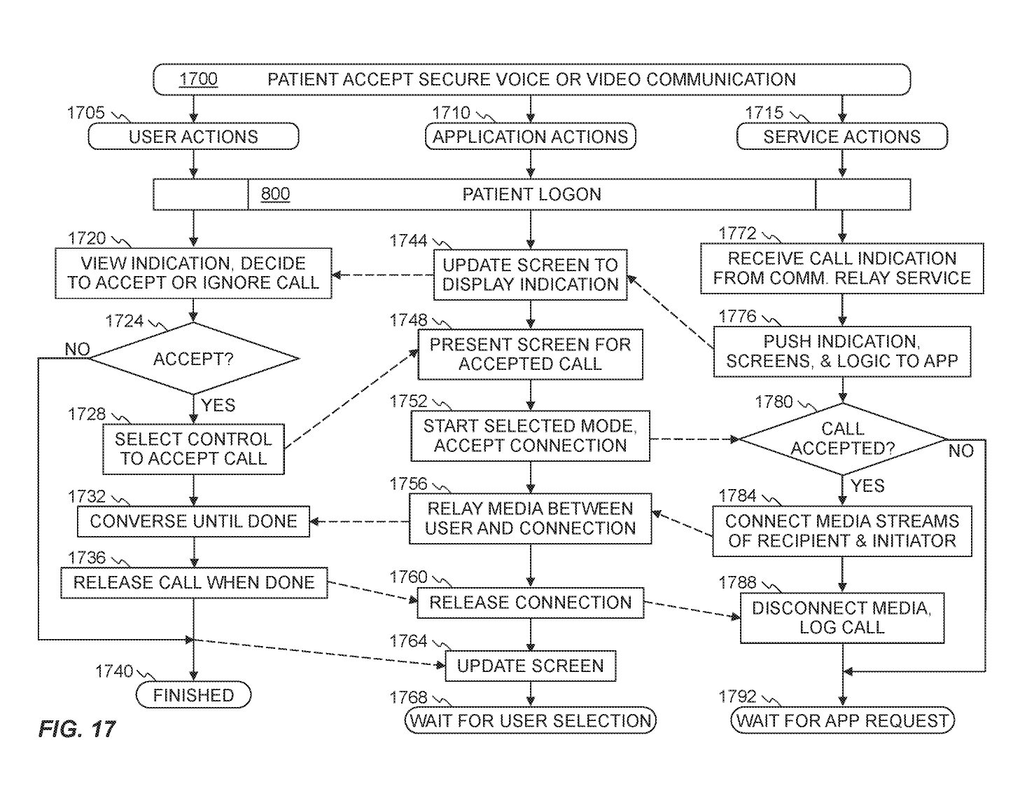

The fourth communication process is patient accept secure voice or video communication process 1700, which is depicted in FIG. 17. Again, the three-actor model and temporal/informational interaction conventions apply. The sub-processes in this case are labeled user actions 1705, application actions 1710, and service actions 1715; the primary service actor is secure communication services 243, with help from secure communication relay services 281.

Patient accept secure voice or video communication process 1700 begins with the prerequisite of patient logon process 800, in which all three actors participate. Note that for a voice or video call, this will generally have already occurred such that the portal session is currently active; otherwise the called patient would not have an opportunity to accept the call while it is being offered, and it would have turned into a missed call. Thus in this scenario, right away at operation 1772 secure communication services 243 of patient portal 204 should receive an indication of the offered call from secure communication relay services 281, and at operation 1776 patient portal 204 should push that indication, as well as screens and logic for accepting a voice or video call, to patient application 140. Patient application 140 would in turn at operation 1744 update whatever screen it is displaying to include an indication of the incoming call.

When the patient notices, at operation 1720, the indication thus displayed, he or she may choose at operation 1724 to accept or ignore the call. If the decision is to accept, the patient at operation 1728 may select the necessary control to do so. This may involve explicit selection of a menu command to enter secure communication services 243 and the appropriate communication mode—in this case either voice or video. Preferably, though, it would involve simply touching or clicking on the indication itself, causing patient application 140 to switch to the corresponding service mode automatically. However it arrived in the appropriate state, at operation 1748 patient application 140 presents the appropriate screen for having accepted the call, and at operation 1752 it starts the selected mode and completes the connection. At operation 1780, secure communication services 243 and secure communication relay services 281 also may determine whether the patient accepted the call, and if so proceed at operation 1784 to connect the media streams of the recipient and initiator, while patient application 140 at operation 1756 relays the media streams between the patient and the connection, so that the patient may at operation 1732 converse with the caller. When the patient is finished conversing with the caller, he or she will release the call at operation 1736. This may take the form of clicking or touching a hang-up control on the screen of, or pressing a button on, patient personal computing device 104. Alternatively, patient application 140, secure communication services 243, or secure communication relay services 281 may detect a predetermined spoken command or visible gesture from the patient as indicating the call is over, or detect that a disconnect indication of some sort was detected on the other side of the call. However call termination may be detected, patient application 140 will at operation 1760 release the connection and at operation 1764 update the screen to reflect this change of state. At operation 1788 secure communication services 243 will disconnect the media streams between the initiator's and recipient's portal sessions, and record the event in a conversation history associated with both the calling patient and the recipient, which may preferably be stored in the notes, records, and events 225 section of operational databases 202.

If the patient ignores or rejects the calls, this is detected at operations 1724 and 1780, and the process skips to the end, bypassing all the other operations except 1764 in which patient application 140 updates the screen to reflect the corresponding state change.

Whether answered and disconnected, or ignored, at this point patient accept secure voice or video communication process 1700 is complete, with each sub-process quiescing in its own way. User sub-process 1705 simply finishes at operation 1740, with nothing further for the patient to do. In application sub-process 1710, patient application 140 remains running in patient personal computing device 104, so its quiescent state is waiting for the user's next selection at operation 1768. Similarly, patient portal 204 has an established session for this user, with patient application 140 connected and authenticated, so it too quiesces in an active state waiting for the next application request at operation 1792. Any of the other patient methods may be selected in this state.

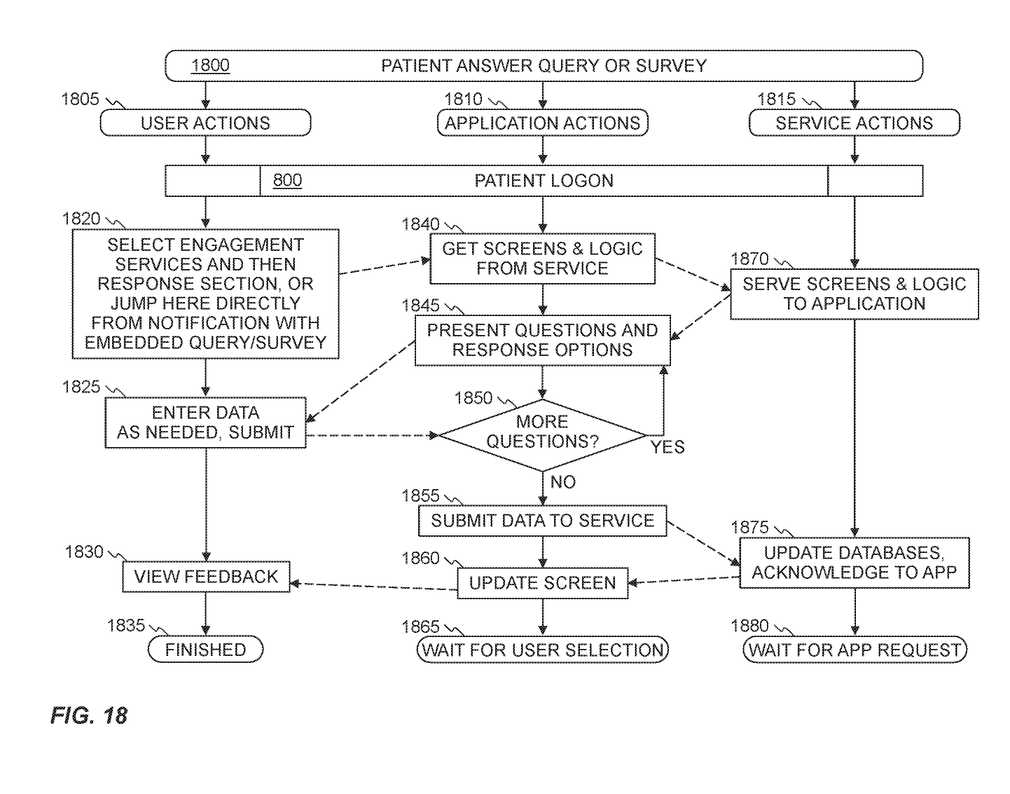

A patient participating in a trial may be prompted periodically or upon certain occurrences to answer surveys and direct questions about trial experiences, health outcomes, and other matters according to the trial protocol. Patient answer query or survey, process 1800 depicted in FIG. 18, supports this activity. Again, the three-actor model and temporal/informational interaction conventions apply. The sub-processes in this case are labeled user actions 1805, application actions 1810, and service actions 1815; the primary service actor is engagement services 244.

Patient answer query or survey process 1800 begins with the usual prerequisite of patient logon process 800, in which all three actors participate. Note that external notification mechanisms described in processes 7400 and 7450, respectively notifications relay service autonomous operation and external notifications gateway autonomous operation, may prompt a patient who is not already logged on to do so. After that, at operation 1820 the patient may select engagement services 244 and the appropriate service—query or survey—from the menu of patient portal services 240. This may include explicit selection of an action to open the diary or journal, or it may occur automatically upon opening a notification that includes a specific query or a survey request. Either way, at operation 1840 patient application 140 may get the corresponding additional screens and logic from patient portal 204 if they are not already loaded, and patient portal 204 would provide them at operation 1870. At operation 1845, then, the application presents the appropriate screens containing the question(s) and response option(s) for the query or survey. Depending on the trial design established by a coordinator, the patient may be presented a number of screens and options and data entry opportunities in order to enter the required observation. Operation 1825 embodies the patient's activity in this regard, which may take the form of several interactions between user and app. After the patient submits each entry in operation 1825, patient application 140 may determine at operation 1850 whether there are more questions in the query or survey, and if so return to operation 1845. When there are no more questions, patient application 140 at operation 1855 may submit the data package to patient portal 204 enrollment services 241, which in turn at operation 1875 would update operational databases 202 with this new information about the patient. Some of the information may be recorded in notes, records, and events not classified as health information 225, while other information may be categorized such that it must be recorded in protected health information 223; if appropriate, some of the latter may be also de-identified and recorded in anonymized health information 224. When all the data is safely stored, still within operation 1875, patient portal 204 enrollment services 241 should provide an acknowledgement to patient application 140 so it may update the screen accordingly at operation 1860. The acknowledgement may include gratitude or encouragement feedback, which may be designed to make the patient feel appreciated and eager to continue participating upon viewing it at operation 1830.

At this point, patient answer query or survey process 1800 is complete, with each sub-process quiescing in its own way. User sub-process 1805 simply finishes at operation 1835, with nothing further for the patient to do. In application sub-process 1810, patient application 140 remains running in patient personal computing device 104, so its quiescent state is waiting for the user's next selection at operation 1865. Similarly, patient portal 204 has an established session for this user, with patient application 140 connected and authenticated, so it too quiesces in an active state waiting for the next application request at operation 1880. Any of the other patient methods may be selected in this state.

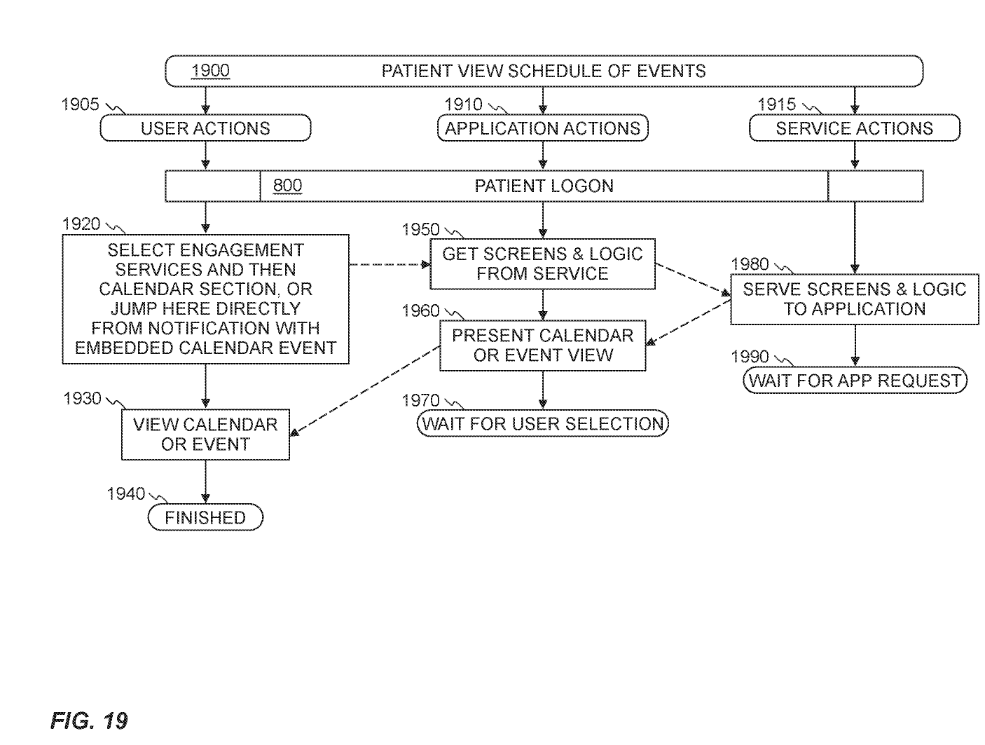

A patient participating in a trial may have a number of clinician appointments, scheduled doses, and other calendar events to attend according to the trial protocol. Patient view schedule of events, process 1900 depicted in FIG. 19, allows the patient to keep up with this schedule. Again, the three-actor model and temporal/informational interaction conventions apply. The sub-processes in this case are labeled user actions 1905, application actions 1910, and service actions 1915; the primary service actor is engagement services 244.

Patient view schedule of events process 1900 begins with the usual prerequisite of patient logon process 800, in which all three actors participate. Note that external notification mechanisms described in processes 7400 and 7450, respectively notifications relay service autonomous operation and external notifications gateway autonomous operation, may prompt a patient who is not already logged on to do so. After that, at operation 1920 the patient may select engagement services 244 and the calendar service from the menu of patient portal services 240. This may include explicit selection of an action to open the calendar, or it may occur automatically upon opening a notification that includes a newly scheduled event or a reminder for a previously scheduled event. Either way, at operation 1950 patient application 140 may get the corresponding additional screens and logic from patient portal 204 if they are not already loaded, and patient portal 204 would provide them at operation 1980. At operation 1960, then, the application presents the appropriate screens for showing the calendar or specific event. At operation 1930, the patient may in turn view this information.

At this point, patient view schedule of events process 1900 is complete, with each sub-process quiescing in its own way. User sub-process 1905 simply finishes at operation 1940, with nothing further for the patient to do. In application sub-process 1910, patient application 140 remains running in patient personal computing device 104, so its quiescent state is waiting for the user's next selection at operation 1970. Similarly, patient portal 204 has an established session for this user, with patient application 140 connected and authenticated, so it too quiesces in an active state waiting for the next application request at operation 1990. Any of the other patient methods may be selected in this state.

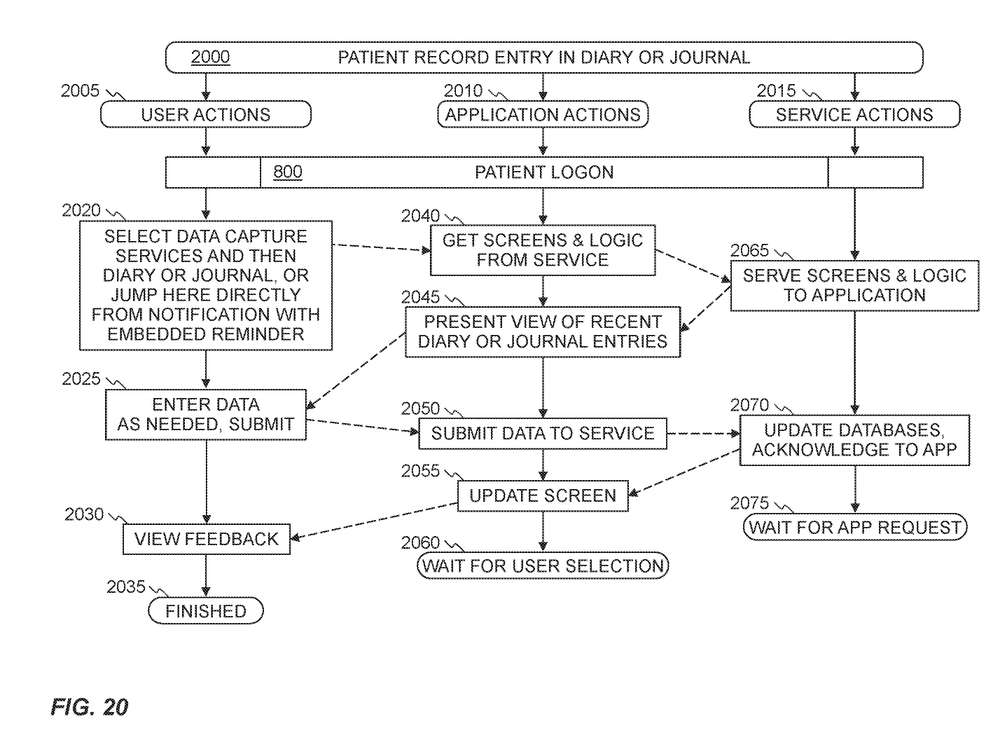

A patient participating in a trial may be expected periodically or upon certain occurrences to record self-observations regarding symptoms, adverse outcomes, drug dosing, and other matters according to the trial protocol. If the trial design requires specific fields with limited values, such as menu selections or checkboxes, the observation may be considered a diary entry. If the trial design requires free-form text or other unconstrained information, the observation may be considered a journal entry. Patient record entry in diary or journal, process 2000 depicted in FIG. 20, supports this activity. Again, the three-actor model and temporal/informational interaction conventions apply. The sub-processes in this case are labeled user actions 2005, application actions 2010, and service actions 2015; the primary service actor is data capture services 245.

Patient record entry in diary or journal process 2000 begins with the usual prerequisite of patient logon process 800, in which all three actors participate. Note that external notification mechanisms described in processes 7400 and 7450, respectively notifications relay service autonomous operation and external notifications gateway autonomous operation, may prompt a patient who is not already logged on to do so. After that, at operation 2020 the patient may select data capture services 245 and the appropriate service—diary or journal—from the menu of patient portal services 240. This may include explicit selection of an action to open the diary or journal, or it may occur automatically upon opening a notification that includes a reminder to make a particular observation or perform an action that should be recorded via an entry. Either way, at operation 2040 patient application 140 may get the corresponding additional screens and logic from patient portal 204 if they are not already loaded, and patient portal 204 would provide them at operation 2065. At operation 2045, then, the application presents the appropriate screens for making the required entry, which may include information from other recent entries to help the patient complete the new entry. Depending on the trial design established by a coordinator, the patient may be presented a number of screens and options and data entry opportunities in order to enter the required observation. Operation 2025 embodies the patient's activity in this regard, which may take the form of several interactions between user and app. In the end when the entry has been completed and submitted, patient application 140 at operation 2050 may submit the data package to patient portal 204 data capture services 245, which in turn at operation 2070 would update operational databases 202 with this new information about the patient. Some of the information may be recorded in notes, records, and events not classified as health information 225, while other information may be categorized such that it must be recorded in protected health information 223; if appropriate, some of the latter may be also de-identified and recorded in anonymized health information 224. When all the data is safely stored, still within operation 2070, patient portal 204 data capture services 245 should provide an acknowledgement to patient application 140 so it may update the screen accordingly at operation 2055. The acknowledgement may include feedback expressing gratitude or encouragement, which may be designed to make the patient feel appreciated and eager to continue participating upon viewing it at operation 2030.

At this point, patient record entry in diary or journal process 2000 is complete, with each sub-process quiescing in its own way. User sub-process 2005 simply finishes at operation 2035, with nothing further for the patient to do. In application sub-process 2010, patient application 140 remains running in patient personal computing device 104, so its quiescent state is waiting for the user's next selection at operation 2060. Similarly, patient portal 204 has an established session for this user, with patient application 140 connected and authenticated, so it too quiesces in an active state waiting for the next application request at operation 2075. Any of the other patient methods may be selected in this state.

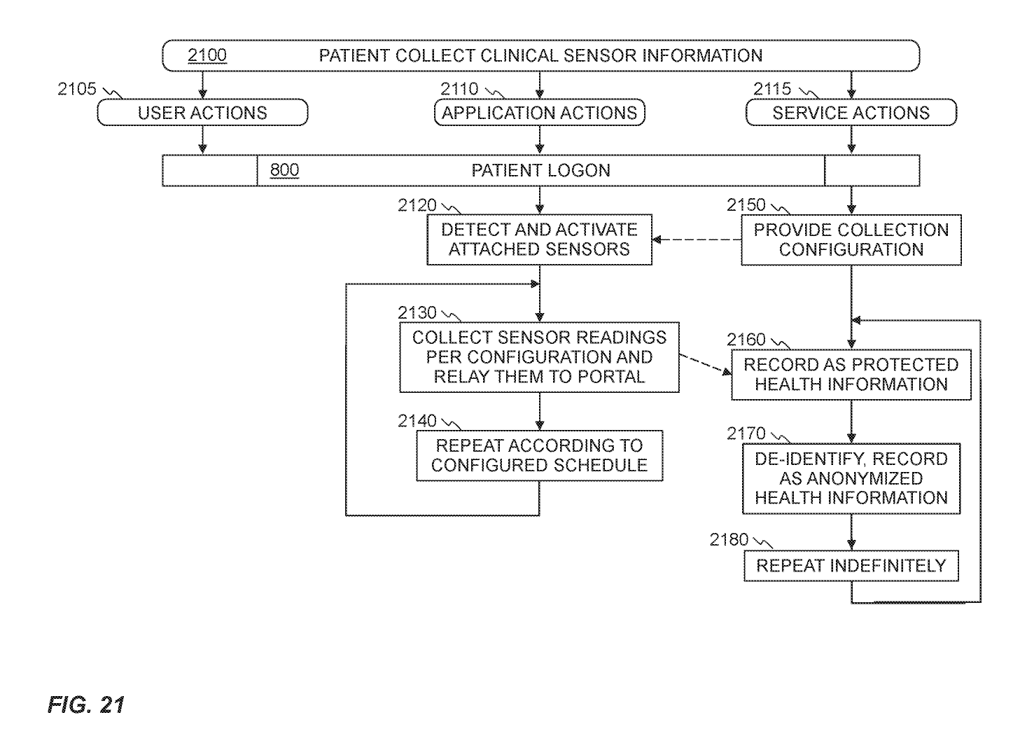

A patient participating in a trial may carry or wear one or more patient sensor(s) 145, which may be attached to or embedded in patient personal computing device 104. Depending on the trial design as established by a coordinator, routine collection of data from patient sensor(s) 145 may be enabled. Patient collect clinical sensor information, process 2100 depicted in FIG. 21, supports this activity. Again, the three-actor model and temporal/informational interaction conventions apply. The sub-processes in this case are labeled user actions 2105, application actions 2110, and service actions 2115; the primary service actor is data capture services 245.

Patient collect clinical sensor information process 2100 begins with the usual prerequisite of patient logon process 800, in which all three actors participate. This may have occurred due to any of the user-driven actions previously described, but depending on trial design it may also occur automatically due to patient application 140 being configured so that it runs at all times when patient personal computing device 104 is on. Either way, no further user interaction is required to enable or execute this process.

Immediately after patient logon process 800 has succeeded, patient portal 204 and data capture services 245 may at operation 2150 provide patient application 140 a configuration for collecting sensor information. This configuration may include a list of sensors to read, along with a periodicity to apply for each one so that readings are collected regularly. Note that such sensor configuration may be provided after every patient logon, not just the one included in this figure; this operation is not shown in the other processes described previously, in order to simplify their depiction since it is not pertinent to those processes. Patient application 140 at operation 2120 uses the provided configuration to detect and activate the configured sensors, then commence a regular cycle of reading and reporting.

Thus at operation 2130, patient application 140 collects sensor readings according to the provided configuration, and provides them to patient portal 204 and its data capture services 245. At operation 2140, patient application 140 sets a timer in order to repeat the cycle according to the configured schedule. In parallel, data capture services 245 at operation 2160 records each received sensor reading in operational databases 202, which stores it in protected health information 223. At operation 2170, operational databases 202 may also de-identify the reading and store it in anonymized health information 224. At operation 2180, data capture services 245 may repeat this process indefinitely within the portal session, terminating only when the portal session ends (not shown).

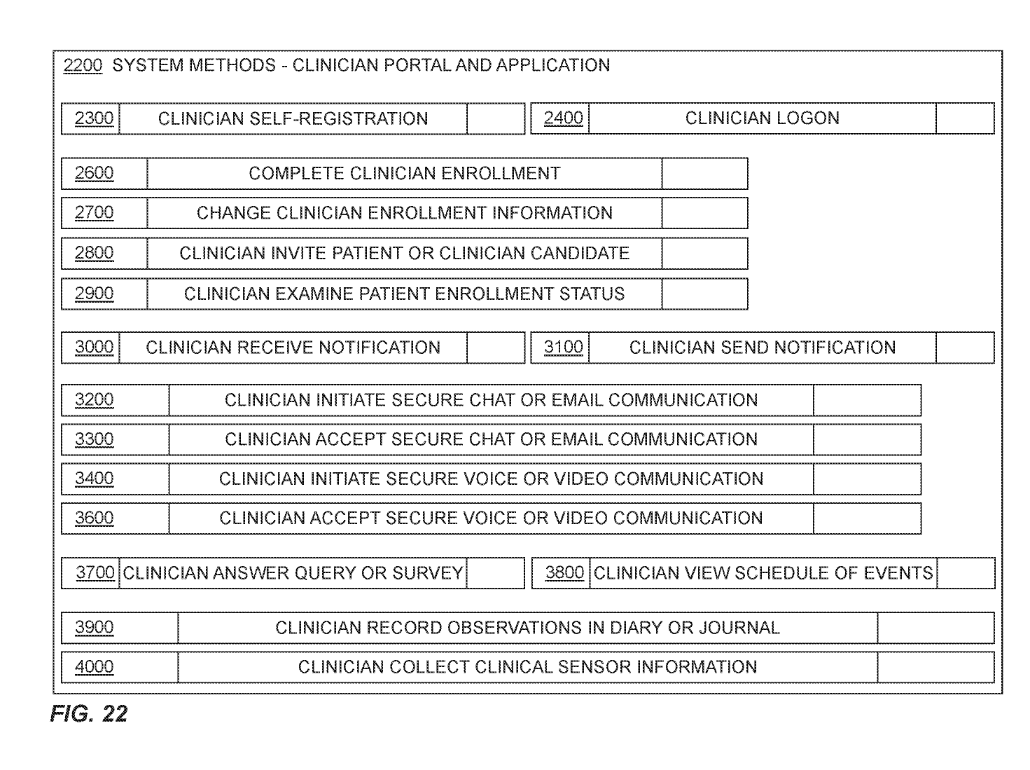

FIG. 22 illustrates a group of processes distributed among a clinician application, clinician portal, and other elements of a trial operations service suite in a clinical trial operations system according to an exemplary embodiment of the invention. The processes may include self-registration, logon, enrollment completion, personal data update, participant invitations, patient enrollment status monitoring, notification sending, notification receipt, secure communication, survey completion, calendar utilization, diary entries, and sensor data collection. FIG. 23-FIG. 40 illustrate additional detail for each of the processes in FIG. 22.

FIG. 23 provides detail of process 2300, clinician self-registration. Three primary actors participate in this process: a user, which in this case is the clinician; an application, which in this case is clinician application 150, a web app and a web browser in which to run it; and a service, which in this case is the various elements of trial operations service suite 200, including in particular clinician portal 205. In the figure, the operations performed by these actors are shown as part of interacting sub-processes: user actions 2305, application actions 2310, and service actions 2315. Solid arrows represent temporal ordering and information flow within a sub-process, and dashed arrows represent temporal ordering and information flow between sub-processes.

Clinician self-registration process 2300 begins at operation 2320, in which the user may activate a web browser installed in clinician workstation 105 and enter a URL to access clinician portal 205. The clinician may have acquired this URL from any of several possible sources advertising the clinical trial of interest, including but not limited to: general news media; a website describing the trial; an invitation email, text message, or fax sent by the trial operations service at the request of a patient, another clinician, or a coordinator using their respective invitation processes 1100, 2800, or 6000. At this point, the web browser should at operation 2344 access clinician portal 205 to download and execute the clinician application 150 web app. Clinician portal 205 facilitates this download at operation 2368. Upon loading, at operation 2348 clinician application 150 prompts the user to logon or register, so at operation 2324 the user selects the register action, whereupon at operation 2352 clinician application 150 prompts the user to enter registration data. Registration data may include identifying information for the clinician, as well as external contact information such as email address or phone number. Registration data may also include initial values for logon credentials such as a user identifier and password. The clinician may enter values for these data in operation 2328, and if so then at operation 2356 clinician application 150 relays them to clinician portal 205 for processing and prompts the clinician to enter the confirmation code to be created at operation 2376. At operation 2372, clinician portal 205 stores the registration data in operational databases 202, creating a new entry for the clinician in provisioning and authorization data 222 and partially populating it. At operation 2376, a notification is constructed to contain a confirmation code the clinician will enter to demonstrate access to the provided contact address; clinician portal 205 submits it to notifications relay service 282 so that it may be sent to the clinician's external contact address, facilitated by process 7400 notifications relay service autonomous operation and process 7450 external notifications gateway autonomous operation. Note that operation 2376 refers to that notification as an email message, but it may also be an SMS text message, fax, voice call, or other contact medium according to the type of external contact address provided by the clinician at operation 2328.

At operation 2336 the clinician should receive on the provided contact address the confirmation message that was constructed and sent at operation 2376. When ready, at operation 2336 the clinician may enter the confirmation code thus received, allowing clinician application 150 to authenticate the confirmation code with clinician portal 205 at operation 2360. Though not shown in the Figure, if for some reason authentication of the confirmation code fails, the entire process must be restarted. Assuming success, at operation 2380 clinician portal 205 updates databases 202, specifically provisioning and authorization data 222, to indicate that the clinician's user account is now active.

At this point, process 2300 is complete, with each sub-process quiescing in its own way. User sub-process 2305 simply finishes at operation 2340, with nothing further for the clinician to do. Clinician application 150 remains running in clinician workstation 105, so its quiescent state is waiting for the user's next selection at operation 2364. Similarly, clinician portal 205 has an established session for this user, with clinician application 150 connected and authenticated, so it too quiesces in an active state waiting for the next application request at operation 2384.

Note that at any point after registration or any other activity, the clinician may choose to log off by deactivating clinician application 150, which may mean closing it, quitting it, or performing some other specific action on it according to the norms of the operating system and/or web browser for clinician workstation 105. If this happens, in order to perform any other activity in the system the clinician must log back on using process 2400, clinician logon.

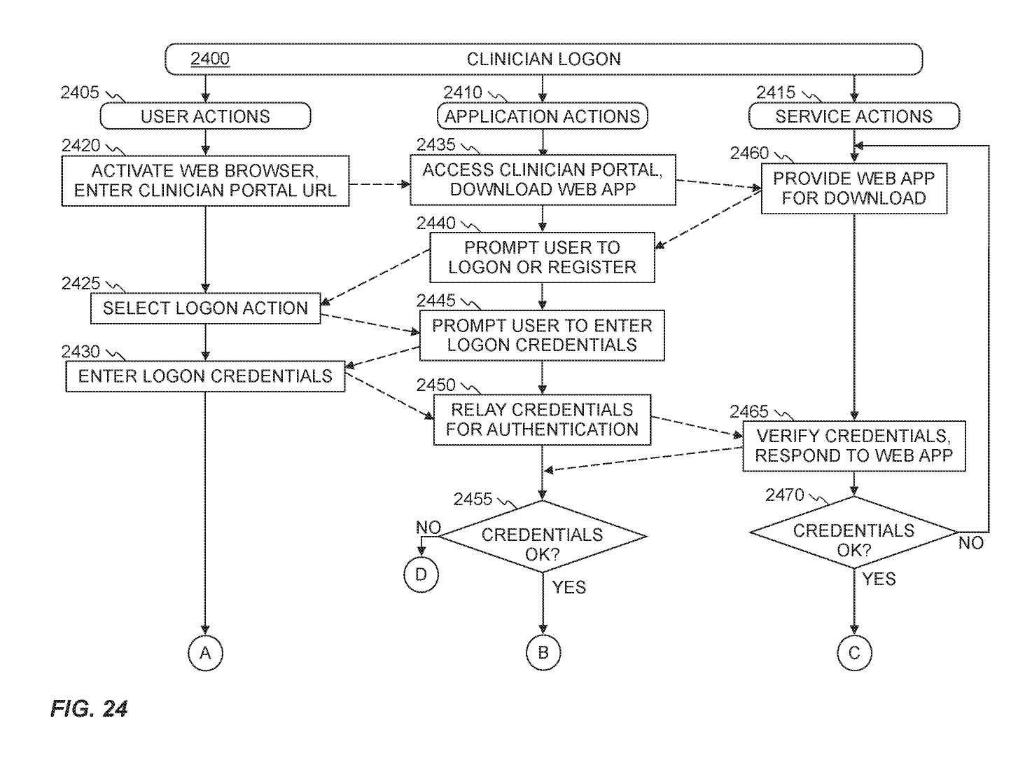

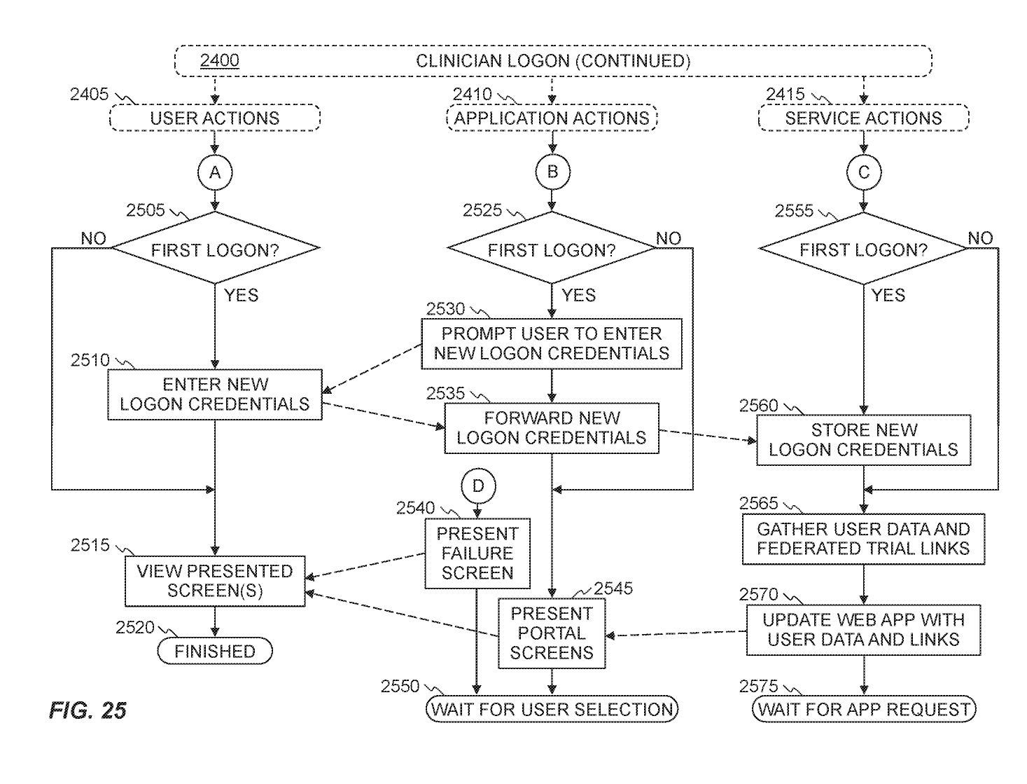

Clinician logon process 2400 is detailed in FIG. 24 and FIG. 25, and is a prerequisite for as well as an embedded operation in every other clinician process that follows. Three primary actors participate in this process: a user, which in this case is the clinician; an application, which in this case is the clinician application 150 web app; and a service, which in this case is the various elements of trial operations service suite 200, including in particular clinician portal 205. In the figure, the operations performed by these actors are shown as part of interacting sub-processes: user actions 2405, application actions 2410, and service actions 2415. Solid arrows represent temporal ordering and information flow within a sub-process, and dashed arrows represent temporal ordering and information flow between sub-processes. Note that the sub-process flows cross from FIG. 24 to FIG. 25 via the connection points shown as labeled circles.

Clinician logon process 2400 begins at operation 2420, in which the user may activate a web browser installed in clinician workstation 105 and enter a URL to access clinician portal 205. At operation 2435, the web browser should access clinician portal 205 to download and execute the clinician application 150 web app. Clinician portal 205 facilitates this download at operation 2460, having been actively awaiting the access attempt via portals autonomous operation process 7200. Upon loading, at operation 2440 clinician application 150 prompts the user to logon or register, so at operation 2425 the user selects the logon action, whereupon at operation 2445 clinician application 150 prompts the user to enter logon credentials. The clinician enters credentials at operation 2430, and at operation 2450 clinician application 150 relays them to clinician portal 205 for verification and an appropriate response in operation 2465. If the credentials were not accepted, the decision at operation 2470 causes server sub-process 2415 to return to awaiting connection attempts. Similarly, application sub-process 2410 checks at operation 2455 whether the response indicated acceptance of the credentials. If not, clinician application 150 presents a failure notice to the user at operation 2540. If the credentials were accepted, each sub-process checks whether this is the user's first logon after registration or password reset, at operations 2525, 2505, and 2555. If so, clinician application 150 prompts the user at operation 2530 to enter new logon credentials at operation 2510, and relays the updated credentials at operation 2535 so that clinician portal 205 may store them in provisioning and authorization data 222 at operation 2560. Afterward, or if this is not the first logon, at operation 2565 clinician portal 205 may retrieve from identity federation gateway 289 a list of federated trial links, and gather from operational databases 202 relevant data to populate the user's portal view, then at operation 2570 update clinician application 150 with this information. Clinician application 150 would in turn at operation 2545 present this information structured as portal screens according to the trial design. The clinician then may view the presented screens, whether the failure notice of operation 2540 or the portal view of operation 2545, at operation 2515.

At this point, clinician logon process 2400 is complete, with each sub-process quiescing in its own way. User sub-process 2405 simply finishes at operation 2520, with nothing further for the clinician to do. In application sub-process 2410, clinician application 150 remains active in clinician workstation 105, so its quiescent state is waiting for the user's next selection at operation 2550. Similarly, clinician portal 205 has an established session for this user, with clinician application 150 connected and authenticated, so it too quiesces in an active state waiting for the next application request at operation 2575. Any of the other clinician methods may be selected in this state.

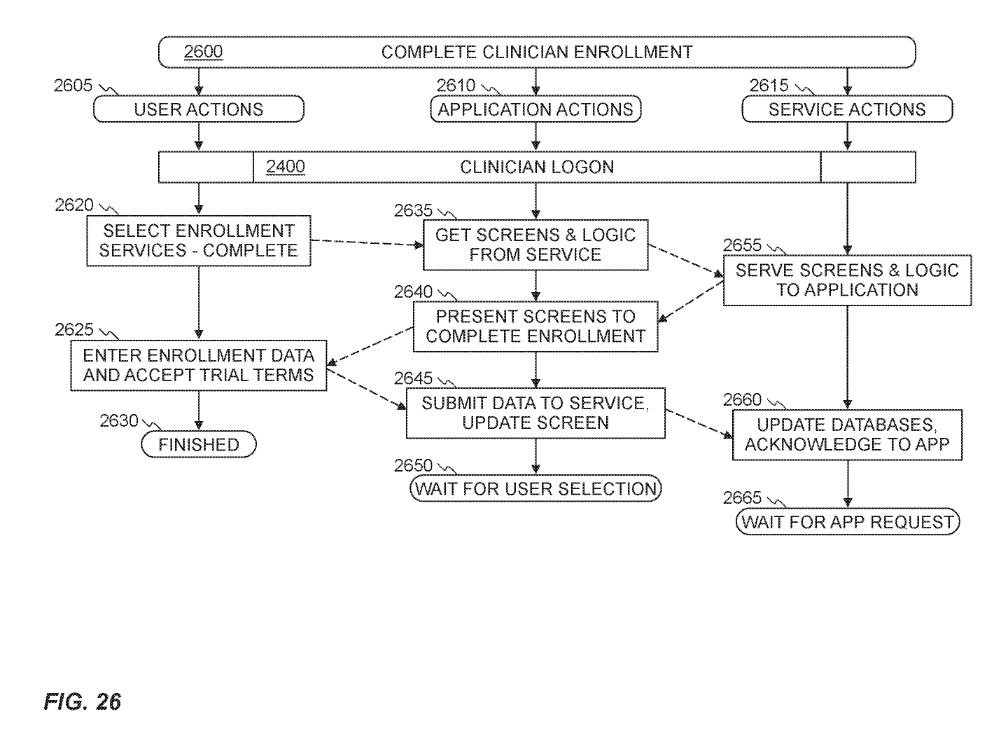

If a clinician has not yet accepted the trial terms and provided all the demographic, patient relationship, and other information required for the trial, one of the first actions to occur after clinician self-registration process 2300 is complete clinician enrollment process 2600, which is depicted in FIG. 26. Three primary actors participate in this process: a user, which in this case is the clinician; an application, which in this case is the clinician application 150 web app; and a service, which in this case is the various elements of trial operations service suite 200, including in particular clinician portal 205 and clinician portal services 250. In the figure, the operations performed by these actors are shown as part of interacting sub-processes: user actions 2605, application actions 2610, and service actions 2615. Solid arrows represent temporal ordering and information flow within a sub-process, and dashed arrows represent temporal ordering and information flow between sub-processes.

Complete clinician enrollment process 2600 begins with the prerequisite of clinician logon process 2400, in which all three actors participate. After that, at operation 2620 the clinician may select enrollment services 251 from the menu of clinician portal services 250. This may include explicit selection of an action to complete enrollment, although in a preferred embodiment clinician application 150 may be aware from its clinician portal services logic that enrollment is incomplete and must be the first action to take. Either way, at operation 2635 clinician application 150 may get the corresponding additional screens and logic from clinician portal 205 if they are not already loaded, and clinician portal 205 would provide them at operation 2655. At operation 2640, then, the application presents the appropriate screens for completing enrollment to the user. Depending on the trial design established by a coordinator, the clinician may be presented a number of screens and options and data entry opportunities in order to qualify for participation in the trial, then review and consent to the terms of the trial, and provide enrollment information required by the trial protocol such as license status, patient relationships, and others. Operation 2625 embodies the clinician's activity of reading this material and entering the requested data. This may take the form of several interactions between user and app, but in the end when all the entries have been made, clinician application 150 at operation 2645 may submit the entire data package to clinician portal 205 enrollment services 251, which in turn at operation 2660 would update operational databases 202 with this new information about the clinician. Some of the information may be recorded in provisioning and authorization data 222, while other information such as patient relationships may be categorized such that it must be recorded in protected health information 223; if appropriate, some of the latter may be also de-identified and recorded in anonymized health information 224. When all the data is safely stored, still within operation 2660, clinician portal 205 enrollment services 251 should provide an acknowledgement to clinician application 150 so it knows enrollment is complete and does not present the option to do so again.

At this point, complete clinician enrollment process 2600 is complete, with each sub-process quiescing in its own way. User sub-process 2605 simply finishes at operation 2630, with nothing further for the clinician to do. In application sub-process 2610, clinician application 150 remains active in clinician workstation 105, so its quiescent state is waiting for the user's next selection at operation 2650. Similarly, clinician portal 205 has an established session for this user, with clinician application 150 connected and authenticated, so it too quiesces in an active state waiting for the next application request at operation 2665. Any of the other clinician methods may be selected in this state.

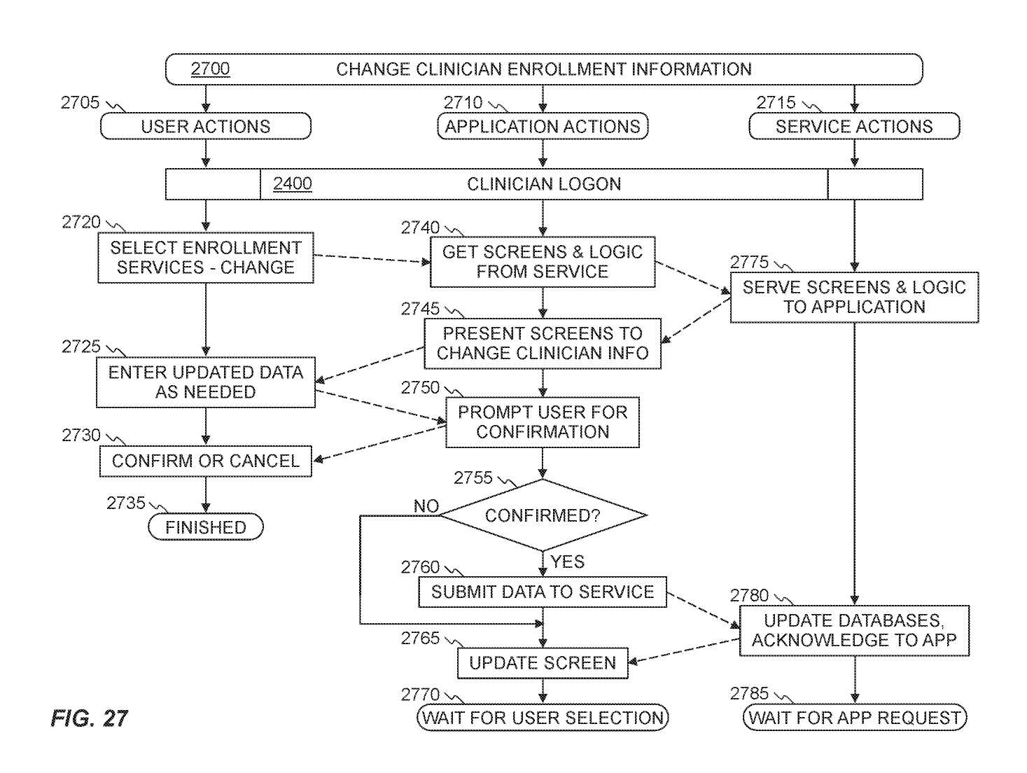

When a clinician needs to change enrollment information, such as contact address or patient relationship information, or even to withdraw from the trial by deleting enrollment, the change clinician enrollment information process 2700 may be used, as depicted in FIG. 27. The same three-actor model applies here, with the same temporal/informational interaction conventions. The sub-processes in this case are labeled user actions 2705, application actions 2710, and service actions 2715.

Change clinician enrollment information process 2700 begins with the prerequisite of clinician logon process 2400, in which all three actors participate. After that, at operation 2720 the clinician may select the change option under enrollment services 251 from the menu of clinician portal services 250. At operation 2740 clinician application 150 may get the corresponding additional screens and logic from clinician portal 205 if they are not already loaded, and clinician portal 205 would provide them at operation 2775. At operation 2745, then, the application presents the appropriate screens for changing enrollment information. Depending on the trial design established by a coordinator, the clinician may be presented one or more screens of options and data entry opportunities in order to update the information as needed, all embodied in operation 2725. This may take the form of several interactions between user and app, but in the end when all the changes have been made, clinician application 150 at operation 2750 may prompt the clinician for confirmation, and the clinician at operation 2730 may choose to confirm or cancel the changes. If clinician application 150 determines at operation 2755 that the clinician confirmed the changes, it may at operation 2760 submit the change package to clinician portal 205 enrollment services 251, which in turn at operation 2780 would update operational databases 202 with this new information about the clinician. Some of the information may be updated in provisioning and authorization data 222, while other information may be categorized such that it must be updated in protected health information 223; if appropriate, some of the latter may also be de-identified and recorded in anonymized health information 224. When all the data is safely stored, still within operation 2780, clinician portal 205 enrollment services 251 should provide an acknowledgement to clinician application 150 so it knows the change was successful. Confirmed or not, changed successfully or not, clinician application 150 updates the screen to reflect the appropriate state of affairs at operation 2765.

At this point, change clinician enrollment information process 2700 is complete, with each sub-process quiescing in its own way. User sub-process 2705 simply finishes at operation 2735, with nothing further for the clinician to do. In application sub-process 2710, clinician application 150 remains running in clinician workstation 105, so its quiescent state is waiting for the user's next selection at operation 2770. Similarly, clinician portal 205 has an established session for this user, with clinician application 150 connected and authenticated, so it too quiesces in an active state waiting for the next application request at operation 2785. Any of the other clinician methods may be selected in this state.

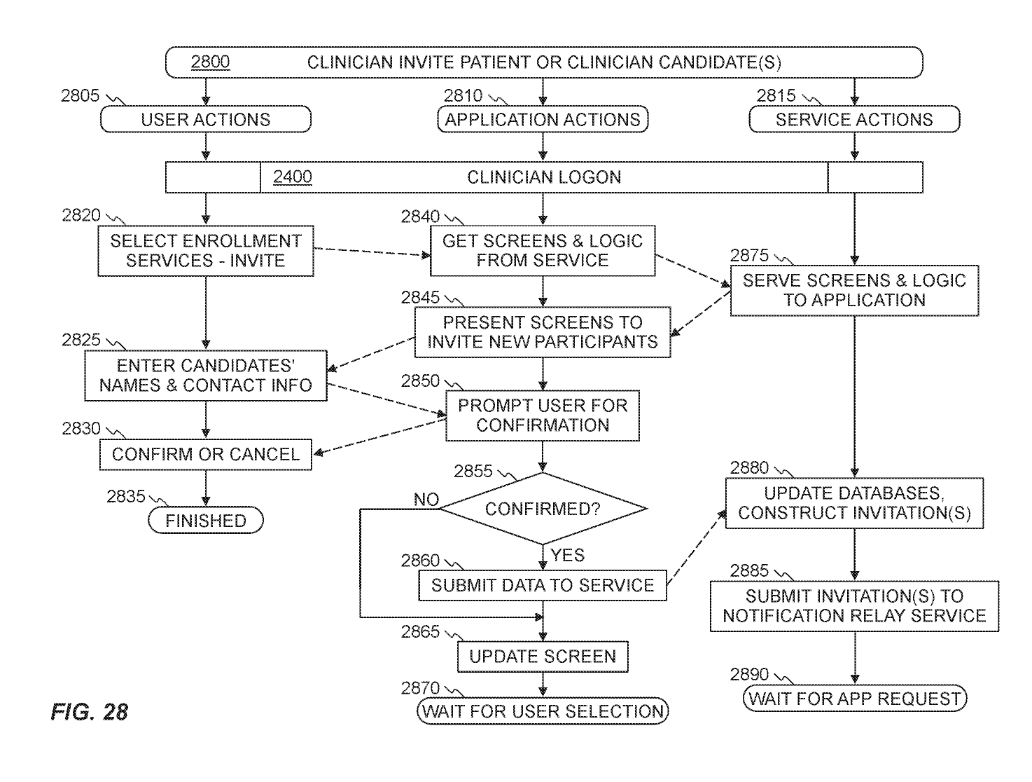

One of the ways a patient or clinician such as a doctor may learn about a particular trial and choose to self-register for it (process 600 for patients, process 2300 for clinicians) is by invitation from a current participant, based on real-life interactions. This “social networking” aspect of trial participation is facilitated by process 2800, clinician invite patient or clinician candidate(s), which is depicted in FIG. 28. The same three-actor model applies here, with the same temporal/informational interaction conventions. The sub-processes in this case are labeled user actions 2805, application actions 2810, and service actions 2815.

Clinician invite patient or clinician candidate process 2800 begins with the prerequisite of clinician logon process 2400, in which all three actors participate. After that, at operation 2820 the clinician may select the invite option under enrollment services 251 from the menu of clinician portal services 250. At operation 2840 clinician application 150 may get the corresponding additional screens and logic from clinician portal 205 if they are not already loaded, and clinician portal 205 would provide them at operation 2875. At operation 2845, then, the application presents the appropriate screens for inviting new participants. Depending on the trial design established by a coordinator, the clinician may be presented one or more screens of options and data entry opportunities in order to provide name(s) and contact information for the patient or clinician candidate(s) to be invited, all embodied in operation 2825. This may take the form of several interactions between user and app, including for example entering each name/contact pair one by one or selecting a file containing several name/contact pairs, but in the end when the information has been entered, clinician application 150 at operation 2850 may prompt the clinician for confirmation, and the clinician at operation 2830 may choose to confirm or cancel the invitation(s). If clinician application 150 determines at operation 2855 that the clinician confirmed the invitation, it may at operation 2860 submit the invitation package to clinician portal 205 enrollment services 251, which in turn at operation 2880 would update operational databases 202 with this new information about the invitee(s), and construct the actual invitation message(s). In general, only provisioning and authorization data 222 would be created at this operation, because the invitee(s) would have to provide the remaining enrollment data in their own respective executions of self-registration process 600/2300 and enrollment completion process 900/2600. When all the data is safely stored, clinician portal 205 enrollment services 251 should submit, at operation 2885, each invitation separately to notification relay services 282 within trial operations service suite 200, in order to have the invitation(s) relayed externally; refer to process 7400, notifications relay service autonomous operation, for details of how this occurs. Finally, whether the invitation set was confirmed or not, clinician application 150 may update the screen to reflect the appropriate state of affairs at operation 2865.

At this point, clinician invite patient or clinician candidate process 2800 is complete, with each sub-process quiescing in its own way. User sub-process 2805 simply finishes at operation 2835, with nothing further for the clinician to do. In application sub-process 2810, clinician application 150 remains running in clinician workstation 105, so its quiescent state is waiting for the user's next selection at operation 2870. Similarly, clinician portal 205 has an established session for this user, with clinician application 150 connected and authenticated, so it too quiesces in an active state waiting for the next application request at operation 2890. Any of the other clinician methods may be selected in this state.

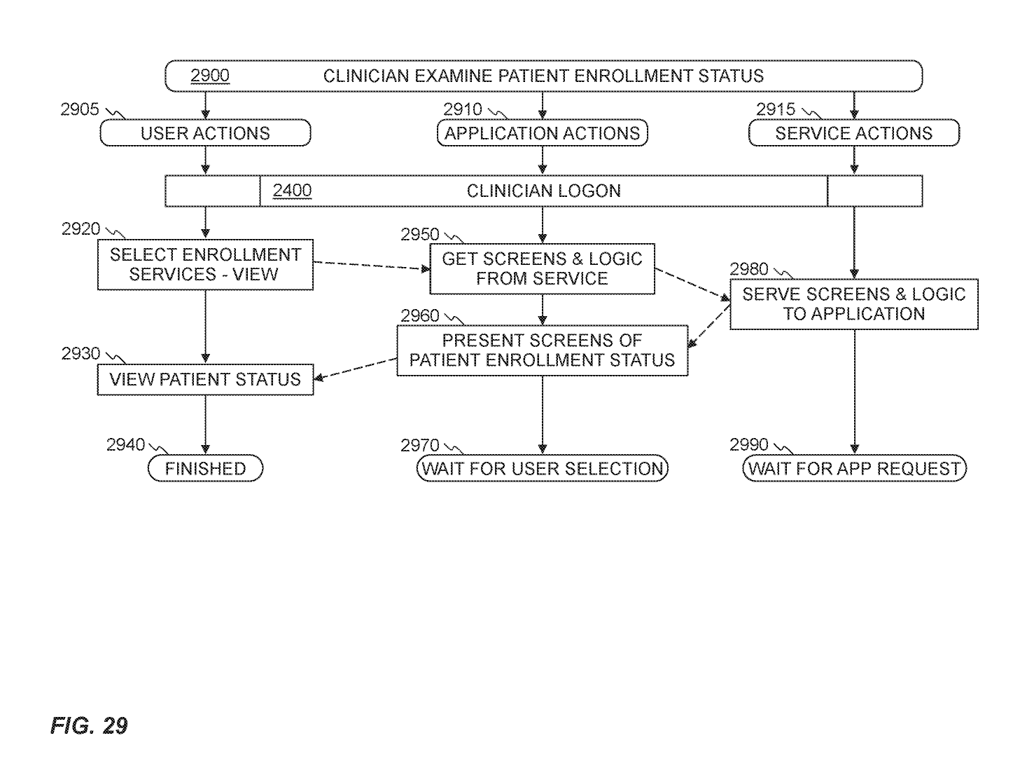

A clinician may have invited a number of patients and need to check the status of their enrollments in order to gauge progress toward meeting trial participation goals. The clinician examine patient enrollment status process 2900 may be used for this activity, as depicted in FIG. 29. The same three-actor model applies here, with the same temporal/informational interaction conventions. The sub-processes in this case are labeled user actions 2905, application actions 2910, and service actions 2915.

Clinician examine patient enrollment status process 2900 begins with the prerequisite of clinician logon process 2400, in which all three actors participate. After that, at operation 2920 the clinician may select the view patients option under enrollment services 251 from the menu of clinician portal services 250. At operation 2950 clinician application 150 may get the corresponding additional screens and logic from clinician portal 205 if they are not already loaded, and clinician portal 205 would provide them at operation 2980. At operation 2960, then, the application presents the appropriate screens for showing patient enrollment status, and the clinician may view this information at operation 2930.

At this point, clinician examine patient enrollment status process 2900 is complete, with each sub-process quiescing in its own way. User sub-process 2905 simply finishes at operation 2940, with nothing further for the clinician to do. In application sub-process 2910, clinician application 150 remains running in clinician workstation 105, so its quiescent state is waiting for the user's next selection at operation 2970. Similarly, clinician portal 205 has an established session for this user, with clinician application 150 connected and authenticated, so it too quiesces in an active state waiting for the next application request at operation 2990. Any of the other clinician methods may be selected in this state.

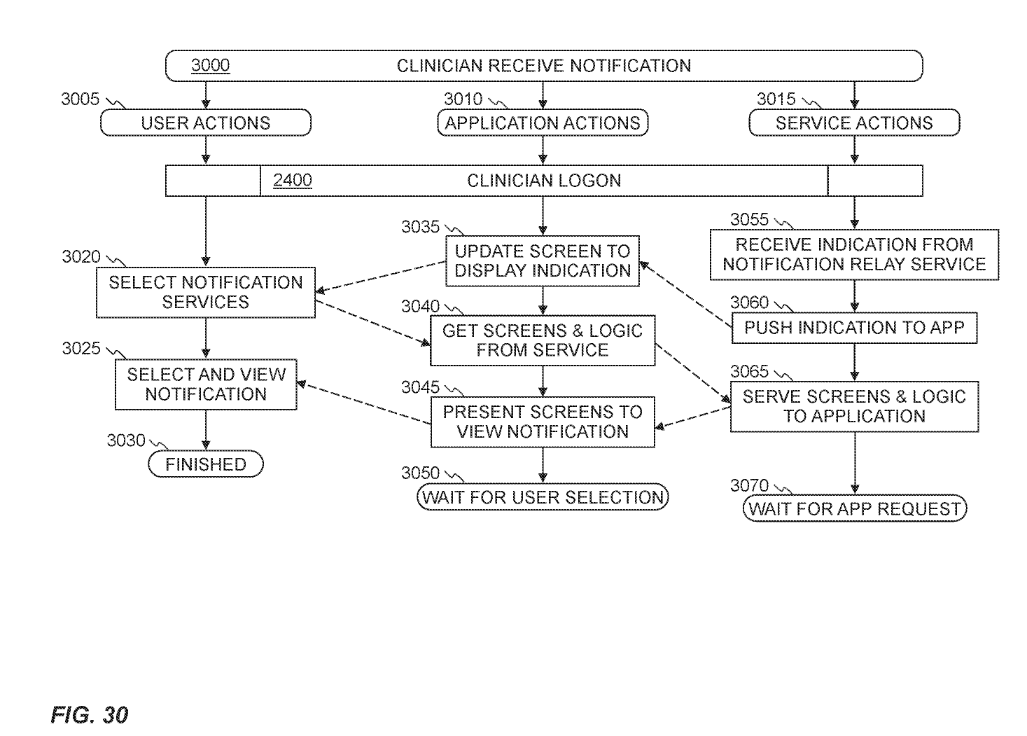

A coordinator may send clinicians a notification conveying, for example, news about the trial; see the corresponding send notification process 6800 described later. Clinician receive notification process 3000, which is depicted in FIG. 30, enables the clinician to view such notifications. Once again, the three-actor model and temporal/informational interaction conventions apply. The sub-processes in this case are labeled user actions 3005, application actions 3010, and service actions 3015.

Clinician receive notification process 3000 begins with the prerequisite of clinician logon process 2400, in which all three actors participate. Note that external notification mechanisms described in processes 7400 and 7450, respectively notifications relay service autonomous operation and external notifications gateway autonomous operation, may prompt a clinician who is not already logged on to do so. Once clinician logon process 2400 has established a portal session for the clinician, whether prompted anew or already in place from previous activity, clinician portal 205 may at operation 3055 receive an indication of the notification from notification relay service 282, and then at operation 3060 push that indication to clinician application 150. Clinician application 150 in turn may at operation 3035 update whatever screen it is displaying to include a form of the indication observable by the user.

After that, at operation 3020 the clinician may select notification service 252 from the menu of clinician portal services 250. This may involve explicit selection of a command to view notifications, or simply touching or clicking on the indication itself. Either way, at operation 3040 clinician application 150 may get the corresponding additional screens and logic from clinician portal 205 if they are not already loaded, and clinician portal 205 would provide them at operation 3065. At operation 3045, then, the application presents the appropriate screens for viewing the notification to the user. Operation 3025 embodies the clinician's activity of viewing the presented notification, which may also include selecting a specific notification from a list of notifications that may have been sent to the clinician. Note that, although not shown in FIG. 30, if the selected notification includes a survey or status query, or is an event reminder, clinician application 150 may also in this operation switch automatically to the corresponding section of engagement services 254, as specified in clinician answer query or survey process 3700 and clinician view schedule of events process 3800.

At this point, clinician receive notification process 3000 is complete, with each sub-process quiescing in its own way. User sub-process 3005 simply finishes at operation 3030, with nothing further for the clinician to do. In application sub-process 3010, clinician application 150 remains running in clinician workstation 105, so its quiescent state is waiting for the user's next selection at operation 3050. Similarly, clinician portal 205 has an established session for this user, with clinician application 150 connected and authenticated, so it too quiesces in an active state waiting for the next application request at operation 3070. Any of the other clinician methods may be selected in this state.

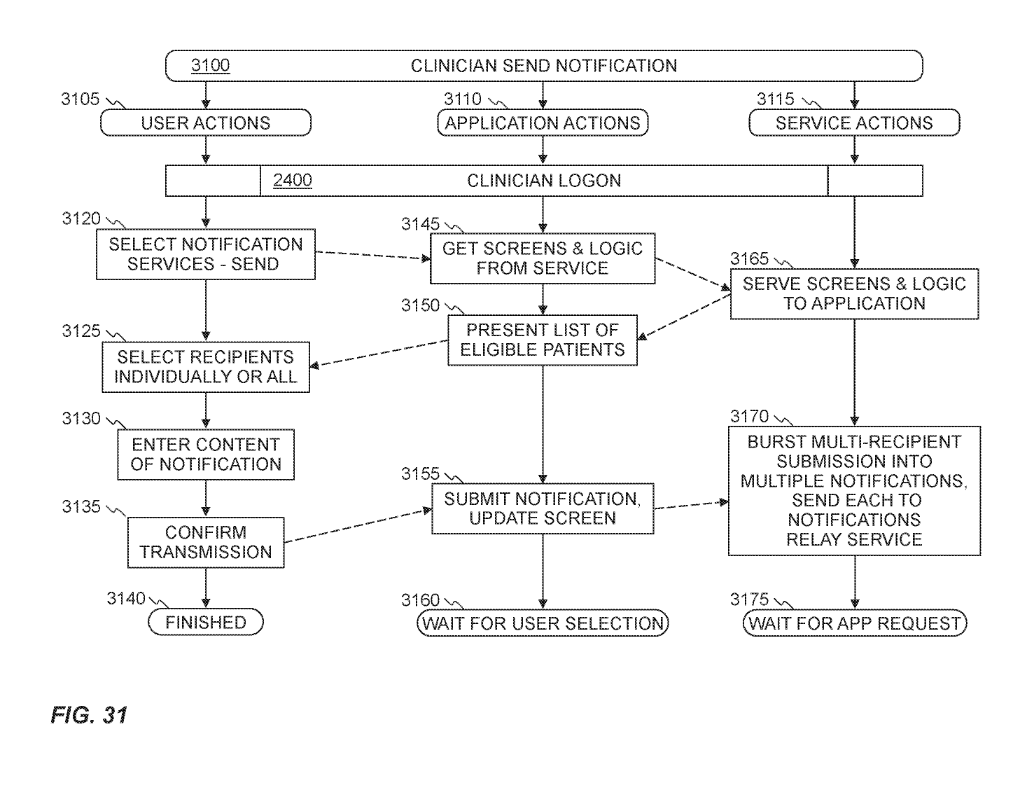

A clinician may be permitted to send a notification to one or more of the patients in the clinician's care, containing for example appointment or treatment reminders or trial status information. The clinician send notification process 3100 may be used for this activity, as depicted in FIG. 31. Once again, the three-actor model applies here, with the same temporal/informational interaction conventions. The sub-processes in this case are labeled user actions 3105, application actions 3110, and service actions 3115.

Clinician send notification process 3100 begins with the prerequisite of clinician logon process 2400, in which all three actors participate. After that, at operation 3120 the clinician may select the send option under notification services 252 from the menu of clinician portal services 250. At operation 3145 clinician application 150 may get the corresponding additional screens and logic from clinician portal 205 if they are not already loaded, and clinician portal 205 would provide them at operation 3165. At operation 3150, then, the application may present a list for selecting the patient or patients to whom the notification should be sent, and the clinician may select one, several, or all recipients from this list at operation 3125. The clinician may then enter the notification content at operation 3130, and confirm transmission of the notification at operation 3135, at which point clinician application 150 would at operation 3155 submit the notification data package to clinician portal 205 notification services 252 and update the screen to reflect that this had been done. At operation 3170, notification services 252 would parse the submitted notification data package into one or more notification requests, each addressed to a single patient from the list of recipients selected by the clinician at operation 3125 and containing a copy of the content provided by the clinician at operation 3130, and then submit each notification individually to notifications relay service 282 for handling according to notifications relay service autonomous operation process 7400.

At this point, clinician send notification process 3100 is complete, with each sub-process quiescing in its own way. User sub-process 3105 simply finishes at operation 3140, with nothing further for the clinician to do. In application sub-process 3110, clinician application 150 remains running in clinician workstation 105, so its quiescent state is waiting for the user's next selection at operation 3160. Similarly, clinician portal 205 has an established session for this user, with clinician application 150 connected and authenticated, so it too quiesces in an active state waiting for the next application request at operation 3175. Any of the other clinician methods may be selected in this state.

Trial operations service suite 200 may include support for communication between a clinician and other trial participants, potentially including patients, other clinicians, investigators, and coordinators. Which other participants a clinician is allowed to select for communication may be constrained by the trial design as established by a coordinator. For example, a clinician may be permitted to communicate only with patients under the clinician's care, or a clinician may be permitted to communicate with other clinicians in the trial. This capability, if permitted by the trial design, may enable a social networking aspect across the community of trial participants, thereby enhancing participants' engagement in the trial protocol. Four modes of communication may be supported, which are divided into two categories for the purpose of explaining their operation. Communication that is oriented toward exchanging written messages, documents, images, and other related information is categorized as messaging, and typified as chat mode or email mode. Communication that is oriented toward verbal or visual conversation over a live full-duplex connection is categorized as calling, and typified as voice mode or video mode. The processes supporting these capabilities are specified for each category, and within each category for initiating and accepting communications of the corresponding category, yielding four separate processes to be described hereinafter.

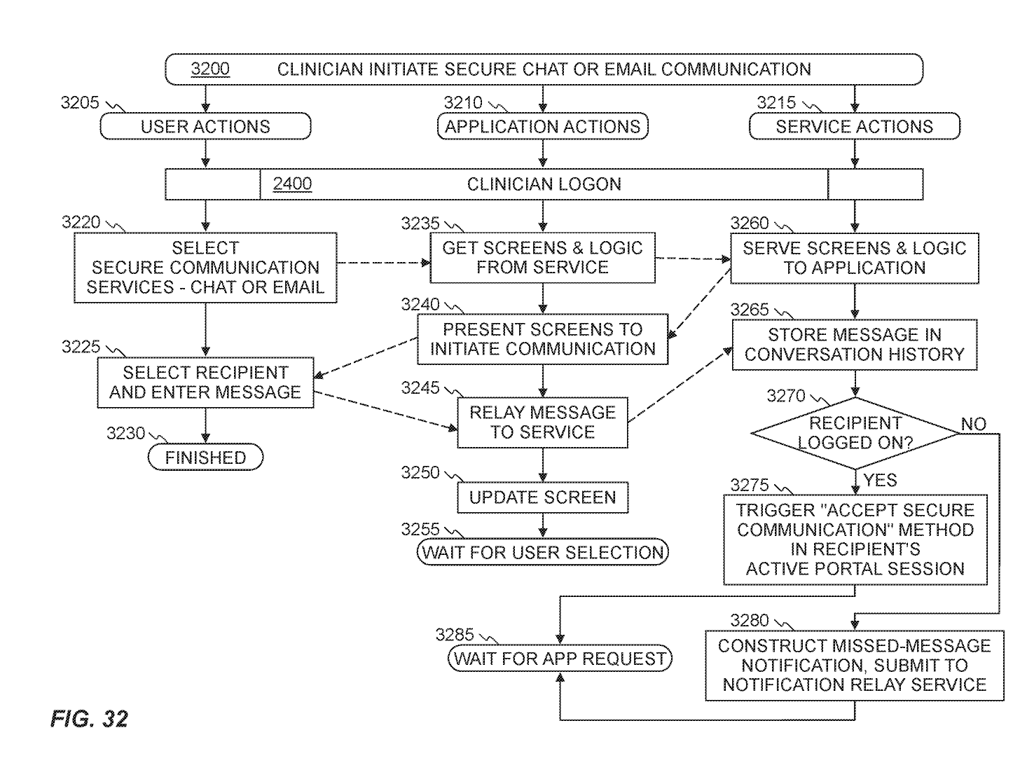

The first communication process is clinician initiate secure chat or email communication process 3200, which is depicted in FIG. 32. As usual, the three-actor model and temporal/informational interaction conventions apply. The sub-processes in this case are labeled user actions 3205, application actions 3210, and service actions 3215; the primary service actor is secure communication services 253, with help from secure communication relay services 281.

Clinician initiate secure chat or email communication process 3200 begins with the prerequisite of clinician logon process 2400, in which all three actors participate. After that, at operation 3220 the clinician may select secure communication services 253 and the appropriate communication mode—in this case either chat or email—from the menu of clinician portal services 250. At operation 3235 clinician application 150 may get the corresponding additional screens and logic from clinician portal 205 if they are not already loaded, and clinician portal 205 would provide them at operation 3260. At operation 3240, then, the application presents the appropriate screens for initiating communication. Depending on the trial design established by a coordinator, the clinician may select one or more recipients to which a chat or email message should be sent, with the selection mechanism and message entry embodied in operation 3225, whereupon clinician application 150 should at operation 3245 relay the entered message to secure communication services 253 of clinician portal 205, and at operation 3250 update the presented screen accordingly. Meanwhile, secure communication relay services 281 at operation 3265 may record the message in a conversation history associated with both the sending clinician and the recipient, which may preferably be stored in the notes, records, and events 225 section of operational databases 202. Note that while only one recipient is depicted in the figure, this and following operations may be repeated for each of multiple recipients if the sender selected more than one. Then, at operation 3270 secure communication relay services 281 may determine whether the recipient is currently logged on in an active portal session. If so, at operation 3275 secure communication services 253 may trigger the recipient's accept secure chat or email communication process 1400, 3300, 4800, or 6300 depending on the recipient's role in the system, respectively a patient, another clinician, an investigator, or a coordinator. If the recipient does not have a current active portal session, then at operation 3280 secure communication relay services 281 may construct a missed-message notification and submit it to notifications relay service 282 so that it may be sent to the recipient's external contact address, facilitated by process 7400 notifications relay service autonomous operation and process 7450 external notifications gateway autonomous operation.

At this point, clinician initiate secure chat or email communication process 3200 is complete, with each sub-process quiescing in its own way. User sub-process 3205 simply finishes at operation 3230, with nothing further for the clinician to do. In application sub-process 3210, clinician application 150 remains running in clinician workstation 105, so its quiescent state is waiting for the user's next selection at operation 3255. Similarly, clinician portal 205 has an established session for this user, with clinician application 150 connected and authenticated, so it too quiesces in an active state waiting for the next application request at operation 32150. Any of the other clinician methods may be selected in this state.

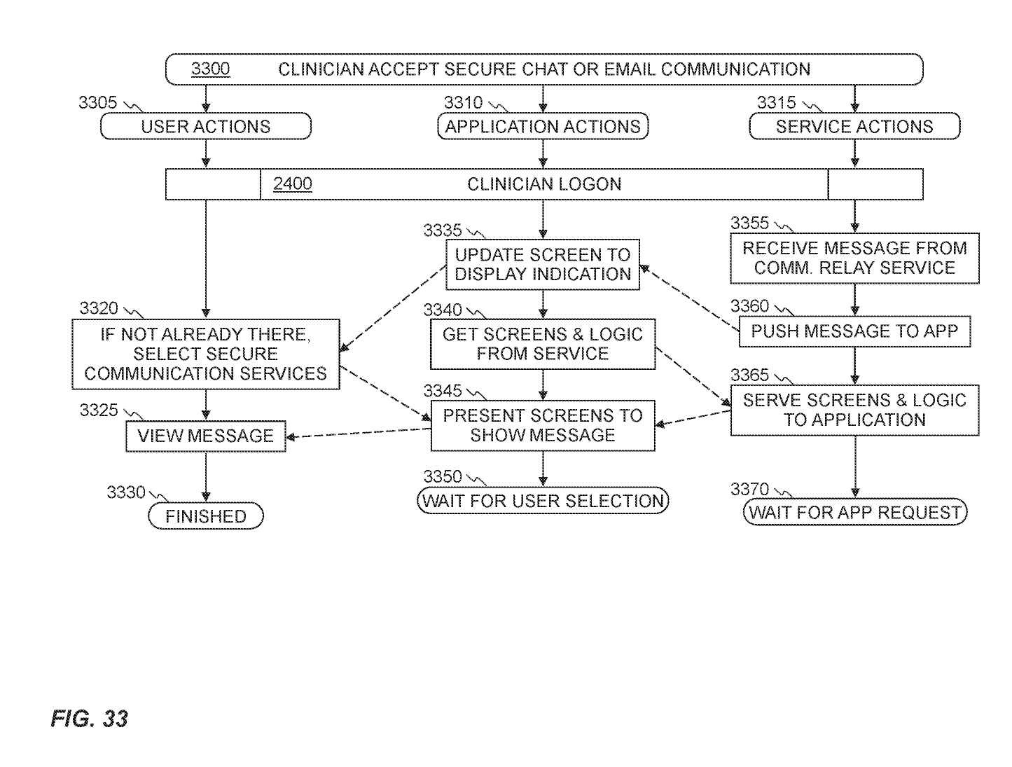

The next communication process is clinician accept secure chat or email communication process 3300, which is depicted in FIG. 33. As usual, the three-actor model and temporal/informational interaction conventions apply. The sub-processes in this case are labeled user actions 3305, application actions 3310, and service actions 3315; the primary service actor is secure communication services 253, with help from secure communication relay services 281.

Clinician accept secure chat or email communication process 3300 begins with the prerequisite of clinician logon process 2400, in which all three actors participate. Note that external notification mechanisms described in processes 7400 and 7450, respectively notifications relay service autonomous operation and external notifications gateway autonomous operation, may prompt a clinician who is not already logged on to do so. Once clinician logon process 2400 has established a portal session for the clinician, whether prompted anew or already in place from previous activity, at operation 3355 secure communication services 253 of clinician portal 205 may receive a copy of the message from secure communication relay services 281. After that, at operation 3360 the clinician portal 205 should push the message to clinician application 150, which would in turn at operation 3335 update whatever screen it is displaying to include an indication of the message's arrival. At operation 3340 clinician application 150 may predictively retrieve the corresponding additional screens and logic from clinician portal 205 if they are not already loaded, and clinician portal 205 would provide them at operation 3365.

When the clinician notices the indication thus displayed, at operation 3320 the clinician may select secure communication services 253 and the appropriate communication mode—in this case either chat or email—from the menu of clinician portal services 250. This may involve explicit selection of a command to enter the corresponding service, or simply touching or clicking on the indication itself; alternatively, clinician application 150 may switch to the corresponding service mode automatically, or already be in the corresponding service mode due to previous activity. However it arrived in the appropriate state, at operation 3345 clinician application 150 presents the appropriate screens for viewing the received message. Operation 3325 embodies the clinician's activity of viewing the presented message. If the communication mode is email, this may also include selecting a specific message from a list of messages that may have been sent to the clinician. If the communication mode is chat, this may also include viewing other messages both sent and received in the context of a conversation.

At this point, clinician accept secure chat or email communication process 3300 is complete, with each sub-process quiescing in its own way. User sub-process 3305 simply finishes at operation 3330, with nothing further for the clinician to do. In application sub-process 3310, clinician application 150 remains running in clinician workstation 105, so its quiescent state is waiting for the user's next selection at operation 3350. Similarly, clinician portal 205 has an established session for this user, with clinician application 150 connected and authenticated, so it too quiesces in an active state waiting for the next application request at operation 3370. Any of the other clinician methods may be selected in this state.

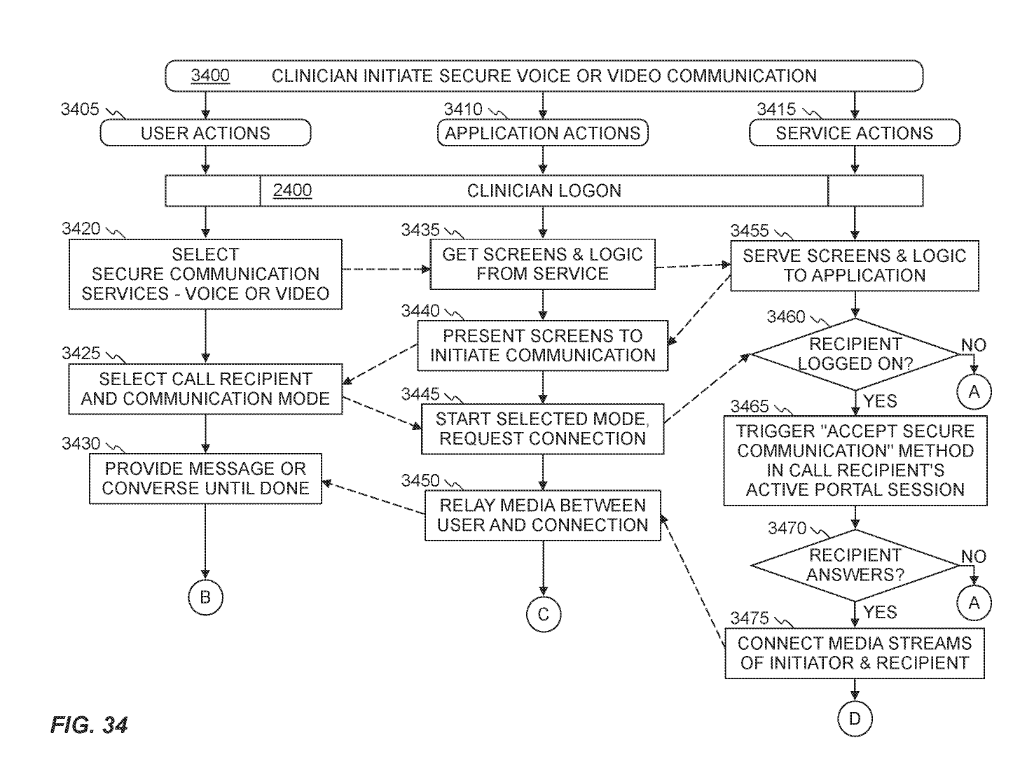

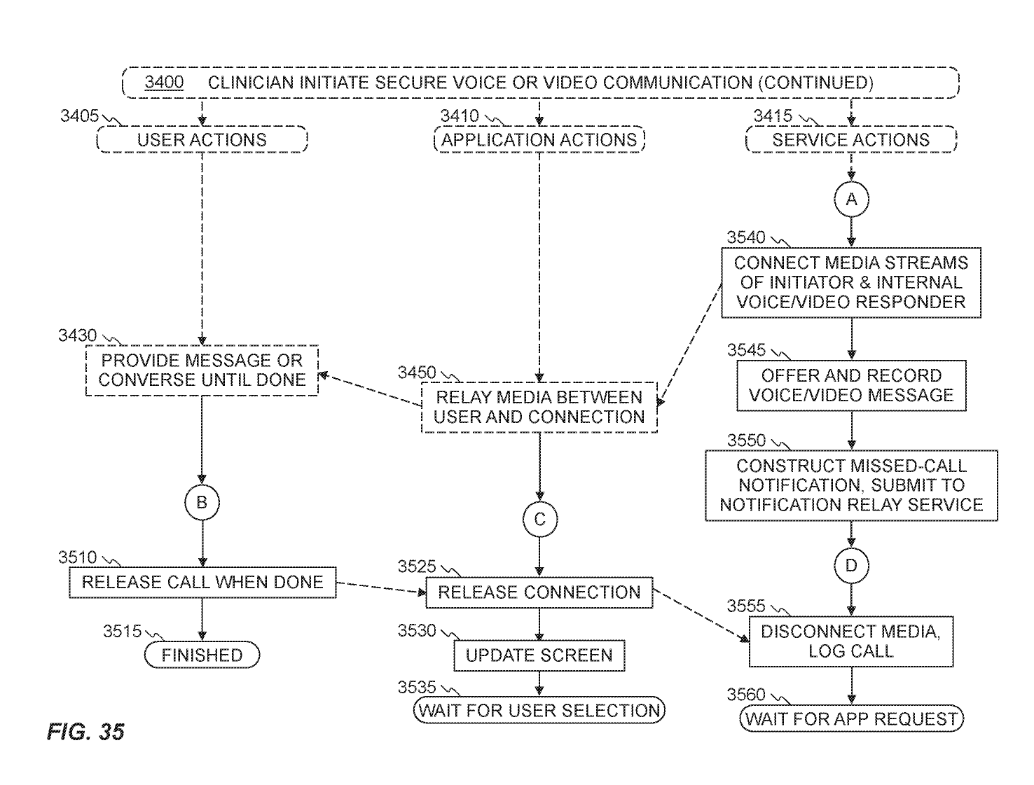

The next communication process is clinician initiate secure voice or video communication process 3400, which is depicted in FIG. 34 and FIG. 35. As usual, the three-actor model and temporal/informational interaction conventions apply. The sub-processes in this case are labeled user actions 3405, application actions 3410, and service actions 3415; the primary service actor is secure communication services 253, with help from secure communication relay services 281. Note that the sub-process flows cross from FIG. 34 to FIG. 35 via the connection points shown as labeled circles.

Clinician initiate secure voice or video communication process 3400 begins with the prerequisite of clinician logon process 2400, in which all three actors participate. After that, at operation 3420 the clinician may select secure communication services 253 and the appropriate communication mode—in this case either voice or video—from the menu of clinician portal services 250. At operation 3435 clinician application 150 may get the corresponding additional screens and logic from clinician portal 205 if they are not already loaded, and clinician portal 205 would provide them at operation 3455. At operation 3440, then, the application presents the appropriate screens for initiating communication. Depending on the trial design established by a coordinator, the clinician may select another trial participant with which a voice or video call should be placed, with the selection mechanism and message entry embodied in operation 3425, whereupon clinician application 150 should at operation 3445 start the selected communication mode and request secure communication services 253 to have secure communication relay services 281 make a connection supporting the call. At operation 3460 secure communication relay services 281 may determine whether the called participant, referred to as recipient in the Figure, is currently logged on in an active portal session. If so, at operation 3465 secure communication relay services 281 may trigger the recipient's accept secure voice or video communication process 1700, 3600, 5100, or 6600 depending on the recipient's role in the system, respectively a patient, another clinician, an investigator, or a coordinator. During that process, the recipient may choose to accept (answer), reject, or ignore the call. At operation 3470, secure communication relay services 281 determines this outcome. If the recipient answers, at operation 3475 secure communication relay services 281 connects the media streams of the initiator and the recipient so that they may converse.

Whether the call is answered by the recipient or the message recorder, at operation 3450 clinician application 150 relays the media streams between the calling user and the connection, so that the clinician may at operation 3430 either converse with the called participant or leave a voice or video message, as appropriate. Note that these two operations appear on both FIG. 34 and FIG. 35, and represent the same actions in both. When the clinician is finished conversing with or providing a voice/video message for the recipient, he or she will release the call at operation 3510. This may take the form of clicking or touching a hang-up control on the screen of, or pressing a button on, clinician workstation 105.

If the recipient does not have a current active portal session (operation 3460), or if the recipient does not answer the call (operation 3470), then at operation 3540 secure communication services 253 may connect the initiator's media streams to those of an internal voice or video responder—effectively an answering machine—provided by secure communication relay services 281, which at operation 3545 may offer prompts to record a voice or video message and then do so if one is provided by the initiator. After the voice or video message is finished, which may be determined by detecting a configured period of silence, or by detecting an explicit indication from the calling user such as a key word, tone, or disconnect, or if the caller chooses not to leave a message at all, at operation 3550 the secure communication relay services 281 may construct a missed-call notification and submit it to notifications relay service 282 so that it may be sent to the recipient's external contact address, facilitated by process 7400 notifications relay service autonomous operation and process 7450 external notifications gateway autonomous operation.

Whether the call is answered by the recipient or the message recorder, at operation 3450 clinician application 150 relays the media streams between the calling user and the connection, so that the clinician may at operation 3430 either converse with the called participant or leave a voice or video message, as appropriate. Note that these two operations appear on both FIG. 34 and FIG. 35 and represent the same actions in both. When the clinician is finished conversing with or providing a voice/video message for the recipient, he or she will release the call at operation 3510. This may take the form of clicking or touching a hang-up control on the screen of, or pressing a button on, clinician workstation 105. Alternatively, clinician application 150, secure communication services 253, or secure communication relay services 281 may detect a predetermined spoken command or visible gesture from the clinician as indicating the call is over, or detect that a disconnect indication of some sort was detected on the other side of the call. However call termination may be detected, clinician application 150 will at operation 3525 release the connection and at operation 3530 update the screen to reflect this change of state. At operation 3555 secure communication relay services 281 will disconnect the media streams between the initiator's and recipient's portal sessions, and record the event in a conversation history associated with both the calling clinician and the recipient, which may preferably be stored in the notes, records, and events 225 section of operational databases 202.

At this point, clinician initiate secure voice or video communication process 3400 is complete, with each sub-process quiescing in its own way. User sub-process 3405 simply finishes at operation 3515, with nothing further for the clinician to do. In application sub-process 3410, clinician application 150 remains running in clinician workstation 105, so its quiescent state is waiting for the user's next selection at operation 3535. Similarly, clinician portal 205 has an established session for this user, with clinician application 150 connected and authenticated, so it too quiesces in an active state waiting for the next application request at operation 3560. Any of the other clinician methods may be selected in this state.

Note that clinician initiate secure voice or video communication process 3400 may also be used to listen to a voice message or view a video message left by another participant when a call from that other participant was missed by the clinician. The details of this usage are not depicted, but they are effectively identical if the “answering machine” function of secure communication relay services 281 is considered the call recipient and the clinician's interactions with that function are considered the conversation.

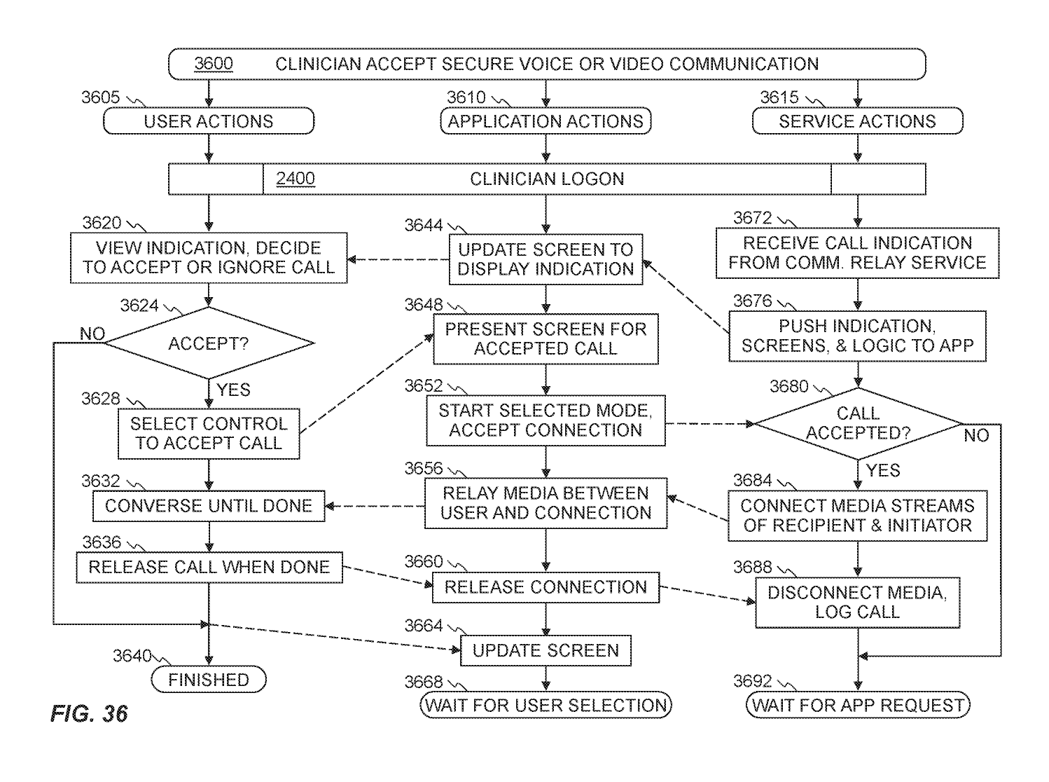

The fourth communication process is clinician accept secure voice or video communication process 3600, which is depicted in FIG. 36. Again, the three-actor model and temporal/informational interaction conventions apply. The sub-processes in this case are labeled user actions 3605, application actions 3610, and service actions 3615; the primary service actor is secure communication services 253, with help from secure communication relay services 281.

Clinician accept secure voice or video communication process 3600 begins with the prerequisite of clinician logon process 2400, in which all three actors participate. Note that for a voice or video call, this will generally have already occurred such that the portal session is currently active; otherwise the called clinician would not have an opportunity to accept the call while it is being offered, and it would have turned into a missed call. Thus in this scenario, right away at operation 3672 secure communication services 253 of clinician portal 205 should receive an indication of the offered call from secure communication relay services 281, and at operation 3676 clinician portal 205 should push that indication, as well as screens and logic for accepting a voice or video call, to clinician application 150. Clinician application 150 would in turn at operation 3644 update whatever screen it is displaying to include an indication of the incoming call.

When the clinician notices, at operation 3620, the indication thus displayed, he or she may choose at operation 3624 to accept or ignore the call. If the decision is to accept, the clinician at operation 3628 may select the necessary control to do so. This may involve explicit selection of a menu command to enter secure communication services 253 and the appropriate communication mode—in this case either voice or video. Preferably, though, it would involve simply touching or clicking on the indication itself, causing clinician application 150 to switch to the corresponding service mode automatically. However it arrived in the appropriate state, at operation 3648 clinician application 150 presents the appropriate screen for having accepted the call, and at operation 3652 it starts the selected mode and completes the connection. At operation 3680, secure communication services 253 and secure communication relay services 281 also may determine whether the clinician accepted the call, and if so proceed at operation 3684 to connect the media streams of the recipient and initiator, while clinician application 150 at operation 3656 relays the media streams between the clinician and the connection, so that the clinician may at operation 3632 converse with the caller. When the clinician is finished conversing with the caller, he or she will release the call at operation 3636. This may take the form of clicking or touching a hang-up control on the screen of, or pressing a button on, clinician workstation 105. Alternatively, clinician application 150, secure communication services 253, or secure communication relay services 281 may detect a predetermined spoken command or visible gesture from the clinician as indicating the call is over, or detect that a disconnect indication of some sort was detected on the other side of the call. However call termination may be detected, clinician application 150 will at operation 3660 release the connection and at operation 3664 update the screen to reflect this change of state. At operation 3688 secure communication services 253 will disconnect the media streams between the initiator's and recipient's portal sessions, and record the event in a conversation history associated with both the calling clinician and the recipient, which may preferably be stored in the notes, records, and events 225 section of operational databases 202.

If the clinician ignores or rejects the calls, this is detected at operations 3624 and 3680, and the process skips to the end, bypassing all the other operations except 3664 in which clinician application 150 updates the screen to reflect the corresponding state change.

Whether answered and disconnected, or ignored, at this point clinician accept secure voice or video communication process 3600 is complete, with each sub-process quiescing in its own way. User sub-process 3605 simply finishes at operation 3640, with nothing further for the clinician to do. In application sub-process 3610, clinician application 150 remains running in clinician workstation 105, so its quiescent state is waiting for the user's next selection at operation 3668. Similarly, clinician portal 205 has an established session for this user, with clinician application 150 connected and authenticated, so it too quiesces in an active state waiting for the next application request at operation 3692. Any of the other clinician methods may be selected in this state.

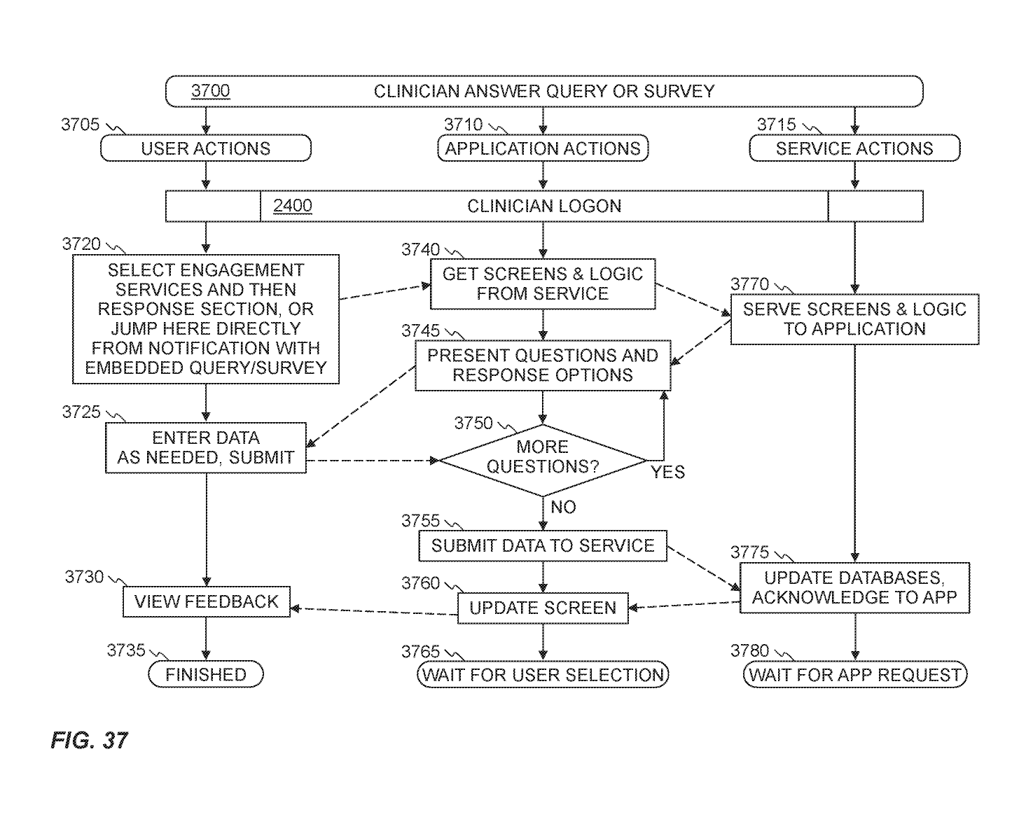

A clinician participating in a trial may be prompted periodically or upon certain occurrences to answer surveys and direct questions about trial experiences, health outcomes, and other matters according to the trial protocol. Clinician answer query or survey, process 3700 depicted in FIG. 37, supports this activity. Again, the three-actor model and temporal/informational interaction conventions apply. The sub-processes in this case are labeled user actions 3705, application actions 3710, and service actions 3715; the primary service actor is engagement services 254.

Clinician answer query or survey process 3700 begins with the usual prerequisite of clinician logon process 2400, in which all three actors participate. Note that external notification mechanisms described in processes 7400 and 7450, respectively notifications relay service autonomous operation and external notifications gateway autonomous operation, may prompt a clinician who is not already logged on to do so. After that, at operation 3720 the clinician may select engagement services 254 and the appropriate service—query or survey—from the menu of clinician portal services 250. This may include explicit selection of an action to open the diary or journal, or it may occur automatically upon opening a notification that includes a specific query or a survey request. Either way, at operation 3740 clinician application 150 may get the corresponding additional screens and logic from clinician portal 205 if they are not already loaded, and clinician portal 205 would provide them at operation 3770. At operation 3745, then, the application presents the appropriate screens containing the question(s) and response option(s) for the query or survey.

Depending on the trial design established by a coordinator, the clinician may be presented a number of screens and options and data entry opportunities in order to enter the required observation. Operation 3725 embodies the clinician's activity in this regard, which may take the form of several interactions between user and app. After the clinician submits each entry in operation 3725, clinician application 150 may determine at operation 3750 whether there are more questions in the query or survey, and if so return to operation 3745. When there are no more questions, clinician application 150 at operation 3755 may submit the data package to clinician portal 205 enrollment services 251, which in turn at operation 3775 would update operational databases 202 with this new information about the clinician. Some of the information may be recorded in notes, records, and events not classified as health information 225, while other information may be categorized such that it must be recorded in protected health information 223; if appropriate, some of the latter may be also de-identified and recorded in anonymized health information 224. When all the data is safely stored, still within operation 3775, clinician portal 205 enrollment services 251 should provide an acknowledgement to clinician application 150 so it may update the screen accordingly at operation 3760. The acknowledgement may include gratitude or encouragement feedback, which may be designed to make the clinician feel appreciated and eager to continue participating upon viewing it at operation 3730.

At this point, clinician answer query or survey process 3700 is complete, with each sub-process quiescing in its own way. User sub-process 3705 simply finishes at operation 3735, with nothing further for the clinician to do. In application sub-process 3710, clinician application 150 remains running in clinician workstation 105, so its quiescent state is waiting for the user's next selection at operation 3765. Similarly, clinician portal 205 has an established session for this user, with clinician application 150 connected and authenticated, so it too quiesces in an active state waiting for the next application request at operation 3780. Any of the other clinician methods may be selected in this state.

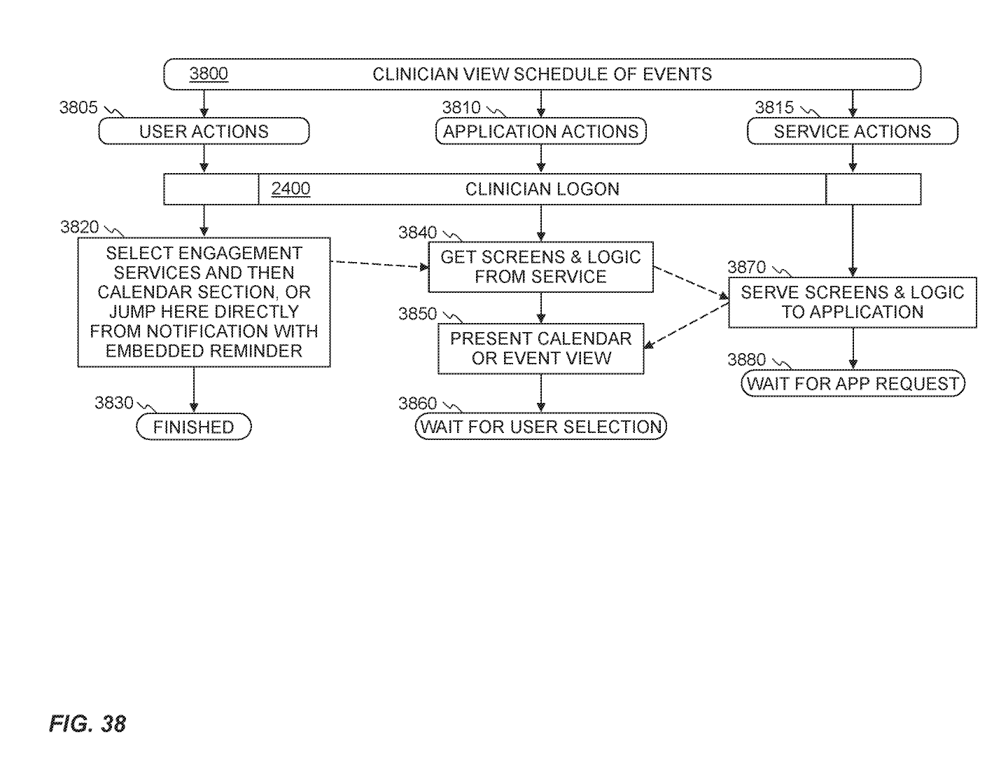

A clinician participating in a trial may have a number of patient appointments, status meetings, and other calendar events to attend according to the trial protocol. Clinician view schedule of events, process 3800 depicted in FIG. 38, allows the clinician to keep up with this schedule. Again, the three-actor model and temporal/informational interaction conventions apply. The sub-processes in this case are labeled user actions 3805, application actions 3810, and service actions 3815; the primary service actor is engagement services 254.

Clinician view schedule of events process 3800 begins with the usual prerequisite of clinician logon process 2400, in which all three actors participate. Note that external notification mechanisms described in processes 7400 and 7450, respectively notifications relay service autonomous operation and external notifications gateway autonomous operation, may prompt a clinician who is not already logged on to do so. After that, at operation 3820 the clinician may select engagement services 254 and the calendar service from the menu of clinician portal services 250. This may include explicit selection of an action to open the calendar, or it may occur automatically upon opening a notification that includes a newly scheduled event or a reminder for a previously scheduled event. Either way, at operation 3840 clinician application 150 may get the corresponding additional screens and logic from clinician portal 205 if they are not already loaded, and clinician portal 205 would provide them at operation 3870. At operation 3850, then, the application presents the appropriate screens for showing the calendar or specific event. At operation 3850, the clinician may in turn view this information.

At this point, clinician view schedule of events process 3800 is complete, with each sub-process quiescing in its own way. User sub-process 3805 simply finishes at operation 3830, with nothing further for the clinician to do. In application sub-process 3810, clinician application 150 remains running in clinician workstation 105, so its quiescent state is waiting for the user's next selection at operation 3860. Similarly, clinician portal 205 has an established session for this user, with clinician application 150 connected and authenticated, so it too quiesces in an active state waiting for the next application request at operation 3880. Any of the other clinician methods may be selected in this state.

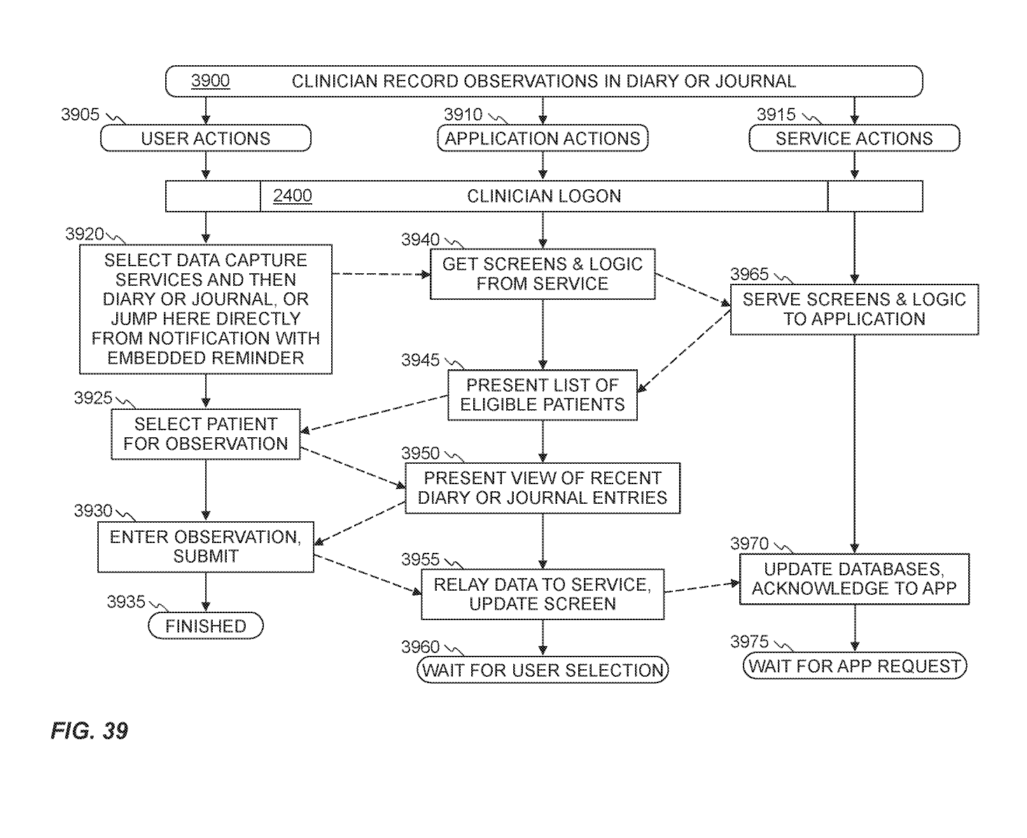

A clinician participating in a trial may be expected periodically or upon certain occurrences to record observations about patients under the clinician's care regarding symptoms, adverse outcomes, drug dosing, and other matters according to the trial protocol. If the trial design requires specific fields with limited values, such as menu selections or checkboxes, the observation may be considered a diary entry. If the trial design requires free-form text or other unconstrained information, the observation may be considered a journal entry. Clinician record observations in diary or journal, process 3900 depicted in FIG. 39, supports this activity. Again, the three-actor model and temporal/informational interaction conventions apply. The sub-processes in this case are labeled user actions 3905, application actions 3910, and service actions 3915; the primary service actor is data capture services 255.

Clinician record observations in diary or journal process 3900 begins with the usual prerequisite of clinician logon process 2400, in which all three actors participate. Note that external notification mechanisms described in processes 7400 and 7450, respectively notifications relay service autonomous operation and external notifications gateway autonomous operation, may prompt a clinician who is not already logged on to do so. After that, at operation 3920 the clinician may select data capture services 255 and the appropriate service—diary or journal—from the menu of clinician portal services 250. This may include explicit selection of an action to open the diary or journal, or it may occur automatically upon opening a notification that includes a reminder to make a particular observation or perform an action that should be recorded via an entry. Either way, at operation 3940 clinician application 150 may get the corresponding additional screens and logic from clinician portal 205 if they are not already loaded, and clinician portal 205 would provide them at operation 3965. At operation 3945, clinician application 150 may present a list of patients under the clinician's care and who are eligible for diary or journal entry. The clinician would at operation 3925 select the patient for whom observations are to be entered. At operation 3950, then, the application may present the appropriate screens for making the required entry, which may include information from other recent entries regarding the selected patient, to help the clinician complete the new entry. Depending on the trial design established by a coordinator, the clinician may be presented a number of screens and options and data entry opportunities in order to enter the required observation. Operation 3930 embodies the clinician's activity in this regard, which may take the form of several interactions between user and app. In the end when the entry has been completed and submitted, clinician application 150 at operation 3955 may submit the data package to clinician portal 205 data capture services 255, which in turn at operation 3970 would update operational databases 202 with this new information about the patient. Some of the information may be recorded in notes, records, and events not classified as health information 225, while other information may be categorized such that it must be recorded in protected health information 223; if appropriate, some of the latter may be also de-identified and recorded in anonymized health information 224. When all the data is safely stored, still within operation 3970, clinician portal 205 data capture services 255 should provide an acknowledgement to clinician application 150 so it may update the screen accordingly.

At this point, clinician record observations in diary or journal process 3900 is complete, with each sub-process quiescing in its own way. User sub-process 3905 simply finishes at operation 3935, with nothing further for the clinician to do. In application sub-process 3910, clinician application 150 remains running in clinician workstation 105, so its quiescent state is waiting for the user's next selection at operation 3960. Similarly, clinician portal 205 has an established session for this user, with clinician application 150 connected and authenticated, so it too quiesces in an active state waiting for the next application request at operation 3975. Any of the other clinician methods may be selected in this state.

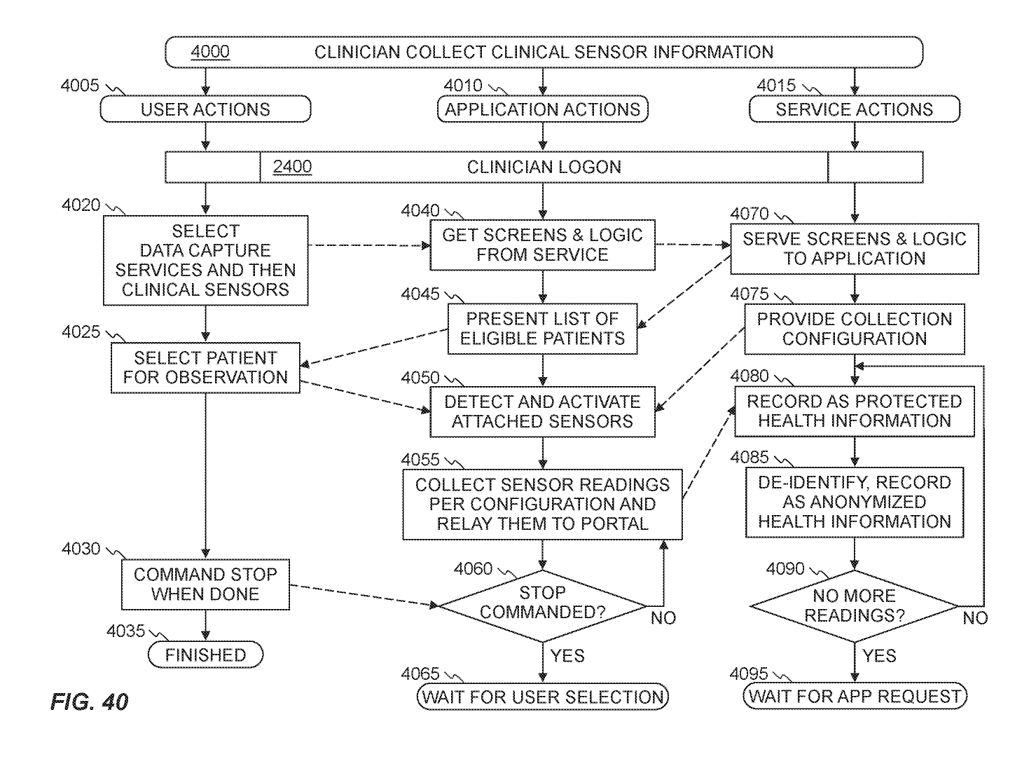

A clinician participating in a trial may have one or more relevant clinical sensor(s) 155, which may be attached to or embedded in clinician workstation 105 for use with visiting patients. Depending on the trial design as established by a coordinator, collection of data from clinical sensor(s) 155 during a patient appointment may be enabled. Clinician collect clinical sensor information, process 4000 depicted in FIG. 40, supports this activity. Again, the three-actor model and temporal/informational interaction conventions apply. The sub-processes in this case are labeled user actions 4005, application actions 4010, and service actions 4015; the primary service actor is data capture services 255.

Clinician collect clinical sensor information process 4000 begins with the usual prerequisite of clinician logon process 2400, in which all three actors participate. Note that external notification mechanisms described in processes 7400 and 7450, respectively notifications relay service autonomous operation and external notifications gateway autonomous operation, may prompt a clinician who is not already logged on to do so. After that, at operation 4020 the clinician may select data capture services 255 and the appropriate service—clinical sensors in this case—from the menu of clinician portal services 250. This may include explicit selection of an action to open the sensor function, or it may occur automatically upon opening a notification that includes a reminder to take sensor readings for a particular patient. Either way, at operation 4040 clinician application 150 may get the corresponding additional screens and logic from clinician portal 205 if they are not already loaded, and clinician portal 205 would provide them at operation 4070. At operation 4045, clinician application 150 may present a list of patients under the clinician's care and who are eligible for clinical sensor data collection. If so, the clinician would at operation 4025 select the patient for whom sensor readings are to be collected. Alternatively, the appropriate patient may be selected automatically by clinician application 150 if it has calendar information indicating an appointment with that patient, thereby skipping operations 4045 and 4025.

Immediately after providing application screens and logic, clinician portal 205 data capture services 255 may at operation 4075 provide clinician application 150 a configuration for collecting sensor information. This configuration may include a list of sensors to read if available, along with a periodicity and duration to apply for each one so that readings are collected regularly over a span of time as specified by the trial design. Note that such sensor configuration may be provided after every clinician logon, not just the one included in this figure; this operation is not shown in the other processes described previously, in order to simplify their depiction since it is not pertinent to those processes. Clinician application 150 at operation 4050 uses the provided configuration to detect and activate the configured sensors, then commence reading and reporting.

Thus at operation 4055, clinician application 150 collects sensor readings according to the provided configuration, and provides them to clinician portal 205 and its data capture services 255. At operation 4060, clinician application 150 checks whether the clinician has at operation 4030 commanded that readings stop, which may occur at any time; if not, operation 4055 is repeated. The procedure may also be stopped after a period of time or number of repetitions in the collection configuration, although this is not depicted in the figure. In parallel, data capture services 255 at operation 4080 records each received sensor reading in operational databases 202, which stores it in protected health information 223. At operation 4085, operational databases 202 may also de-identify the reading and store it in anonymized health information 224. At operation 4090, data capture services 255 may detect whether the final reading has been collected, which may occur by interpreting a count or time in the collection configuration previously sent to clinician application 150, by noting an indication in a particular reading that it is the last one, or by having not received a reading for a certain duration. If more readings are expected, sub-process 4015 loops back to repeat operations 4080 and 4085 on a subsequent reading.

When one of the conditions for terminating this process occurs, clinician collect clinical sensor information process 4000 is complete, with each sub-process quiescing in its own way. User sub-process 4005 simply finishes at operation 4035, with nothing further for the clinician to do. In application sub-process 4010, clinician application 150 remains running in clinician workstation 105, so its quiescent state is waiting for the user's next selection at operation 4065. Similarly, clinician portal 205 has an established session for this user, with clinician application 150 connected and authenticated, so it too quiesces in an active state waiting for the next application request at operation 4095. Any of the other clinician methods may be selected in this state.

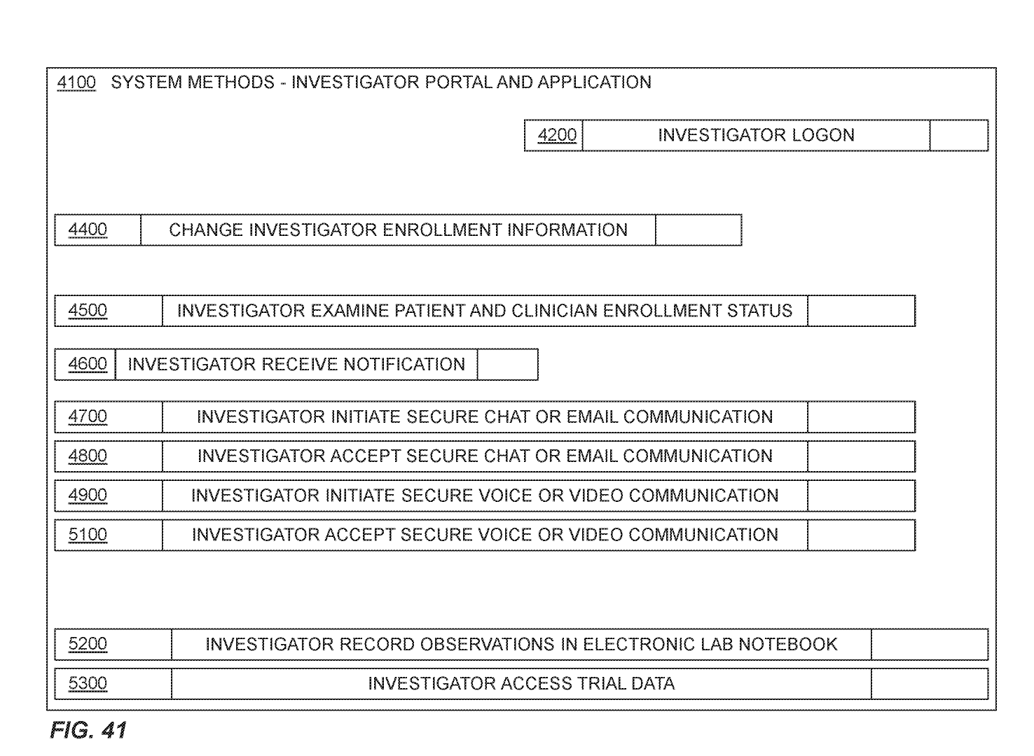

FIG. 41 illustrates a group of processes distributed among an investigator application, investigator portal, and other elements of a trial operations service suite in a clinical trial operations system according to an exemplary embodiment of the invention. The processes may include logon, personal data update, participant enrollment status monitoring, notification receipt, electronic lab notebook entry, trial data access, and secure communication. FIG. 42-FIG. 53 illustrate additional detail for each of the processes in FIG. 41.

Note that investigators and their contact information are known to the trial manager; they are not invited and they may not self-register, as patients and clinicians may. Therefore, investigator accounts are registered explicitly by a coordinator using process 5800 or process 5900, described later.

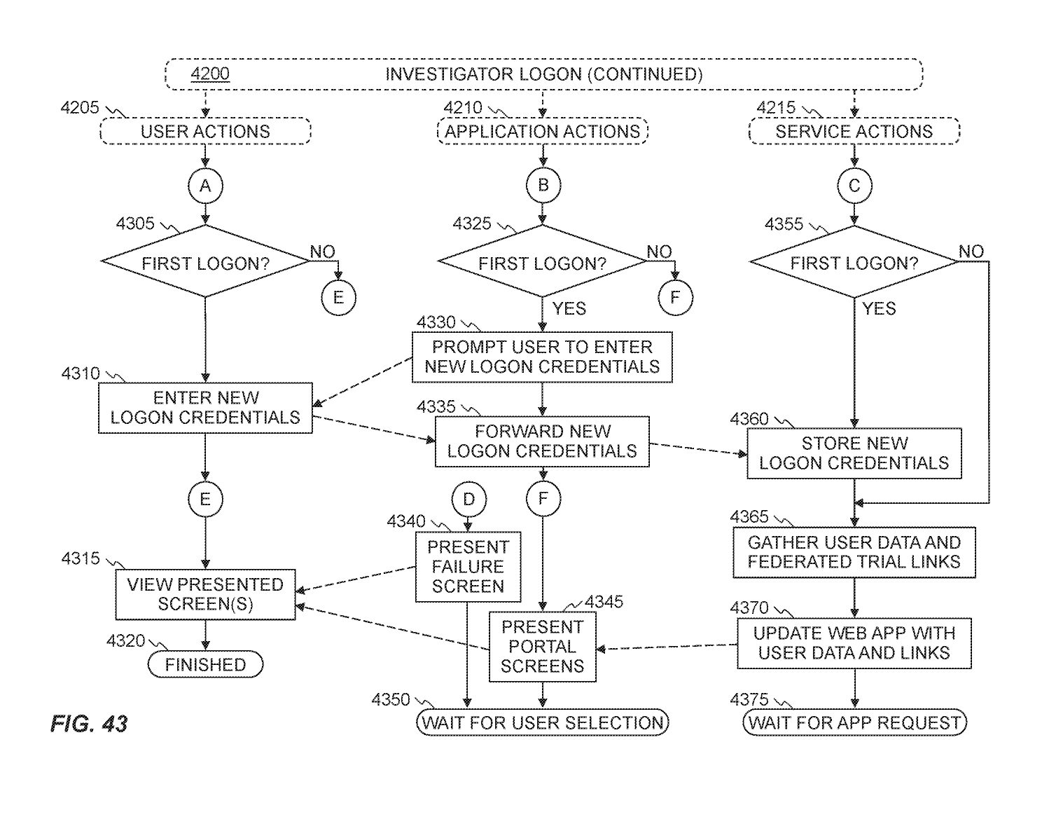

Investigator logon process 4200 is detailed in FIG. 42 and FIG. 43, and is a prerequisite for as well as an embedded operation in every other investigator process that follows. Three primary actors participate in this process: a user, which in this case is the investigator; an application, which in this case is the investigator application 160 web app; and a service, which in this case is the various elements of trial operations service suite 200, including in particular investigator portal 206. In the Figure, the operations performed by these actors are shown as part of interacting sub-processes: user actions 4205, application actions 4210, and service actions 4215. Solid arrows represent temporal ordering and information flow within a sub-process, and dashed arrows represent temporal ordering and information flow between sub-processes. Note that the sub-process flows cross from FIG. 42 to FIG. 43, via the connection points shown as labeled circles.

Investigator logon process 4200 begins at operation 4220, in which the user may activate a web browser installed in investigator workstation 106 and enter a URL to access investigator portal 206. At operation 4230, the web browser should access investigator portal 206 to download and execute the investigator application 160 web app. Investigator portal 206 facilitates this download at operation 4250, having been actively awaiting the access attempt via portals autonomous operation process 7200. Upon loading, at operation 4235 investigator application 160 prompts the user to enter logon credentials. The investigator enters credentials at operation 4225, and at operation 4240 investigator application 160 relays them to investigator portal 206 for verification and an appropriate response in operation 4255. If the credentials were not accepted, the decision at operation 4260 causes server sub-process 4215 to return to awaiting connection attempts. Similarly, application sub-process 4210 checks at operation 4245 whether the response indicated acceptance of the credentials. If not, investigator application 160 presents a failure notice to the user at operation 4340. If the credentials were accepted, each sub-process checks whether this is the user's first logon after registration or password reset, at operations 4325, 4305, and 4355. If so, investigator application 160 prompts the user at operation 4330 to enter new logon credentials at operation 4310, and relays the updated credentials at operation 4335 so that investigator portal 206 may store them in provisioning and authorization data 222 at operation 4360. Afterward, or if this is not the first logon, at operation 4365 investigator portal 206 may retrieve from identity federation gateway 289 a list of federated trial links, and gather from operational databases 202 relevant data to populate the user's portal view, then at operation 4370 update investigator application 160 with this information. Investigator application 160 would in turn at operation 4345 present this information structured as portal screens according to the trial design. The investigator then may view the presented screens, whether the failure notice of operation 4345 or the portal view of operation 4345, at operation 4315.

At this point, investigator logon process 4200 is complete, with each sub-process quiescing in its own way. User sub-process 4205 simply finishes at operation 4320, with nothing further for the investigator to do. In application sub-process 4210, investigator application 160 remains active in investigator workstation 106, so its quiescent state is waiting for the user's next selection at operation 4350. Similarly, investigator portal 206 has an established session for this user, with investigator application 160 connected and authenticated, so it too quiesces in an active state waiting for the next application request at operation 4375. Any of the other investigator methods may be selected in this state.

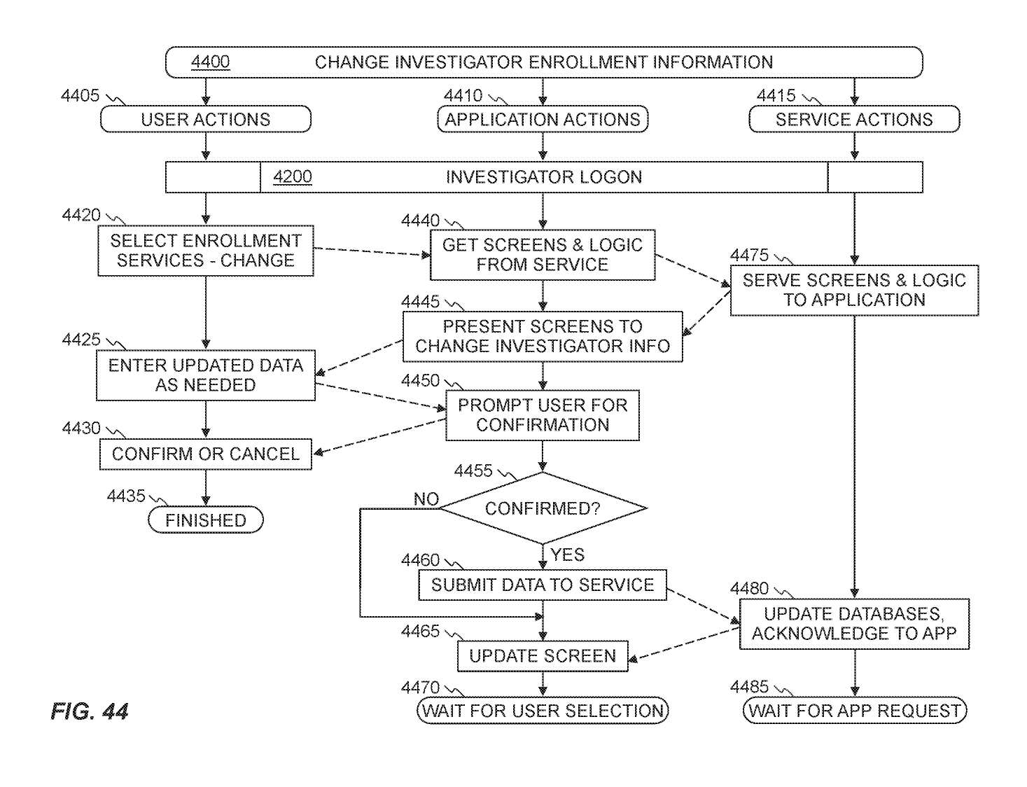

When an investigator needs to change enrollment information, such as contact address or other information, the change investigator enrollment information process 4400 may be used, as depicted in FIG. 44. The same three-actor model applies here, with the same temporal/informational interaction conventions. The sub-processes in this case are labeled user actions 4405, application actions 4410, and service actions 4415.

Change investigator enrollment information process 4400 begins with the prerequisite of investigator logon process 4200, in which all three actors participate. After that, at operation 4420 the investigator may select the change option under enrollment services 261 from the menu of investigator portal services 260. At operation 4440 investigator application 160 may get the corresponding additional screens and logic from investigator portal 206 if they are not already loaded, and investigator portal 206 would provide them at operation 4475. At operation 4445, then, the application presents the appropriate screens for changing enrollment information. Depending on the trial design established by a coordinator, the investigator may be presented one or more screens of options and data entry opportunities in order to update the information as needed, all embodied in operation 4425. This may take the form of several interactions between user and app, but in the end when all the changes have been made, investigator application 160 at operation 4450 may prompt the investigator for confirmation, and the investigator at operation 4430 may choose to confirm or cancel the changes. If investigator application 160 determines at operation 4455 that the investigator confirmed the changes, it may at operation 4460 submit the change package to investigator portal 206 enrollment services 261, which in turn at operation 4480 would update operational databases 202 with this new information about the investigator, specifically in provisioning and authorization data 222; investigators would have no protected health information in the system. When the data is safely stored, still within operation 4480, investigator portal 206 enrollment services 261 should provide an acknowledgement to investigator application 160 so it knows the change was successful. Confirmed or not, changed successfully or not, investigator application 160 updates the screen to reflect the appropriate state of affairs at operation 4465.

At this point, change investigator enrollment information process 4400 is complete, with each sub-process quiescing in its own way. User sub-process 4405 simply finishes at operation 4435, with nothing further for the investigator to do. In application sub-process 4410, investigator application 160 remains running in investigator workstation 106, so its quiescent state is waiting for the user's next selection at operation 4470. Similarly, investigator portal 206 has an established session for this user, with investigator application 160 connected and authenticated, so it too quiesces in an active state waiting for the next application request at operation 4485. Any of the other investigator methods may be selected in this state.

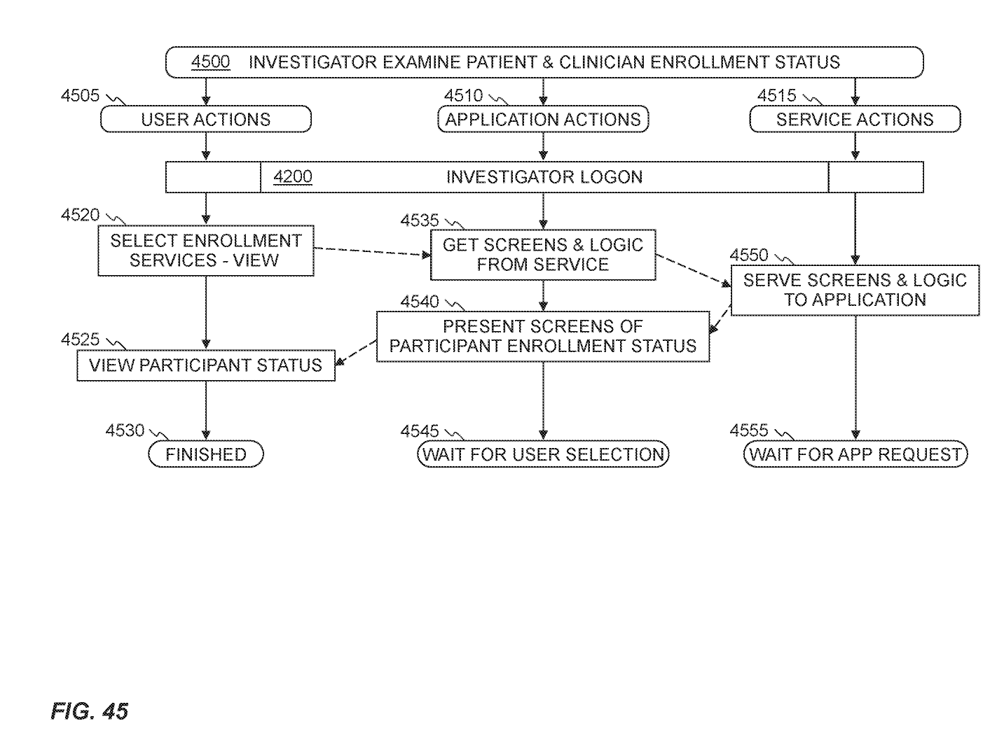

An investigator may need to check the status of patient and clinician enrollments in order to gauge progress toward meeting trial participation goals. The investigator examine patient & clinician enrollment status process 4500 may be used for this activity, as depicted in FIG. 45. The same three-actor model applies here, with the same temporal/informational interaction conventions. The sub-processes in this case are labeled user actions 4505, application actions 4510, and service actions 4515.

Investigator examine patient & clinician enrollment status process 4500 begins with the prerequisite of investigator logon process 4200, in which all three actors participate. After that, at operation 4520 the investigator may select the view participants option under enrollment services 261 from the menu of investigator portal services 260. At operation 4535 investigator application 160 may get the corresponding additional screens and logic from investigator portal 206 if they are not already loaded, and investigator portal 206 would provide them at operation 4550. At operation 4540, then, the application presents the appropriate screens for showing patient & clinician enrollment status, and the investigator may view this information at operation 4525. Depending on investigator preference settings, the default screen may be a list of patients and their statuses, with a summary showing the number of patients in each status, a list of clinicians and their statuses, along with a summary showing the number of patients in each status for each clinician, or some combination. Any format that is not automatically shown may be selected explicitly for display from a menu.

Note that all patient and clinician identifying information is removed by operational databases 202 when data is accessed by investigator portal 206, so that only anonymized (also known as de-identified) information is available.

At this point, investigator examine patient enrollment status process 4500 is complete, with each sub-process quiescing in its own way. User sub-process 4505 simply finishes at operation 4530, with nothing further for the investigator to do. In application sub-process 4510, investigator application 160 remains running in investigator workstation 106, so its quiescent state is waiting for the user's next selection at operation 4545. Similarly, investigator portal 206 has an established session for this user, with investigator application 160 connected and authenticated, so it too quiesces in an active state waiting for the next application request at operation 4555. Any of the other investigator methods may be selected in this state.

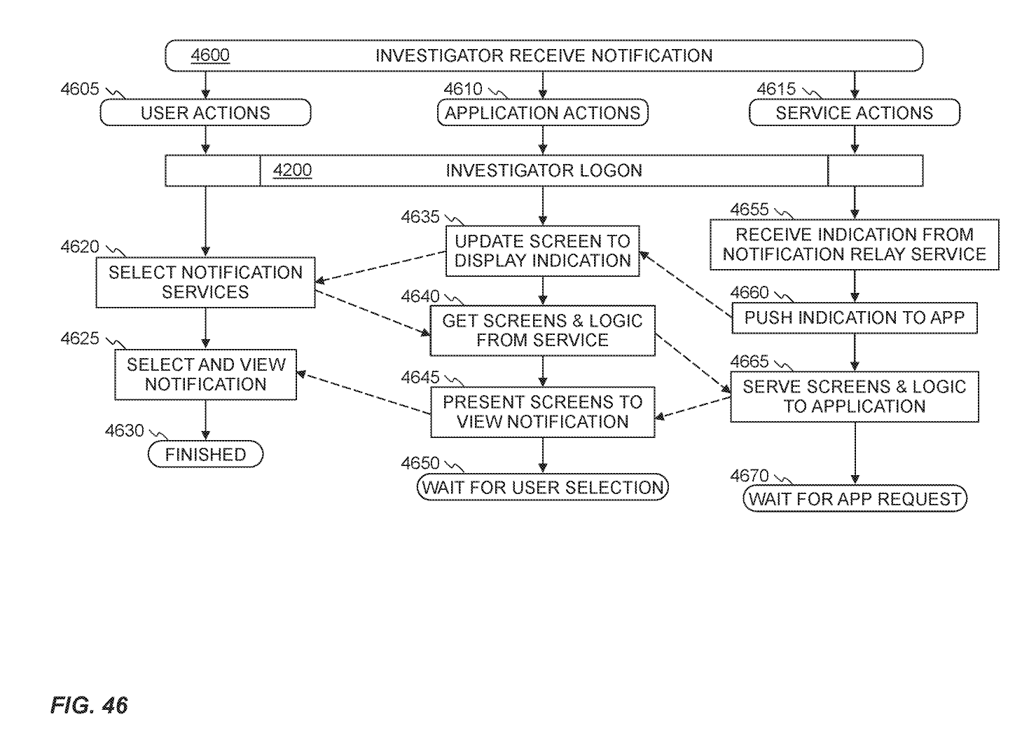

A coordinator may send investigators a notification conveying, for example, news about the trial; see the corresponding send notification process 6800 described later. Investigator receive notification process 4600, which is depicted in FIG. 46, enables the investigator to view such notifications. Once again, the three-actor model and temporal/informational interaction conventions apply. The sub-processes in this case are labeled user actions 4605, application actions 4610, and service actions 4615.

Investigator receive notification process 4600 begins with the prerequisite of investigator logon process 4200, in which all three actors participate. Note that external notification mechanisms described in processes 7400 and 7450, respectively notifications relay service autonomous operation and external notifications gateway autonomous operation, may prompt an investigator who is not already logged on to do so. Once investigator logon process 4200 has established a portal session for the investigator, whether prompted anew or already in place from previous activity, investigator portal 206 may at operation 4655 receive an indication of the notification from notification relay service 282, and then at operation 4660 push that indication to investigator application 160. Investigator application 160 in turn may at operation 4635 update whatever screen it is displaying to include a form of the indication observable by the user.

After that, at operation 4620 the investigator may select notification service 262 from the menu of investigator portal services 260. This may involve explicit selection of a command to view notifications, or simply touching or clicking on the indication itself. Either way, at operation 4640 investigator application 160 may get the corresponding additional screens and logic from investigator portal 206 if they are not already loaded, and investigator portal 206 would provide them at operation 4665. At operation 4645, then, the application presents the appropriate screens for viewing the notification to the user. Operation 4625 embodies the investigator's activity of viewing the presented notification, which may also include selecting a specific notification from a list of notifications that may have been sent to the investigator.

At this point, investigator receive notification process 4600 is complete, with each sub-process quiescing in its own way. User sub-process 4605 simply finishes at operation 4630, with nothing further for the investigator to do. In application sub-process 4610, investigator application 160 remains running in investigator workstation 106, so its quiescent state is waiting for the user's next selection at operation 4650. Similarly, investigator portal 206 has an established session for this user, with investigator application 160 connected and authenticated, so it too quiesces in an active state waiting for the next application request at operation 4670. Any of the other investigator methods may be selected in this state.

Trial operations service suite 200 may include support for communication between an investigator and other trial participants, potentially including patients, clinicians, other investigators, and coordinators. Which other participants an investigator is allowed to select for communication may be constrained by the trial design as established by a coordinator. For example, an investigator may be permitted to communicate clinicians but not patients, or an investigator may be permitted to communicate with other investigators in the trial. This capability, if permitted by the trial design, may enable a social networking aspect across the community of trial participants, thereby enhancing participants' engagement in the trial protocol. Four modes of communication may be supported, which are divided into two categories for the purpose of explaining their operation. Communication that is oriented toward exchanging written messages, documents, images, and other related information is categorized as messaging, and typified as chat mode or email mode. Communication that is oriented toward verbal or visual conversation over a live full-duplex connection is categorized as calling, and typified as voice mode or video mode. The processes supporting these capabilities are specified for each category, and within each category for initiating and accepting communications of the corresponding category, yielding four separate processes to be described hereinafter.

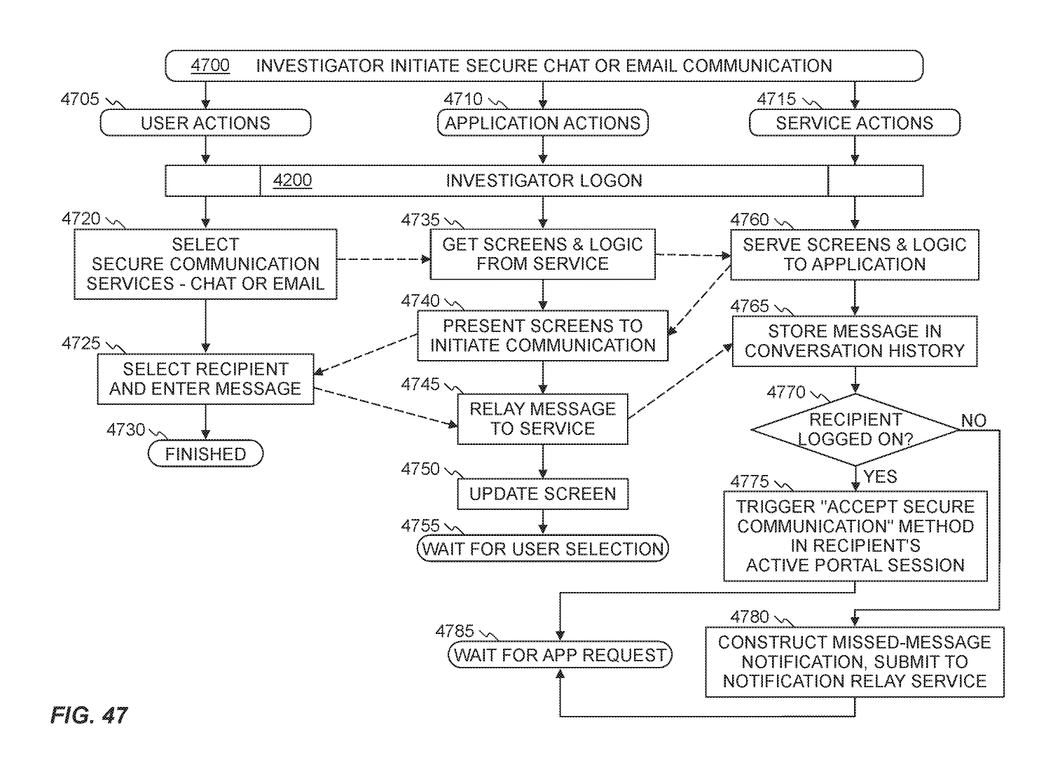

The first communication process is investigator initiate secure chat or email communication process 4700, which is depicted in FIG. 47. As usual, the three-actor model and temporal/informational interaction conventions apply. The sub-processes in this case are labeled user actions 4705, application actions 4710, and service actions 4715; the primary service actor is secure communication services 263, with help from secure communication relay services 281.

Investigator initiate secure chat or email communication process 4700 begins with the prerequisite of investigator logon process 4200, in which all three actors participate. After that, at operation 4720 the investigator may select secure communication services 263 and the appropriate communication mode—in this case either chat or email—from the menu of investigator portal services 260. At operation 4735 investigator application 160 may get the corresponding additional screens and logic from investigator portal 206 if they are not already loaded, and investigator portal 206 would provide them at operation 4760. At operation 4740, then, the application presents the appropriate screens for initiating communication. Depending on the trial design established by a coordinator, the investigator may select one or more recipients to which a chat or email message should be sent, with the selection mechanism and message entry embodied in operation 4725, whereupon investigator application 160 should at operation 4745 relay the entered message to secure communication services 263 of investigator portal 206, and at operation 4750 update the presented screen accordingly. Meanwhile, secure communication relay services 281 at operation 4765 may record the message in a conversation history associated with both the sending investigator and the recipient, which may preferably be stored in the notes, records, and events 225 section of operational databases 202. Note that while only one recipient is depicted in the Figure, this and following operations may be repeated for each of multiple recipients if the sender selected more than one. Then, at operation 4770 secure communication relay services 281 may determine whether the recipient is currently logged on in an active portal session. If so, at operation 4775 secure communication relay services 281 may trigger the recipient's accept secure chat or email communication process 1400, 3300, 4800, or 6300 depending on the recipient's role in the system, respectively a patient, a clinician, another investigator, or a coordinator. If the recipient does not have a current active portal session, then at operation 4780 secure communication relay services 281 may construct a missed-message notification and submit it to notifications relay service 282 so that it may be sent to the recipient's external contact address, facilitated by process 7400 notifications relay service autonomous operation and process 7450 external notifications gateway autonomous operation.

At this point, investigator initiate secure chat or email communication process 4700 is complete, with each sub-process quiescing in its own way. User sub-process 4705 simply finishes at operation 4730, with nothing further for the investigator to do. In application sub-process 4710, investigator application 160 remains running in investigator workstation 106, so its quiescent state is waiting for the user's next selection at operation 4755. Similarly, investigator portal 206 has an established session for this user, with investigator application 160 connected and authenticated, so it too quiesces in an active state waiting for the next application request at operation 4785. Any of the other investigator methods may be selected in this state.

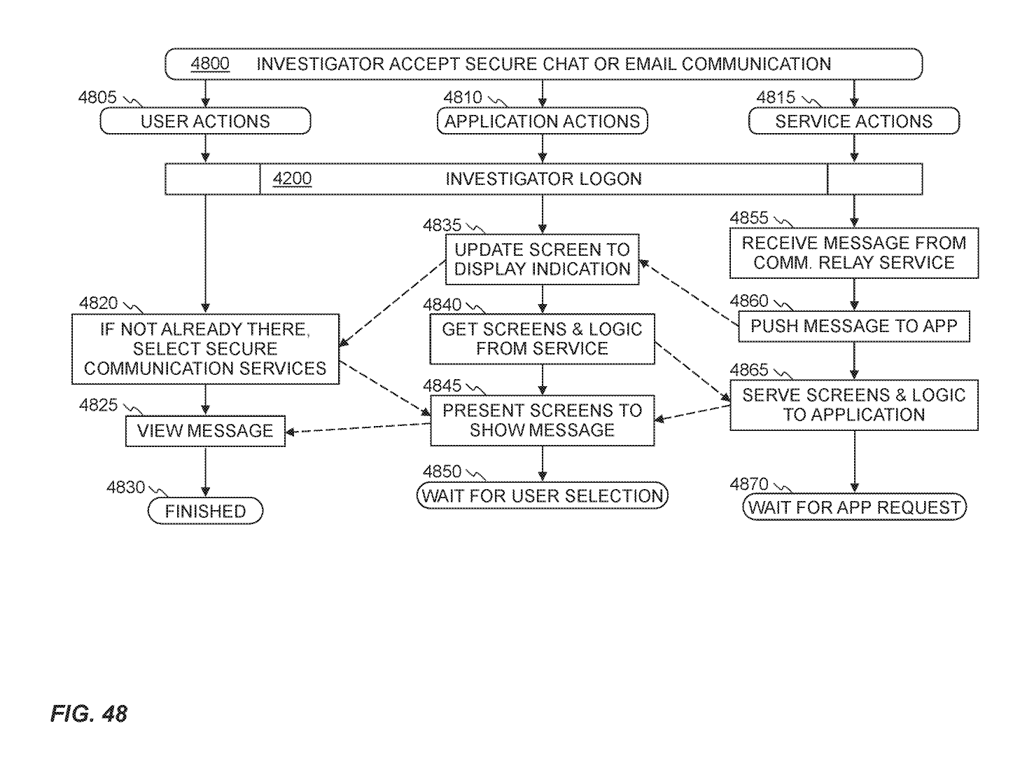

The next communication process is investigator accept secure chat or email communication process 4800, which is depicted in FIG. 48. As usual, the three-actor model and temporal/informational interaction conventions apply. The sub-processes in this case are labeled user actions 4805, application actions 4810, and service actions 4815; the primary service actor is secure communication services 263, with help from secure communication relay services 281.

Investigator accept secure chat or email communication process 4800 begins with the prerequisite of investigator logon process 4200, in which all three actors participate. Note that external notification mechanisms described in processes 7400 and 7450, respectively notifications relay service autonomous operation and external notifications gateway autonomous operation, may prompt an investigator who is not already logged on to do so. Once investigator logon process 4200 has established a portal session for the investigator, whether prompted anew or already in place from previous activity, at operation 4855 secure communication services 263 of investigator portal 206 may receive a copy of the message from secure communication relay services 281. After that, at operation 4860 the investigator portal 206 should push the message to investigator application 160, which would in turn at operation 4835 update whatever screen it is displaying to include an indication of the message's arrival. At operation 4840 investigator application 160 may predictively retrieve the corresponding additional screens and logic from investigator portal 206 if they are not already loaded, and investigator portal 206 would provide them at operation 4865.

When the investigator notices the indication thus displayed, at operation 4820 the investigator may select secure communication services 263 and the appropriate communication mode—in this case either chat or email—from the menu of investigator portal services 260. This may involve explicit selection of a command to enter the corresponding service, or simply touching or clicking on the indication itself; alternatively, investigator application 160 may switch to the corresponding service mode automatically, or already be in the corresponding service mode due to previous activity. However it arrived in the appropriate state, at operation 4845 investigator application 160 presents the appropriate screens for viewing the received message. Operation 4825 embodies the investigator's activity of viewing the presented message. If the communication mode is email, this may also include selecting a specific message from a list of messages that may have been sent to the investigator. If the communication mode is chat, this may also include viewing other messages both sent and received in the context of a conversation.

At this point, investigator accept secure chat or email communication process 4800 is complete, with each sub-process quiescing in its own way. User sub-process 4805 simply finishes at operation 4830, with nothing further for the investigator to do. In application sub-process 4810, investigator application 160 remains running in investigator workstation 106, so its quiescent state is waiting for the user's next selection at operation 4850. Similarly, investigator portal 206 has an established session for this user, with investigator application 160 connected and authenticated, so it too quiesces in an active state waiting for the next application request at operation 4870. Any of the other investigator methods may be selected in this state.

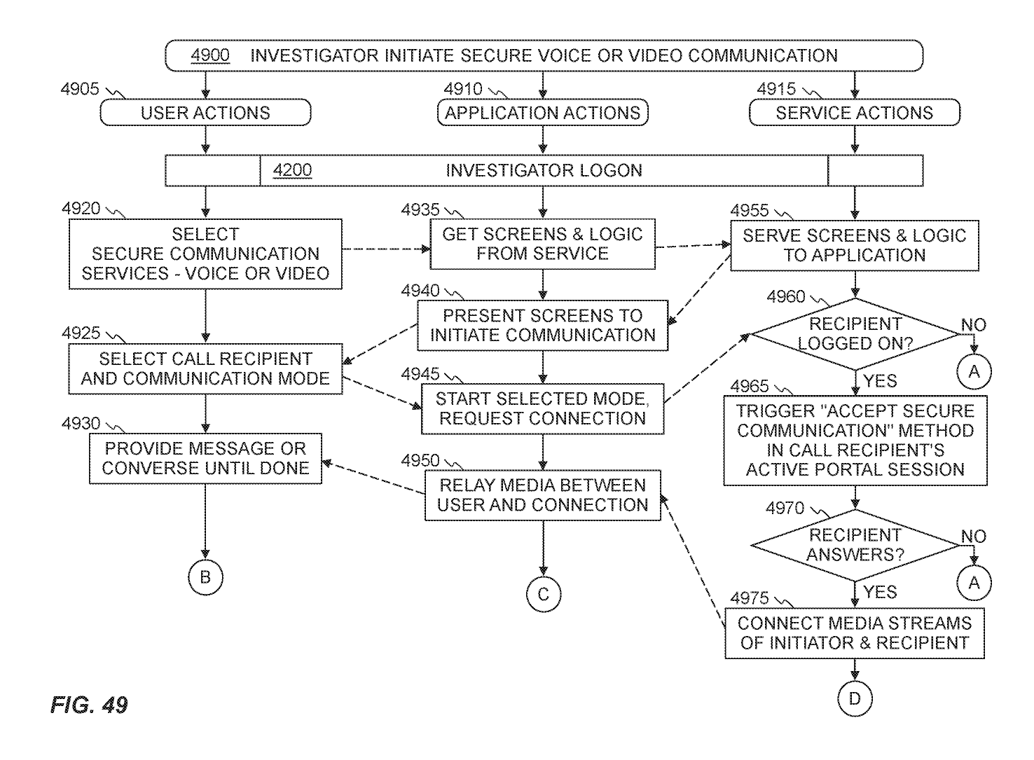

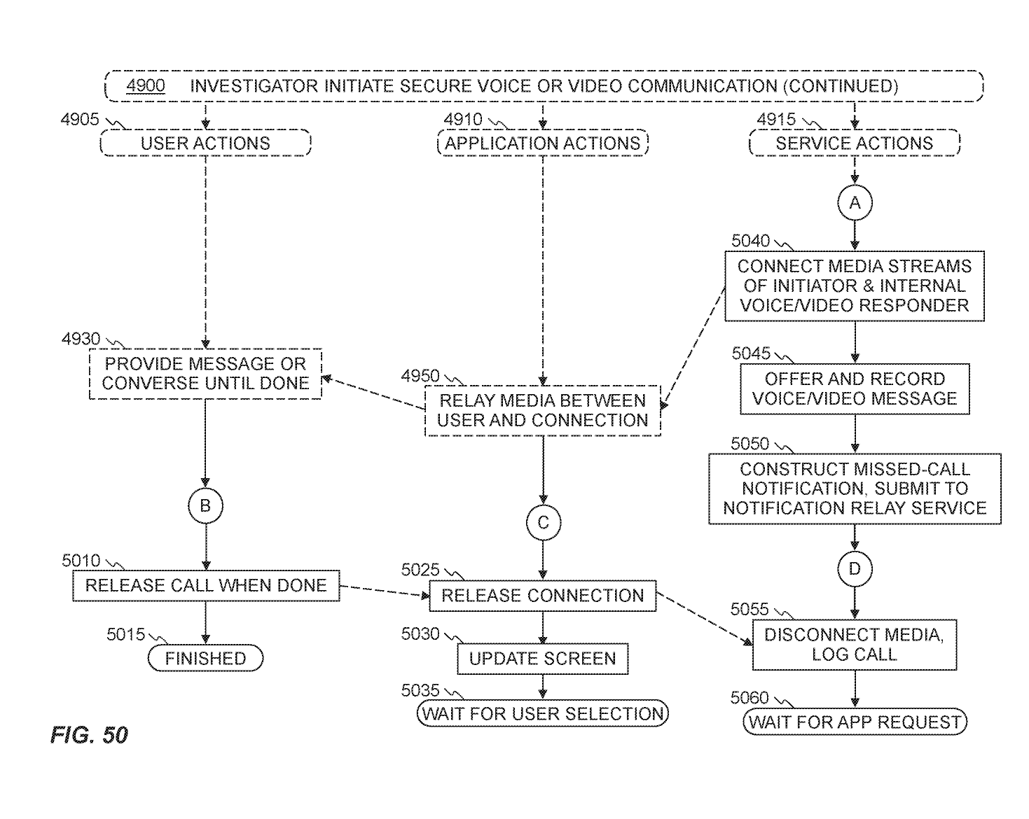

The next communication process is investigator initiate secure voice or video communication process 4900, which is depicted in FIG. 49 and FIG. 50. As usual, the three-actor model and temporal/informational interaction conventions apply. The sub-processes in this case are labeled user actions 4905, application actions 4910, and service actions 4915; the primary service actor is secure communication services 263, with help from secure communication relay services 281. Note that the sub-process flows cross from FIG. 49 to FIG. 50 via the connection points shown as labeled circles.

Investigator initiate secure voice or video communication process 4900 begins with the prerequisite of investigator logon process 4200, in which all three actors participate. After that, at operation 4920 the investigator may select secure communication services 263 and the appropriate communication mode—in this case either voice or video—from the menu of investigator portal services 260. At operation 4935 investigator application 160 may get the corresponding additional screens and logic from investigator portal 206 if they are not already loaded, and investigator portal 206 would provide them at operation 4955. At operation 4940, then, the application presents the appropriate screens for initiating communication. Depending on the trial design established by a coordinator, the investigator may select another trial participant with which a voice or video call should be placed, with the selection mechanism and message entry embodied in operation 4925, whereupon investigator application 160 should at operation 4945 start the selected communication mode and request secure communication services 263 to have secure communication relay services 281 make a connection supporting the call. At operation 4960 secure communication relay services 281 may determine whether the called participant, referred to as recipient in the Figure, is currently logged on in an active portal session. If so, at operation 4965 secure communication relay services 281 may trigger the recipient's accept secure voice or video communication process 1700, 3600, 5100, or 6600 depending on the recipient's role in the system, respectively a patient, a clinician, another investigator, or a coordinator. During that process, the recipient may choose to accept (answer), reject, or ignore the call. At operation 4970, secure communication relay services 281 determines this outcome. If the recipient answers, at operation 4975 secure communication services 263 and secure communication relay services 281 connect the media streams of the initiator and the recipient so that they may converse.

Whether the call is answered by the recipient or the message recorder, at operation 4950 investigator application 160 relays the media streams between the calling user and the connection, so that the investigator may at operation 4930 either converse with the called participant or leave a voice or video message, as appropriate. Note that these two operations appear on both FIG. 49 and FIG. 50, and represent the same actions in both. When the investigator is finished conversing with or providing a voice/video message for the recipient, he or she will release the call at operation 5010. This may take the form of clicking or touching a hang-up control on the screen of, or pressing a button on, investigator workstation 106.

If the recipient does not have a current active portal session (operation 4960), or if the recipient does not answer the call (operation 4970), then at operation 5040 secure communication services 263 may connect the initiator's media streams to those of an internal voice or video responder—effectively an answering machine—provided by secure communication relay services 281, which at operation 5045 may offer prompts to record a voice or video message and then do so if one is provided by the initiator. After the voice or video message is finished, which may be determined by detecting a configured period of silence, or by detecting an explicit indication from the calling user such as a key word, tone, or disconnect, or if the caller chooses not to leave a message at all, at operation 5050 the secure communication relay services 281 may construct a missed-call notification and submit it to notifications relay service 282 so that it may be sent to the recipient's external contact address, facilitated by process 7400 notifications relay service autonomous operation and process 7450 external notifications gateway autonomous operation.

Whether the call is answered by the recipient or the message recorder, at operation 4950 investigator application 160 relays the media streams between the calling user and the connection, so that the investigator may at operation 4930 either converse with the called participant or leave a voice or video message, as appropriate. Note that these two operations appear on both FIG. 49 and FIG. 50 and represent the same actions in both. When the investigator is finished conversing with or providing a voice/video message for the recipient, he or she will release the call at operation 5010. This may take the form of clicking or touching a hang-up control on the screen of, or pressing a button on, investigator workstation 106. Alternatively, investigator application 160, secure communication services 263, or secure communication relay services 281 may detect a predetermined spoken command or visible gesture from the investigator as indicating the call is over, or detect that a disconnect indication of some sort was detected on the other side of the call. However call termination may be detected, investigator application 160 will at operation 5025 release the connection and at operation 5030 update the screen to reflect this change of state. At operation 5055 secure communication relay services 281 will disconnect the media streams between the initiator's and recipient's portal sessions, and record the event in a conversation history associated with both the calling investigator and the recipient, which may preferably be stored in the notes, records, and events 225 section of operational databases 202.

At this point, investigator initiate secure voice or video communication process 4900 is complete, with each sub-process quiescing in its own way. User sub-process 4905 simply finishes at operation 5015, with nothing further for the investigator to do. In application sub-process 4910, investigator application 160 remains running in investigator workstation 106, so its quiescent state is waiting for the user's next selection at operation 5035. Similarly, investigator portal 206 has an established session for this user, with investigator application 160 connected and authenticated, so it too quiesces in an active state waiting for the next application request at operation 5060. Any of the other investigator methods may be selected in this state.

Note that investigator initiate secure voice or video communication process 4900 may also be used to listen to a voice message or view a video message left by another participant when a call from that other participant was missed by the investigator. The details of this usage are not depicted, but they are effectively identical if the “answering machine” function of secure communication relay services 281 is considered the call recipient and the investigator's interactions with that function are considered the conversation.

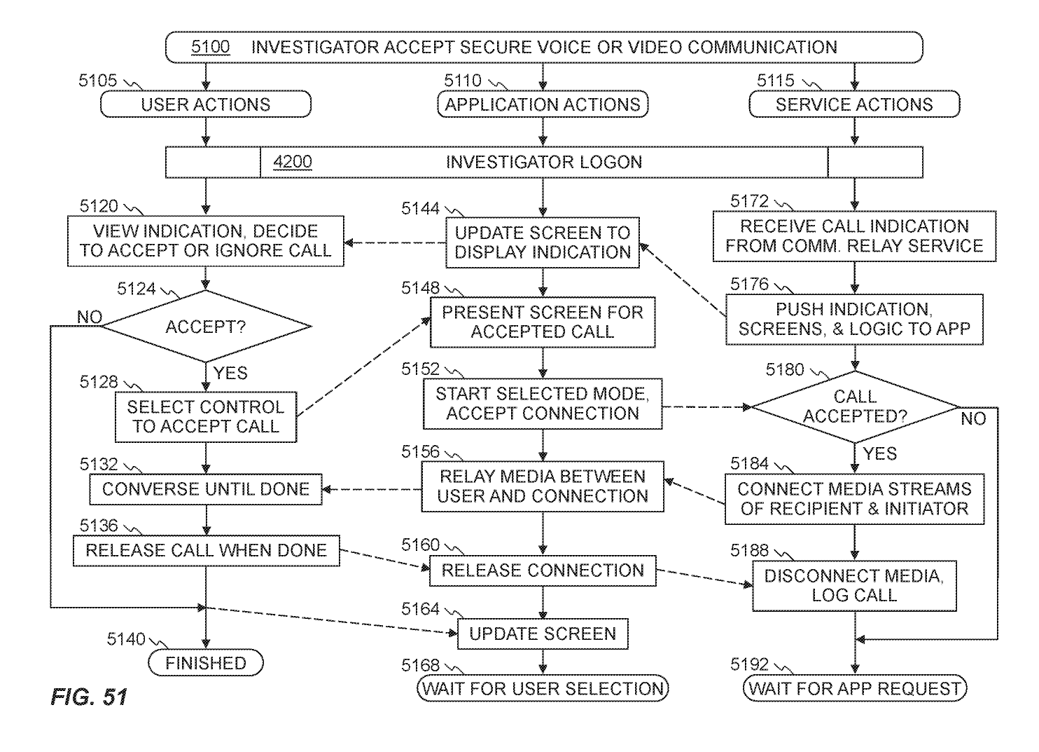

The fourth communication process is investigator accept secure voice or video communication process 5100, which is depicted in FIG. 51. Again, the three-actor model and temporal/informational interaction conventions apply. The sub-processes in this case are labeled user actions 5105, application actions 5110, and service actions 5115; the primary service actor is secure communication services 263, with help from secure communication relay services 281.

Investigator accept secure voice or video communication process 5100 begins with the prerequisite of investigator logon process 4200, in which all three actors participate. Note that for a voice or video call, this will generally have already occurred such that the portal session is currently active; otherwise the called investigator would not have an opportunity to accept the call while it is being offered, and it would have turned into a missed call. Thus in this scenario, right away at operation 5172 secure communication services 263 of investigator portal 206 should receive an indication of the offered call from secure communication relay services 281, and at operation 5176 investigator portal 206 should push that indication, as well as screens and logic for accepting a voice or video call, to investigator application 160. Investigator application 160 would in turn at operation 5144 update whatever screen it is displaying to include an indication of the incoming call.

When the investigator notices, at operation 5120, the indication thus displayed, he or she may choose at operation 5124 to accept or ignore the call. If the decision is to accept, the investigator at operation 5128 may select the necessary control to do so. This may involve explicit selection of a menu command to enter secure communication services 263 and the appropriate communication mode—in this case either voice or video. Preferably, though, it would involve simply touching or clicking on the indication itself, causing investigator application 160 to switch to the corresponding service mode automatically. However it arrived in the appropriate state, at operation 5148 investigator application 160 presents the appropriate screen for having accepted the call, and at operation 5152 it starts the selected mode and completes the connection. At operation 5180, secure communication services 263 and secure communication relay services 281 also may determine whether the investigator accepted the call, and if so proceed at operation 5184 to connect the media streams of the recipient and initiator, while investigator application 160 at operation 5156 relays the media streams between the investigator and the connection, so that the investigator may at operation 5132 converse with the caller. When the investigator is finished conversing with the caller, he or she will release the call at operation 5136. This may take the form of clicking or touching a hang-up control on the screen of, or pressing a button on, investigator workstation 106. Alternatively, investigator application 160, secure communication services 263, or secure communication relay services 281 may detect a predetermined spoken command or visible gesture from the investigator as indicating the call is over, or detect that a disconnect indication of some sort was detected on the other side of the call. However call termination may be detected, investigator application 160 will at operation 5160 release the connection and at operation 5164 update the screen to reflect this change of state. At operation 5188 secure communication services 263 will disconnect the media streams between the initiator's and recipient's portal sessions, and record the event in a conversation history associated with both the calling investigator and the recipient, which may preferably be stored in the notes, records, and events 225 section of operational databases 202.

If the investigator ignores or rejects the calls, this is detected at operations 5124 and 5180, and the process skips to the end, bypassing all the other operations except 5164 in which investigator application 160 updates the screen to reflect the corresponding state change.

Whether answered and disconnected, or ignored, at this point investigator accept secure voice or video communication process 5100 is complete, with each sub-process quiescing in its own way. User sub-process 5105 simply finishes at operation 5140, with nothing further for the investigator to do. In application sub-process 5110, investigator application 160 remains running in investigator workstation 106, so its quiescent state is waiting for the user's next selection at operation 5168. Similarly, investigator portal 206 has an established session for this user, with investigator application 160 connected and authenticated, so it too quiesces in an active state waiting for the next application request at operation 5192. Any of the other investigator methods may be selected in this state.

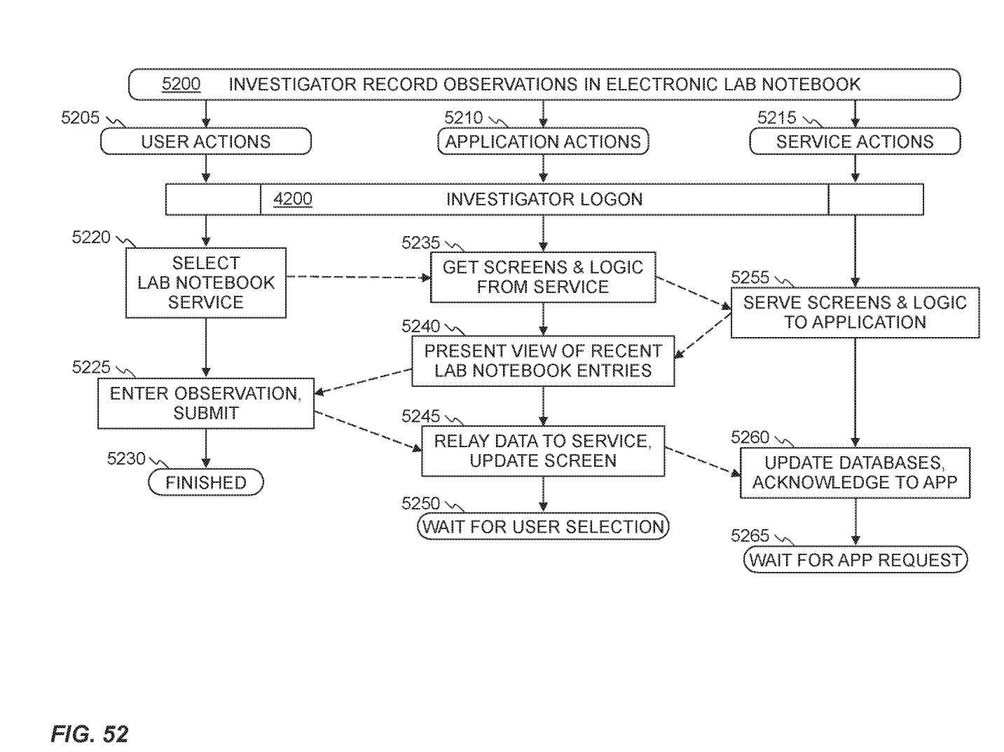

An investigator participating in a trial may be expected to record observations, deductions, and analyses in an auditable medium, such as the electronic lab notebook embodied in lab notebook service 264. Investigator record observations in electronic lab notebook, process 5200 depicted in FIG. 52, supports this activity. As usual, the same three-actor model and temporal/informational interaction conventions used throughout this specification apply. The sub-processes in this case are labeled user actions 5205, application actions 5210, and service actions 5215; the primary service actor is lab notebook service 264.

Investigator record observations in electronic lab notebook process 5200 begins with the usual prerequisite of investigator logon process 4200, in which all three actors participate. After that, at operation 5220 the investigator may select lab notebook service 264 from the menu of investigator portal services 260. At operation 5235 investigator application 160 may get the corresponding additional screens and logic from investigator portal 206 if they are not already loaded, and investigator portal 206 would provide them at operation 5255. At operation 5240, investigator application 160 may present the appropriate screens for making an entry in the electronic lab notebook, which may include information from other recent entries to help the investigator complete the new entry. Depending on the trial design established by a coordinator, the investigator may be presented a number of screens and options and data entry opportunities in order to enter the observation. Operation 5225 embodies the investigator's activity in this regard, which may take the form of several interactions between user and app. In the end when the entry has been completed and submitted, investigator application 160 at operation 5245 may submit the data package to lab notebook service 264, which in turn at operation 5260 would update operational databases 202 with the new information. In general, this information may be recorded in a subsection (not explicitly depicted in FIG. 2) of anonymized health information 224, since it may have been derived primarily from that database, although some may also be eligible for storage in notes, records, and events not classified as health information 225. The specific storage location for a particular entry may be selected explicitly by the investigator, or filters in operational databases 202 or lab notebook service 264 may detect and apply the appropriate treatment automatically. When all the data is safely stored, still within operation 5260, lab notebook service 264 should provide an acknowledgement to investigator application 160 so it may update the screen accordingly.

At this point, investigator record observations in electronic lab notebook process 5200 is complete, with each sub-process quiescing in its own way. User sub-process 5205 simply finishes at operation 5230, with nothing further for the investigator to do. In application sub-process 5210, investigator application 160 remains running in investigator workstation 106, so its quiescent state is waiting for the user's next selection at operation 5250. Similarly, investigator portal 206 has an established session for this user, with investigator application 160 connected and authenticated, so it too quiesces in an active state waiting for the next application request at operation 5265. Any of the other investigator methods may be selected in this state.

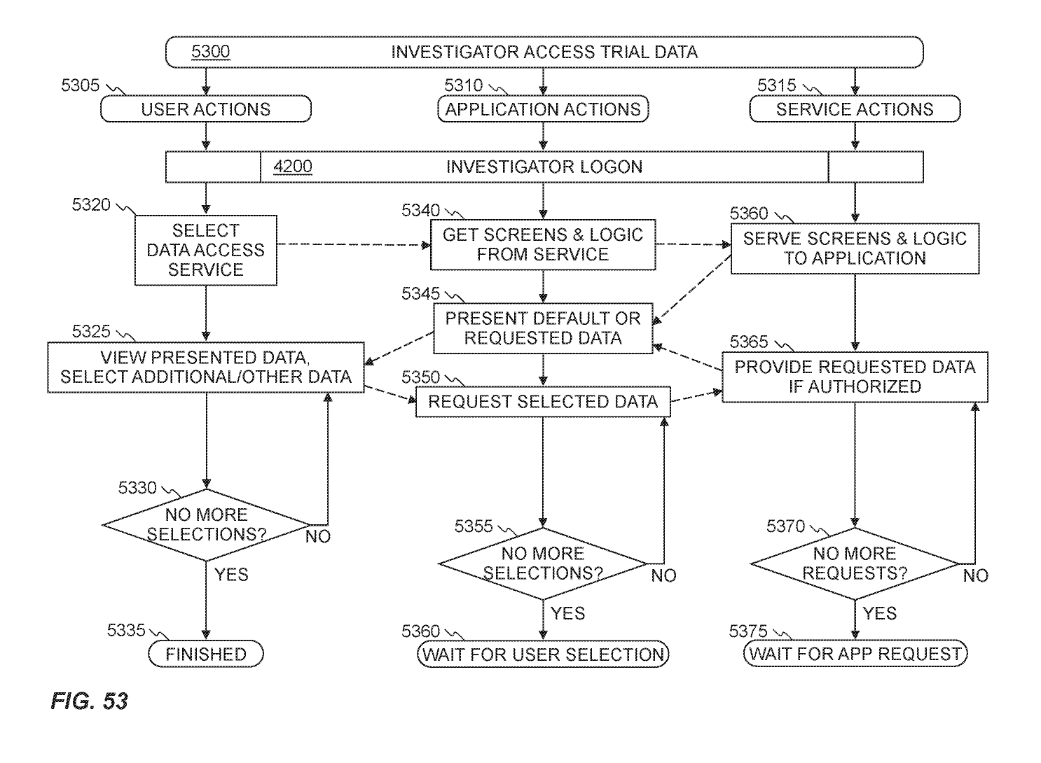

An investigator participating in a trial may be expected to access trial data such as anonymized health information 224 and notes, records, and events not classified as health information 225, in order to synthesize observations, deductions, and analyses that support the scientific process of the trial. Investigator access trial data, process 5300 depicted in FIG. 53, supports this activity. As usual, the same three-actor model and temporal/informational interaction conventions used throughout this specification apply. The sub-processes in this case are labeled user actions 5305, application actions 5310, and service actions 5315; the primary service actor is data access service 265.

Investigator access trial data process 5300 begins with the usual prerequisite of investigator logon process 4200, in which all three actors participate. After that, at operation 5320 the investigator may select data access service 265 from the menu of investigator portal services 260. At operation 5335 investigator application 160 may get the corresponding additional screens and logic from investigator portal 206 if they are not already loaded, and investigator portal 206 would provide them at operation 5360. At operation 5340, investigator application 160 may present the results of a preconfigured or default database query, which may also be the same data being viewed at the end of a previous portal session. At operation 5320, the investigator may view the presented data, and may select additional or other data to request. The user interface mechanism for presenting data may take any form appropriate to the nature of the data itself, including formats commonly used in data analysis tools such as tables, graphs, spreadsheets, and others. The user interface mechanism for selecting data may also take any form appropriate to the nature of the data, including forms commonly used in data analysis tools such as column or row filters for selection, criteria checkboxes, textual database query languages such as SQL, and others. When a data selection has been formulated and submitted, investigator application 160 at operation 5345 may request the data from data access service 265, which in turn at operation 5365 would provide the requested data back to investigator application 160 if the investigator is authorized to see the requested data. Note that all health information is de-identified—that is, patient and clinician identifying information is removed—for all data presented by investigator portal 206. Presentation at operation 5340 and viewing at operation 5320 would then be repeated. At operation 5325, the user would decide whether additional data should be selected, and if so return to operation 5320 to repeat the cycle. Similarly, investigator application 160 decides at operation 5350 whether the user has provided another selection, and data access service 265 decides at operation 5370 whether the application has requested another selection, and if so both sub-processes return to their respective operations 5345 and 5365 to handle them.

Once all three sub-processes have determined that there are no more selections, perhaps via an explicit indication from the user or via a portal session activity timeout, investigator access trial data process 5300 is complete, with each sub-process quiescing in its own way. User sub-process 5305 simply finishes at operation 5330, with nothing further for the investigator to do. In application sub-process 5310, investigator application 160 remains running in investigator workstation 106, so its quiescent state is waiting for the user's next selection at operation 5355. Similarly, investigator portal 206 has an established session for this user, with investigator application 160 connected and authenticated, so it too quiesces in an active state waiting for the next application request at operation 5375. Any of the other investigator methods may be selected in this state.

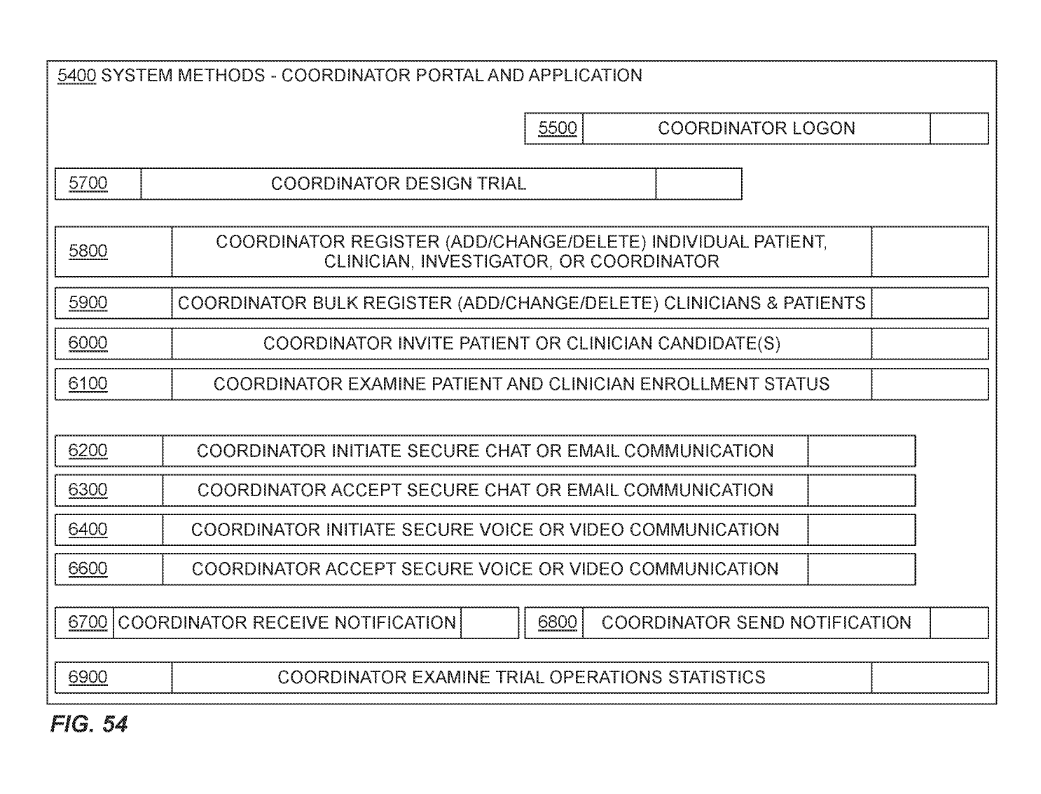

FIG. 54 illustrates a group of processes distributed among a coordinator application, coordinator portal, and other elements of a trial operations service suite in a clinical trial operations system according to an exemplary embodiment of the invention. The processes may include logon, trial design, participant enrollment status monitoring, individual and bulk user registration, participant invitations, secure communication, notification sending, notification receipt, and operational data monitoring. FIG. 55-FIG. 69 illustrate additional detail for each of the processes in FIG. 54.

Note that coordinators and their contact information are known to the trial manager; they are not invited and they may not self-register, as patients and clinicians may. Therefore, coordinator accounts are registered explicitly by a coordinator using process 5800 or process 5900, described later. Note also that a single default coordinator account is preconfigured in each trial operations service suite 200 in order to have a starting point for these processes. That user's credentials, including both logon name and password, are to be changed upon first logon as described in the following paragraphs.

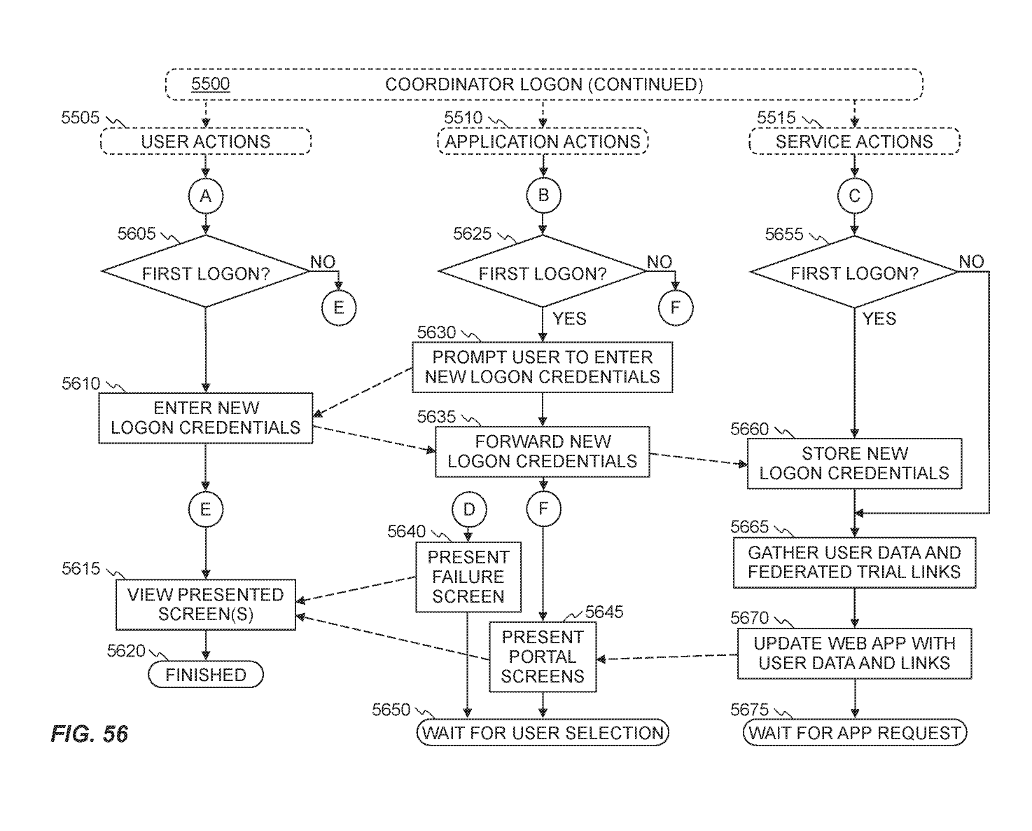

Coordinator logon process 5500 is detailed in FIG. 55 and FIG. 56 and is a prerequisite for as well as an embedded operation in every other coordinator process that follows. Three primary actors participate in this process: a user, which in this case is the coordinator; an application, which in this case is the coordinator application 170 web app; and a service, which in this case is the various elements of trial operations service suite 200, including in particular coordinator portal 207. In the Figure, the operations performed by these actors are shown as part of interacting sub-processes: user actions 5505, application actions 5510, and service actions 5515. Solid arrows represent temporal ordering and information flow within a sub-process, and dashed arrows represent temporal ordering and information flow between sub-processes. Note that the sub-process flows cross from FIG. 55 to FIG. 56 via the connection points shown as labeled circles.

Coordinator logon process 5500 begins at operation 5520, in which the user may activate a web browser installed in coordinator workstation 107 and enter a URL to access coordinator portal 207. At operation 5530, the web browser should access coordinator portal 207 to download and execute the coordinator application 170 web app. Coordinator portal 207 facilitates this download at operation 5550, having been actively awaiting the access attempt via portals autonomous operation process 7200. Upon loading, at operation 5535 coordinator application 170 prompts the user to enter logon credentials. The coordinator enters credentials at operation 5525, and at operation 5540 coordinator application 170 relays them to coordinator portal 207 for verification and an appropriate response in operation 5555. If the credentials were not accepted, the decision at operation 5560 causes server sub-process 5515 to return to awaiting connection attempts. Similarly, application sub-process 5510 checks at operation 5545 whether the response indicated acceptance of the credentials. If not, coordinator application 170 presents a failure notice to the user at operation 5640. If the credentials were accepted, each sub-process checks whether this is the user's first logon after registration or password reset, or whether it's the first logon to the default coordinator account, at operations 5625, 5605, and 5655. If so, coordinator application 170 prompts the user at operation 5630 to enter new logon credentials at operation 5610, and relays the updated credentials at operation 5635 so that coordinator portal 207 may store them in provisioning and authorization data 222 at operation 5660. Afterward, or if this is not the first logon, at operation 5665 coordinator portal 207 may retrieve from identity federation gateway 289 a list of federated trial links, and gather from operational databases 202 relevant data to populate the user's portal view, then at operation 5670 update coordinator application 170 with this information. Coordinator application 170 would in turn at operation 5645 present this information structured as portal screens according to the trial design. The coordinator then may view the presented screens, whether the failure notice of operation 5645 or the portal view of operation 5645, at operation 5615.

At this point, coordinator logon process 5500 is complete, with each sub-process quiescing in its own way. User sub-process 5505 simply finishes at operation 5620, with nothing further for the coordinator to do. In application sub-process 5510, coordinator application 170 remains active in coordinator workstation 107, so its quiescent state is waiting for the user's next selection at operation 5650. Similarly, coordinator portal 207 has an established session for this user, with coordinator application 170 connected and authenticated, so it too quiesces in an active state waiting for the next application request at operation 5675. Any of the other coordinator methods may be selected in this state.

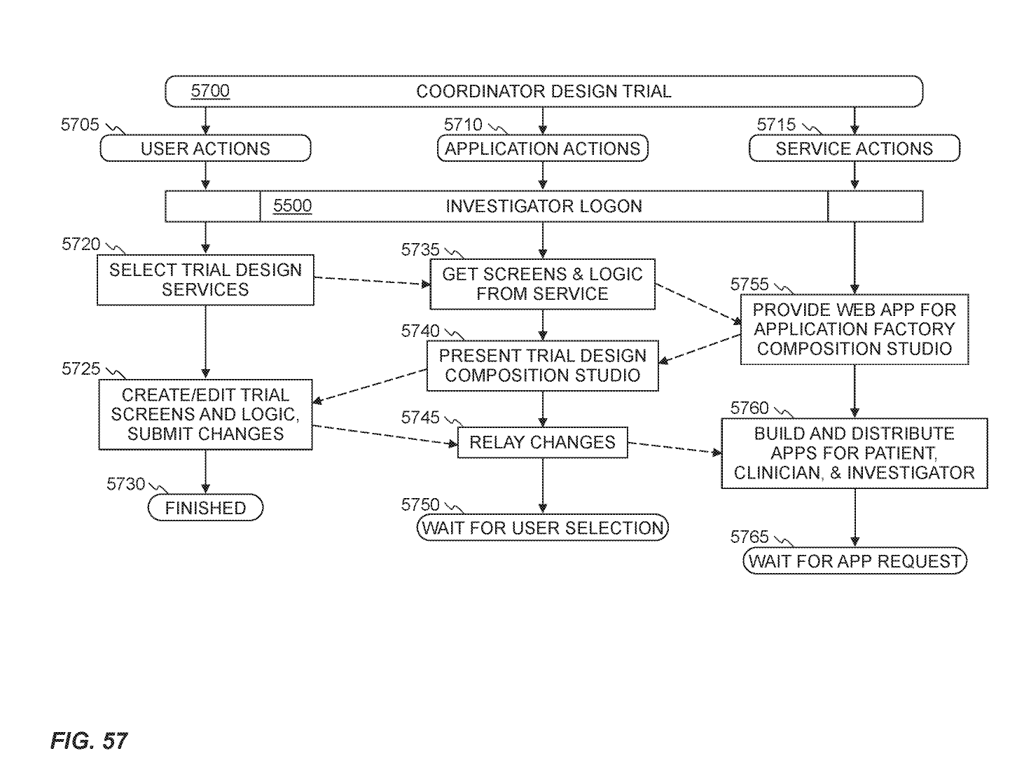

One of the most important activities a coordinator may perform is to design the aspects of participant qualification and consent, data collection, and participant interaction that are unique to the particular trial supported by its specific instance of trial operations service suite 200. Coordinator design trial, process 5700 depicted in FIG. 57, supports this activity. Once again, the three-actor model and temporal/informational interaction conventions used throughout this specification apply. The sub-processes in this case are labeled user actions 5705, application actions 5710, and service actions 5715; the primary service actor is trial design services 271.

Coordinator design trial process 5700 begins with the usual prerequisite of coordinator logon process 5500, in which all three actors participate. After that, at operation 5720 the coordinator may select trial design services 271 from the menu of coordinator portal services 270. At operation 5735 coordinator application 170 may get the corresponding additional screens and logic from coordinator portal 207, which would at operation 5755 provide the Composition Studio web app of the enhanced Application Factory System described previously. At operation 5740, coordinator application 170 may present the screens and controls constituting the user interface of said Composition Studio, enabling the coordinator at operation 5725 to create or edit screens and logic of the various aspects of the supported clinical trial as previously described in the context of FIG. 2 and FIG. 3 with respect to trial design services 271. To recap, such aspects may include without limitation the permissions and constraints for interaction, communication, and data access; database structures; look and feel as well as behaviors for applications 140, 150, 160, and even 170, along with their counterparts in portal services 240, 250, 260, and 270; participant qualification and consent protocols; structure, content, and frequency of surveys, queries, and notifications; multi-trial identity federation relationships; or any other trial-specific option including those cited in other sections throughout this specification. When the new or changed trial design is ready, the coordinator may submit the changes, at which point coordinator application 170 at operation 5745 may relay the changes to trial design services 271, which in turn at operation 5760 would build and distribute the various data structures, applications, portals, and service configuration settings that comprise the trial operations service suite 200, using build and distribution capabilities of the enhanced Application Factory System that is incorporated within trial design services 271. Note that the coordinator may also choose to create the design in pieces, saving intermediate or incomplete versions without building and distributing, and to test/verify aspects of the design prior to distribution, using corresponding capabilities of the enhanced Application Factory System that is incorporated within trial design services 271.

At this point, coordinator design trial process 5700 is complete, with each sub-process quiescing in its own way. User sub-process 5705 simply finishes at operation 5730, with nothing further for the coordinator to do. In application sub-process 5710, coordinator application 760 remains running in coordinator workstation 107, so its quiescent state is waiting for the user's next selection at operation 5750. Similarly, coordinator portal 207 has an established session for this user, with coordinator application 170 connected and authenticated, so it too quiesces in an active state waiting for the next application request at operation 5765. Any of the other coordinator methods may be selected in this state.

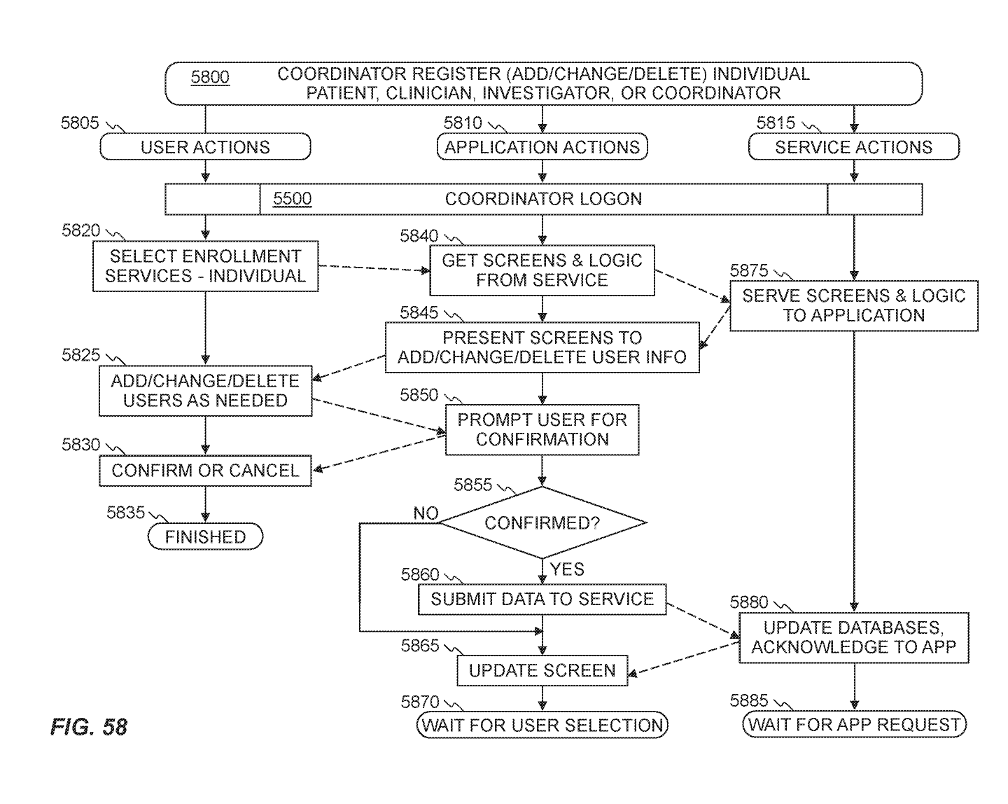

A coordinator may add, change, or delete registration information for a trial participant, if permitted. Attributes that a coordinator may be allowed to change include identifying, demographic, and contact information, as well as data access and communication permissions, but not protected health information. Note that at least one coordinator must always have this permission so that accounts for investigators and other coordinators can be managed; the default coordinator will start out with this permission, and may delegate it to one or more other coordinators after creating their accounts. Since patients and clinicians may self-register, it is not absolutely necessary for them to be managed this way, but depending on the specific requirements of a particular trial, it may be appropriate to pre-register them instead and manage them individually. A coordinator may also need to change his or her own contact or other information. The coordinator register (add/change/delete) individual patient, clinician, investigator, or coordinator process 5800 may be used for this, as depicted in FIG. 58. The three-actor model applies here, with the usual temporal/informational interaction conventions. The sub-processes in this case are labeled user actions 5805, application actions 5810, and service actions 5815; the primary service actor is enrollment services 272.

Coordinator register (add/change/delete) individual patient, clinician, investigator, or coordinator process 5800 begins with the prerequisite of coordinator logon process 5500, in which all three actors participate. After that, at operation 5820 the coordinator may select the individual option under enrollment services 272 from the menu of coordinator portal services 270. At operation 5840 coordinator application 170 may get the corresponding additional screens and logic from coordinator portal 207 if they are not already loaded, and coordinator portal 207 would provide them at operation 5875. At operation 5845, then, the application presents the appropriate screens for adding, deleting, or changing enrollment information about participants. Depending on the trial design, either the default approach or an updated one established by a coordinator using trial design services 271, the coordinator may be presented one or more screens of options and data entry opportunities in order to select the desired action, to select the appropriate user if the action is a change or deletion, and to update the information as needed, all embodied in operation 5825. This may take the form of several interactions between user and app, but in the end when all the changes have been made, coordinator application 170 at operation 5850 may prompt the coordinator for confirmation, and the coordinator at operation 5830 may choose to confirm or cancel the changes. If coordinator application 170 determines at operation 5855 that the coordinator confirmed the changes, it may at operation 5860 submit the change package to coordinator portal 207 enrollment services 272, which in turn at operation 5880 would update operational databases 202 with this new information about the participant, specifically in provisioning and authorization data 222; coordinators would have no ability to enter protected health information directly via this mechanism. When the data is safely stored, still within operation 5880, coordinator portal 207 enrollment services 272 should provide an acknowledgement to coordinator application 170 so it knows the change was successful. Confirmed or not, updated successfully or not, coordinator application 170 updates the screen to reflect the appropriate state of affairs at operation 5865.

At this point, coordinator register (add/change/delete) individual patient, clinician, investigator, or coordinator process 5800 is complete, with each sub-process quiescing in its own way. User sub-process 5805 simply finishes at operation 5835, with nothing further for the coordinator to do. In application sub-process 5810, coordinator application 170 remains running in coordinator workstation 107, so its quiescent state is waiting for the user's next selection at operation 5870. Similarly, coordinator portal 207 has an established session for this user, with coordinator application 170 connected and authenticated, so it too quiesces in an active state waiting for the next application request at operation 5885. Any of the other coordinator methods may be selected in this state.

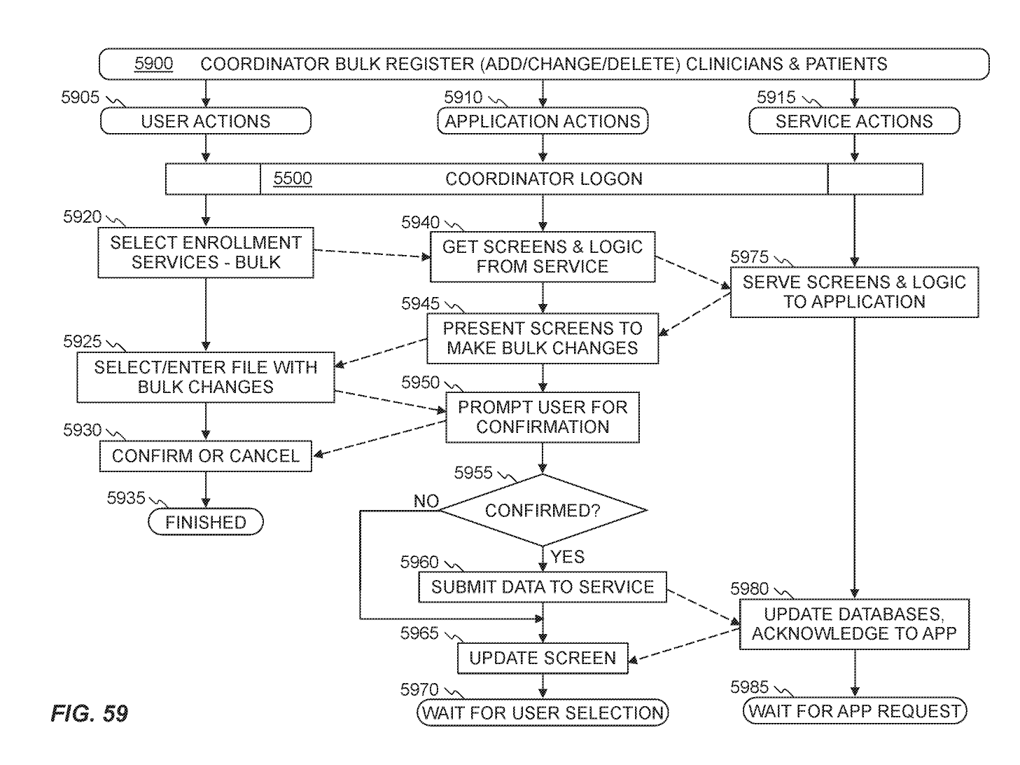

A coordinator may add, change, or delete registration information for multiple trial participants, if permitted. Attributes that a coordinator may be allowed to change include identifying, demographic, and contact information, as well as data access and communication permissions, but not protected health information. Since patients and clinicians may self-register, it is not absolutely necessary for them to be managed this way, but depending on the specific requirements of a particular trial, it may be appropriate to pre-register them in bulk instead or in addition. Updating registration data for multiple participants, also known as bulk registration, eases the burden of managing patient and clinician accounts individually if self-registration is not permitted for a particular trial. The coordinator bulk register (add/change/delete) clinicians and patients process 5900 may be used for this, as depicted in FIG. 59. The three-actor model applies here, with the usual temporal/informational interaction conventions. The sub-processes in this case are labeled user actions 5905, application actions 5910, and service actions 5915; the primary service actor is enrollment services 272.

Coordinator bulk register (add/change/delete) clinicians and patients process 5900 begins with the prerequisite of coordinator logon process 5500, in which all three actors participate. After that, at operation 5920 the coordinator may select the bulk option under enrollment services 272 from the menu of coordinator portal services 270. At operation 5940 coordinator application 170 may get the corresponding additional screens and logic from coordinator portal 207 if they are not already loaded, and coordinator portal 207 would provide them at operation 5975. At operation 5945, then, the application presents the appropriate screens for making bulk changes to clinician and patient registration information. Depending on the trial design, either the default approach or an updated one established by a coordinator using trial design services 271, the coordinator may be presented one or more screens of options and data entry opportunities in order to select a file containing individual add/delete/change actions for multiple patients and clinicians at operation 5925. This may take the form of several interactions between user and app, but in the end when the file has been selected, coordinator application 170 at operation 5950 may prompt the coordinator for confirmation, and the coordinator at operation 5930 may choose to confirm or cancel the changes. If coordinator application 170 determines at operation 5955 that the coordinator confirmed the changes, it may at operation 5960 submit the bulk change package to coordinator portal 207 enrollment services 272, which in turn at operation 5980 would process each add/change/delete action in the file and update operational databases 202 with the new participant registration information for each participant, specifically in provisioning and authorization data 222; coordinators would have no ability to enter protected health information directly via this mechanism. When the data is safely stored, still within operation 5980, coordinator portal 207 enrollment services 272 should provide an acknowledgement to coordinator application 170 so it knows the change was successful. Confirmed or not, updated successfully or not, coordinator application 170 updates the screen to reflect the appropriate state of affairs at operation 5965. Though not explicitly shown in the Figure, while processing the bulk registration file enrollment services 272 may provide coordinator application 170 with intermediate or partial acknowledgements that indicate progress, and coordinator application 170 may update the screen to reflect this state of affairs.

Once the final acknowledgement is delivered and reflected on screen, coordinator bulk register (add/change/delete) clinicians and patients process 5900 is complete, with each sub-process quiescing in its own way. User sub-process 5905 simply finishes at operation 5935, with nothing further for the coordinator to do. In application sub-process 5910, coordinator application 170 remains running in coordinator workstation 107, so its quiescent state is waiting for the user's next selection at operation 5970. Similarly, coordinator portal 207 has an established session for this user, with coordinator application 170 connected and authenticated, so it too quiesces in an active state waiting for the next application request at operation 5985. Any of the other coordinator methods may be selected in this state.

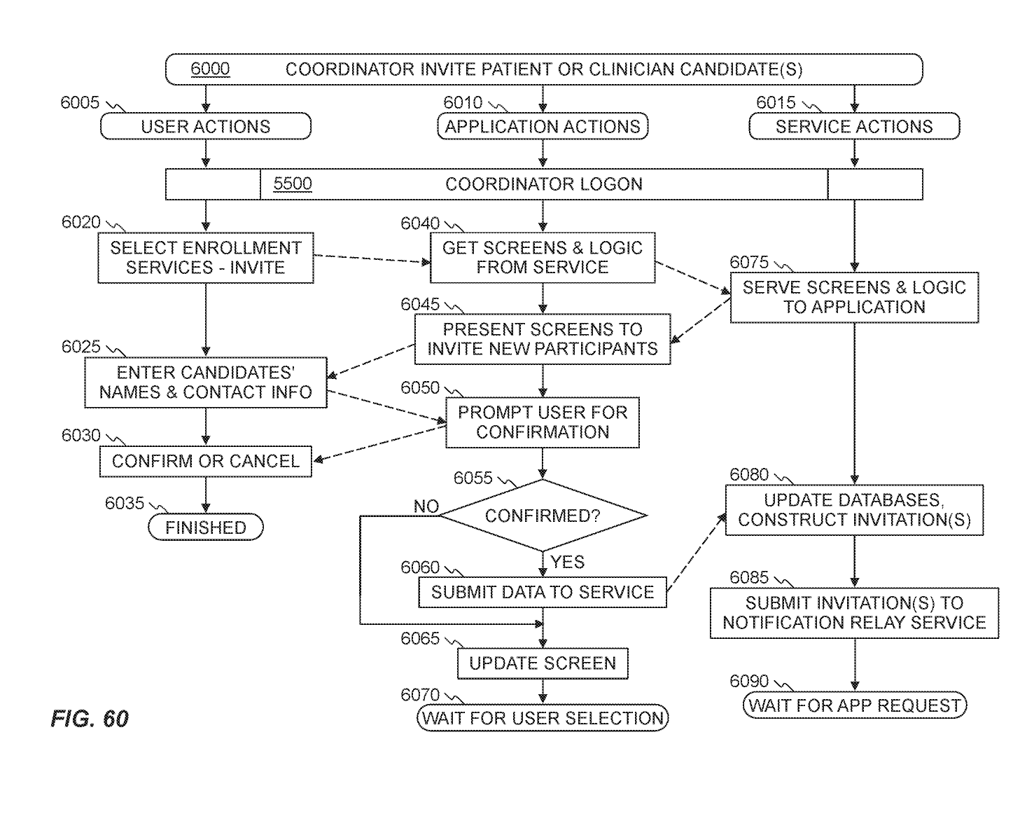

One of the ways a patient or clinician such as a doctor may learn about a particular trial and choose to self-register for it (process 600 for patients, process 2300 for clinicians) is by invitation from a coordinator, based on external information regarding potential suitability for the trial. This aspect of trial participation is facilitated by process 6000, coordinator invite patient or clinician candidate(s), which is depicted in FIG. 60. The three-actor model applies here, with the usual temporal/informational interaction conventions. The sub-processes in this case are labeled user actions 6005, application actions 6010, and service actions 6015; the primary service actor is enrollment services 272.

Coordinator invite patient or clinician candidate(s) process 6000 begins with the prerequisite of coordinator logon process 5500, in which all three actors participate. After that, at operation 6020 the coordinator may select the invite option under enrollment services 272 from the menu of coordinator portal services 270. At operation 6040 coordinator application 170 may get the corresponding additional screens and logic from coordinator portal 207 if they are not already loaded, and coordinator portal 207 would provide them at operation 6075. At operation 6045, then, the application presents the appropriate screens for inviting new participants. Depending on the trial design, either the default approach or an updated one established by a coordinator using trial design services 271, the coordinator may be presented one or more screens of options and data entry opportunities in order to provide name and contact information for the patient or clinician candidate or candidates to be invited, all embodied in operation 6025. This may take the form of several interactions between user and app, including for example entering each name/contact pair one by one, selecting a file containing several name/contact pairs, or selecting one or more candidates from a list of names or list of groups already registered using process 5800 or process 5900. When the information has been entered or selected, coordinator application 170 at operation 6050 may prompt the coordinator for confirmation, and the coordinator at operation 6030 may choose to confirm or cancel the invitation(s). If coordinator application 170 determines at operation 6055 that the coordinator confirmed the invitation(s), it may at operation 6060 submit the invitation package to coordinator portal 207 enrollment services 272, which in turn at operation 6080 would update operational databases 202 with this new information about the invitee(s), and construct the actual invitation message(s). In general, only provisioning and authorization data 222 would be created or altered at this operation, because the invitee(s) would have to provide the remaining enrollment data in their own respective executions of self-registration process 600/2300 and enrollment completion process 900/2600. When all the data is safely stored, coordinator portal 207 enrollment services 272 should submit, at operation 6085, each invitation separately to notification relay services 282 within trial operations service suite 200, in order to have the invitation(s) relayed externally; refer to process 7400, notifications relay service autonomous operation, for details of how this occurs. Finally, whether the invitation set was confirmed or not, coordinator application 170 may update the screen to reflect the appropriate state of affairs at operation 6065. Though not explicitly shown in the figure, while processing an invitation file enrollment services 272 may provide coordinator application 170 with intermediate or partial acknowledgements that indicate progress, and coordinator application 170 may update the screen to reflect this state of affairs.

At this point, coordinator invite patient or clinician candidate(s) process 6000 is complete, with each sub-process quiescing in its own way. User sub-process 6005 simply finishes at operation 6035, with nothing further for the coordinator to do. In application sub-process 6010, coordinator application 170 remains running in coordinator workstation 107, so its quiescent state is waiting for the user's next selection at operation 6070. Similarly, coordinator portal 207 has an established session for this user, with coordinator application 170 connected and authenticated, so it too quiesces in an active state waiting for the next application request at operation 6090. Any of the other coordinator methods may be selected in this state.

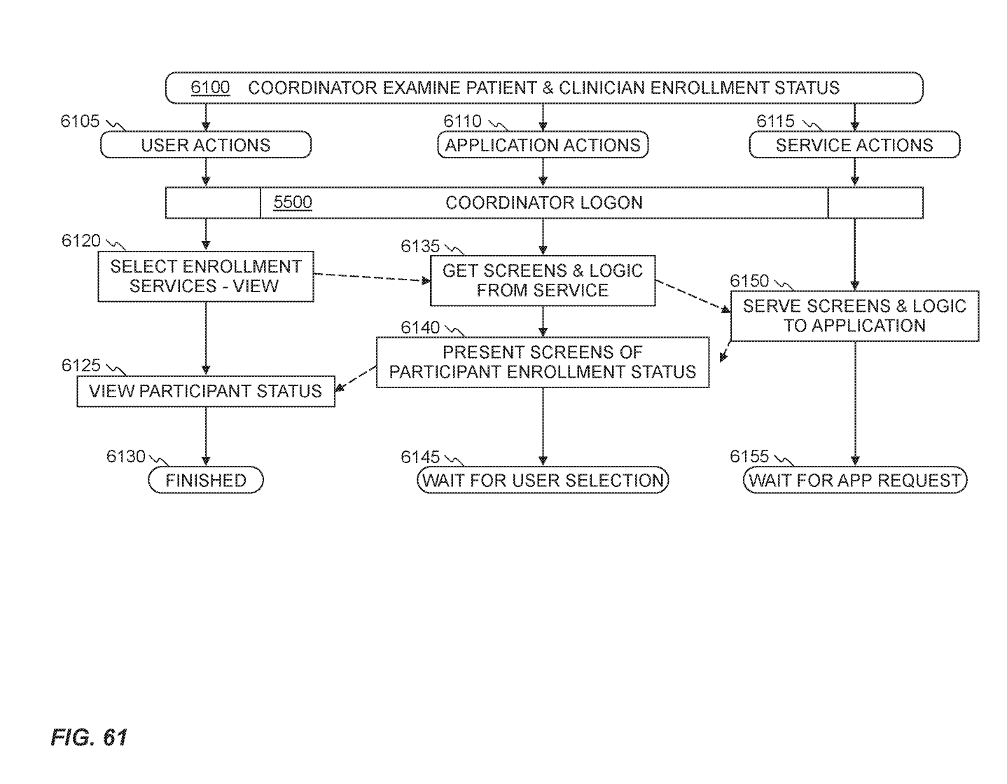

A coordinator may need to check the status of patient and clinician enrollments in order to gauge progress toward meeting trial participation goals. The coordinator examine patient & clinician enrollment status process 6100 may be used for this activity, as depicted in FIG. 61. The same three-actor model applies here, with the same temporal/informational interaction conventions. The sub-processes in this case are labeled user actions 6105, application actions 6110, and service actions 6115.

Coordinator examine patient & clinician enrollment status process 6100 begins with the prerequisite of coordinator logon process 5500, in which all three actors participate. After that, at operation 6120 the coordinator may select the view participants option under enrollment services 272 from the menu of coordinator portal services 270. At operation 6135 coordinator application 170 may get the corresponding additional screens and logic from coordinator portal 207 if they are not already loaded, and coordinator portal 207 would provide them at operation 6150. At operation 6140, then, the application presents the appropriate screens for showing patient & clinician enrollment status, and the coordinator may view this information at operation 6125. Depending on coordinator preference settings, the default screen may be a list of patients and their statuses, with a summary showing the number of patients in each status, a list of clinicians and their statuses, along with a summary showing the number of patients in each status for each clinician, or some combination. Any format that is not automatically shown may be selected explicitly for display from a menu.

Note that all patient and clinician identifying information is removed by operational databases 202 when data is accessed by coordinator portal 207, so that only anonymized (also known as de-identified) information is available.

At this point, coordinator examine patient enrollment status process 6100 is complete, with each sub-process quiescing in its own way. User sub-process 6105 simply finishes at operation 6130, with nothing further for the coordinator to do. In application sub-process 6110, coordinator application 170 remains running in coordinator workstation 107, so its quiescent state is waiting for the user's next selection at operation 6145. Similarly, coordinator portal 207 has an established session for this user, with coordinator application 170 connected and authenticated, so it too quiesces in an active state waiting for the next application request at operation 6155. Any of the other coordinator methods may be selected in this state.

Trial operations service suite 200 may include support for communication between a coordinator and other trial participants, potentially including patients, clinicians, investigators, and other coordinators. Which other participants a particular coordinator is allowed to select for communication may be constrained by the trial design as established by a coordinator. For example, a coordinator may be permitted to communicate only with investigators and other coordinators, or a coordinator may be permitted to communicate with clinicians in the trial. This capability, if permitted by the trial design, may enable a social networking aspect across the community of trial participants, thereby enhancing participants' engagement in the trial protocol. Four modes of communication may be supported, which are divided into two categories for the purpose of explaining their operation. Communication that is oriented toward exchanging written messages, documents, images, and other related information is categorized as messaging, and typified as chat mode or email mode. Communication that is oriented toward verbal or visual conversation over a live full-duplex connection is categorized as calling, and typified as voice mode or video mode. The processes supporting these capabilities are specified for each category, and within each category for initiating and accepting communications of the corresponding category, yielding four separate processes to be described hereinafter.

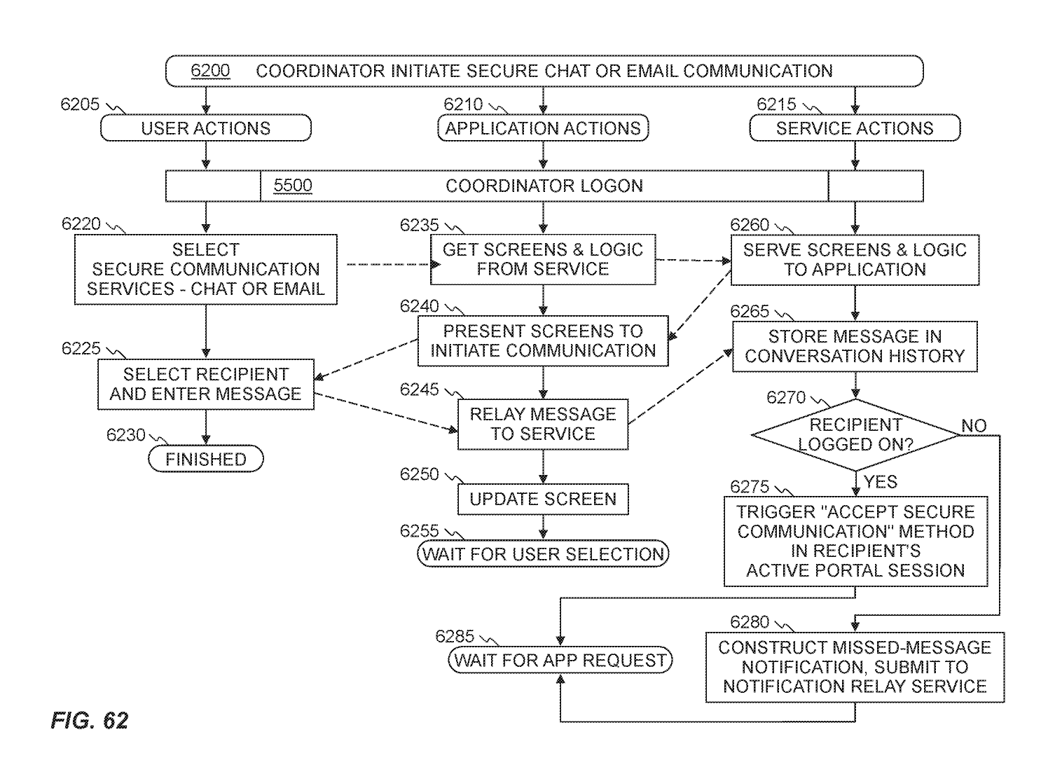

The first communication process is coordinator initiate secure chat or email communication process 6200, which is depicted in FIG. 62. As usual, the three-actor model and temporal/informational interaction conventions apply. The sub-processes in this case are labeled user actions 6205, application actions 6210, and service actions 6215; the primary service actor is secure communication services 273, with help from secure communication relay services 281.

Coordinator initiate secure chat or email communication process 6200 begins with the prerequisite of coordinator logon process 5500, in which all three actors participate. After that, at operation 6220 the coordinator may select secure communication services 273 and the appropriate communication mode—in this case either chat or email—from the menu of coordinator portal services 270. At operation 6235 coordinator application 170 may get the corresponding additional screens and logic from coordinator portal 207 if they are not already loaded, and coordinator portal 207 would provide them at operation 6260. At operation 6240, then, the application presents the appropriate screens for initiating communication. Depending on the trial design established by a coordinator, the coordinator may select one or more recipients to which a chat or email message should be sent, with the selection mechanism and message entry embodied in operation 6225, whereupon coordinator application 170 should at operation 6245 relay the entered message to secure communication services 273 of coordinator portal 207, and at operation 6250 update the presented screen accordingly. Meanwhile, secure communication relay services 281 at operation 6265 may record the message in a conversation history associated with both the sending coordinator and the recipient, which may preferably be stored in the notes, records, and events 225 section of operational databases 202. Note that while only one recipient is depicted in the figure, this and following operations may be repeated for each of multiple recipients if the sender selected more than one. Then, at operation 6270 secure communication relay services 281 may determine whether the recipient is currently logged on in an active portal session. If so, at operation 6275 secure communication relay services 281 may trigger the recipient's accept secure chat or email communication process 1400, 3300, 4800, or 6300 depending on the recipient's role in the system, respectively a patient, a clinician, an investigator, or another coordinator. If the recipient does not have a current active portal session, then at operation 6280 secure communication relay services 281 may construct a missed-message notification and submit it to notifications relay service 282 so that it may be sent to the recipient's external contact address, facilitated by process 7400 notifications relay service autonomous operation and process 7450 external notifications gateway autonomous operation.

At this point, coordinator initiate secure chat or email communication process 6200 is complete, with each sub-process quiescing in its own way. User sub-process 6205 simply finishes at operation 6230, with nothing further for the coordinator to do. In application sub-process 6210, coordinator application 170 remains running in coordinator workstation 107, so its quiescent state is waiting for the user's next selection at operation 6255. Similarly, coordinator portal 207 has an established session for this user, with coordinator application 170 connected and authenticated, so it too quiesces in an active state waiting for the next application request at operation 6285. Any of the other coordinator methods may be selected in this state.

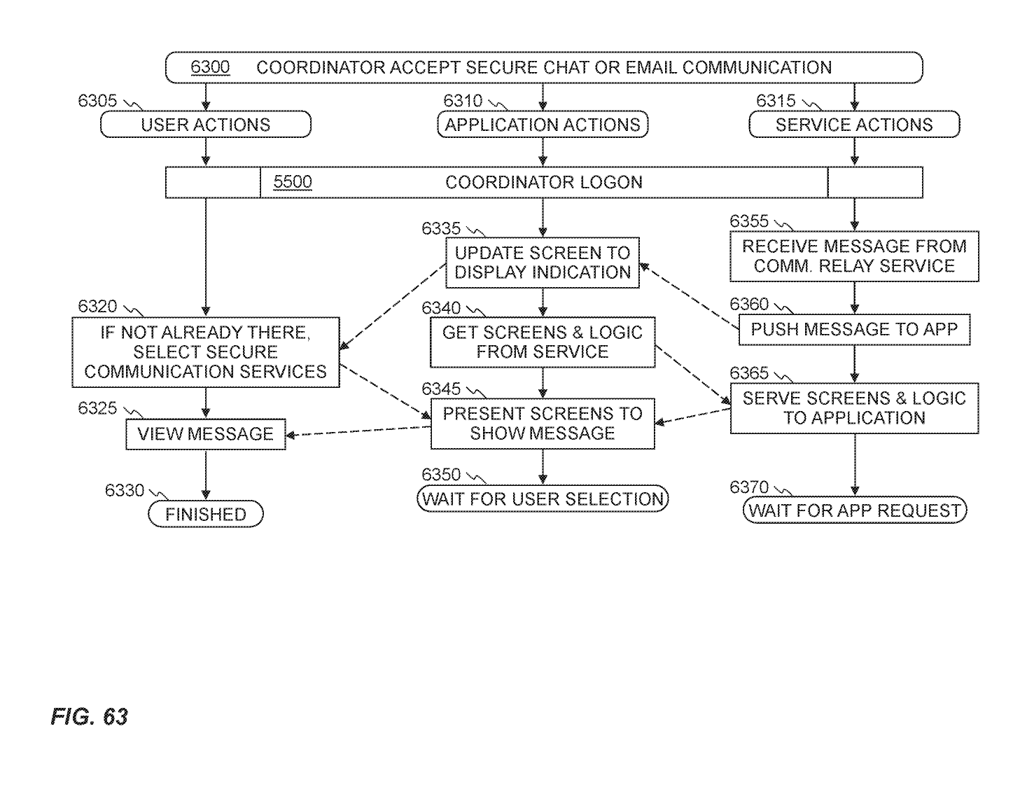

The next communication process is coordinator accept secure chat or email communication process 6300, which is depicted in FIG. 63. As usual, the three-actor model and temporal/informational interaction conventions apply. The sub-processes in this case are labeled user actions 6305, application actions 6310, and service actions 6315; the primary service actor is secure communication services 273, with help from secure communication relay services 281.

Coordinator accept secure chat or email communication process 6300 begins with the prerequisite of coordinator logon process 5500, in which all three actors participate. Note that external notification mechanisms described in processes 7400 and 7450, respectively notifications relay service autonomous operation and external notifications gateway autonomous operation, may prompt a coordinator who is not already logged on to do so. Once coordinator logon process 5500 has established a portal session for the coordinator, whether prompted anew or already in place from previous activity, at operation 6355 secure communication services 273 of coordinator portal 207 may receive a copy of the message from secure communication relay services 281. After that, at operation 6360 the coordinator portal 207 should push the message to coordinator application 170, which would in turn at operation 6335 update whatever screen it is displaying to include an indication of the message's arrival. At operation 6340 coordinator application 170 may predictively retrieve the corresponding additional screens and logic from coordinator portal 207 if they are not already loaded, and coordinator portal 207 would provide them at operation 6365.

When the coordinator notices the indication thus displayed, at operation 6320 the coordinator may select secure communication services 273 and the appropriate communication mode—in this case either chat or email—from the menu of coordinator portal services 270. This may involve explicit selection of a command to enter the corresponding service, or simply touching or clicking on the indication itself; alternatively, coordinator application 170 may switch to the corresponding service mode automatically, or already be in the corresponding service mode due to previous activity. However it arrived in the appropriate state, at operation 6345 coordinator application 170 presents the appropriate screens for viewing the received message. Operation 6325 embodies the coordinator's activity of viewing the presented message. If the communication mode is email, this may also include selecting a specific message from a list of messages that may have been sent to the coordinator. If the communication mode is chat, this may also include viewing other messages both sent and received in the context of a conversation.

At this point, coordinator accept secure chat or email communication process 6300 is complete, with each sub-process quiescing in its own way. User sub-process 6305 simply finishes at operation 6330, with nothing further for the coordinator to do. In application sub-process 6310, coordinator application 170 remains running in coordinator workstation 107, so its quiescent state is waiting for the user's next selection at operation 6350. Similarly, coordinator portal 207 has an established session for this user, with coordinator application 170 connected and authenticated, so it too quiesces in an active state waiting for the next application request at operation 6370. Any of the other coordinator methods may be selected in this state.

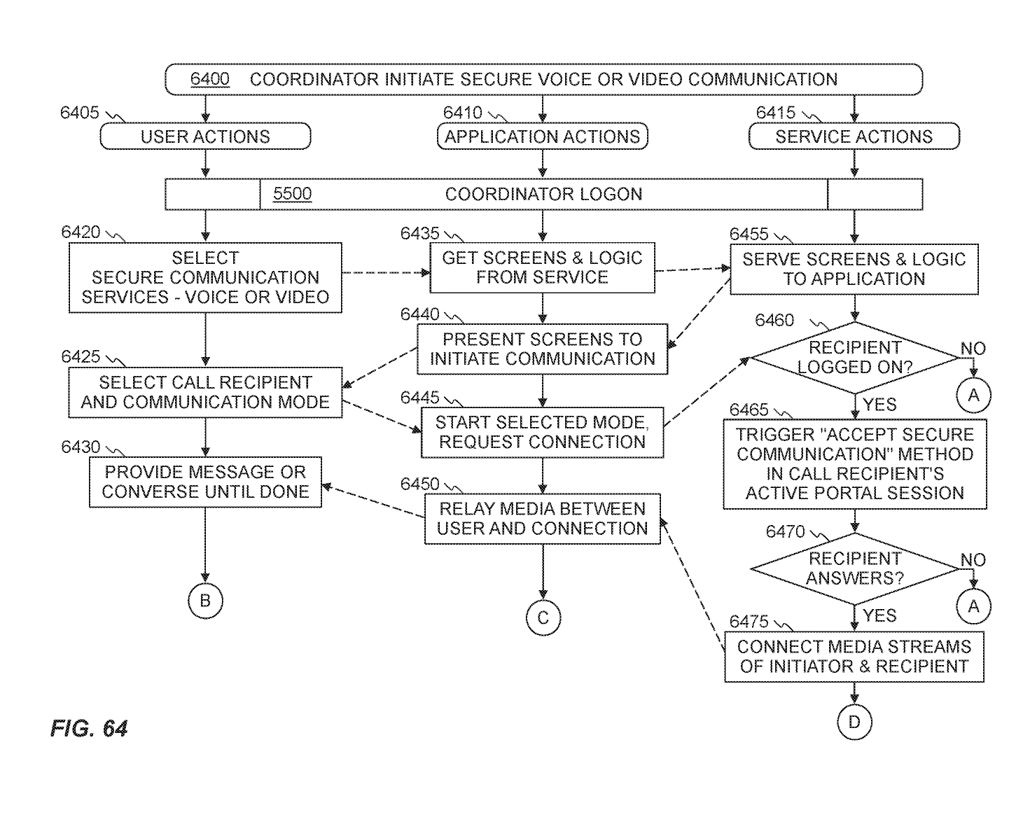

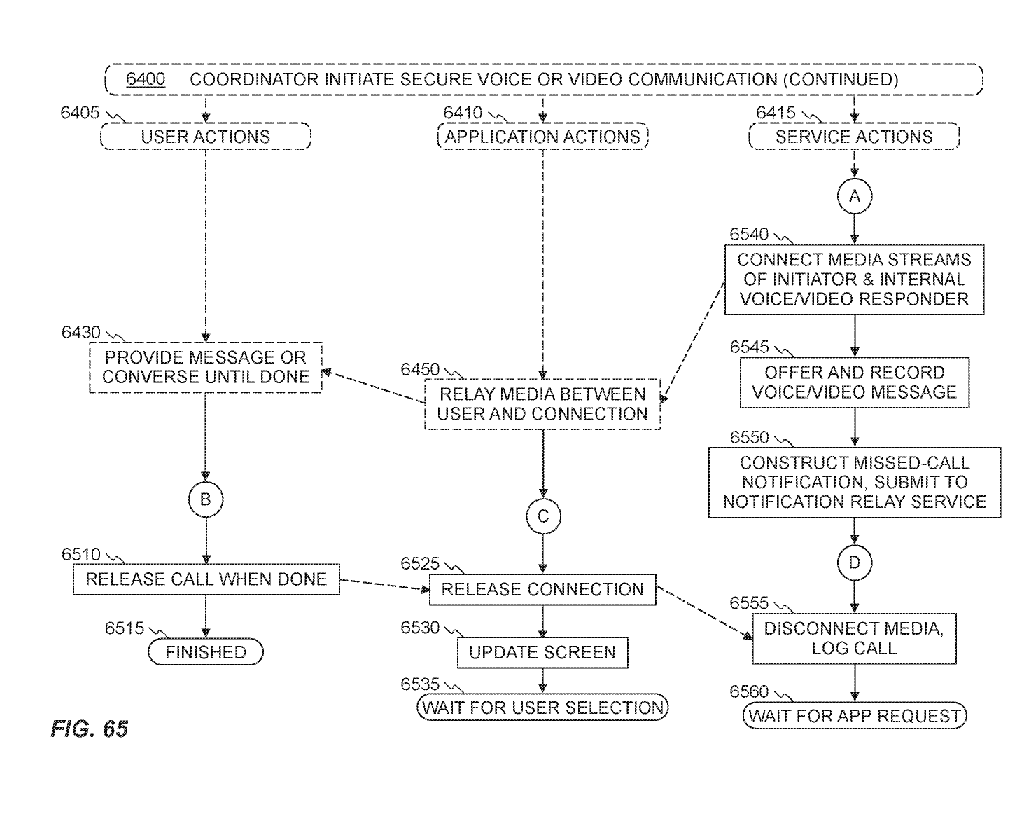

The next communication process is coordinator initiate secure voice or video communication process 6400, which is depicted in FIG. 64 and FIG. 65. As usual, the three-actor model and temporal/informational interaction conventions apply. The sub-processes in this case are labeled user actions 6405, application actions 6410, and service actions 6415; the primary service actor is secure communication services 273, with help from secure communication relay services 281. Note that the sub-process flows cross from FIG. 64 to FIG. 65 via the connection points shown as labeled circles.

Coordinator initiate secure voice or video communication process 6400 begins with the prerequisite of coordinator logon process 5500, in which all three actors participate. After that, at operation 6420 the coordinator may select secure communication services 273 and the appropriate communication mode—in this case either voice or video—from the menu of coordinator portal services 270. At operation 6435 coordinator application 170 may get the corresponding additional screens and logic from coordinator portal 207 if they are not already loaded, and coordinator portal 207 would provide them at operation 6455. At operation 6440, then, the application presents the appropriate screens for initiating communication. Depending on the trial design established by a coordinator, the coordinator may select another trial participant with which a voice or video call should be placed, with the selection mechanism and message entry embodied in operation 6425, whereupon coordinator application 170 should at operation 6445 start the selected communication mode and request secure communication services 273 to have secure communication relay services 281 make a connection supporting the call. At operation 6460 secure communication relay services 281 may determine whether the called participant, referred to as recipient in the figure, is currently logged on in an active portal session. If so, at operation 6465 secure communication relay services 281 may trigger the recipient's accept secure voice or video communication process 1700, 3600, 5100, or 6600 depending on the recipient's role in the system, respectively a patient, a clinician, an investigator, or another coordinator. During that process, the recipient may choose to accept (answer), reject, or ignore the call. At operation 6470, secure communication relay services 281 determines this outcome. If the recipient answers, at operation 6475 secure communication services 273 and secure communication relay services 281 connect the media streams of the initiator and the recipient so that they may converse.

At operation 6450 coordinator application 170 relays the media streams between the calling user and the connection, so that the coordinator may at operation 6430 either converse with the called participant or leave a voice or video message, as appropriate. Note that these two operations appear on both FIG. 64 and FIG. 65, and represent the same actions in both. When the coordinator is finished conversing with or providing a voice/video message for the recipient, he or she will release the call at operation 6510. This may take the form of clicking or touching a hang-up control on the screen of, or pressing a button on, coordinator workstation 107.

If the recipient does not have a current active portal session (operation 6460), or if the recipient does not answer the call (operation 6470), then at operation 6540 secure communication services 273 may connect the initiator's media streams to those of an internal voice or video responder—effectively an answering machine—provided by secure communication relay services 281, which at operation 6545 may offer prompts to record a voice or video message and then do so if one is provided by the initiator. After the voice or video message is finished, which may be determined by detecting a configured period of silence, or by detecting an explicit indication from the calling user such as a key word, tone, or disconnect, or if the caller chooses not to leave a message at all, at operation 6550 the secure communication relay services 281 may construct a missed-call notification and submit it to notifications relay service 282 so that it may be sent to the recipient's external contact address, facilitated by process 7400 notifications relay service autonomous operation and process 7450 external notifications gateway autonomous operation.

Whether the call is answered by the recipient or the message recorder, at operation 6450 coordinator application 170 relays the media streams between the calling user and the connection, so that the coordinator may at operation 6430 either converse with the called participant or leave a voice or video message, as appropriate. Note that these two operations appear on both FIG. 64 and FIG. 65, and represent the same actions in both. When the coordinator is finished conversing with or providing a voice/video message for the recipient, he or she will release the call at operation 6510. This may take the form of clicking or touching a hang-up control on the screen of, or pressing a button on, coordinator workstation 107. Alternatively, coordinator application 170, secure communication services 273, or secure communication relay services 281 may detect a predetermined spoken command or visible gesture from the coordinator as indicating the call is over, or detect that a disconnect indication of some sort was detected on the other side of the call. However call termination may be detected, coordinator application 170 will at operation 6525 release the connection and at operation 6530 update the screen to reflect this change of state. At operation 6555 secure communication relay services 281 will disconnect the media streams between the initiator's and recipient's portal sessions, and record the event in a conversation history associated with both the calling coordinator and the recipient, which may preferably be stored in the notes, records, and events 225 section of operational databases 202.

At this point, coordinator initiate secure voice or video communication process 6400 is complete, with each sub-process quiescing in its own way. User sub-process 6405 simply finishes at operation 6515, with nothing further for the coordinator to do. In application sub-process 6410, coordinator application 170 remains running in coordinator workstation 107, so its quiescent state is waiting for the user's next selection at operation 6535. Similarly, coordinator portal 207 has an established session for this user, with coordinator application 170 connected and authenticated, so it too quiesces in an active state waiting for the next application request at operation 6560. Any of the other coordinator methods may be selected in this state.

Note that coordinator initiate secure voice or video communication process 6400 may also be used to listen to a voice message or view a video message left by another participant when a call from that other participant was missed by the coordinator. The details of this usage are not depicted, but they are effectively identical if the “answering machine” function of secure communication relay services 281 is considered the call recipient and the coordinator's interactions with that function are considered the conversation.

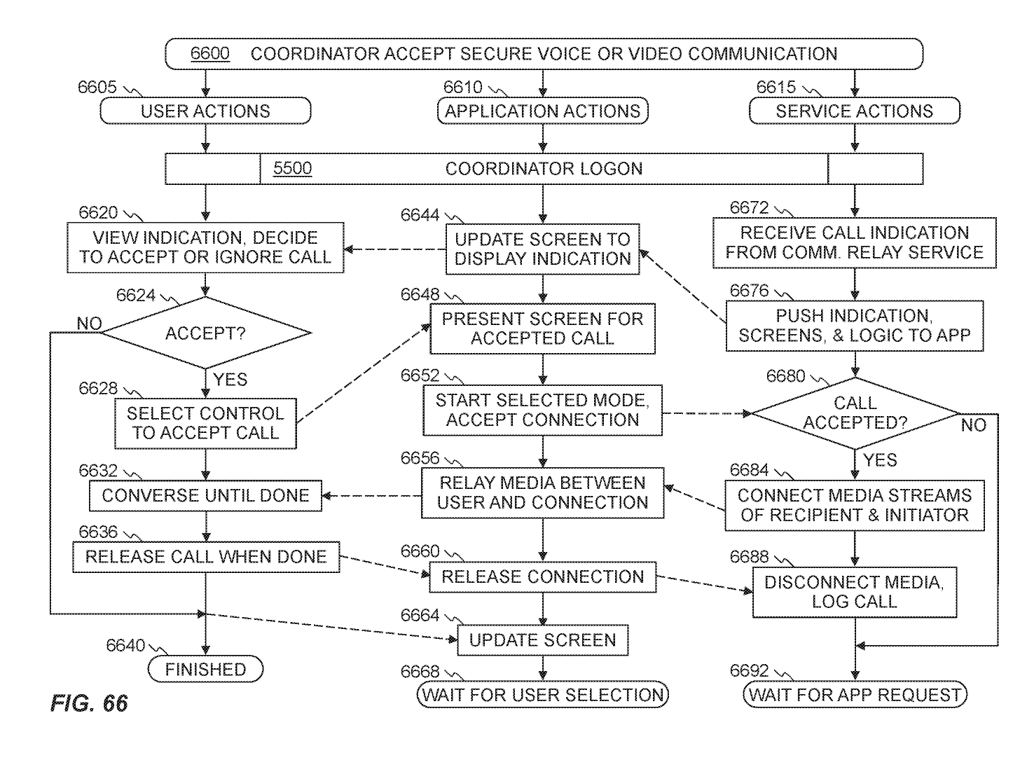

The fourth communication process is coordinator accept secure voice or video communication process 6600, which is depicted in FIG. 66 Again, the three-actor model and temporal/informational interaction conventions apply. The sub-processes in this case are labeled user actions 6605, application actions 6610, and service actions 6615; the primary service actor is secure communication services 273, with help from secure communication relay services 281.

Coordinator accept secure voice or video communication process 6600 begins with the prerequisite of coordinator logon process 5500, in which all three actors participate. Note that for a voice or video call, this will generally have already occurred such that the portal session is currently active; otherwise the called coordinator would not have an opportunity to accept the call while it is being offered, and it would have turned into a missed call. Thus in this scenario, right away at operation 6672 secure communication services 273 of coordinator portal 207 should receive an indication of the offered call from secure communication relay services 281, and at operation 6676 coordinator portal 207 should push that indication, as well as screens and logic for accepting a voice or video call, to coordinator application 170. Coordinator application 170 would in turn at operation 6644 update whatever screen it is displaying to include an indication of the incoming call.

When the coordinator notices, at operation 6620, the indication thus displayed, he or she may choose at operation 6624 to accept or ignore the call. If the decision is to accept, the coordinator at operation 6628 may select the necessary control to do so. This may involve explicit selection of a menu command to enter secure communication services 273 and the appropriate communication mode—in this case either voice or video. Preferably, though, it would involve simply touching or clicking on the indication itself, causing coordinator application 170 to switch to the corresponding service mode automatically. However it arrived in the appropriate state, at operation 6648 coordinator application 170 presents the appropriate screen for having accepted the call, and at operation 6652 it starts the selected mode and completes the connection. At operation 6680, secure communication services 273 and secure communication relay services 281 also may determine whether the coordinator accepted the call, and if so proceed at operation 6684 to connect the media streams of the recipient and initiator, while coordinator application 170 at operation 6656 relays the media streams between the coordinator and the connection, so that the coordinator may at operation 6632 converse with the caller. When the coordinator is finished conversing with the caller, he or she will release the call at operation 6636. This may take the form of clicking or touching a hang-up control on the screen of, or pressing a button on, coordinator workstation 107. Alternatively, coordinator application 170, secure communication services 273, or secure communication relay services 281 may detect a predetermined spoken command or visible gesture from the coordinator as indicating the call is over, or detect that a disconnect indication of some sort was detected on the other side of the call. However call termination may be detected, coordinator application 170 will at operation 6660 release the connection and at operation 6664 update the screen to reflect this change of state. At operation 6688 secure communication services 273 will disconnect the media streams between the initiator's and recipient's portal sessions, and record the event in a conversation history associated with both the calling coordinator and the recipient, which may preferably be stored in the notes, records, and events 225 section of operational databases 202.

If the coordinator ignores or rejects the calls, this is detected at operations 6624 and 6680, and the process skips to the end, bypassing all the other operations except 6664 in which coordinator application 170 updates the screen to reflect the corresponding state change.

Whether answered and disconnected, or ignored, at this point coordinator accept secure voice or video communication process 6600 is complete, with each sub-process quiescing in its own way. User sub-process 6605 simply finishes at operation 6640, with nothing further for the coordinator to do. In application sub-process 6610, coordinator application 170 remains running in coordinator workstation 107, so its quiescent state is waiting for the user's next selection at operation 6668. Similarly, coordinator portal 207 has an established session for this user, with coordinator application 170 connected and authenticated, so it too quiesces in an active state waiting for the next application request at operation 6692. Any of the other coordinator methods may be selected in this state.

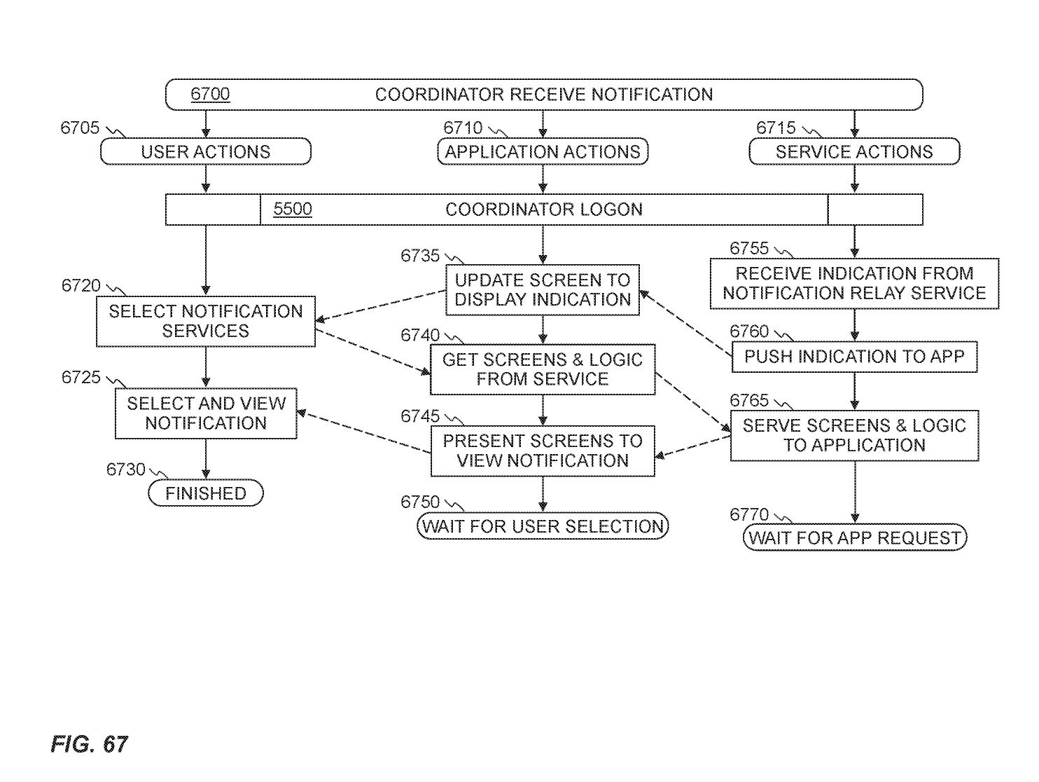

A coordinator may send other coordinators a notification conveying, for example, news about the trial; see the corresponding send notification process 6800 described later. Coordinator receive notification process 6700, which is depicted in FIG. 67, enables the coordinator to view such notifications. Once again, the three-actor model and temporal/informational interaction conventions apply. The sub-processes in this case are labeled user actions 6705, application actions 6710, and service actions 6715.

Coordinator receive notification process 6700 begins with the prerequisite of coordinator logon process 5500, in which all three actors participate. Note that external notification mechanisms described in processes 7400 and 7450, respectively notifications relay service autonomous operation and external notifications gateway autonomous operation, may prompt a coordinator who is not already logged on to do so. Once coordinator logon process 5500 has established a portal session for the coordinator, whether prompted anew or already in place from previous activity, coordinator portal 207 may at operation 6755 receive an indication of the notification from notification relay service 282, and then at operation 6760 push that indication to coordinator application 170. Coordinator application 170 in turn may at operation 6735 update whatever screen it is displaying to include a form of the indication observable by the user.

After that, at operation 6720 the coordinator may select notification service 274 from the menu of coordinator portal services 270. This may involve explicit selection of a command to view notifications, or simply touching or clicking on the indication itself. Either way, at operation 6740 coordinator application 170 may get the corresponding additional screens and logic from coordinator portal 207 if they are not already loaded, and coordinator portal 207 would provide them at operation 6765. At operation 6745, then, the application presents the appropriate screens for viewing the notification to the user. Operation 6725 embodies the coordinator's activity of viewing the presented notification, which may also include selecting a specific notification from a list of notifications that may have been sent to the coordinator.

At this point, coordinator receive notification process 6700 is complete, with each sub-process quiescing in its own way. User sub-process 6705 simply finishes at operation 6730, with nothing further for the coordinator to do. In application sub-process 6710, coordinator application 170 remains running in coordinator workstation 107, so its quiescent state is waiting for the user's next selection at operation 6750. Similarly, coordinator portal 207 has an established session for this user, with coordinator application 170 connected and authenticated, so it too quiesces in an active state waiting for the next application request at operation 6770. Any of the other coordinator methods may be selected in this state.

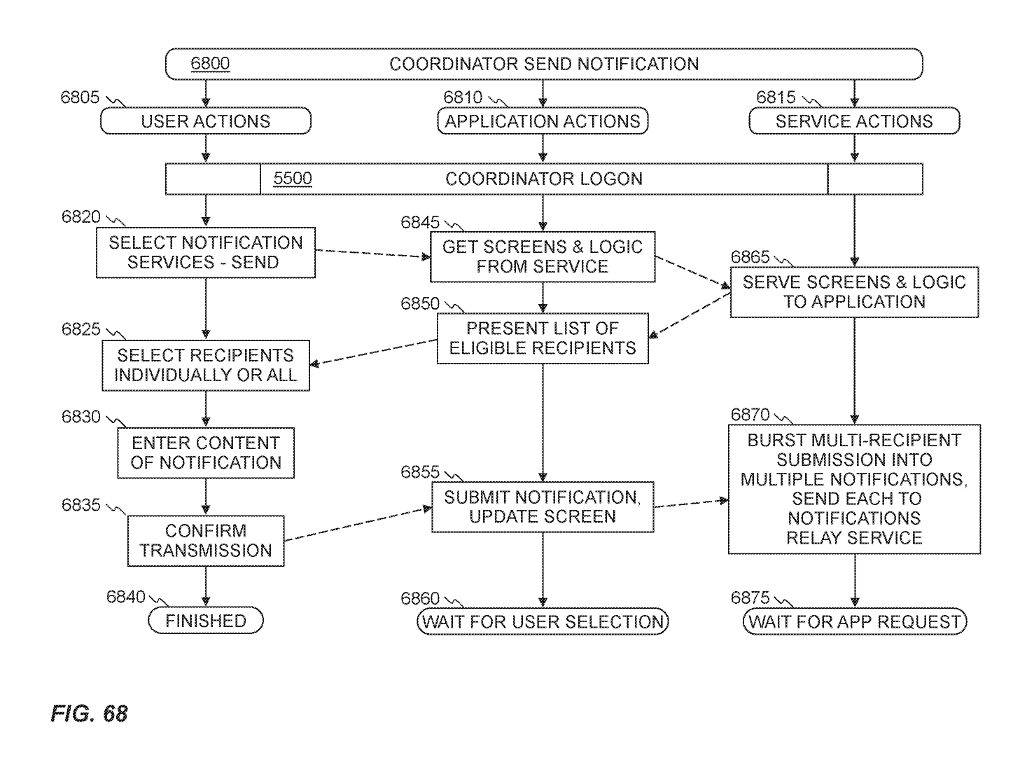

A coordinator may be permitted to send a notification, containing for example trial status information, to one or more trial participants, including all participants of a user type or all participants associated with a particular site, all patients associated with a particular clinician, or other combinations. The coordinator send notification process 6800 may be used for this activity, as depicted in FIG. 68. Once again, the three-actor model applies here, with the same temporal/informational interaction conventions. The sub-processes in this case are labeled user actions 6805, application actions 6810, and service actions 6815.

Coordinator send notification process 6800 begins with the prerequisite of coordinator logon process 5500, in which all three actors participate. After that, at operation 6820 the coordinator may select the send option under notification services 274 from the menu of coordinator portal services 270. At operation 6845 coordinator application 170 may get the corresponding additional screens and logic from coordinator portal 207 if they are not already loaded, and coordinator portal 207 would provide them at operation 6865. At operation 6850, then, the application may present a list for selecting the participant(s) to whom the notification should be sent, and the coordinator may select one, several, or all recipients from this list at operation 6825. The coordinator may then enter the notification content at operation 6830, and confirm transmission of the notification at operation 6835, at which point coordinator application 170 would at operation 6855 submit the notification data package to coordinator portal 207 notification services 274 and update the screen to reflect that this had been done. At operation 6870, notification services 274 would parse the submitted notification data package into one or more notification requests, each addressed to a single patient from the list of recipients selected by the coordinator at operation 6825 and containing a copy of the content provided by the coordinator at operation 6830, and then submit each notification individually to notifications relay service 282 for handling according to notifications relay service autonomous operation process 7400.

At this point, coordinator send notification process 6800 is complete, with each sub-process quiescing in its own way. User sub-process 6805 simply finishes at operation 6840, with nothing further for the coordinator to do. In application sub-process 6810, coordinator application 170 remains running in coordinator workstation 107, so its quiescent state is waiting for the user's next selection at operation 6860. Similarly, coordinator portal 207 has an established session for this user, with coordinator application 170 connected and authenticated, so it too quiesces in an active state waiting for the next application request at operation 6875. Any of the other coordinator methods may be selected in this state.

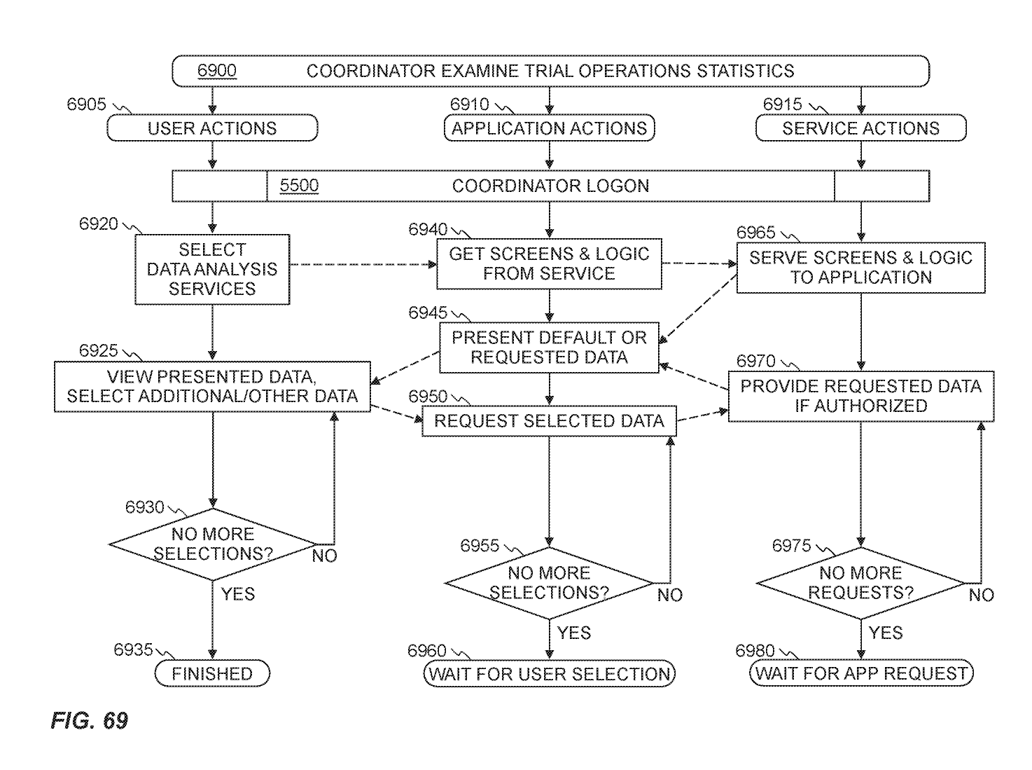

A coordinator may be expected to access trial data such as aggregate statistics from provisioning and authorization data 222, anonymized health information 224, and notes, records, and events not classified as health information 225, in order to track the administrative and scientific progress of the trial. Coordinator examine trial operations statistics, process 6900 depicted in FIG. 69, supports this activity. As usual, the same three-actor model and temporal/informational interaction conventions used throughout this specification apply. The sub-processes in this case are labeled user actions 6905, application actions 6910, and service actions 6915; the primary service actor is data analysis services 275.

Coordinator examine trial operations statistics process 6900 begins with the usual prerequisite of coordinator logon process 5500, in which all three actors participate. After that, at operation 6920 the coordinator may select data analysis services 275 from the menu of coordinator portal services 270. At operation 6940 coordinator application 170 may get the corresponding additional screens and logic from coordinator portal 207 if they are not already loaded, and coordinator portal 207 would provide them at operation 6965. At operation 6945, coordinator application 170 may present the results of a preconfigured or default database query, which may also be the same data being viewed at the end of a previous portal session. At operation 6925, the coordinator may view the presented data, and may select additional or other data to request. The user interface mechanism for presenting data may take any form appropriate to the nature of the data itself, including formats commonly used in data analysis tools such as tables, graphs, spreadsheets, and others. The user interface mechanism for selecting data may also take any form appropriate to the nature of the data, including forms commonly used in data analysis tools such as column or row filters for selection, criteria checkboxes, textual database query languages such as SQL, and others. When a data selection has been formulated and submitted, coordinator application 170 at operation 6950 may request the data from data analysis services 275, which in turn at operation 6970 would provide the requested data back to coordinator application 170 if the coordinator is authorized to see the requested data. Note that all health information is de-identified—that is, patient and clinician identifying information is removed—for all data presented by coordinator portal 207. Presentation at operation 6945 and viewing at operation 6925 would then be repeated. At operation 6930, the user would decide whether additional data should be selected, and if so return to operation 6925 to repeat the cycle. Similarly, coordinator application 170 decides at operation 6955 whether the user has provided another selection, and data analysis services 275 decides at operation 6975 whether the application has requested another selection, and if so both sub-processes return to their respective operations 6950 and 6970 to handle them.

Once all three sub-processes have determined that there are no more selections, perhaps via an explicit indication from the user or via a portal session activity timeout, coordinator examine trial operations statistics process 6900 is complete, with each sub-process quiescing in its own way. User sub-process 6905 simply finishes at operation 6935, with nothing further for the coordinator to do. In application sub-process 6910, coordinator application 170 remains running in coordinator workstation 107, so its quiescent state is waiting for the user's next selection at operation 6960. Similarly, coordinator portal 207 has an established session for this user, with coordinator application 170 connected and authenticated, so it too quiesces in an active state waiting for the next application request at operation 6980. Any of the other coordinator methods may be selected in this state.

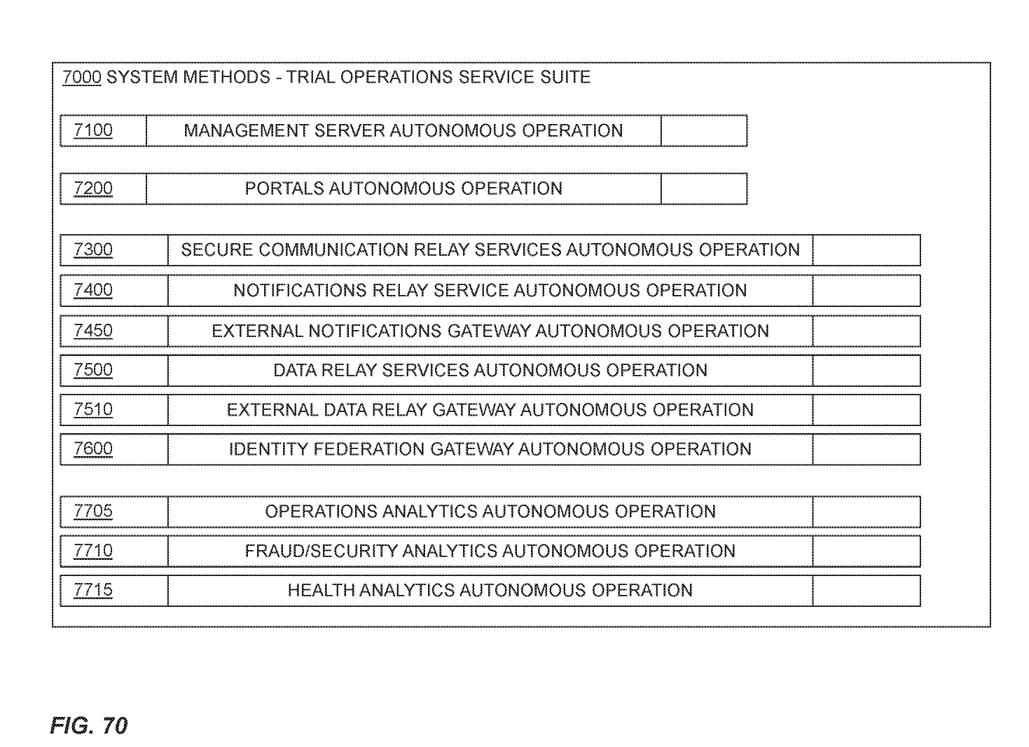

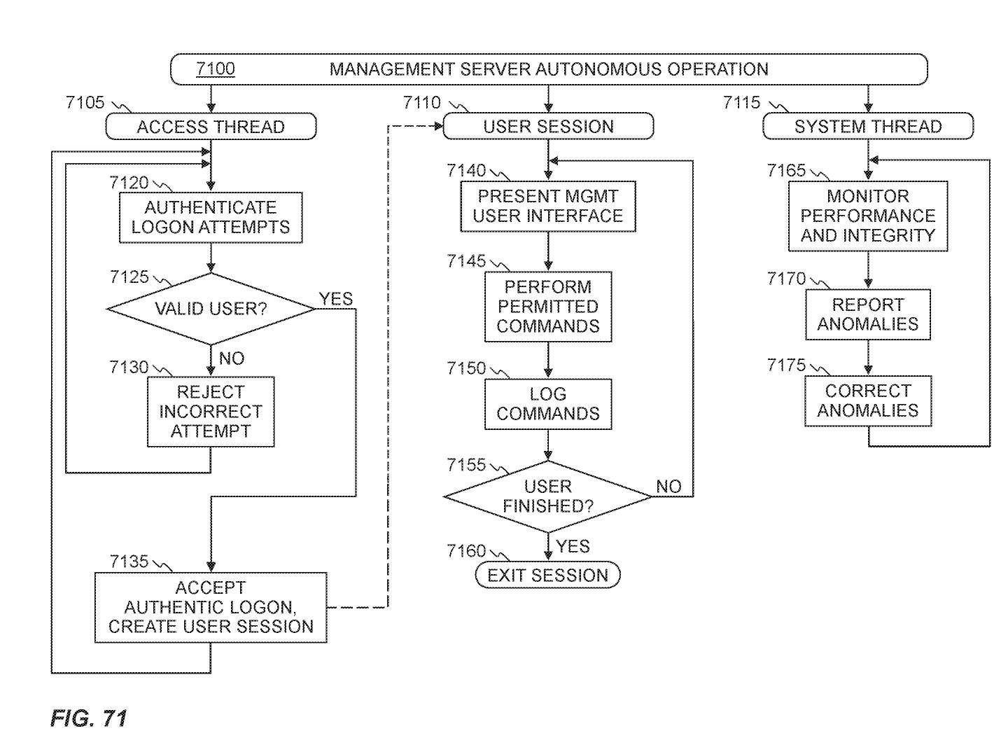

FIG. 70 illustrates a group of autonomous processes performed by elements of a trial operations service suite in a clinical trial operations system according to an exemplary embodiment of the invention. The processes may include support for system management, portal operation, secure communication, notification, data access, identity federation, and automatic data analytics. FIG. 71-FIG. 77 illustrate additional detail for each of the processes in FIG. 70.

Management server autonomous operation process 7100, depicted in FIG. 71, encompasses the activities performed by management server 203. Two primary threads are executed continuously by this process, access thread 7105 and system thread 7115. Zero, one, or more than one user session 7110 may also execute temporarily while a management user is logged on. As in the previous figures, temporal flow within a thread is represented by solid arrows, while temporal relationships between threads are represented by dashed arrows.

System thread 7115 executes continuously throughout the life of trial operations service suite 200. In operation 7165, it is responsible for monitoring system performance and integrity using conventional self-management techniques common in the field of computer server system and software system management. If a problem is detected at any time, management server 203 may at operation 7170 report anomalies according to its configuration; reporting techniques will include at least logging the anomaly using management log service 231, and may also include sending a notification using notifications relay service 282, as well as any mechanism commonly used in the field such as notifying an external system using standard mechanisms according to the Simple Network Management Protocol (SNMP), or causing a physical alarm to be activated using a programmable relay. At operation 7175, management server 203 may attempt to correct a detected anomaly if it is programmed and configured to do so; this correction may take any of numerous forms depending on the details of the anomaly, including such common actions as restarting a service, auditing and recovering corrupted information in a database, or releasing hung sessions.

Access thread 7105 executes continuously throughout the life of trial operations service suite 200. In operation 7120, it detects logon attempts purporting to be from authorized management users, and authenticates them. If it determines at operation 7125 that the logon attempt does not represent a valid user, then at operation 7130 management server 203 rejects the incorrect attempt and continues the process from the beginning. Otherwise, if the logon attempt represents a valid user, then at operation 7135 management server 203 will accept the authentic logon and create a user session 7110.

A user session 7110 thus created provides a context in which the management user may operate on the system, and executes independently of any other user session as well as the continuous access and system threads. At operation 7140, management server 203 would present the management user interface, which may include command-line management service 235 and graphical control management service 236. Commands requested by the management user would, if permitted according to the configuration of management server 203 with respect to the logged-on management user, be performed at operation 7145 and logged at operation 7150, updating the user interface as appropriate. If management server 203 detects at operation 7155 that the management user is finished, perhaps via an explicit logoff command or an inactivity timer that causes an implicit logoff, the session terminates at operation 7160. Otherwise, user session 7110 may repeat until then.

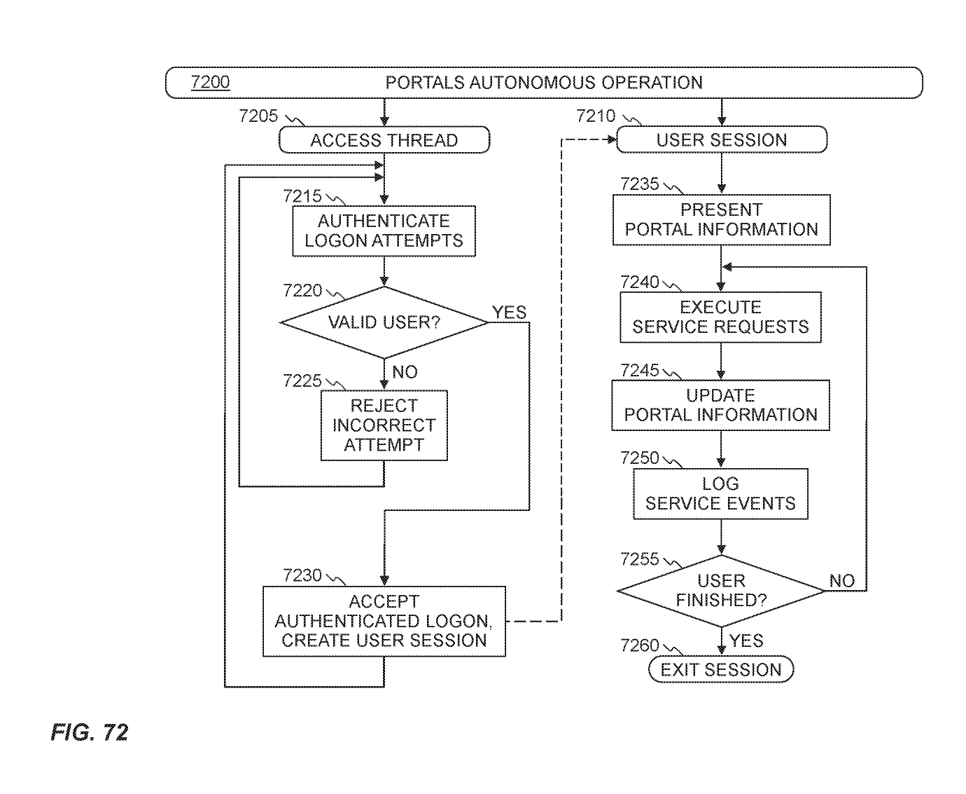

Each of the four user portals—patient portal 204, clinician portal 205, investigator portal 206, and coordinator portal 207—independently instantiates its own copy of the portals autonomous operation process 7200, which is depicted in FIG. 72. One primary thread is executed continuously by this process, access thread 7205. Zero, one, or more than one user session 7210 may also execute temporarily while a portal user is logged on. As in the previous figures, temporal flow within a thread is represented by solid arrows, while temporal relationships between threads are represented by dashed arrows.

Access thread 7205 executes continuously throughout the life of trial operations service suite 200. In operation 7215, it detects logon attempts purporting to be from authorized trial participants, and authenticates them. If it determines at operation 7220 that the logon attempt does not represent a valid user, then at operation 7225 the portal rejects the incorrect attempt and continues the process from the beginning. Otherwise, if the logon attempt represents a valid user, then at operation 7230 the portal will accept the authentic logon and create a user session 7210. This is the actual manifestation of what has been called a portal session in describing other processes, and aligns with the service sub-process in each of those processes.

A user session 7210 thus created provides a context in which the trial participant may perform the various processes described previously, and executes independently of any other user session as well as the continuous access thread. At operation 7235, the portal would present the portal user interface, which is equivalent to the several “serve screens and logic to application” operations in other processes, and may include the portal's dashboard as well as a list of other federated trials to which the user also has access if appropriate. Service requests from the corresponding application would, if permitted according to provisioning and authorization data 222 with respect to the logged-on user, may be performed at operation 7240 and logged at operation 7250, updating the portal user interface as appropriate at operation 7245. If the portal detects at operation 7255 that the user is finished, perhaps via an explicit logoff command or an inactivity timer that causes an implicit logoff, the session terminates at operation 7260. Otherwise, user session 7210 may repeat until then.

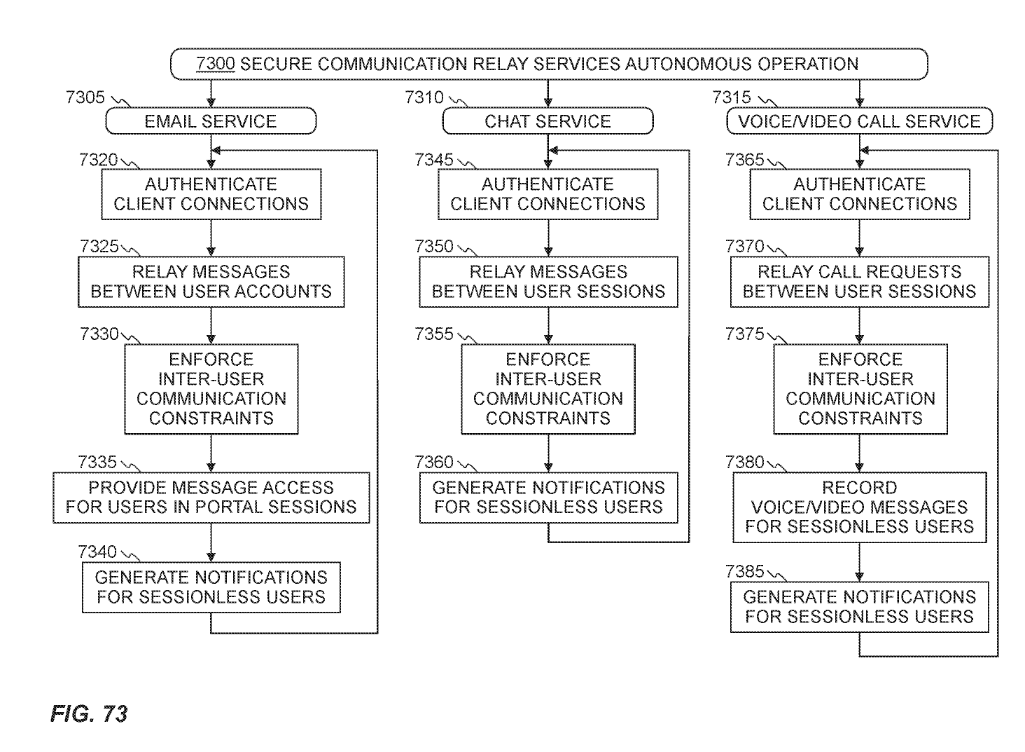

Secure communication relay services 281 provides capabilities for messaging and call connectivity among trial participants as previously described. The method of its behavior, secure communication relay services autonomous operation process 7300, is depicted in FIG. 73 as having three threads of execution. The first thread, email service 7305, supports email messaging among trial participants by acting as a standard email server with configuration and specialization that constrain it to the closed environment of trial operations service suite 200. The second thread, chat service 7310, supports instant messaging among trial participants by acting as a standard chat server, again with configuration and specialization that constrain it to the closed environment of trial operations service suite 200. The third thread, voice/video call service 7315, supports phone calls and video conferences among trial participants by acting as a standard IP PBX, once again configured and specialized so it is constrained to the closed environment of trial operations service suite 200. Being closed means each thread serves only the user community of trial participants and does not support external communication with outside systems. Email service 7305 is configured to disable external relay capability, so that messages cannot be sent to or received from other mail servers. Similarly, chat service 7310 has no configuration for associating with other chat servers, and call service 7315 is configured to have no trunk connections to other call servers. Other specializations are discussed in the descriptions that follow for each thread.

Email service 7305 operates as a continuous cycle covering several action operations and repeating them indefinitely. In operation 7320, the service authenticates client connections from portal sessions, whose secure communication services 243, 253, 263, and 273 may use standard email protocols including the message submission variant of simple message transport protocol (SMTP) to send and internet message access protocol (IMAP) to receive email messages from and to the participants they represent. Implicit in this operation is rejection of connections that fail authentication, although since server and clients are all internal to trial operations service suite 200 any such failure would represent a general operational fault to be dealt with by management services 230.

In operation 7325, email service 7305 performs the usual behavior of relaying messages between user accounts, relying on addressing information in each message to place copies in appropriate folders. This is modified by operation 7330, in which the service enforces inter-user communication constraints so that messages addressed improperly are not delivered, an important consideration in this system due to the sensitivity of and the regulations covering information flowing within it. Note that, since the portals' secure communication services should not present unpermitted addresses for selection when composing a message, this is a second layer of enforcement that if exercised would represent an operational fault and possibly a security breach to be dealt with by management services 230.

Operation 7335 expands upon the email service aspects of operation 7325 by providing message access for users in portal sessions. This is the standard function of an IMAP server, in which the portal secure communication services acting as client may retrieve lists of incoming messages both read and unread, retrieve complete messages for display to the participant/user, and manipulate messages for the purpose of organizing them into folders or deleting them.

Finally, in operation 7340 the service may generate notifications for users not logged on, referred to in the figure as sessionless, informing them of the existence of an incoming message or other important and pertinent occurrence in the secure communication relay services 281. This is effectively a message filter in which a new message is created and handed to notifications relay service 282 for separate delivery, specifically when something happens while the recipient user is not logged on. The notification message would not include the content of the original message, in order to avoid leaking private information in case the notification has to be delivered externally via external notifications gateway 288. Depending on trial design, the notification may or may not include the name of the message sender; if that is too sensitive, it may identify the sender only by relationship, such as “your doctor” or “another trial participant.” Only one notification may be generated within a configured period of time, regardless the number of messages missed, in order to avoid excessive notification traffic.

Chat service 7310 operates as a continuous cycle covering several action operations and repeating them indefinitely. In operation 7345, the service authenticates client connections from portal sessions, whose secure communication services 243, 253, 263, and 273 may use standard instant messaging protocols such as XMPP to register presence and exchange chat messages on behalf of the participants they represent. Implicit in this operation is rejection of connections that fail authentication, although since server and clients are all internal to trial operations service suite 200 any such failure would represent a general operational fault to be dealt with by management services 230.

In operation 7350, chat service 7310 performs the usual behavior of relaying instant messages between user accounts, relying on addressing information in each message to place copies in appropriate queues. This is modified by operation 7355, in which the service enforces inter-user communication constraints so that messages addressed improperly are not delivered, an important consideration in this system due to the sensitivity of and the regulations covering information flowing within it. Note that, since the portals' secure communication services should not present unpermitted addresses for selection when initiating a chat, this is a second layer of enforcement that if exercised would represent an operational fault and possibly a security breach to be dealt with by management services 230.

Finally, in operation 7360 the service may generate notifications for users not logged on, referred to in the figure as sessionless, informing them of the existence of an incoming chat message or other important and pertinent occurrence in the secure communication relay services 281. This is effectively a message filter in which a new message is created and handed to notifications relay service 282 for separate delivery, specifically when something happens while the recipient user is not logged on. The notification message would not include the content of the original message, in order to avoid leaking private information in case the notification has to be delivered externally via external notifications gateway 288. Depending on trial design, the notification may or may not include the name of the initiating participant; if that is too sensitive, it may identify the sender only by relationship, such as “your doctor” or “another trial participant.” Only one notification may be generated within a configured period of time, regardless the number of messages missed, in order to avoid excessive notification traffic.

Voice/video call service 7315 operates as a continuous cycle covering several action operations and repeating them indefinitely. In operation 7365, the service authenticates client connections from portal sessions, whose secure communication services 243, 253, 263, and 273 may use common VOIP protocols including SIP, SDP, RTP, and related standards to place and accept calls to and from the participants they represent. Implicit in this operation is rejection of connections that fail authentication, although since server and clients are all internal to trial operations service suite 200 any such failure would represent a general operational fault to be dealt with by management services 230.

In operation 7370, call service 7315 performs the usual behavior of relaying call requests and media descriptors between user accounts, relying on addressing information in each message to determine the appropriate participating session or sessions. This is modified by operation 7375, in which the service enforces inter-user communication constraints so that call requests addressed improperly are not delivered, an important consideration in this system due to the sensitivity of and the regulations covering information flowing within it. Note that, since the portals' secure communication services should not present unpermitted addresses for selection when initiating a call, this is a second layer of enforcement that if exercised would represent an operational fault and possibly a security breach to be dealt with by management services 230.

Operation 7380 performs the additional capability of recording voice or video messages from a calling participant when the called participant is not logged on, or sessionless. The details of this operation have been previously described in the context of the various initiate secure voice or video communication processes 1500, 3400, 4900, and 6400.

Finally, in operation 7385 the service may generate notifications for users not logged on, referred to in the figure as sessionless, informing them of the missed call or other important and pertinent occurrence in the secure communication relay services 281. This operation has also been described in the context of the various initiate secure voice or video communication processes 1500, 3400, 4900, and 6400. The notification message would not include the content of the voice or video message, even if there is one, in order to avoid leaking private information in case the notification has to be delivered externally via external notifications gateway 288. Depending on trial design, the notification may or may not include the name of the calling participant; if that is too sensitive, it may identify the caller only by relationship, such as “your doctor” or “another trial participant.” Depending on configuration, only one notification may be generated within a configured period of time, regardless the number of calls missed, in order to avoid excessive notification traffic; alternatively, since calls typically do not occur in high numbers, a separate notification may be sent for every missed call in order to make the participant aware of all communication attempts.

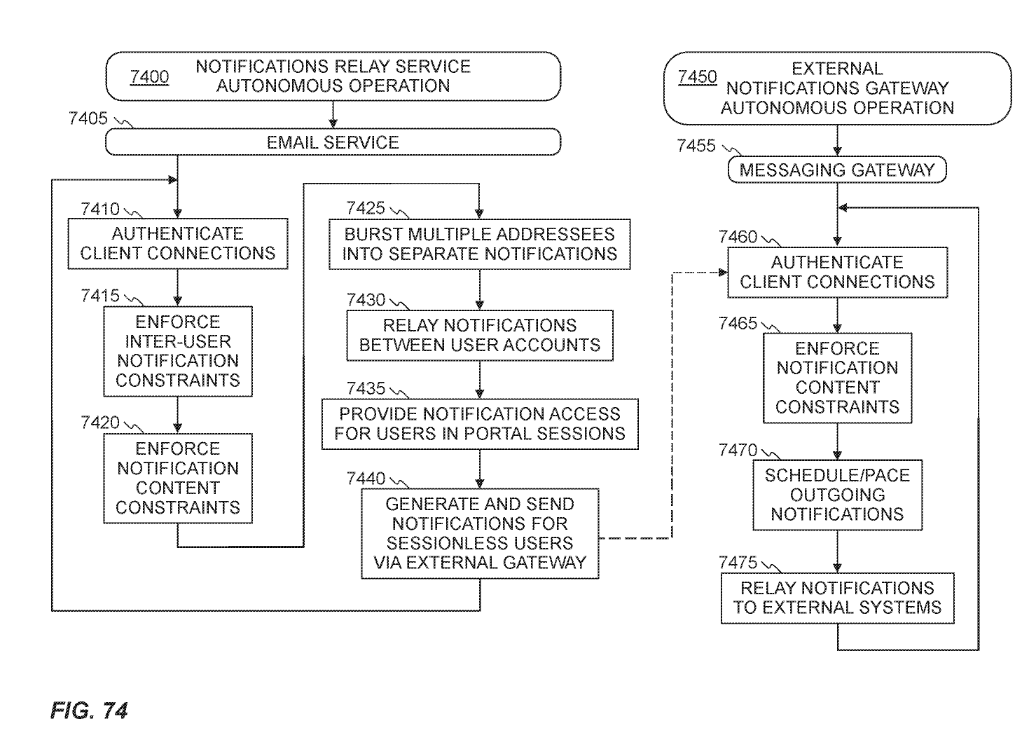

Notifications relay service 282 provides capabilities for restricted messaging among trial participants as previously described. The method of its behavior, notifications relay service autonomous operation process 7400, is depicted in FIG. 74. The process features a single execution thread, email service 7405, which supports notifications as email messaging among trial participants by acting as a standard email server with configuration and specialization that constrain it to the closed environment of trial operations service suite 200 and its notifications capability. Being closed means the thread serves only the user community of trial participants and does not support external communication with outside systems. Email service 7405 is configured to disable external relay capability, so that notification messages cannot be sent to or received from other mail servers outside trial operations service suite 200. Other specializations are discussed in the descriptions that follow.

Email service 7405 operates as a continuous cycle covering several action operations and repeating them indefinitely. In operation 7410, the service authenticates client connections from portal sessions, whose notification services 242, 252, 262, and 274 may use standard email protocols including the internet message access protocol (IMAP) to receive notification messages as email to the participants they represent, and whose notification services 252 and 274 as well as enrollment services 241, 251, and 272 may if permitted user standard email protocols including the message submission variant of simple message transport protocol (SMTP) to send notification messages as email from the participants they represent. Other clients include the secure communication relay services 281, which may send notifications as previously described, and analytics framework 290, which may send notifications as described later. Implicit in this operation is rejection of connections that fail authentication, although since server and clients are all internal to trial operations service suite 200 any such failure would represent a general operational fault to be dealt with by management services 230.

In operation 7430, email service 7405 performs the usual behavior of relaying messages between user accounts, relying on addressing information in each message to place copies in appropriate folders. This is modified by the preceding operation 7415, in which the service enforces inter-user communication constraints so that notification messages addressed improperly are not delivered, an important consideration in this system due to the sensitivity of and the regulations covering information flowing within it. Note that, since the portals' secure communication services should not present unpermitted addresses for selection when composing a message, nor even support the option to send a notification for users not allowed to do so (patients and investigators), this is a second layer of enforcement that if exercised would represent an operational fault and possibly a security breach to be dealt with by management services 230. Notification message relay is further modified by operation 7420, in which the service examines message text to enforce notification content constraints. This filter is particularly tuned to prevent leakage of protected health information via notifications, which generally are sent to multiple users and therefore must not contain information specific to any of them; note however that certain notification recipients may be authorized to view protected health information, in which case the filter may permit a notification to such users containing such information. Since notification services 252 and 274 may also include measures to avoid including protected health information in a notification message as it is composed, operation 7420 may be considered a second layer of enforcement; however, client measures and relay filters have access to inherently different technical approaches, making them complementary rather than duplicative. Notification message relay is modified yet again at operation 7425, in which a sending client's notification message addressed to multiple recipients is burst into multiple separate notification messages each addressed to a single recipient. This operation ensures that recipients are not made aware of one another's identities by removing other addresses from the notification message. This operation may produce an identical copy of the notification content to every addressed recipient, or it may incorporate capabilities for personalizing the content of each separate notification message by including recipient name or other non-health information at appropriate locations in the text (commonly called “mail-merge”).

Operation 7435 expands upon the email service aspects of operation 7430 by providing message access for users in portal sessions. This is the standard function of an IMAP server, in which the portal notification services acting as client may retrieve lists of incoming notification messages both read and unread, retrieve complete notification messages for display to the participant/user, and delete notification messages that have been read. Though an IMAP server typically also provides the ability to manipulate messages for the purpose of organizing them into folders, email service 7405 may be configured to constrain this function to a small set of predefined folders, or even to eliminate it altogether, for an optimal match to the semantics of notifications.

Finally, in operation 7440 the service may generate external notifications for users not logged on, referred to in the figure as sessionless, submitting copies of the notification to external notifications gateway 288 so that it may relay the notification to each user's external contact address. This is effectively another message filter, in which a new message is created and handed to external notifications gateway 288 for separate delivery. Depending on trial design, the external notification may or may not include the name or relationship of the message sender; it may instead institutionalize the sender as “the trial” or some variation thereof.

Being closed means notifications relay service 282 does not support external communication with outside systems. However, notifications may be forwarded to outside systems in a controlled manner via external notifications gateway 288. External notifications gateway 288 is an internal mail server, satisfying the internal-only requirement of notifications relay service 282, but it has the ability to relay notification messages externally as well. The method of its behavior, external notifications gateway autonomous operation process 7500, is also depicted in FIG. 74. The process features a single execution thread, messaging gateway 7455, which supports relaying notifications submitted as email messages from notifications relay service 282 to external systems.

Messaging gateway 7455 operates as a continuous cycle covering several action operations and repeating them indefinitely. In operation 7460, the service authenticates client connections from notifications relay service 282. It may use standard email protocols including the message submission variant of simple message transport protocol (SMTP) to authenticate the client and accept notification messages as email. Implicit in this operation is rejection of connections that fail authentication, although since server and client are both internal to trial operations service suite 200 any such failure would represent a general operational fault to be dealt with by management services 230.

In operation 7475, messaging gateway 7455 performs the usual behavior of relaying messages to the appropriate external service, depending on the addressed recipient's external contact information. External notifications gateway 288 may support interoperation with multiple kinds of external service, such as email servers, chat servers, short message service gateways, voice telephony networks and corresponding voicemail systems, using the corresponding protocols and making the appropriate media translations (text to speech, for example, in the case of a voice-only external contact address).

External relay is modified by the preceding operation 7465, in which the service examines message text to enforce notification content constraints. This filter is particularly tuned to prevent leakage of protected health information via external notifications, which escape the secure environment of trial operations service suite 200 and therefore must not contain any such information. Since notifications relay service 282 also includes measures to avoid the presence of protected health information in the content of a notification message, operation 7465 may be considered an additional layer of enforcement; however, given the criticality of the information, multiple layers of protection are appropriate. Content constraints may also include conformance to administratively approved templates, and operation 7465 may including filtering the notification for this as well. In any case, if a notification gets to this point with any inappropriate content, including protected health information or unapproved structure, the notification is dropped and the event is logged.

External relay at operation 7475 is also modified by the preceding operation 7470, in which outgoing notifications are scheduled and paced so as not to overload external systems or be considered excessive by them, and so as not to overload the external network access capacity of the computing platform hosting trial operations service suite 200.

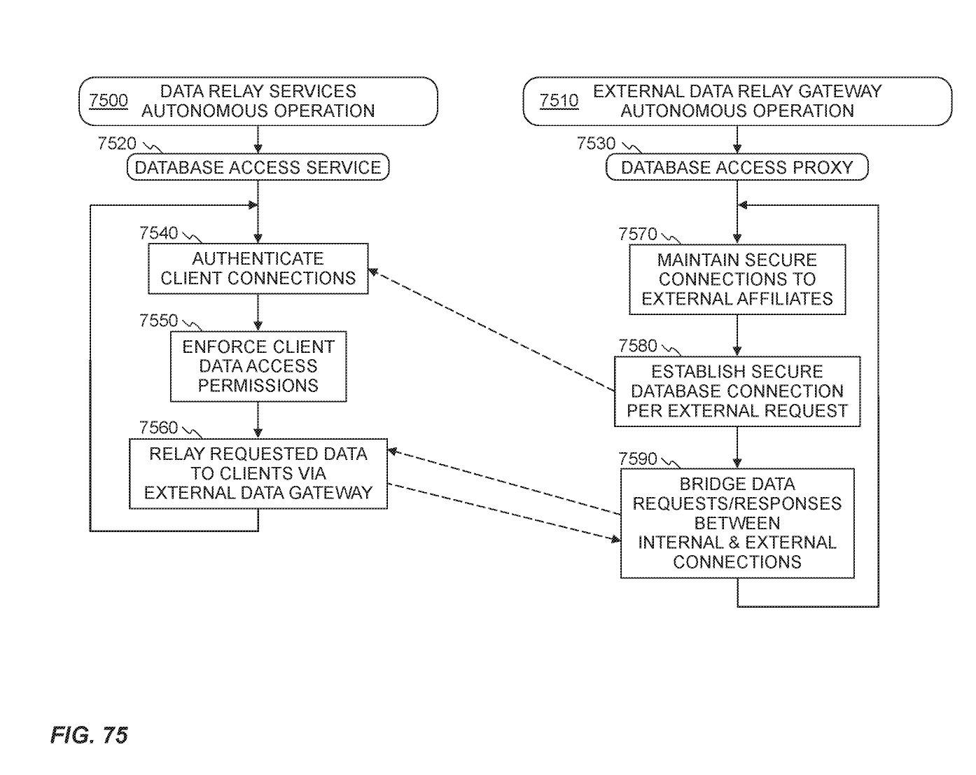

Data relay services 283 and external data relay gateway 287 provide capabilities for access to operational databases 202 by external partners as previously described. The methods of their behavior in this respect, data relay services autonomous operation process 7500 and external data relay gateway autonomous operation process 7510, are depicted in FIG. 75. Each process features a single execution thread, respectively database access service 7520 and database access proxy 7530. This function is separated into two distinct processes for security of the data in operational databases 202. Of the pair, only data relay services 283 may access operational databases 202 directly, while only external data relay gateway 287 has external connections. This isolation, and the structured interaction between the two processes, requires an attacker to penetrate two distinct layers of execution before gaining access (in addition to the usual firewalls and other defenses that are not shown because they are not unique to trial operations service suite 200).

On the internal side, database access service 7520 operates as a continuous cycle covering several action operations and repeating them indefinitely. In operation 7540, the service authenticates client connections from external data relay gateway 287, which may use an internal-only database access protocol specific to the structure of data in operational databases 202 to communicate with data relay services 283. Implicit in this operation is rejection of connections that fail authentication. Two layers of authentication may be employed. The first layer authenticates external data relay gateway 287 itself, which should not fail since it is internal so any such failure would represent a general operational fault to be dealt with by management services 230. The second layer authenticates the external system or user on whose behalf data is being accessed. This second layer uses a subset of provisioning and authorization data 222 that has not been previously discussed, which may be configured by a coordinator as part of trial design or set directly by a management user as part of system installation.

In operation 7560, database access service 7520 performs the usual behavior of looking up requested data and relaying it to external data relay gateway 287 for transmission to the requesting external client. This is modified by the preceding operation 7550, in which the service enforces client data access permissions so each external client may only receive data that client is permitted to receive, thus filtering data results according to the trial protocol as established by a coordinator during trial design. These access permissions, which may be represented as authorizations in provisioning and authorization data 222, serve to protect the sensitive information held in operational databases 202.

Database access proxy 7530, which in common networking terms may be considered an application-layer gateway or proxy firewall specifically for the database access function, also operates as a continuous cycle covering several action operations and repeating them indefinitely. In operation 7570 the gateway maintains secure connections to external affiliated systems. Such connections may be authenticated at both ends using separate credentials for each, which should be stored in security configuration data 201. Here, too, the credentials may be established by a coordinator through trial design or by a management user through maintenance tools. In operation 7580, external data relay gateway 287 may establish a secure internal connection to data relay services 283 for each external client request, providing the external client's credentials to support the second layer of authentication described above for operation 7550. Note that in alternate embodiments, the external client authentication may be performed in operation 7580 using credentials in security configuration data 201, instead of in operation 7540 using credentials in provisioning and authorization data 222, such that data relay services 283 trusts the judgment of external data relay gateway 287 (probably a less secure approach), or it may be performed in both with the authentication performed at 7580 followed by a substitution of internal proxy credentials for the external user's external credentials (possibly a more secure approach due to the redirection, but also possibly less secure if the redirection link in security configuration data 201 is vulnerable). External client requests may use a standard data access protocol appropriate to the purpose of the external affiliated system, such as a standard EHR protocol if the external system is permitted to access protected health information.

Operation 7590 then performs the act of bridging data requests from the external user to the internal access service, and in turn bridging the responses from the internal access service to the external user, translating between the internal and external protocols for each.

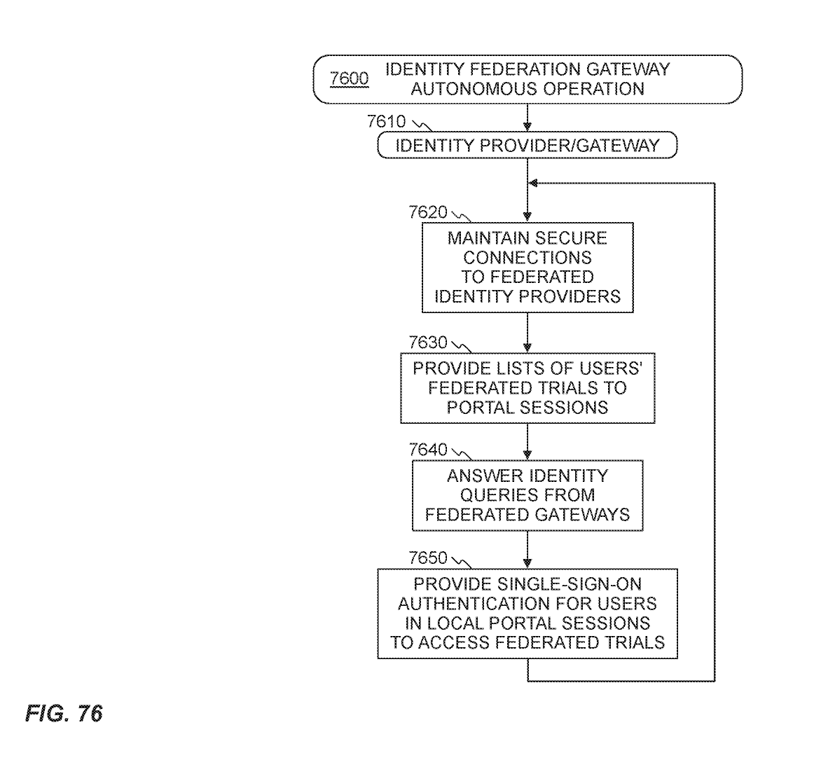

Identity federation gateway 289 provides capabilities that allow trial participants (i.e., users) to translate a single logon in one trial operations service suite 200 instance into an authorized logon in one or more other trial operations service suite 200 instances, without manually entering credentials for each one multiple times, as previously described. The method of its behavior, identity federation gateway autonomous operation process 7600, is depicted in FIG. 76. The process features a single execution thread, identity provider/gateway 7610, which supports single-sign-on identity federation by acting as a standard identity federation server with configuration and specialization that constrain it to the closed environment of affiliated trial operations service suites 200. Being closed in this case means the gateway connects only those trial operations service suites 200 that are explicitly configured to interact with one another.

Identity provider/gateway 7610 operates as a continuous cycle covering several action operations and repeating them indefinitely. In operation 7620, the service maintains secure connections to external affiliated systems, that is federated identity providers. Such connections may be authenticated at both ends using separate credentials for each, which should be stored in security configuration data 201. As with the external data relay gateway, the credentials may be established by a coordinator through trial design or by a management user through maintenance tools. In operation 7630, upon request from a local portal session, gateway 289 may poll its federated identity federation gateways 289 in other trial operation service suites 200 to determine in which of those trials the corresponding participant/user also exists and may logon through single-sign-on, then return a list of such trials to the requesting portal session. Operation 7640 in this gateway 289 is the counterpart of operation 7630 in other gateways 289. That is, in operation 7640 this identity federation gateway 289 answers polls from its federated identity federation gateways 289 to inform them whether a particular participant/user exists in and may logon to this trial through single-sign-on at the other. Finally in operation 7650, upon request from a local portal session in which the corresponding participant/user has selected a federated trial to access, gateway 289 may authenticate that participant/user in the selected federated trial using standard single-sign-on techniques.

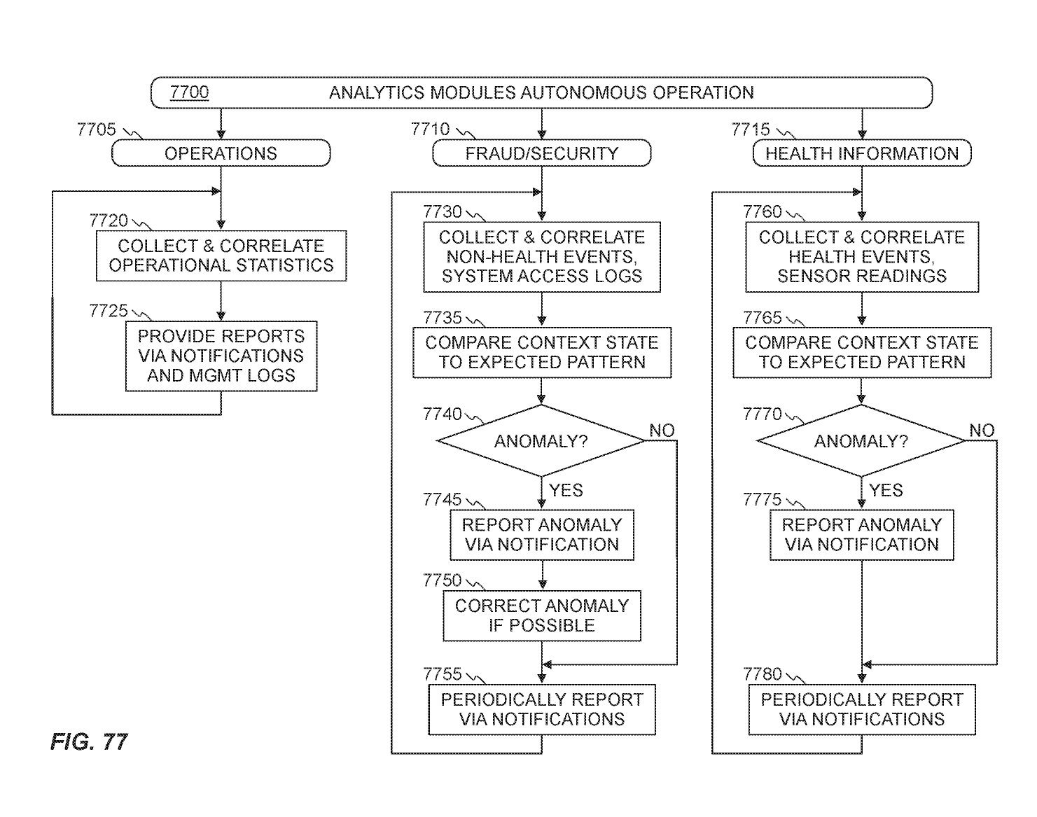

The modules operating within autonomous data analytics framework 290 may provide ongoing analysis of data flow across trial operations service suite 200, as previously described. FIG. 77 illustrates an analytics modules autonomous operation process 7700, which is depicted having three threads 7705, 7710, and 7715 of execution mapping to the three classes of analytics module shown in FIG. 2. The first thread, operations class 7705, may correspond to operations analytic(s) 291 and may examine management event flow to detect operational anomalies. The second thread, fraud/security class 7710, may correspond to fraud/security analytic(s) 292 and may examine participants' non-health event flow to detect patterns of usage or correlations that may indicate fraud or security intrusions. The third thread, health information class 7715, may correspond to health analytic(s) 293 and may examine participants' health event flow to detect patterns or correlations that may indicate health trends or predict health outcomes. Each analytic module may be programmed individually, within its class, to examine specific data and make specific inferences, details of which may change over time due to experience both within a trial and in the relevant technical fields at large. Those details are not captured here; instead, these processes provide a general behavioral framework followed by each analytic module in a class.

Operations class 7705 may function as a continuous cycle covering several action operations and repeating them indefinitely. In operation 7720, a module may collect and correlate operational statistics by monitoring security configuration and management authentication database 201 as well as events recorded by management log service 231. When an instance of the module's programmed correlation is detected, or periodically according to a configured timeframe, at operation 7725 the module may construct a report of its results and submit that report for distribution to coordinators via notifications relay service 282. It may also log the report via management log service 231 to inform management users.

Fraud/security class 7710 may operate as a continuous cycle covering several action operations and repeating them indefinitely. In operation 7730, a module may collect and correlate non-health events as they are stored in operational databases 202, monitoring activity in notes, records, and events not classified as health information 225 as well as access logs 221. At operation 7735, the module may add each received event (that is, database update) to one or more matching correlation contexts, such as per user or per user group or per event type, and then compare the context's state with its expected pattern for the monitored condition. The specific procedure for adding the event to the context and making the comparison may differ from module to module, and may include application of algorithms such as those commonly used for intrusion detection, data leakage detection, and other aspects of system security and user fraud. If at operation 7740 an instance of the module's programmed correlation is detected as an anomaly, at operation 7745 the module may construct a report of its results and submit that report for distribution to coordinators via notifications relay service 282. It may also at operation 7750 attempt to correct the anomaly if possible and appropriate. Whether an anomaly is detected or not, at operation 7755 the module may also report periodically according to a configured timeframe.

Health information class 7715 may operate as a continuous cycle covering several action operations and repeating them indefinitely. In operation 7760, a module may collect and correlate health events such as patient responses, clinician observations, sensor readings, and others as they are stored in operational databases 202, monitoring activity in anonymized health information 224 or, if permitted, protected health information 223. Depending on the condition being matched, the module may also monitor corresponding activity in notes, records, and events not classified as health information 225, as well as access logs 221 if appropriate. At operation 7765, the module may add each received event (that is, database update) to one or more matching correlation contexts, such as per user or per user group or per event type, and then compare the context's state with its expected pattern for the monitored condition. The specific procedure for adding the event to the context and making the comparison may differ from module to module, and may include application of algorithms appropriate for interpreting the event's relationship to the condition being matched. If at operation 7770 an instance of the module's programmed correlation is detected as an anomaly, at operation 7775 the module may construct a report of its results and submit that report for distribution to coordinators and other appropriate participants via notifications relay service 282. For example, a match to a specific medical condition for a particular patient may also be distributed to that patient's clinician, while a match to a health trend affecting multiple patients may also be distributed to a designated investigator. Whether an anomaly is detected or not, at operation 7780 the module may also report periodically according to a configured timeframe.

The processes of FIG. 5-FIG. 77 may be implemented in various different ways without departing from the scope of the disclosure. For instance, the operations may be performed in different orders than shown. As another example, some processes may include additional operations and/or omit various listed operations.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

### IV. Computer System

Many of the processes and modules described above may be implemented as software processes that are specified as one or more sets of instructions recorded on a non-transitory storage medium. When these instructions are executed by one or more computational element(s) (e.g., microprocessors, microcontrollers, digital signal processors (DSPs), application-specific integrated circuits (ASICs), field programmable gate arrays (FPGAs), etc.) the instructions cause the computational element(s) to perform actions specified in the instructions.

In some embodiments, various processes and modules described above may be implemented completely using electronic circuitry that may include various sets of devices or elements (e.g., sensors, logic gates, analog to digital converters, digital to analog converters, comparators, etc.). Such circuitry may be able to perform functions and/or features that may be associated with various software elements described throughout.

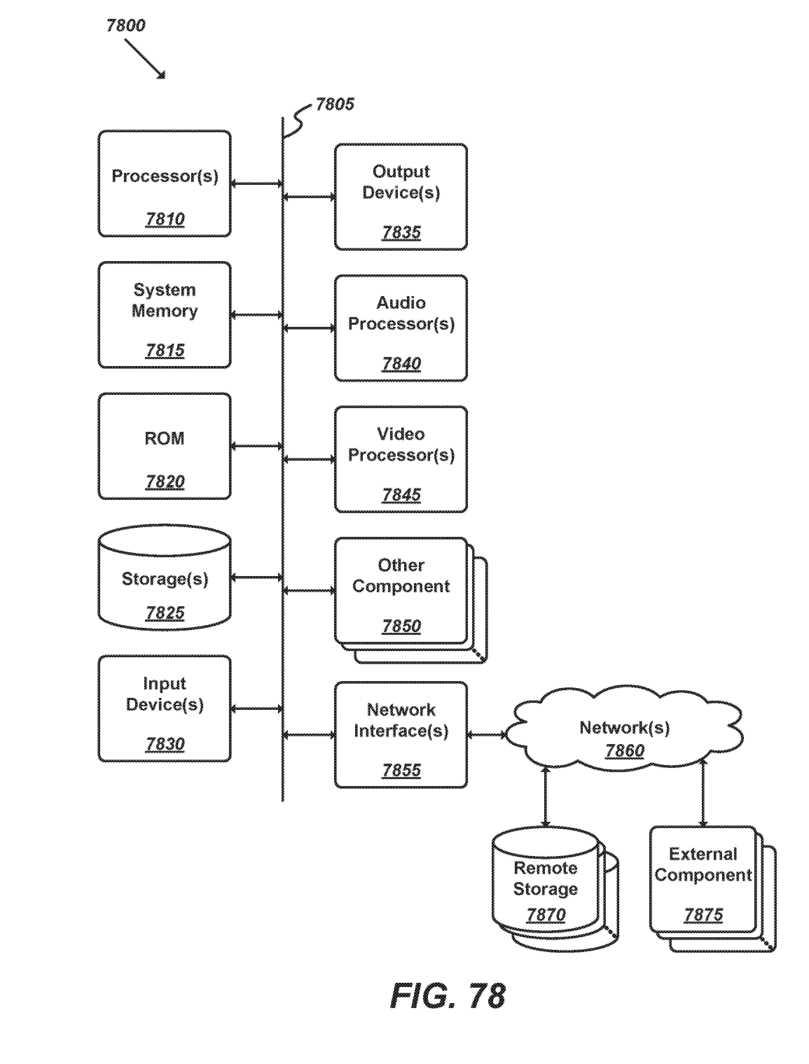

FIG. 78 illustrates a schematic block diagram of an exemplary computer system 7800 used to implement some embodiments. For example, the systems described above in reference to FIG. 1-FIG. 4 may be at least partially implemented using computer system 7800. As another example, the processes described in reference to FIG. 5-FIG. 77 may be at least partially implemented using sets of instructions that are executed using computer system 7800.

Computer system 7800 may be implemented using various appropriate devices. For instance, the computer system may be implemented using one or more personal computers (PCs), servers, mobile devices (e.g., a smartphone), tablet devices, and/or any other appropriate devices. The various devices may work alone (e.g., the computer system may be implemented as a single PC) or in conjunction (e.g., some components of the computer system may be provided by a mobile device while other components are provided by a tablet device).

As shown, computer system 7800 may include at least one communication bus 7805, one or more processors 7810, a system memory 7815, a read-only memory (ROM) 7820, permanent storage devices 7825, input devices 7830, output devices 7835, audio processors 7840, video processors 7845, various other components 7850, and one or more network interfaces 7855.

Bus 7805 represents all communication pathways among the elements of computer system 7800. Such pathways may include wired, wireless, optical, and/or other appropriate communication pathways. For example, input devices 7830 and/or output devices 7835 may be coupled to the system 7800 using a wireless connection protocol or system.

The processor 7810 may, in order to execute the processes of some embodiments, retrieve instructions to execute and/or data to process from components such as system memory 7815, ROM 7820, and permanent storage device 7825. Such instructions and data may be passed over bus 7805.

System memory 7815 may be a volatile read-and-write memory, such as a random access memory (RAM). The system memory may store some of the instructions and data that the processor uses at runtime. The sets of instructions and/or data used to implement some embodiments may be stored in the system memory 7815, the permanent storage device 7825, and/or the read-only memory 7820. ROM 7820 may store static data and instructions that may be used by processor 7810 and/or other elements of the computer system.

Permanent storage device 7825 may be a read-and-write memory device. The permanent storage device may be a non-volatile memory unit that stores instructions and data even when computer system 7800 is off or unpowered. Computer system 7800 may use a removable storage device and/or a remote storage device as the permanent storage device.

Input devices 7830 may enable a user to communicate information to the computer system and/or manipulate various operations of the system. The input devices may include keyboards, cursor control devices, audio input devices and/or video input devices. Output devices 7835 may include printers, displays, audio devices, etc. Some or all of the input and/or output devices may be wirelessly or optically connected to the computer system 7800.

Audio processor 7840 may process and/or generate audio data and/or instructions. The audio processor may be able to receive audio data from an input device 7830 such as a microphone. The audio processor 7840 may be able to provide audio data to output devices 7840 such as a set of speakers. The audio data may include digital information and/or analog signals. The audio processor 7840 may be able to analyze and/or otherwise evaluate audio data (e.g., by determining qualities such as signal to noise ratio, dynamic range, etc.). In addition, the audio processor may perform various audio processing functions (e.g., equalization, compression, etc.).

The video processor 7845 (or graphics processing unit) may process and/or generate video data and/or instructions. The video processor may be able to receive video data from an input device 7830 such as a camera. The video processor 7845 may be able to provide video data to an output device 7840 such as a display. The video data may include digital information and/or analog signals. The video processor 7845 may be able to analyze and/or otherwise evaluate video data (e.g., by determining qualities such as resolution, frame rate, etc.). In addition, the video processor may perform various video processing functions (e.g., contrast adjustment or normalization, color adjustment, etc.). Furthermore, the video processor may be able to render graphic elements and/or video.

Other components 7850 may perform various other functions including providing storage, interfacing with external systems or components, etc.

Finally, as shown in FIG. 78, computer system 7800 may include one or more network interfaces 7855 that are able to connect to one or more networks 7860. For example, computer system 7800 may be coupled to a web server on the Internet such that a web browser executing on computer system 7800 may interact with the web server as a user interacts with an interface that operates in the web browser. Computer system 7800 may be able to access one or more remote storages 7870 and one or more external components 7875 through the network interface 7855 and network 7860. The network interface(s) 7855 may include one or more application programming interfaces (APIs) that may allow the computer system 7800 to access remote systems and/or storages and also may allow remote systems and/or storages to access computer system 7800 (or elements thereof).

As used in this specification and any claims of this application, the terms “computer”, “server”, “processor”, and “memory” all refer to electronic devices. These terms exclude people or groups of people. As used in this specification and any claims of this application, the term “non-transitory storage medium” is entirely restricted to tangible, physical objects that store information in a form that is readable by electronic devices. These terms exclude any wireless or other ephemeral signals.

It should be recognized by one of ordinary skill in the art that any or all of the components of computer system 7800 may be used in conjunction with some embodiments. Moreover, one of ordinary skill in the art will appreciate that many other system configurations may also be used in conjunction with some embodiments or components of some embodiments.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

The foregoing relates to illustrative details of exemplary embodiments and modifications may be made without departing from the scope of the disclosure as defined by the following claims.

We claim:

1. A trial operations service suite in a clinical trial operations system comprising:
patient, clinician, investigator, and coordinator portals supporting corresponding applications and providing specific portal services for each type of participant, wherein the coordinator portal allows a coordinator user to develop, build, customize, and establish trial protocol and participant interaction using an application factory system embedded within a trial design aspect of the coordinator portal;
security configuration and management authentication databases;
operational databases, partitioned for security, comprising trial data including provisioning and authorization data describing trial participants, access logs recording participant activity, protected and de-identified health information provided by patients, clinicians, and associated sensors, and non-health information provided by patients, clinicians, and associated sensors;
a notification relay service and corresponding external notifications gateway providing essential communication between the system and users, between clinicians and patients, and between coordinators and all participants; and
a secure communication relay service providing multi-media communication among participants, constrained by permissions as established by a particular coordinator user.

2. The trial operations service suite of claim 1, wherein the patient portal provides capabilities supporting a patient application whereby:
a patient participating in a clinical trial may self-register for and complete enrollment in the clinical trial, invite other patient and clinician candidates to join the clinical trial, communicate with other clinical trial participants via various media, and provide health information specific to goals of the clinical trial as established by a particular coordinator user; and
the patient application provides information automatically collected from a patient sensor.

3. The trial operations service suite of claim 1, wherein the clinician portal provides capabilities supporting a clinician application whereby:
a clinician participating in a clinical trial may self-register for and complete enrollment in the clinical trial, invite other patient and clinician candidates to join the clinical trial, track status of those invitations, communicate with other trial participants, and provide health information specific to goals of the clinical trial as established by a particular coordinator user; and
the clinician application provides information automatically collected from a clinical sensor.

4. The trial operations service suite of claim 1, wherein the investigator portal provides capabilities supporting an investigator application whereby an investigator participating in a clinical trial may track status of trial participant enrollment, communicate with other trial participants via various media, access and analyze de-identified health information collected via patient and clinician portals, and record analysis results and observations in an electronic lab notebook.

5. The trial operations service suite of claim 1, wherein the coordinator portal provides capabilities supporting a coordinator application whereby a coordinator managing a clinical trial may register other participants individually or in bulk, invite patient or clinician candidates to join the clinical trial, track status of trial participant enrollment and other operational statistics, and communicate with other trial participants.

6. The trial operations service suite of claim 1 further comprising a data relay service and corresponding external data relay gateway providing access to clinical trial data for affiliated external medical information systems.

7. The trial operations service suite of claim 1 further comprising an identity federation gateway providing single-sign-on capability across multiple affiliated trial operations service suite instances supporting a plurality of clinical trials operated under common management.

8. The trial operations service suite of claim 1, implemented using shared computing elements in a private cloud associated with a trial manager corporate data center, and accessed via a virtual private network carried over the Internet.

9. The trial operations service suite of claim 1, implemented as a macroservice in a virtual private data center on computing elements associated with a Health Insurance Portability and Accountability Act (HIPAA)-compliant public cloud service, and accessed via the Internet.

10. The trial operations service suite of claim 1, implemented as a set of serverless microservices in a virtual private cloud on shared computing elements associated with a Health Insurance Portability and Accountability Act (HIPAA)-compliant public cloud service, and accessed via the Internet.

11. The trial operations service suite of claim 1, implemented using dedicated computing elements associated with a trial manager facility, and accessed via at least one of a local area network and the Internet.

12. The trial operations service suite of claim 1 further comprising an autonomous data analytics framework supporting individually programmed analytic modules that each examines specific data to detect and report a specific condition.

13. The trial operations service suite of claim 12, wherein the individually programmed analytic modules focus on operational data and detect conditions related to system operations.

14. The trial operations service suite of claim 12, wherein the individually programmed analytic modules focus on non-health data and detect conditions related to fraud and security.

15. The trial operations service suite of claim 12, wherein the individually programmed analytic modules focus on health data and detect conditions related to health outcomes.

16. A method of operating a trial operations service suite in a clinical trial operations system, the method comprising:
serving a mobile application factory composition studio to a coordinator application via a coordinator portal;
accepting from the coordinator application a set of customizations of trial protocol and participant interaction screens and logic;
building and distributing customized patient, clinician, and investigator applications for use with corresponding patient, clinician, and investigator portals of the trial operations service suite;
registering and enrolling patient, clinician, investigator, and coordinator users;
inviting patients and clinicians to register and enroll as users;
sending and receiving notifications among users;
recording patient health information provided by patients, clinicians, and associated sensors;
protecting patient health information according to Health Insurance Portability and Accountability Act (HIPAA) regulations;
presenting de-identified patient health information to investigator users for analysis; and
supporting secure multi-media communication among participants, constrained by permissions as established by a particular coordinator user.

17. The method of claim 16 further comprising providing access to trial data for affiliated external medical information systems.

18. The method of claim 16 further comprising providing single-sign-on capability across multiple affiliated trial operations service suite instances supporting multiple clinical trials operated under common management, using a standard identity federation protocol.

19. The method of claim 16 further comprising supporting individually programmed analytic modules that each examines specific data to detect and report a specific condition related to one of system operations, fraud and security, or health outcomes.

20. The method of claim 16, wherein the associated sensors include at least one patient sensor and at least one clinician sensor.
